(19) United States
(12) Patent Application Publication
(10) Pub. No.: US 2026/0308156 A1
(43) Pub. Date: Oct. 8, 2026

(54) PYRAZOLOTRIAZINECARBONITRILES USEFUL AS KINASE INHIBITORS

(71) Applicant: BRISTOL-MYERS SQUIBB COMPANY, Princeton, NJ (US)

(72) Inventors: LI-QIANG SUN, NEWTOWN, PA (US); ZHIZHEN BARBARA ZHENG, SKILLMAN, NJ (US); MURALI T.G. DHAR, NEWTOWN, PA (US); ASHVINIKUMAR V. GAVAI, PRINCETON JUNCTION, NJ (US); JENNIFER X. QIAO, ARLINGTON, MA (US); DAVID K. WILLIAMS, DELRAN, NJ (US); PATRICE GILL, LEVITTOWN, PA (US); JOHN S. TOKARSKI, Princeton, NJ (US); SREEKANTHA RATNA KUMAR, BANGALORE (IN); HIMA KIRAN POTTURI, BENGALURU (IN); SOUMEN CHAKROBORTY, BENGALURU (IN); PITANI VEERA VENKATA SRINIVAS, ANDHRA PRADESH (IN)

(73) Assignee: BRISTOL-MYERS SQUIBB COMPANY, Princeton, NJ (US)

(21) Appl. No.: 19/474,825

(22) PCT Filed: Apr. 16, 2024
(86) PCT No.: PCT/US2024/024743

§ 371 Date: Oct. 13, 2025

Related U.S. Application Data
(60) Provisional application No. 63/496,432, filed on Apr. 17, 2023.

(51) Int. Cl. C07D 487/04; A61K 31/53; A61K 31/5377; A61K 31/55; C07B 59/00; C07D 519/00

(52) U.S. Cl. CPC C07D 487/04; A61K 31/53; A61K 31/5377; A61K 31/55; C07B 59/002; C07D 519/00; C07B 2200/05

(57) ABSTRACT

The invention provides compounds of Formula (I) or stereoisomers, tautomers, or pharmaceutically acceptable salts thereof. The Formula I imidazotriazines inhibit protein Formula (I) kinase activity thereby making them useful as anticancer agents.

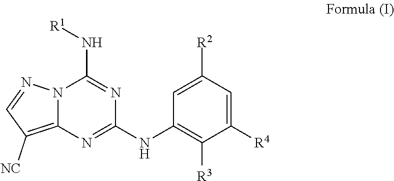

### CROSS-REFERENCE TO RELATED APPLICATIONS

This application claims the priority benefit of U.S. Provisional Application No. 63/496,432, filed Apr. 17, 2023; the content of which is herein incorporated by reference in its entirety.

### FIELD OF THE INVENTION

The invention relates to novel substituted pyrazolotriazine compounds useful as protein kinase inhibitors. The invention also relates to methods of using the compounds in the treatment of proliferative and other types of diseases and to pharmaceutical compositions containing the compounds.

### BACKGROUND OF THE INVENTION

The invention relates to fused heterocyclic compounds which inhibit protein kinase enzymes, compositions which contain protein kinase inhibiting compounds and methods of using inhibitors of protein kinase enzymes to treat diseases which are characterized by an overexpression or upregulation of protein kinases. Protein kinases mediate intracellular signal transduction. They do this by affecting a phosphoryl transfer from a nucleoside triphosphate to a protein acceptor that is involved in a signaling pathway. There are a number of kinases and pathways through which extracellular and other stimuli cause a variety of cellular responses to occur inside the cell. An extracellular stimulus may affect one or more cellular responses related to cell growth, migration, differentiation, secretion of hormones, activation of transcription factors, muscle contraction, glucose metabolism, and control of protein synthesis and regulation of cell cycle.

Many diseases are associated with abnormal cellular responses triggered by protein kinase-mediated events. These diseases include autoimmune diseases, inflammatory diseases, neurological and neurodegenerative diseases, cancer, cardiovascular diseases, allergies and asthma, Alzheimer's disease, or hormone-related diseases. Accordingly, there has been a substantial effort in medicinal chemistry to find protein kinase inhibitors that are effective as therapeutic agents.

Protein kinase CK2 (formerly known as casein kinase II) is a highly conserved serine/threonine kinase. Protein kinase CK2 is ubiquitously distributed and constitutively active in eukaryotes. In mammals, the enzyme exists in two isozymic forms due to variations in the catalytic subunits of the enzyme. The CK2 holoenzyme is a heterotetrameric complex composed of two catalytic α (CK2A1) subunits or α′ (CK2A2) subunits and two regulatory β-subunits. The formation of CK2 complexes containing the catalytic subunits requires dimerization of the regulatory β-subunits. CK2 interacts with a variety of cellular proteins and has been implicated in cell replication such as cell proliferation and differentiation, cellular survival, and tumorigenesis. With respect to tumorigenesis, protein kinase CK2 has been implicated in kidney tumors (Stalter et al., “Asymmetric expression of protein kinase CK2 subunits in human kidney tumors”, Biochem. Biophys. Res. Commun., 202:141-147 (1994)), mammary gland tumors (Landesman-Bollag et al., “Protein kinase CK2 in mammary gland tumorigenesis”, Oncology, 20:3247-3257 (2001)), lung carcinoma (Daya-Makin et al., “Activation of a tumor-associated protein kinase (p40TAK) and casein kinase II in human squamous cell carcinomas and adenocarcinomas of the lung”, Cancer Res., 54:2262-2268 (1994)), head and neck carcinoma (Faust et al., “Antisense oligonucleotides against protein kinase CK2-α inhibit growth of squamous cell carcinoma of the head and neck in vitro”, Head Neck, 22:341-346 (2000)), and prostate cancer (Wang et al., “Role of protein kinase CK2 in the regulation of tumor necrosis factor-related apoptosis inducing ligand-induced apoptosis in prostate cancer cells”, Cancer Res., 66:2242-2249 (2006)).

### SUMMARY OF THE INVENTION

The invention encompasses compounds of Formula (I) including stereoisomers, tautomers, pharmaceutically acceptable salts, solvates or prodrugs thereof, which inhibit protein kinase enzymes, especially protein kinase CK2 for the treatment of cancer such as non-small cell lung cancer.

The invention also provides processes and intermediates for making the compounds of the present invention.

The invention also provides pharmaceutical compositions comprising at least a pharmaceutically acceptable carrier and at least one of the compounds of the present invention or stereoisomers, tautomers, pharmaceutically acceptable salts, solvates, or prodrugs thereof.

The invention also provides methods for inhibiting the activity of protein kinase CK2 comprising administering to a host in need of such treatment a therapeutically effective amount of at least one of the compounds of the present invention or stereoisomers, tautomers, pharmaceutically acceptable salts, solvates, or prodrugs thereof.

The invention also provides methods for inhibiting angiogenesis or treating cancers comprising administering to a host in need of such treatment a therapeutically effective amount of at least one of the compounds of the present invention or stereoisomers, tautomers, pharmaceutically acceptable salts, solvates, or prodrugs thereof.

The invention also provides the compounds of the present invention or stereoisomers, tautomers, pharmaceutically acceptable salts, solvates, or prodrugs thereof, for use in therapy or the manufacture of a medicament.

The invention also provides the use of the compounds of the present invention or stereoisomers, tautomers, pharmaceutically acceptable salts, solvates, or prodrugs thereof, in preparing a medicament for the treatment of cancer in a human patient, particularly a cancer such as non-small cell lung cancer receptive to treatment via inhibition of the CK2 enzyme.

These and other features of the invention will be set forth in the expanded form as the disclosure continues.

### DETAILED DESCRIPTION OF THE INVENTION

In a first aspect, the present invention provides, inter alia, a compound of Formula (I):

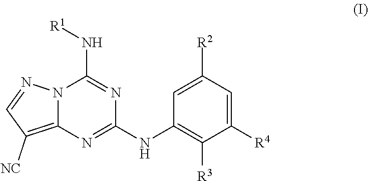

or a stereoisomer, a tautomer, or a pharmaceutically acceptable salt thereof, wherein:
R¹ is independently C₁₋₄ alkyl, C₃₋₄ cycloalkyl substituted with 0-1 R^{1a};
R^{1a} is independently F or C₁₋₄ alkyl;
R² and R³ are independently Cl, cyano, C₁₋₄ alkoxy, C₁₋₄ fluoroalkoxy, or C₃₋₄ cycloalkyl;
R⁴ is independently

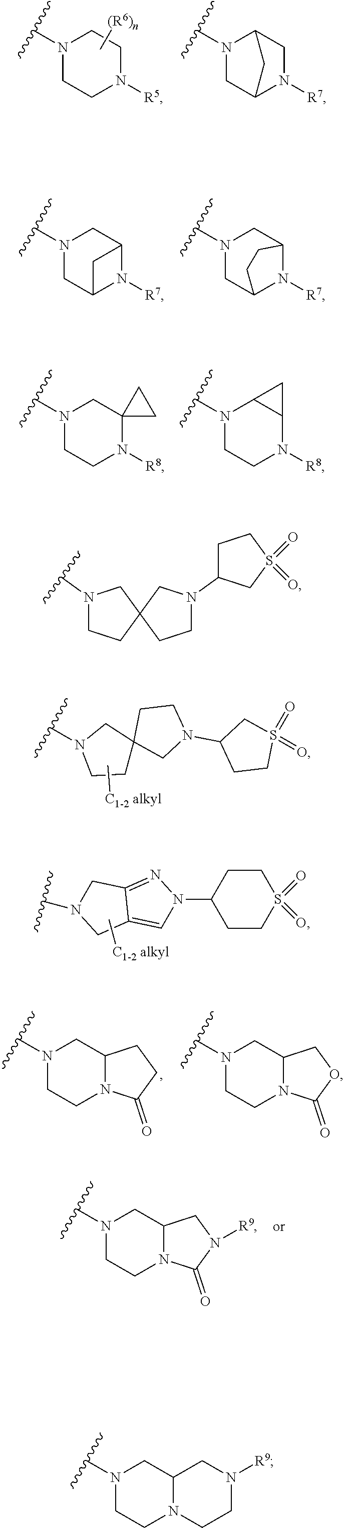

R⁵ is independently H, C₁₋₄ fluoroalkyl, —C(═O)(C₁₋₄ alkyl), —C(═O)(C₁₋₄ fluoroalkyl), —C(═O)C(C₁₋₂ alkyl)(C₁₋₂ fluoroalkyl)(OH), —C(═O)O(C₁₋₄ alkyl), —C(═O)CH₂O(C₁₋₂ alkyl), —C(═O)CH₂OC(═O)(C₁₋₂ alkyl), —C(═O)NHR¹⁰, —C(═O)C(═O)NHR¹¹, —SO₂(C₁₋₄alkyl), C₁₋₄alkyl substituted with 0-2 R^{a}, —(CH₂)₀₋₁—C₃₋₆ cycloalkyl substituted with 0-2 R^{d}, —X-(4- to 7-membered heterocyclic ring including 1 to 2 ring heteroatoms which are independently N, N(R^{b}), 0, or S(O)ₚ, and wherein the heterocyclic ring is partially saturated or fully saturated, and is substituted with 0 to 2 R^{e}), or —X-(5-membered heteroaryl wherein from 1 to 4 ring atoms are each independently N, or N(R^{b}), wherein the heteroaryl is substituted with 0 to 1 R^{d});
X is independently a bond, —CH₂—, —C(═O)—, —C(═O)CH₂—, —CH₂C(═O)—, or —C(═O)O—;
R⁶ is independently C₁₋₄ alkyl substituted with 0-1 OH, C₁₋₄ fluoroalkyl, or C₃₋₄ cycloalkyl;
R⁷ and R⁸ are independently H, C₁₋₄alkyl substituted with 0-2 R^{e}, —C(═O)(C₁₋₄ alkyl), —C(═O)O(C₁₋₄ alkyl), —CH₂C(═O)NH₂, —SO₂(C₁₋₂ alkyl),

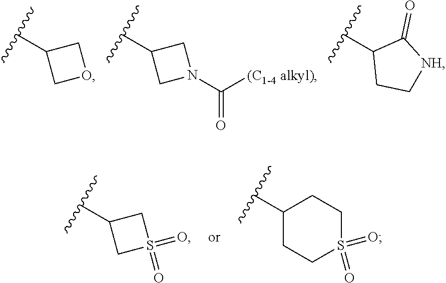

R⁹ is independently H, C₁₋₄ alkyl substituted with 0-2 R^{e}, —C(═O)(C₁₋₄ alkyl), —C(═O)O(C₁₋₄ alkyl), or

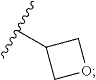 [SMILES: *C1COC1]

R¹⁰ is independently C₁₋₄ alkyl substituted with 0-2 R^{c}, C₁₋₄ fluoroalkyl, or C₃₋₄ cycloalkyl;
R¹¹ is independently H, C₁₋₄ alkyl, or C₃₋₄ cycloalkyl;
R^{a} is independently halogen, OH, C₁₋₄alkoxy, —C(═O)NH₂, —C(═O)O(C₁₋₄ alkyl), C₃₋₄ cycloalkyl substituted with 0-1 R^{d}, or

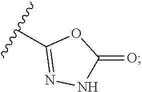 [SMILES: *c1n[nH]c(=O)o1]

R^{b} is independently H, C₁₋₄ alkyl substituted with 0-2 R^{e}, —C(═O)(C₁₋₄ alkyl), —C(═O)C(C₁₋₂ alkyl)(C₁₋₂ fluoroalkyl)(OH), —C(═O)O(C₁₋₄ alkyl), —C(═O)C(═O)NH₂, —SO₂(C₁₋₄ alkyl substituted with 0-1 R^{e}), C₃₋₄ cycloalkyl substituted with 0-1 R^{d},

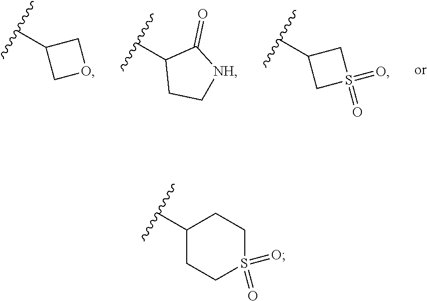 [SMILES: *C1COC1 ; *C1CCNC1=O ; *C1CS(=O)(=O)C1 ; "or" ; *C1CCS(=O)(=O)CC1]

R^{c} is independently C₁₋₂ alkoxy;
R^{d} is independently D, halogen, OH, NH₂, C₁₋₄alkyl, or —SO₂(C₁₋₄alkyl);
R^{e} is independently oxo or R^{d};
R^{f} is independently F, OH, C₁₋₄ alkoxy, or —C(═O)NH₂.
n is independently 0, 1 or 2; and
p is independently 0, 1 or 2.

In a second aspect, within the scope of the first aspect, the present invention provides a compound of Formula (I), or a stereoisomer, a tautomer, or pharmaceutically acceptable salt thereof, wherein:
R¹ is independently C₁₋₂ alkyl, C₁₋₂ fluoroalkyl, or C₃₋₄ cycloalkyl substituted with 0-1 R^{1a};
R^{1a} is independently F or C₁₋₂ alkyl;
R² is independently cyano, C₁₋₄ fluoroalkoxy, or C₃₋₄ cycloalkyl;
R³ is independently Cl, C₁₋₄ alkoxy, C₁₋₄ fluoroalkoxy, or C₃₋₄ cycloalkyl;
R⁶ is independently C₁₋₄ alkyl substituted with 0-1 OH, CF₃, or C₃₋₄ cycloalkyl;
R¹⁰ is independently C₁₋₄ alkyl substituted with 0-2 R^{c}, C₁₋₄ fluoroalkyl, or C₃₋₄ cycloalkyl; and
R¹¹ is independently H, C₁₋₄ alkyl, or C₃₋₄ cycloalkyl.

In a third aspect, within the scope of the first aspect or the second aspect, the present invention provides a compound of Formula (I), or a stereoisomer, a tautomer, or pharmaceutically acceptable salt thereof, wherein:
R⁴ is independently

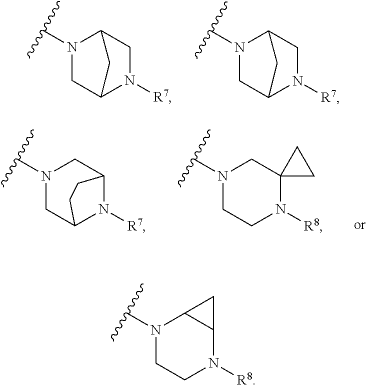

In a fourth aspect, within the scope of the first aspect or the second aspect, the present invention provides a compound of Formula (I), or a stereoisomer, a tautomer, or pharmaceutically acceptable salt thereof, wherein:
R⁴ is independently

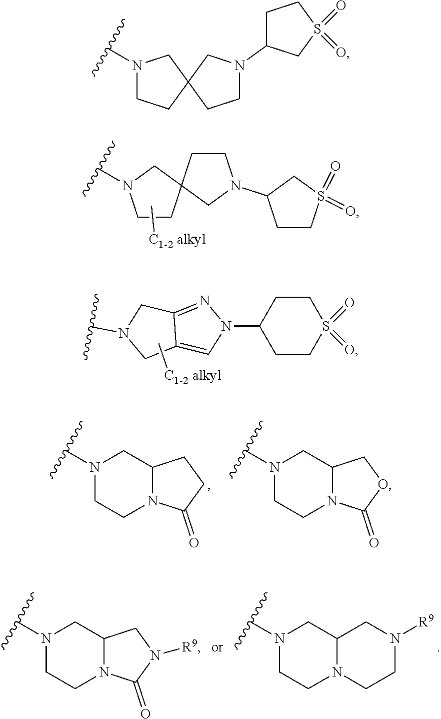

In a fifth aspect, within the scope of the first aspect or the second aspect, the present invention provides a compound of Formula (II):

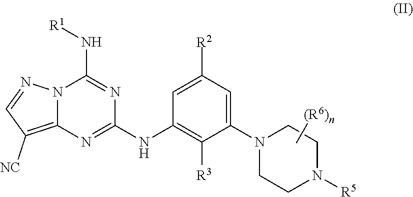

or a stereoisomer, a tautomer, or pharmaceutically acceptable salt thereof.

In a sixth aspect, the present invention provides a compound of Formula (III):

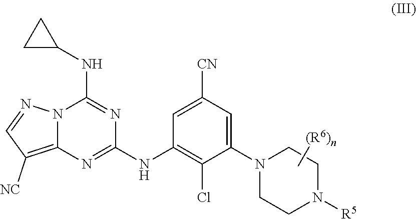

or a stereoisomer, a tautomer, or pharmaceutically acceptable salt thereof, wherein:
R⁵ is independently H, C₁₋₄ fluoroalkyl, —C(═O)(C₁₋₂ alkyl), —C(═O)(C₁₋₂ fluoroalkyl), —C(═O)C(CH₃)(CF₃)(OH), —C(═O)O(C₁₋₄ alkyl), —C(═O)CH₂O(C₁₋₂ alkyl), —C(═O)CH₂OC(═O)(C₁₋₂ alkyl), —C(═O)NHR¹⁰, —C(═O)C(═O)NHR¹¹, —SO₂(C₁₋₂alkyl), C₁₋₄alkyl substituted with 0-2 R^{a}, —(CH₂)₀₋₁—C₃₋₄ cycloalkyl substituted with 0-1 R^{d},

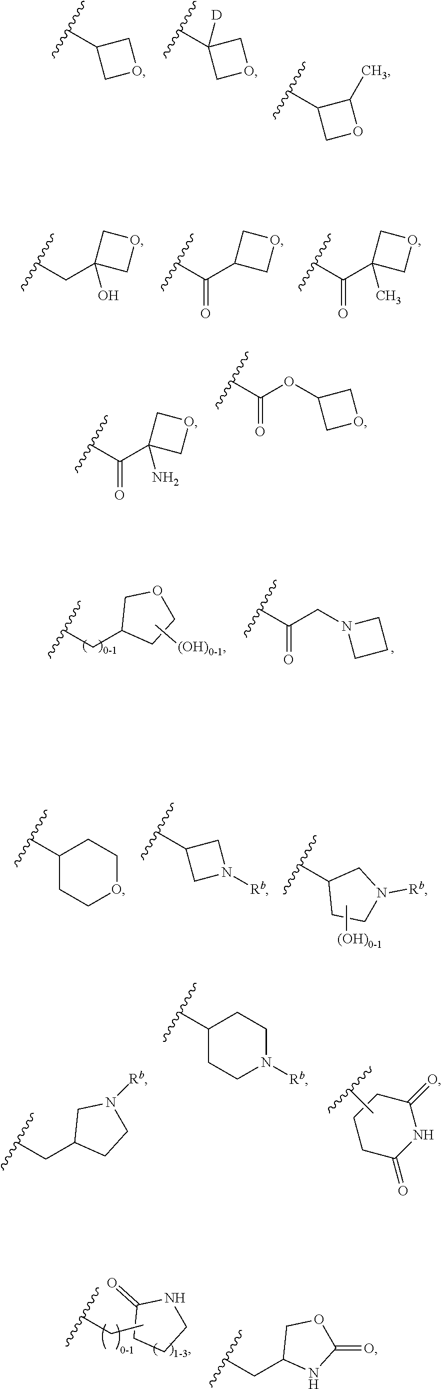

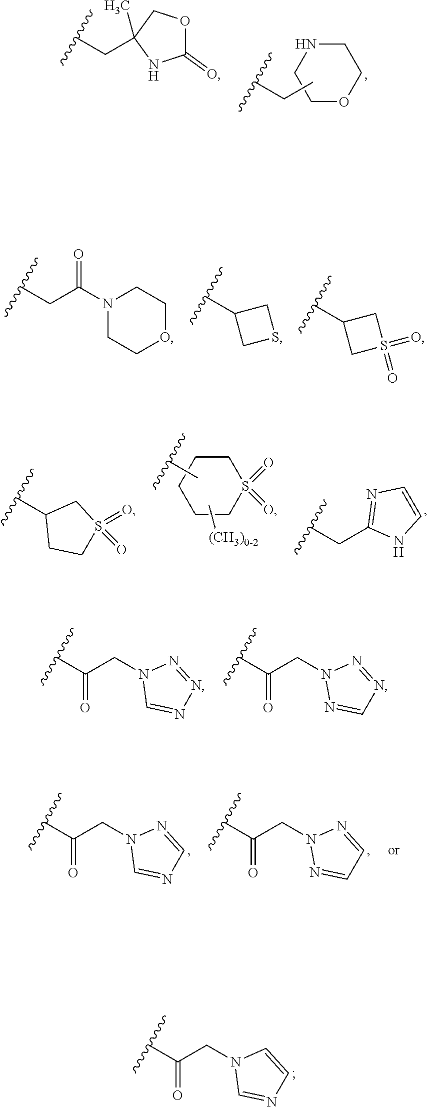

R⁶ is independently C₁₋₂ alkyl substituted with 0-1 OH, CF₃, or cyclopropyl;
R¹⁰ is independently C₁₋₄ alkyl substituted with 0-2 R^{c}, C₁₋₄ fluoroalkyl, or C₃₋₄ cycloalkyl;
R¹¹ is independently H, C₁₋₄ alkyl, or C₃₋₄ cycloalkyl;
R^{a} is independently F, OH, C₁₋₂alkoxy, —C(═O)NH₂, —C(═O)O(C₁₋₂ alkyl), or C₃₋₄ cycloalkyl substituted with 0-1 R^{d};
R^{b} is independently H, C₁₋₄ alkyl substituted with 0-2 R^{f}, —C(═O)(C₁₋₂ alkyl), —C(═O)C(CH₃)(CF₃)(OH), —C(═O)O(C₁₋₂ alkyl), —C(═O)C(═O)NH₂, —SO₂(C₁₋₂ alkyl substituted with 0-1 R^{e}), C₃₋₄ cycloalkyl substituted with 0-1 R^{d},

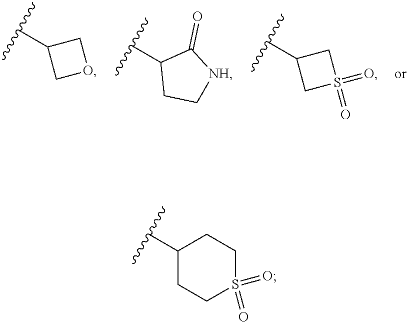 [SMILES: *C1COC1 ; *C1CCNC1=O ; *C1CS(=O)(=O)C1 ; *C1CCS(=O)(=O)CC1]

R^{c} is independently C₁₋₂ alkoxy;
R^{d} is independently F, OH, or —SO₂(C₁₋₂ alkyl);
R^{f} is independently F, OH, C₁₋₂ alkoxy, or —C(═O)NH₂; and
n is independently 0, 1 or 2.

In a seventh aspect, within the scope of the sixth aspect, the present invention provides a compound of Formula (IV):

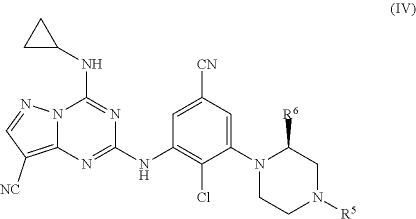

or a stereoisomer, a tautomer, or pharmaceutically acceptable salt thereof.

In an embodiment, within the scope of the sixth aspect or the seventh aspect, R⁶ is independently methyl or ethyl.

In another aspect, the invention provides a compound selected from the exemplified examples or a stereoisomer, a tautomer, a pharmaceutically acceptable salt, or a solvate thereof.

In another aspect, the invention provides a compound selected from any subset list of compounds within the scope of the exemplified examples or a stereoisomer, a tautomer, a pharmaceutically acceptable salt, or a solvate thereof.

All aspects of the compounds, including individual variable definitions, may be combined with other aspects to form additional compounds.

The compounds of the invention may form salts with alkali metals such as sodium, potassium and lithium, with alkaline earth metals such as calcium and magnesium, with organic bases such as dicyclohexylamine, tributylamine, pyridine and amino acids such as arginine, lysine and the like. Such salts can be formed as known to those skilled in the art.

The compounds of the invention may form salts with a variety of organic and inorganic acids. Such salts include those formed with hydrogen chloride, hydrogen bromide, methanesulfonic acid, sulfuric acid, acetic acid, trifluoroacetic acid, oxalic acid, maleic acid, benzenesulfonic acid, toluenesulfonic acid and various others (e.g., nitrates, phosphates, borates, tartrates, citrates, succinates, benzoates, ascorbates, salicylates and the like). Such salts can be formed as known to those skilled in the art.

In addition, zwitterions (“inner salts”) may be formed.

The invention is also intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can be prepared by conventional techniques known to those skilled in the art or by processes analogous to those described herein, using an appropriate isotopically-labeled reagent in place of the non-labeled reagent otherwise employed.

Compounds of the invention may also have prodrug forms. Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

Various forms of prodrugs are well known in the art. For examples of such prodrug derivatives, see:
Design of Prodrugs, H. Bundgaard, ed., Elsevier (1985), and
Methods in Enzymology, 112:309-396, K. Widder et al., eds., Academic Press (1985);
Bundgaard, H., Chapter 5, “Design and Application of Prodrugs,” A Textbook of Drug Design and Development, pp. 113-191, P. Krosgaard-Larsen et al., eds., Harwood Academic Publishers (1991); and
Bundgaard, H., Adv. Drug Deliv. Rev., 8:1-38 (1992).

It should further be understood that solvates (e.g., hydrates) of the compounds of the invention are also within the scope of the invention. Methods of solvation are generally known in the art. The inventive compounds may either be in the free or hydrate form.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C═N double bonds, and the like can also be present in the compounds, and all such stable isomers are contemplated in the present invention. Cis and trans geometric isomers of the compounds of the present invention are described and may be isolated as a mixture of isomers or as separated isomeric forms. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated. When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. All processes used to prepare compounds of the present invention and intermediates made therein are considered to be part of the present invention. When enantiomeric or diastereomeric products are prepared, they can be separated by conventional methods, for example, by chromatography or fractional crystallization. Compounds of the present invention, and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

### Definitions

The following are definitions of terms used in this specification and appended claims. The initial definition provided for a group or term herein applies to that group or term throughout the specification and claims, individually or as part of another group, unless otherwise indicated.

In accordance with a convention used in the art,

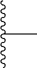

is used in structural formulas herein to depict the bond that is the point of attachment of the moiety or substituent to the core or backbone structure.

A dash “-” that is not between two letters or symbols is used to indicate a point of attachment for a substituent. For example, —CONH₂ is attached through the carbon atom.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

“Haloalkyl” is intended to include both branched and straight-chain saturated aliphatic hydrocarbon groups having the specified number of carbon atoms, substituted with 1 or more halogen. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl. Examples of haloalkyl also include “fluoroalkyl” which is intended to include both branched and straight-chain saturated aliphatic hydrocarbon groups having the specified number of carbon atoms, substituted with 1 or more fluorine atoms.

The term “halogen” or “halo” refers to fluorine (F), chlorine (Cl), bromine (Br) and iodine (I).

“Haloalkoxy” or “haloalkyloxy” represents a haloalkyl group as defined above with the indicated number of carbon atoms attached through an oxygen bridge. For example, “C₁₋₆ haloalkoxy”, is intended to include C₁, C₂, C₃, C₄, C₅, and C₆ haloalkoxy groups. Examples of haloalkoxy include, but are not limited to, trifluoromethoxy, 2,2,2-trifluoroethoxy, pentafluorothoxy, and the like. Similarly, “haloalkylthio” or “thiohaloalkoxy” represents a haloalkyl group as defined above with the indicated number of carbon atoms attached through a sulphur bridge; for example, trifluoromethyl-S—, pentafluoroethyl-S—, and the like.

As used herein, “carbocycle,” “carbocyclic residue,” or “carbocyclyl” is intended to mean any stable 3-, 4-, 5-, 6-, or 7-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, phenyl, and indanyl. When the term “carbocycle,” “carbocyclic residue,” or “carbocyclyl” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge.

The term “aryl” refers to monocyclic, bicyclic, tricyclic aromatic hydrocarbon groups having 6 to 15 carbon atoms in the ring portion, such as phenyl, naphthyl, biphenyl and diphenyl groups, each of which may be substituted. Aryl groups which are bicyclic or tricyclic must include at least one fully aromatic ring, but the other fused ring or rings may be aromatic or non-aromatic. When an aryl is substituted with a further heterocyclic ring, said ring may be attached to the aryl through a carbon atom or a heteroatom and said ring in turn is optionally substituted with one to two substituents as valence allows.

The terms “aryloxy”, “arylamino”, “arylalkylamino”, “arylthio”, “arylalkanoylamino”, “arylsulfonyl”, “arylalkoxy”, “arylsulfinyl”, “arylheteroaryl”, “arylalkylthio”, “arylcarbonyl”, “arylalkenyl”, or “arylalkylsulfonyl” refer to an aryl or substituted aryl bonded to an oxygen; an amino; an alkylamino; a thio; an alkanoylamino; a sulfonyl; an alkoxy; a sulfinyl; a heteroaryl or substituted heteroaryl; an alkylthio; a carbonyl; an alkenyl; or an alkylsulfonyl, respectively.

The term “alkenyl” refers to straight or branched chain hydrocarbon groups of 2 to 20 carbon atoms, preferably 2 to 15 carbon atoms, and most preferably 2 to 8 carbon atoms, having one to four double bonds.

The term “alkynyl” refers to straight or branched chain hydrocarbon groups of 2 to 20 carbon atoms, preferably 2 to 15 carbon atoms, and most preferably 2 to 8 carbon atoms, having one to four triple bonds.

An “alkylidene” group refers to an alkylene group consisting of at least two carbon atoms and at least one carbon-carbon double bond. Substituents on this group include those in the definition of “substituted alkyl”.

The term “cycloalkyl” refers to an optionally substituted, saturated cyclic hydrocarbon ring systems, preferably containing 1 to 3 rings and 3 to 7 carbons per ring. Exemplary groups include cyclopropyl, cyclobutyl, cyclopentyl, cyclohexyl, cycloheptyl, cyclooctyl, cyclodecyl, cyclododecyl, and adamantyl. Exemplary substituents include one or more alkyl groups as described above, or one or more groups described above as alkyl substituents.

As used herein, the term “heterocycle,” “heterocyclyl,” “heterocyclic ring” or “heterocyclic group” is intended to mean a stable 4-, 5-, 6-, or 7-membered monocyclic or bicyclic or 7-, 8-, 9-, 10-, 11-, 12-, 13-, or 14-membered bicyclic heterocyclic ring which is saturated, partially unsaturated or fully unsaturated or aromatic, and which consists of carbon atoms and 1, 2, 3 or 4 heteroatoms independently selected from the group consisting of N, O and S; and including any bicyclic group in which any of the above-defined heterocyclic rings is fused to a benzene ring. The nitrogen and sulfur heteroatoms may optionally be oxidized (i.e., N→O and S(O)ₚ). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. When the term “heterocycle,” “heterocyclyl,” “heterocyclic ring” or “heterocyclic group” is used, it is intended to include heteroaryl.

Examples of heterocycles include, but are not limited to, acridinyl, azocinyl, benzimidazolyl, benzofuranyl, benzothiofuranyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

Preferred 5- to 10-membered heterocycles include, but are not limited to, pyridinyl, furanyl, thienyl, pyrrolyl, pyrazolyl, pyrazinyl, piperazinyl, piperidinyl, imidazolyl, imidazolidinyl, indolyl, tetrazolyl, isoxazolyl, morpholinyl, oxazolyl, oxadiazolyl, oxazolidinyl, tetrahydrofuranyl, thiadiazinyl, thiadiazolyl, thiazolyl, triazinyl, triazolyl, benzimidazolyl, 1H-indazolyl, benzofuranyl, benzothiofuranyl, benztetrazolyl, benzotriazolyl, benzisoxazolyl, benzoxazolyl, oxindolyl, benzoxazolinyl, benzthiazolyl, benzisothiazolyl, isatinoyl, isoquinolinyl, octahydroisoquinolinyl, tetrahydroisoquinolinyl, tetrahydroquinolinyl, isoxazolopyridinyl, quinazolinyl, quinolinyl, isothiazolopyridinyl, thiazolopyridinyl, oxazolopyridinyl, imidazolopyridinyl, and pyrazolopyridinyl.

Preferred 5- to 6-membered heterocycles include, but are not limited to, pyridinyl, furanyl, thienyl, pyrrolyl, pyrazolyl, pyrazinyl, piperazinyl, piperidinyl, imidazolyl, imidazolidinyl, indolyl, tetrazolyl, isoxazolyl, morpholinyl, oxazolyl, oxadiazolyl, oxazolidinyl, tetrahydrofuranyl, thiadiazinyl, thiadiazolyl, thiazolyl, triazinyl, and triazolyl. Also included are fused ring and spiro compounds containing, for example, the above heterocycles.

Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge.

The term “heteroaryl” refers to substituted and unsubstituted aromatic 5- or 6-membered monocyclic groups, 9- or 10-membered bicyclic groups, and 11- to 14-membered tricyclic groups which have at least one heteroatom (O, S or N) in at least one of the rings, said heteroatom-containing ring preferably having 1, 2, or 3 heteroatoms selected from the group consisting of O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups can be substituted or unsubstituted. The nitrogen atom may be substituted or unsubstituted (i.e., N or NR wherein R is H or another substituent, if defined). The nitrogen and sulfur heteroatoms may optionally be oxidized (i.e., N→O and S(O)ₚ) and the nitrogen atoms may optionally be quaternized.

Heteroaryl groups which are bicyclic or tricyclic must include at least one fully aromatic ring, but the other fused ring or rings may be aromatic or non-aromatic. The heteroaryl group may be attached at any available nitrogen or carbon atom of any ring. The heteroaryl ring system may contain zero, one, two or three substituents.

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxazolyl, benzothienyl, quinolinyl, dihydroisoquinolinyl, tetrahydroquinolinyl, isoquinolinyl, benzimidazolyl, benzopyranyl, benzoxazinyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, and the like.

Exemplary tricyclic heteroaryl groups include carbazolyl, benzidolyl, phenanthrollinyl, acridinyl, phenanthridinyl, xanthenyl and the like.

The term “heteroatoms” shall include oxygen, sulfur and nitrogen.

As referred to herein, the term “substituted” means that one or more hydrogen atoms is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties.

When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 1-3 Rₑ, then said group may optionally be substituted with up to three Rₑ groups and Rₑ at each occurrence is selected independently from the definition of Rₑ. Also, combinations of substituents and/or variables are permissible only if such combinations result in stable compounds.

When a dotted ring is used within a ring structure, this indicates that the ring structure may be saturated, partially saturated or unsaturated.

### Utility

The compounds of the invention may be used to modulate kinase activities.

Applicants have discovered that compounds of the invention have particular utility in treating proliferative conditions associated with the modulation of kinase activity, and particularly the inhibition of serine/threonine kinase activities. The compounds of the present invention can be used to treat proliferative disorders associated with abnormal kinase activity. As used herein, the terms “treating” and “treatment” encompass either or both responsive and prophylaxis measures, e.g., measures designed to inhibit or delay the onset of the disease or disorder, achieve a full or partial reduction of the symptoms or disease state, and/or to alleviate, ameliorate, lessen, or cure the disease or disorder and/or its symptoms.

Accordingly, one aspect of the invention is the use of a compound of the invention, or a pharmaceutically acceptable salt thereof in the manufacture of a medicament for use in the production of an antiproliferative effect in a warm-blooded animal such as a human being.

According to a further feature of the invention there is provided a method for producing an antiproliferative effect in a warm-blooded animal, such as a human being, in need of such treatment which comprises administering to said animal an effective amount of a compound of the invention or a pharmaceutically acceptable salt thereof as defined herein before.

The anti-proliferative treatment defined herein before may be applied as a sole therapy or may involve, in addition to a compound of the invention, one or more other substances and/or treatments. Such treatment may be achieved by way of the simultaneous, sequential or separate administration of the individual components of the treatment. The compounds of this invention may also be useful in combination with known anti-cancer and cytotoxic agents and treatments, including radiation. Compounds of the invention may be used sequentially with known anticancer or cytotoxic agents and treatment, including radiation when a combination formulation is inappropriate.

In the field of medical oncology, it is normal practice to use a combination of different forms of treatment to treat each patient with cancer. In medical oncology the other component(s) of such treatment in addition to the antiproliferative treatment defined herein may be surgery, radiotherapy or chemotherapy.

As stated above, the compounds of the invention are of interest for their antiproliferative effects. Such compounds of the invention are expected to be useful in a wide range of disease states including cancer, psoriasis, and rheumatoid arthritis.

Due to the key role of kinases in the regulation of cellular proliferation in general, inhibitors could act as reversible cytostatic agents which may be useful in the treatment of any disease process which features abnormal cellular proliferation, e.g., benign prostate hyperplasia, familial adenomatosis polyposis, neurofibromatosis, pulmonary fibrosis, arthritis, psoriasis, glomerulonephritis, restenosis following angioplasty or vascular surgery, hypertrophic scar formation and inflammatory bowel disease.

Compounds of the invention are especially useful in treatment of tumors having a high incidence of serine/threonine kinase activity, such as prostate, colon, brain, thyroid and pancreatic tumors. Additionally, the compounds of the invention may be useful in treatment of sarcomas and pediatric sarcomas. By the administration of a composition (or a combination) of the compounds of this invention, development of tumors in a mammalian host is reduced.

Compounds of the invention may also be useful in the treatment of other cancerous diseases (such as acute myelogenous leukemia) that may be associated with signal transduction pathways operating through kinases such as DYRK1a, CDK, and GSK3β. The inventive compositions may contain other therapeutic agents as described above and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

Accordingly, the present invention further includes compositions comprising one or more compounds of the invention and a pharmaceutically acceptable carrier.

A “pharmaceutically acceptable carrier” refers to media generally accepted in the art for the delivery of biologically active agents to animals, in particular, mammals. Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Remington's Pharmaceutical Sciences, 17th ed. (1985), which is incorporated herein by reference in its entirety.

The pharmaceutical compositions of the invention containing the active ingredient may be in a form suitable for oral use, for example, as Tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

The pharmaceutical compositions may be in the form of sterile injectable aqueous solutions. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS® Model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above.

Compounds of the invention may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of Tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aq. or non-aq. solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Exemplary compositions for topical administration include a topical carrier such as Plastibase (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release Tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried Tablets.

Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., Gantrez); and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

When a compound according to this invention is administered into a human subject, the daily dosage will normally be determined by the prescribing physician with the dosage generally varying according to the age, weight, sex and response of the individual patient, as well as the severity of the patient's symptoms. Exemplary dosage amounts for a mammal may include from about 0.05 to 1000 mg/kg; 1-1000 mg/kg; 1-50 mg/kg; 5-250 mg/kg; 250-1000 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species, which are affected by mediation of protein kinase enzyme levels.

If formulated as a fixed dose, a combination product can, for example, utilize a dosage of the compound of the invention within the dosage range described above and the dosage of another anti-cancer agent/treatment within the approved dosage range for such known anti-cancer agent/treatment. If a combination product is inappropriate, the compounds of the invention and the other anti-cancer agent/treatment can, for example, are administered simultaneously or sequentially. If administered sequentially, the present invention is not limited to any particular sequence of administration. For example, compounds of the invention can be administered either prior to, or after, administration of the known anti-cancer agent or treatment.

### Methods of Preparation

As can be appreciated by the skilled artisan, methods of synthesizing compounds of the invention herein will be evident to those of ordinary skill in the art. Synthetic chemistry transformations and protecting group methodologies (protection and deprotection) useful in synthesizing the compounds described herein are known in the art and include, for example, those such as described in Larock, R. C., Comprehensive Organic Transformations, 2^{nd} Edition, Wiley-VCH, New York, NY (1999); Wuts, P. G. M., Greene's Protective Groups in Organic Synthesis, 5th Edition, Wiley (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g., diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

Compounds of the invention can be prepared in a number of ways well known to one skilled in the art of organic synthesis. The compounds of the present invention can be synthesized using the methods described below, together with synthetic methods known in the art of synthetic organic chemistry, or variations thereon as appreciated by those skilled in the art. Preferred methods include, but are not limited to, those described below.

Compounds of this invention may be prepared using the reactions and techniques described in this section. The reactions are performed in solvents appropriate to the reagents and materials employed and are suitable for the transformations being effected. Also, in the description of the synthetic methods described below, it is to be understood that all proposed reaction conditions, including choice of solvent, reaction atmosphere, reaction temperature, duration of the experiment and work up procedures, are chosen to be the conditions standard for that reaction, which should be readily recognized by one skilled in the art. It is understood by one skilled in the art of organic synthesis that the functionality present on various portions of the molecule must be compatible employing the reagents and reactions proposed. Such restrictions to the substituents that are compatible employing the reaction conditions will be readily apparent to one skilled in the art and alternate methods must then be used. This will sometimes require a judgment to modify the order of the synthetic steps or to select one particular process scheme over another in order to obtain a desired compound of the invention. It will also be recognized that another major consideration in the planning of any synthetic route in this field is the judicious choice of the protecting group used for protection of the reactive functional groups present in the compounds described in this invention.

The compounds of this invention may be prepared using the reactions and techniques described in this section and the accompanying Schemes.

Compounds of Formula (I) may be prepared by as described in Scheme 1. Commercially available 5-amino-1H-pyrazole-4-carbonitrile 1 was first treated with ethoxycarbonyl-isothiocyanate at elevated temperature, for example at 70° C., in an appropriate solvent such as ethyl acetate to offer thiourea 2. Cyclization of 2 in the presence of base such as ammonia afforded the pyrazolotriazine 3. Selective S-alkylation of 3 with one equivalent electrophile such as methyl iodide in an appropriate solvent such as DMF provides 4. The intermediate 4 can be converted to the corresponding chloride 5 by treatment with phosphorous oxychloride at elevated temperature, for example a 125° C., in the presence of amines such as diisopropylethylamine. Treatment of compound 5 with an amine (R₂R₃NH) such as N-(4-methoxybenzyl)cyclopropanamine or N-(4-methoxybenzyl) ethylamine in the presence of a base such as diisopropylethylamine in an aprotic solvent such as THF affords compound 6. Treatment of pyrazolotriazine 6 with a suitable oxidizing agent such as MCPBA in a suitable solvent such as DCM yields pyrazolotriazine 7. Displacement of sulfone 7 with a nucleophile such as an amine in the presence of a base such as potassium tert-butoxide or LiHMDS in a suitable solvent such as THF provides pyrazolotriazine 8. In cases where R3 is an amine protecting group such as p-methoxybenzyl, removal of the protecting group can be affected through known methods. For example, treatment with trifluoroacetic acid with an appropriate solvent such as dichloroethane in the presence of a cation trap such as anisole affords general formula 9.

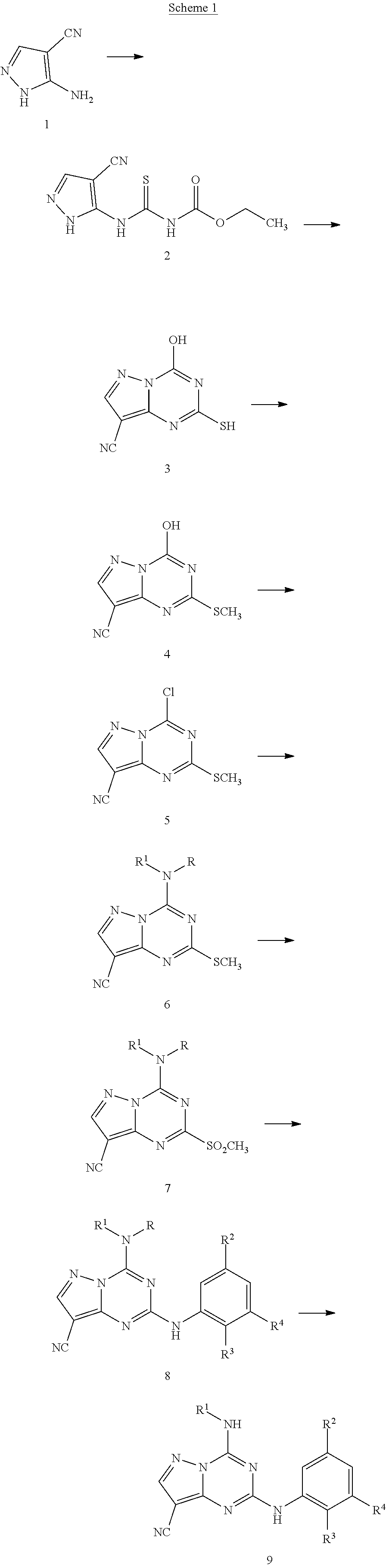

Alternatively, the compounds of general formula 9 could also be prepared according to Scheme 2. Displacement of sulfone 7 with a nucleophile such as an ammonia in a suitable solvent such as dioxane at elevated temperature, for example at 80° C., provides pyrazolotriazine 10. Palladium mediated coupling of 10 with aryl halide at elevated temperature, for example above 90° C., in the presence of a palladium catalyst such as Pd₂(dba)₃, a ligand such as 4,5-bis(diphenyl-phosphino)-9,9-dimethylx-25 xanthene (Xantphos) and a base such as cesium carbonate yields 8. In cases where R is an amine protecting groups such as p-methoxybenzyl, removal of the protecting group can be affected through known methods. For example, treatment with trifluoroacetic acid with an appropriate solvent such as dichloroethane in the presence of a cation trap such as anisole affords general formula 9.

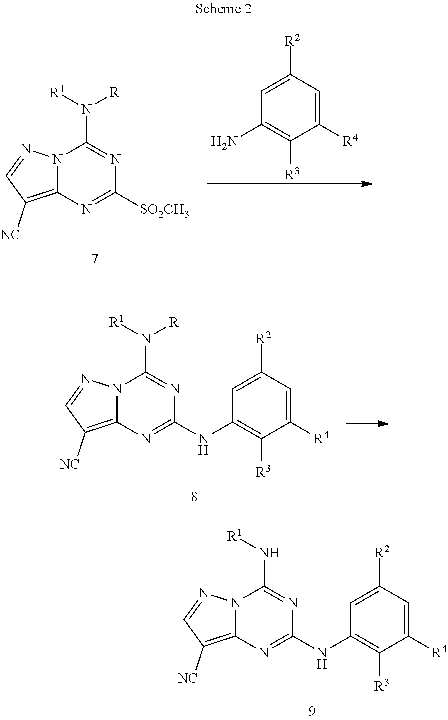

The following abbreviations have the indicated meanings:
ACN=acetonitrile
AcOH=acetic acid
BOP=(Benzotriazol-1-yloxy)tris(dimethylamino)phosphonium
hexafluorophosphate
CDCl₃=chloroform-d
CD₃OD=methanol-d₄
CH₂Cl₂=dichloromethane
Cs₂CO₃=cesium carbonate
CuI=copper (I) iodide
d=doublet
DBU=1,8-diazabicycloundec-7-ene
DCM=dichloromethane
DCE=dichloroethane
DIEA=N,N-diisopropylethylamine
DMF=N,N-dimethylformamide
DMSO=dimethylsulfoxide
ES=electrospray ionization
Et₂O=diethyl ether
EtOAc=ethyl acetate
EtOH=ethanol
equiv=equivalents
g=gram(s)
h=hour(s)
HCl=hydrogen chloride (usually as a solution)
H₂O=water
H₂O₂=hydrogen peroxide
HATU=1-[Bis(dimethylamino)methylene]-1H-1,2,3-triazolo[4,5-b]pyridinium 3-oxide hexafluorophosphate
HPLC=high-performance liquid chromatography
Hunig's base=N,N-Diisopropylethylamine
ISCO=silica gel cartridge, unless specified otherwise
I₂=iodine
K₂CO₃=potassium carbonate
K₂HPO₄=potassium phosphate, dibasic
KI=potassium iodide
kg=kilogram(s)
LC/MS=liquid chromatography mass spectrometer
LiBH₄=lithium borohydride
m=multiplet
m/z=mass to charge ratio
M=molar
m-CPBA=meta-chloroperoxybenzoic acid
mg=milligram(s)
MeOH=methanol
MHz=megahertz
mL=milliliter(s)
mmol=millimole(s)
min=minute(s)
NaHCO₃=sodium bicarbonate
Na₂CO₃=sodium carbonate
NaOH=sodium hydroxide
Na₂SO₄=sodium sulfate
NEt₃ and TEA=triethylamine
NH₄OH or NH₃H₂O=ammonium hydroxide
NH₄HCO₃=ammonium bicarbonate
nm=nanometer
PdCl₂(PPh₃)₂=bis(triphenylphosphine)palladium (II) dichloride
Pd(dppf)C₂=[1,1′-Bis(diphenylphosphino)ferrocene]dichloropalladium(II)
Pd(dppf)Cl₂DCM=[1,1′-Bis(diphenylphosphino)ferrocene]dichloropalladium(II) dichloromethane complex
Pd(OH)₂=palladium hydroxide
PMB=para-methoxybenzyl
POCl₃=phosphorous oxychloride
ppm=parts per million
ppt=precipitate
Pt=platinum
Pt/C=platinum on carbon
rt=room temperature
RT=retention time
s=singlet
t=triplet
T3P=n-propylphosphonic anhydride
TFA=trifluoroacetic acid
TLC=thin layer chromatography
TsCl=para-toluenesulfonyl chloride
° C.=degrees Celsius
μmol=micromole(s)

### EXAMPLES

The following Examples illustrate embodiments of the inventive compounds and starting materials, and are not intended to limit the scope of the claims.

### Example 1

### (2S)-2-[4-(2-Chloro-5-cyano-3-{[8-cyano-4-(cyclopropylamino)pyrazolo[1,5-a][1,3,5]triazin-2-yl]amino}phenyl)piperazin-1-yl]propanamide

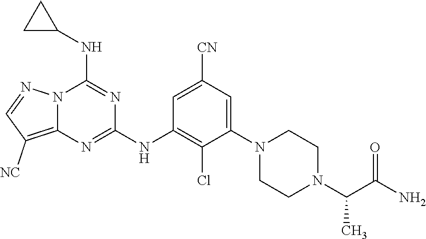 [SMILES: C[C@@H](C(N)=O)N1CCN(c2cc(C#N)cc(Nc3nc(NC4CC4)n4ncc(C#N)c4n3)c2Cl)CC1]

### Step 1. (tert-Butyl 4-(3-((tert-butoxycarbonyl)amino)-2-chloro-5-cyanophenyl)piperazine-1-carboxylate)

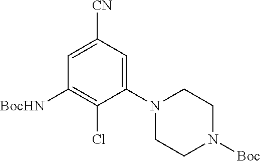 [SMILES: CC(C)(C)OC(=O)Nc1cc(C#N)cc(N2CCN(C(=O)OC(C)(C)C)CC2)c1Cl]

A 1-L three neck flask equipped with mechanic stirrer was charged with tert-butyl (3-bromo-2-chloro-5-cyanophenyl)carbamate (24.25 g, 72.4 mmol), tert-butyl piperazine-1-carboxylate (14.16 g, 76 mmol), Pd₂(dba)₃ (6.63 g, 7.24 mmol), BINAP (6.76 g, 10.86 mmol), Cs₂CO₃ (47.2 g, 145 mmol) and dioxane (483 mL). Nitrogen was bubbled into the reaction mixture for 30 min and the reaction was heated at 105° C. for 16 h under nitrogen. The reaction mixture was cooled to room temperature and celite was added. The reaction mixture was filtered through celite. The filtrate was concentrated in vacuo and the residue was purified by ISCO, eluting with 20% EtOAc/hexane to afford tert-butyl 4-(3-((tert-butoxycarbonyl)amino)-2-chloro-5-cyanophenyl)piperazine-1-carboxylate (25.11 g, 57.5 mmol, 79% yield) as an off-white solid. LC/MS [M+H]⁺=437.3. ¹H NMR (400 MHz, CDCl₃) δ ppm 7.23-7.17 (m, 1H), 7.00-6.96 (m, 1H), 3.72-3.51 (m, 4H), 3.07-2.85 (m, 4H), 1.58-1.55 (m, 9H), 1.54-1.49 (m, 9H).

### Step 2. (tert-Butyl 4-(3-amino-2-chloro-5-cyanophenyl)piperazine-1-carboxylate)

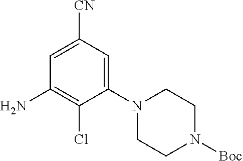 [SMILES: CC(C)(C)OC(=O)N1CCN(c2cc(C#N)cc(N)c2Cl)CC1]

A solution of tert-butyl 4-(3-((tert-butoxycarbonyl)amino)-2-chloro-5 cyanophenyl)piperazine-1-carboxylate (7.8 g, 17.85 mmol) in DCM (60 mL) was treated with TFA (12 mL, 156 mmol), and the contents stirred at rt for 1 h. The reaction mixture was concentrated, the crude material was triturated with Et₂O, and the resulting solid was collected by filtration. The solid was dissolved in 50 mL of DCM, and treated with TEA (7.46 mL, 53.6 mmol), followed by Boc₂O (4.14 mL, 17.85 mmol). It was stirred at rt for 30 min. The reaction mixture was concentrated, and the crude material was purified by ISCO, eluting with 25% EtOAc/hexane to yield tert-butyl 4-(3-amino-2-chloro-5-cyanophenyl)piperazine-1-carboxylate (4.87 g, 14.46 mmol, 81% yield). LC/MS [M+H]⁺=337.1. ¹H NMR (400 MHz, CDCl₃) δ ppm 7.42-7.19 (m, 1H), 6.86-6.51 (m, 2H), 4.43-4.26 (m, 2H), 3.72-3.44 (m, 4H), 3.09-2.80 (m, 4H), 1.53-1.46 (m, 9H).

### Step 3. Ethyl N-[(4-cyano-1H-pyrazol-5-yl)carbamothioyl]carbamate

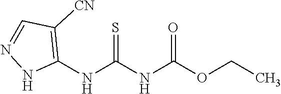 [SMILES: CCOC(=O)NC(=S)Nc1[nH]ncc1C#N]

A mixture of 3-amino-1H-pyrazole-4-carbonitrile (15 g, 139 mmol) and O-ethyl carbonisothiocyanatidate (18.56 g, 142 mmol) in ethyl acetate (250 mL) was heated at 70° C. for 4 h. It was then cooled to rt, filtered and the resulting solid was washed with EtOAc to afford ethyl N-[(4-cyano-1H-pyrazol-5-yl)carbamothioyl]carbamate (27.5 g, 115 mmol, 83% yield). LC/MS [M+H]⁺=240.0. ¹H NMR (400 MHz, DMSO-d₆) δ ppm 13.62 (1H, br. s.), 11.55 (1H, s), 11.20 (1H, s), 8.56 (1H, d, J=1.54 Hz), 4.23 (2H, q, J=7.04 Hz), 1.27 (3H, t, J=7.04 Hz).

### Step 4. (4-Oxo-2-thioxo-1,2,3,4-tetrahydropyrazolo[1,5-a][1,3,5]triazine-8-carbonitrile)

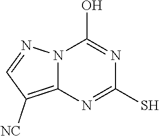 [SMILES: N#Cc1cnn2c(O)nc(S)nc12]

A suspension of ethyl N-[(4-cyano-1H-pyrazol-5-yl)carbamothioyl]carbamate (27.5 g, 115 mmol) in MeOH (230 mL) was treated with ammonium hydroxide (13.43 mL, 345 mmol) and stirred at rt for 2 h. The resulting solid was afforded by filtration. It was then washed with MeOH, and dried to afford 4-oxo-2-thioxo-1,2,3,4-tetrahydropyrazolo[1,5-a][1,3,5]triazine-8-carbonitrile (22 g, 114 mmol, 99% yield). LC/MS [M+H]⁺=194.0. ¹H NMR (400 MHz, DMSO-d₆) δ ppm 8.08 (1H, s), 7.08 (2H, br. s.).

### Step 5. (4-Hydroxy-2-(methylthio)pyrazolo[1,5-a][1,3,5]triazine-8-carbonitrile)

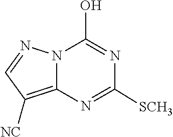 [SMILES: CSc1nc(O)n2ncc(C#N)c2n1]

To a solution of 4-hydroxy-2-mercaptopyrazolo[1,5-a][1,3,5]triazine-8-carbonitrile (10 g, 51.8 mmol) in DMF (104 ml) was added Et₃N (7.22 mL, 51.8 mmol), iodomethane (3.22 mL, 51.8 mmol) and the contents stirred at rt for 2 h. The pH of the reaction was adjusted to ~2 using 1 N HCl. The white solid that separated out was filtered and washed with water. The light-yellow solid was dried. The aqueous solution was extracted with EtOAc. The organic layer was dried over MgSO₄, filtered, and concentrated to obtain a solid. The solids were mixed to yield 4-hydroxy-2-(methylthio)pyrazolo[1,5-a][1,3,5]triazine-8-carbonitrile (10 g, 48.3 mmol, 93% yield). LC/MS [M+H]⁺=208.0. ¹H NMR (499 MHz, DMSO-d₆) δ ppm 8.46-8.32 (m, 1H), 2.62-2.58 (m, 3H).

### Step 6. (4-Chloro-2-(methylthio)pyrazolo[1,5-a][1,3,5]triazine-8-carbonitrile)

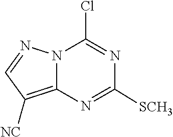 [SMILES: CSc1nc(Cl)n2ncc(C#N)c2n1]

To a mixture of 4-hydroxy-2-(methylthio)pyrazolo[1,5-a][1,3,5]triazine-8-carbonitrile (3.3 g, 15.93 mmol) in POCl₃ (30 mL, 322 mmol) under N₂ was added Hunig's base (3.06 mL, 17.52 mmol). The contents were heated at 125° C. for 2 h. The reaction mixture was cooled to rt, concentrated, DCM was added, and the contents concentrated. This step was repeated 3 times to azeotrope most of the POCl₃. The resulting residue was diluted with EtOAc. It was then poured into a cooled (0° C.) sat. solution of NaHCO₃. The organic layer was separated, washed with brine, dried over MgSO₄, filtered, and concentrated to obtain 4-chloro-2-(methylthio)pyrazolo[1,5-a][1,3,5]triazine-8-carbonitrile (3.5 g, 15.51 mmol, 97% yield) as a yellow solid. It was used as such for the subsequent step without purification. LC/MS [M+H]⁺=225.9. ¹H NMR (499 MHz, DMSO-d₆) δ ppm 8.48-8.42 (m, 1H), 2.62-2.58 (m, 3H).

### Step 7. (4-(Cyclopropyl(4-methoxybenzyl)amino)-2-(methylthio)pyrazolo[1,5-a][1,3,5]triazine-8-carbonitrile)

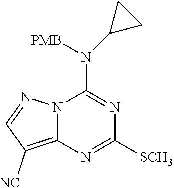

To a mixture of 4-chloro-2-(methylthio)pyrazolo[1,5-a][1,3,5]triazine-8-carbonitrile (1 g, 4.43 mmol) in THF (10 mL) was added N-(4-methoxybenzyl)cyclopropanamine (0.864 g, 4.87 mmol) and Hunig's base (0.929 ml, 5.32 mmol). The mixture was stirred at rt for 2 h. It was then diluted with EtOAc. The organic layer was washed with water, dried over MgSO₄, filtered, and concentrated to obtain a brown solid, which was purified by ISCO, eluting with 30% EtOAc/hexane to obtain a yellow solid. On trituration of the solid with MeOH 4-(cyclopropyl(4-methoxybenzyl)amino)-2-(methylthio)pyrazolo[1,5-a][1,3,5]triazine-8-carbonitrile was obtained (1.1 g, 3.00 mmol, 67.7% yield). LC/MS [M+H]⁺=367.1. ¹H NMR (499 MHz, DMSO-d₆) 5 ppm 8.63-8.53 (m, 1H), 7.32-7.12 (m, 2H), 6.96-6.78 (m, 2H), 5.32-5.08 (m, 2H), 3.75-3.72 (m, 3H), 3.11-3.01 (m, 1H), 2.55-2.52 (m, 3H), 0.90-0.82 (m, 4H).

### Step 8. (4-(Cyclopropyl(4-methoxybenzyl)amino)-2-(methylsulfonyl) pyrazolo[1,5-a][1,3,5]triazine-8-carbonitrile)

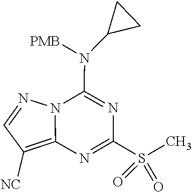

To a mixture of 4-(cyclopropyl(4-methoxybenzyl)amino)-2-(methylthio) pyrazolo[1,5-a][1,3,5]triazine-8-carbonitrile (2.5 g, 6.82 mmol) in CH₂Cl₂ (20 mL) was added mCPBA (5.05 g, 20.47 mmol) at 0° C. It was then stirred at 0° C. for 1 h. The solid formed was filtered and to the DCM layer was added sat. aq. Na₂S₂O₃ solution and the contents stirred at rt for 5 min. The DCM layer was separated, washed with sat. NaS₂O₃ two more times followed by then sat. NaHCO₃ three times and water. The organic layer was dried over MgSO₄, filtered and concentrated. It was then purified by ISCO with 50% EtOAc/hexane to yield 4-(cyclopropyl(4-methoxybenzyl)amino)-2-(methylsulfonyl)pyrazolo[1,5-a][1,3,5]triazine-8-carbonitrile (2.6 g, 6.53 mmol, 96% yield) as a white foam. LC/MS [M+H]⁺=399.1. ¹H NMR (499 MHz, DMSO-d₆) δ ppm 8.90-8.67 (m, 1H), 7.50-7.20 (m, 2H), 6.97-6.73 (m, 2H), 5.73-4.83 (m, 2H), 3.82-3.70 (m, 3H), 3.45-3.33 (m, 3H), 3.24-3.12 (m, 1H), 1.09-0.77 (m, 4H).

### Step 9. (tert-Butyl 4-(2-chloro-5-cyano-3-((8-cyano-4-(cyclopropyl(4-methoxybenzyl)amino)pyrazolo[1,5-a][1,3,5]triazin-2-yl)amino)phenyl)piperazine-1-carboxylate)

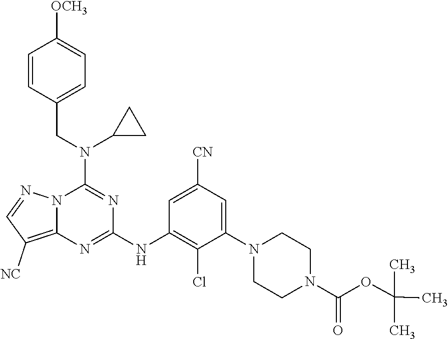 [SMILES: COc1ccc(CN(c2nc(Nc3cc(C#N)cc(N4CCN(C(=O)OC(C)(C)C)CC4)c3Cl)nc3c(C#N)cnn23)C2CC2)cc1]

To a mixture of tert-butyl 4-(3-amino-2-chloro-5-cyanophenyl) piperazine-1-carboxylate (1.1 g, 3.27 mmol) in tetrahydrofuran (15 mL) was added 1 M LiHMDS (6.53 mL, 6.53 mmol) in THF dropwise at −78° C. It was then stirred at −78° C. for 30 min and then 4-(cyclopropyl(4-methoxybenzyl) amino)-2-(methylsulfonyl)pyrazolo[1,5-a][1,3,5]triazine-8-carbonitrile (1.301 g, 3.27 mmol) in tetrahydrofuran (15 mL) was added and stirred at −78° C. for 15 min. The reaction mixture was quenched with water and extracted with EtOAc. The organic layer was washed with water, dried over MgSO₄, filtered and concentrated. It was then purified by ISCO, eluting with 40% EtOAc/hexane to yield tert-butyl 4-(2-chloro-5-cyano-3-((8-cyano-4-(cyclopropyl(4-methoxybenzyl)amino)pyrazolo[1,5-a][1,3,5]triazin-2-yl)amino)phenyl)piperazine-1-carboxylate (1.3 g, 1.984 mmol, 60.8% yield). LC/MS [M+H]⁺=655.2. ¹H NMR (499 MHz, DMSO-d₆) δ ppm 9.85-9.60 (m, 1H), 8.50-8.33 (m, 1H), 8.05-7.81 (m, 1H), 7.45-7.33 (m, 1H), 7.24-7.09 (m, 2H), 6.98-6.80 (m, 2H), 5.26-4.85 (m, 2H), 3.79-3.68 (m, 3H), 3.54-3.39 (m, 4H), 3.03-2.84 (m, 5H), 1.54-1.36 (m, 9H), 0.96-0.71 (m, 4H).

### Step 10. 2-{[2-chloro-5-cyano-3-(piperazin-1-yl)phenyl]amino}-4-(cyclopropylamino)pyrazolo[1,5-a][1,3,5]triazine-8-carbonitrile

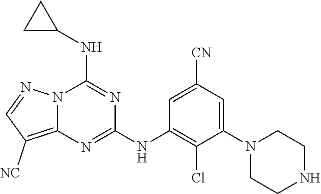 [SMILES: N#Cc1cc(Nc2nc(NC3CC3)n3ncc(C#N)c3n2)c(Cl)c(N2CCNCC2)c1]

A mixture of tert-butyl 4-(2-chloro-5-cyano-3-((8-cyano-4-(cyclopropyl (4-methoxybenzyl)amino)pyrazolo[1,5-a][1,3,5]triazin-2-yl)amino)phenyl) piperazine-1-carboxylate (750 mg, 1.145 mmol) in CH₂Cl₂ (1 mL) was added TFA (3.53 mL, 45.8 mmol) and the contents stirred at rt for 1 h. The reaction mixture was concentrated and to the residue was added ClCH₂CH₂Cl (1 mL), TFA (3.53 mL, 45.8 mmol) and anisole (0.500 mL, 4.58 mmol) and the contents heated at 50° C. for 1 h. The reaction mixture was cooled to RT concentrated and the to the residue was added 1 mL of 7 N NH₃ and the solid was filtered and dried to obtain 2-{[2-chloro-5-cyano-3-(piperazin-1-yl)phenyl]amino}-4-(cyclopropylamino)pyrazolo[1,5-a][1,3,5]triazine-8-carbonitrile (435 mg, 1.00 mmol, 87% yield). LC/MS [M+H]⁺=435.4.

### Step 11: Example 1

A solution of 2-{[2-chloro-5-cyano-3-(piperazin-1-yl)phenyl]amino}-4-(cyclopropylamino)pyrazolo[1,5-a][1,3,5]triazine-8-carbonitrile (80 mg, 0.184 mmol), Hunig's base (0.064 mL, 0.368 mmol) and (R)-1-amino-1-oxopropan-2-yl methanesulfonate (46.1 mg, 0.276 mmol) in DMSO (1 mL) was stirred at 80° C. for 24 h. The reaction mixture was bought to rt and the crude was purified via preparative LC/MS employing the following conditions: Column: XBridge C18, 200 mm×30 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 12% B, 12-42% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 61.0 mg (65%), and its purity by LCMS analysis was 96%. Analytical LC/MS was used to determine the final purity. LC-MS Method A: Purity: 96.1%; Observed Mass: 506.10; RT: 1.66 min. LC-MS Method B: Purity: 100%; Observed Mass: 506.00; RT: 1.31 min. ¹H NMR (499 MHz, DMSO-d₆) b ppm 10.45-9.75 (m, 1H), 9.76-9.46 (m, 1H), 9.38-9.07 (m, 1H), 8.61-8.32 (m, 1H), 8.32-8.14 (m, 1H), 8.14-7.89 (m, 1H), 7.89-7.62 (m, 1H), 7.62-7.35 (m, 1H), 4.11-3.80 (m, 1H), 3.76-2.95 (m, 8H), 2.94-2.77 (m, 1H), 1.64-1.31 (m, 3H), 0.89-0.67 (m, 4H).

The following compounds were prepared in a similar procedure as described for Example 1 employing the R⁵ group identified in the following table:

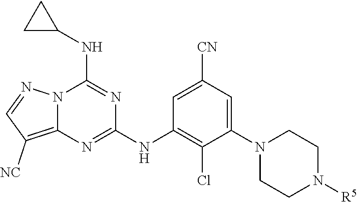

| | | LC/MS [M + H]⁺/ |
|---|---|---|
| | | RT |
| Example # | R⁵ | (Method A) |
| 2 | 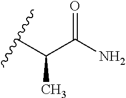 [SMILES: *[C@H](C)C(N)=O] | 506.2 1.16 min |
| 3 | 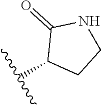 [SMILES: *[C@H]1CCNC1=O] | 518.2 1.63 min |
| 4 | 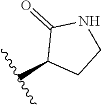 [SMILES: *[C@@H]1CCNC1=O] | 518.2 1.56 min |
| 5 | 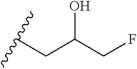 [SMILES: *CC(O)CF] | 511.2 1.82 min |
| 6 | 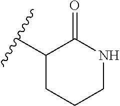 [SMILES: *C1CCCNC1=O] | 532.3 1.81 min |
| 7 | 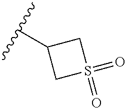 [SMILES: *C1CS(=O)(=O)C1] | 539.1 1.71 min |
| 8 | 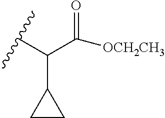 [SMILES: *C(C(=O)OCC)C1CC1] | 561.1 2.31 min |
| 9 | 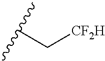 [SMILES: *CC(F)F] | 499.0 2.10 min |
| 10 | 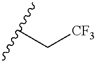 [SMILES: *CC(F)(F)F] | 517.1 2.26 min |

### LC/MC Conditions:

Column: Waters XBridge C18, 2.1 mm×50 mm, 1.7 μm particles; Mobile Phase A: 5:95 acetonitrile: water with 10 mM ammonium acetate; Mobile Phase B: 95:5 acetonitrile: water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 11 and Example 12

### 2-[(2-Chloro-5-cyano-3-{4-[(2S)-3-fluoro-2-hydroxypropyl]piperazin-1-yl}phenyl)amino]-4-(cyclopropylamino)pyrazolo[1,5-a][1,3,5]triazine-8-carbonitrile (Example 11) and 2-[(2-chloro-5-cyano-3-{4-[(2R)-3-fluoro-2-hydroxypropyl]piperazin-1-yl}phenyl)amino]-4-(cyclopropylamino)pyrazolo[1,5-a][1,3,5]triazine-8-carbonitrile (Example 12)

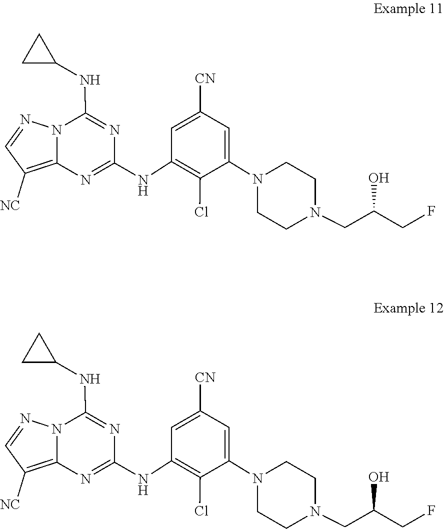 [SMILES: N#Cc1cc(Nc2nc(NC3CC3)n3ncc(C#N)c3n2)c(Cl)c(N2CCN(C[C@H](O)CF)CC2)c1 ; N#Cc1cc(Nc2nc(NC3CC3)n3ncc(C#N)c3n2)c(Cl)c(N2CCN(C[C@@H](O)CF)CC2)c1]

2-({2-Chloro-5-cyano-3-[4-(3-fluoro-2-hydroxypropyl)piperazin-1-yl]phenyl}amino)-4-(cyclopropylamino)pyrazolo[1,5-a][1,3,5]triazine-8-carbonitrile (22.4 mg, 0.044 mmol) was purified via preparative SFC chromatography employing the following conditions: Column: Chiralpak ADH, 30 mm×250 mm, 5 μm particles; Mobile Phase: 70% C_{02/30}% MeOH-ACN 50-50; Flow Rate: 100 mL/min; Column Temperature: 50° C. Fraction collection was triggered by UV (220 nm). Fractions containing the desired product were combined and dried via centrifugal evaporation to give the first eluent, 2-[(2-chloro-5-cyano-3-{4-[(2S)-3-fluoro-2 hydroxypropyl]piperazin-1-yl}phenyl)amino]-4-(cyclopropylamino)pyrazolo[1,5-a][1,3,5]triazine-8-carbonitrile (4.3 mg, 8.42 μmol, 7.32% yield) as Example 11 and the second eluent 2-[(2-chloro-5-cyano-3-{4-[(2R)-3-fluoro-2-hydroxypropyl]piperazin-1-yl}phenyl)amino]-4-(cyclopropylamino)pyrazolo[1,5-a][1,3,5]triazine-8-carbonitrile (3.4 mg, 6.65 μmol, 5.79% yield) as Example 12. The stereochemistry of the hydroxy group in Example 11 and Example 12 was tentatively assigned as shown.

Example 11 analytical data: Analytical LC/MS was used to determine the final purity. LC-MS Method A: Purity: 100%; Observed Mass: 511.10; RT: 1.66 min. LC-MS Method B: Purity: 100%; Observed Mass: 511.20; RT: 1.33 min. The assignment of the stereochemistry is tentative.
Example 12 analytical data: Analytical LC/MS was used to determine the final purity. LC-MS Method A: Purity: 100%; Observed Mass: 511.20; RT: 1.65 min. LC-MS Method B: Purity: 100%; Observed Mass: 511.10; RT: 1.32 min. The assignment of the stereochemistry is tentative.

### Example 13

### Methyl 4-(2-chloro-5-cyano-3-{[8-cyano-4-(cyclopropylamino)pyrazolo[1,5-a][1,3,5]triazin-2-yl]amino}phenyl)piperazine-1-carboxylate

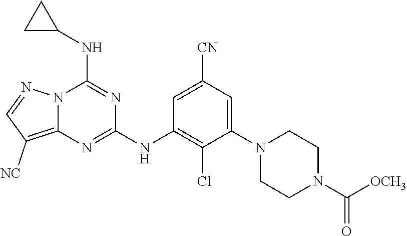 [SMILES: COC(=O)N1CCN(c2cc(C#N)cc(Nc3nc(NC4CC4)n4ncc(C#N)c4n3)c2Cl)CC1]

To a mixture of 2-{[2-chloro-5-cyano-3-(piperazin-1-yl)phenyl]amino}-4-(cyclopropylamino)pyrazolo[1,5-a][1,3,5]triazine-8-carbonitrile·TFA (24 mg, 0.044 mmol) in DMF (1 mL) was added Hunig's base (0.023 mL, 0.131 mmol) and methyl carbonochloridate (4.13 mg, 0.044 mmol) and the contents stirred at rt for 15 min. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 26% B, 26-66% B over 30 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 6.5 mg (30.2%), and its estimated purity by LCMS analysis was 100%. Analytical LC/MS was used to determine the final purity. LC-MS Method A: Purity: 100%; Observed Mass: 493.00; RT: 1.94 min. LC-MS Method B: Purity: 100%; Observed Mass: 493.00; RT: 1.93 min.

The following compounds were prepared employing a similar protocol as described for Example 13 employing the R⁵ group identified in the following table:

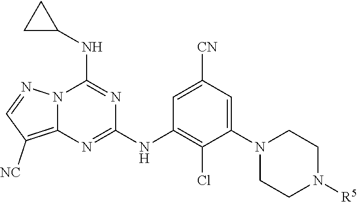

| | | LC/MS [M + H]⁺/ |
|---|---|---|
| | | RT |
| Example # | R⁵ | (Method A) |
| 14 | 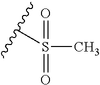 [SMILES: *S(C)(=O)=O] | 513.0 1.78 min |
| 15 | 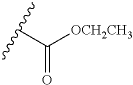 [SMILES: *C(=O)OCC] | 507.3 1.98 min |
| 16 | 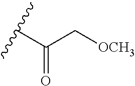 [SMILES: *C(=O)COC] | 507.2 1.64 min |
| 17 | 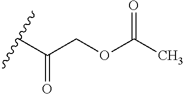 [SMILES: *C(=O)COC(C)=O] | 535.1 1.65 min |
| 18 | 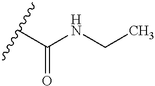 [SMILES: *C(=O)NCC] | 506.2 1.68 min |
| 19 | 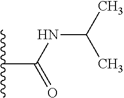 [SMILES: *C(=O)NC(C)C] | 520.3 1.80 min |
| 20 | 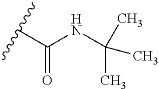 [SMILES: *C(=O)NC(C)(C)C] | 534.4 1.98 min |
| 21 | 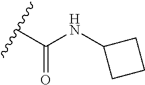 [SMILES: *C(=O)NC1CCC1] | 532.1 1.87 min |
| 22 | 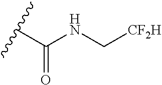 [SMILES: *C(=O)NCC(F)F] | 542.1 1.73 min |
| 23 | 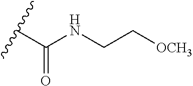 [SMILES: *C(=O)NCCOC] | 536.2 1.63 min |
| 24 | 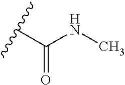 [SMILES: *C(=O)NC] | 492.1 1.59 min |
| 25 | 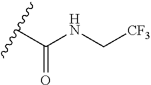 [SMILES: *C(=O)NCC(F)(F)F] | 560.1 1.76 min |
| 26 | 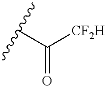 [SMILES: *C(=O)C(F)F] | 513.0 1.79 min |

### Method A:

Column: Waters XBridge C18, 2.1 mm×50 mm, 1.7 μm particles; Mobile Phase A: 5:95 acetonitrile: water with 10 mM ammonium acetate; Mobile Phase B: 95:5 acetonitrile: water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Method B:

Column: Waters Acquity BEH Phenyl 2.1×50 mm 1.7 μm particles; Mobile Phase A: 5:95 acetonitrile: water with 10 mM ammonium acetate; Mobile Phase B: 95:5 acetonitrile: water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 27

### 2-({2-Chloro-5-cyano-3-[4-(oxan-4-yl)piperazin-1-yl]phenyl}amino)-4-(cyclopropylamino)pyrazolo[1,5-a][1,3,5]triazine-8-carbonitrile

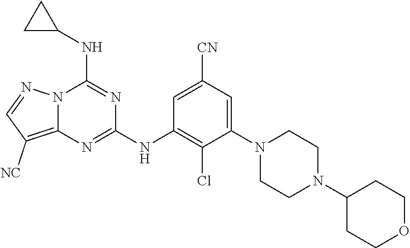 [SMILES: N#Cc1cc(Nc2nc(NC3CC3)n3ncc(C#N)c3n2)c(Cl)c(N2CCN(C3CCOCC3)CC2)c1]

A mixture of 2-{[2-chloro-5-cyano-3-(piperazin-1-yl)phenyl]amino}-4-(cyclopropylamino)pyrazolo[1,5-a][1,3,5]triazine-8-carbonitrile (20 mg, 0.046 mmol), acetic acid (0.013 mL, 0.230 mmol) and tetrahydro-4H-pyran-4-one (13.81 mg, 0.138 mmol) was stirred at rt for 0.5 h, then 1 M sodium cyanoborohydride in THF (0.230 mL, 0.230 mmol) was added. The mixture was stirred at rt for 16 h. The reaction mixture was concentrated, partitioned between DCM and sat. aq. NaHCO₃. The DCM layer was separated, washed with brine, dried over sodium sulfate, concentrated and the crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 10% B, 10-50% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 10.1 (41.3%) mg, and its estimated purity by LCMS analysis was 98%. Analytical LC/MS was used to determine the final purity. LC-MS Method A: Purity: 99.1%; Observed Mass: 519.20; RT: 1.87 min. LC-MS Method B: Purity: 97.6%; Observed Mass: 519.30; RT: 1.41 min.

The following compounds were prepared as described for Example 27 employing the R⁵ group identified in the following table:

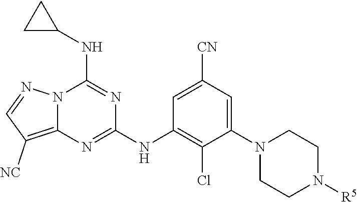

| | | LC/MS [M + H]⁺/ |
|---|---|---|
| | | RT |
| Example # | R⁵ | (Method A) |
| 28 | 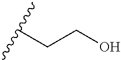 [SMILES: *CCO] | 479.2 1.47 min |
| 29 | 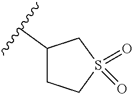 [SMILES: *C1CCS(=O)(=O)C1] | 553.2 1.67 min |
| 30 | 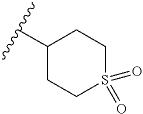 [SMILES: *C1CCS(=O)(=O)CC1] | 567.2 1.67 min |
| 31 | 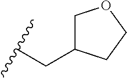 [SMILES: *CC1CCOC1] | 519.2 1.92 min |
| 32 | 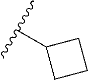 [SMILES: *C1CCC1] | 489.3 2.03 min |
| 33 | 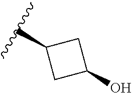 [SMILES: *[C@H]1C[C@@H](O)C1] | 505.2 1.61 min |
| 34 | 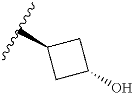 [SMILES: *[C@H]1C[C@H](O)C1] | 505.2 1.47 min |
| 35 | 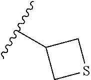 [SMILES: *C1CSC1] | 507.1 2.08 min |
| 36 | 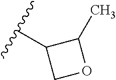 [SMILES: *C1COC1C] | 505.1 1.78 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 37

### 2-[4-(2-Chloro-5-cyano-3-{[8-cyano-4-(cyclopropylamino)pyrazolo[1,5-a][1,3,5]triazin-2-yl]amino}phenyl)piperazin-1-yl]acetamide

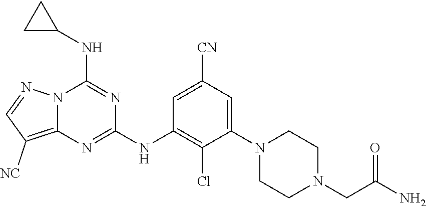 [SMILES: N#Cc1cc(Nc2nc(NC3CC3)n3ncc(C#N)c3n2)c(Cl)c(N2CCN(CC(N)=O)CC2)c1]

To a mixture of 2-{[2-chloro-5-cyano-3-(piperazin-1-yl)phenyl]amino}-4-(cyclopropylamino)pyrazolo[1,5-a][1,3,5]triazine-8-carbonitrile (15 mg, 0.034 mmol) in DMF (1 mL) was added 2-bromoacetamide (4.76 mg, 0.034 mmol) and K₂CO₃ (9.53 mg, 0.069 mmol) and the contents stirred at rt for 2 h. The crude reaction mixture was purified via preparative LC/MS employing the following conditions: Column: XBridge C18, 200 mm×19 mm, 5-μm particles;

Mobile Phase A: 5:95 acetonitrile: water with ammonium acetate; Mobile Phase B: 95:5 acetonitrile: water with ammonium acetate; Gradient: a 0-min hold at 16% B, 16-56% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 7.0 mg (40.4%), and its estimated purity by LCMS analysis was 98%.
Analytical LC/MS was used to determine the final purity. LC-MS Method A: Purity: 98.1%; Observed Mass: 492.20; RT: 1.5 min. LC-MS Method B: Purity: 98%; Observed Mass: 492.10; RT: 1.22 min.

### Example 38

### 2-({2-Chloro-5-cyano-3-[4-(2-methoxyethyl)piperazin-1-yl]phenyl}amino)-4-(cyclopropylamino)pyrazolo[1,5-a][1,3,5]triazine-8-carbonitrile

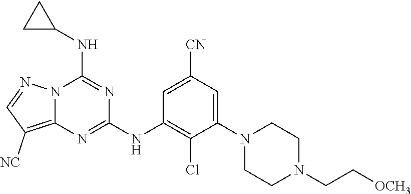 [SMILES: COCCN1CCN(c2cc(C#N)cc(Nc3nc(NC4CC4)n4ncc(C#N)c4n3)c2Cl)CC1]

A mixture of 2-{[2-chloro-5-cyano-3-(piperazin-1-yl)phenyl]amino}-4-(cyclopropylamino)pyrazolo[1,5-a][1,3,5]triazine-8-carbonitrile (20 mg, 0.046 mmol), Et₃N (0.019 mL, 0.138 mmol), 1-bromo-2-methoxyethane (6.39 mg, 0.046 mmol) in DMF (0.5 mL) was heated at 50° C. for 16 h. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 24% B, 24-64% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 11.5 mg (50.7%), and its estimated purity by LCMS analysis was 100%. Analytical LC/MS was used to determine the final purity. LC-MS Method A: 100%; Observed Mass: 493.20; RT: 1.75 min. LC-MS Method B: Purity: 100%; Observed Mass: 493.20; RT: 1.32 min.

### Example 39

### 2-[4-(2-Chloro-5-cyano-3-{[8-cyano-4-(cyclopropylamino)pyrazolo[1,5-a][1,3,5]triazin-2-yl]amino}phenyl)piperazin-1-yl]-2-oxoacetamide

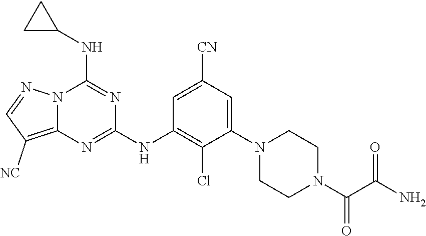 [SMILES: N#Cc1cc(Nc2nc(NC3CC3)n3ncc(C#N)c3n2)c(Cl)c(N2CCN(C(=O)C(N)=O)CC2)c1]

A mixture of 2-{[2-chloro-5-cyano-3-(piperazin-1-yl)phenyl]amino}-4-(cyclopropylamino)pyrazolo[1,5-a][1,3,5]triazine-8-carbonitrile (10 mg, 0.023 mmol), 2-amino-2-oxoacetic acid (2.457 mg, 0.028 mmol), HATU (10.49 mg, 0.028 mmol) in DMF (0.5 mL) was stirred at rt for 2 h. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 14% B, 14-54% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 7.9 mg, and its estimated purity by LCMS analysis was 98%. Analytical LC/MS was used to determine the final purity. LC-MS Method A: Purity: 98.3%; Observed Mass: 506.10; RT: 1.48 min. LC-MS Method B: Purity: 97.8%; Observed Mass: 506.10; RT: 1.47 min.

The following compounds were prepared in a similar procedure as described for Example 39 employing the R⁵ group identified in the following table:

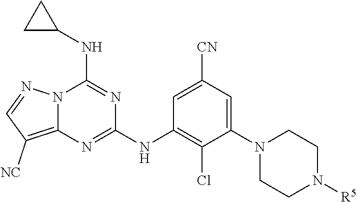

| | | LC/MS [M + H]⁺/ |
|---|---|---|
| | | RT |
| Example # | R⁵ | (Method A) |
| 40 | 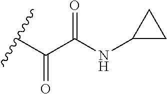 [SMILES: *C(=O)C(=O)NC1CC1] | 546.2 1.65 min |
| 41 | 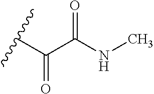 [SMILES: *C(=O)C(=O)NC] | 520.1 1.56 min |
| 42 | 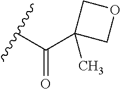 [SMILES: *C(=O)C1(C)COC1] | 533.0. 1.66 min |
| 43 | 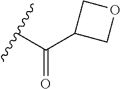 [SMILES: *C(=O)C1COC1] | 519.1 1.58 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 44

### 2-({2-Chloro-5-cyano-3-[(3R)-3-methylpiperazin-1-yl]phenyl}amino)-4-(cyclopropylamino)pyrazolo[1,5-a][1,3,5]triazine-8-carbonitrile

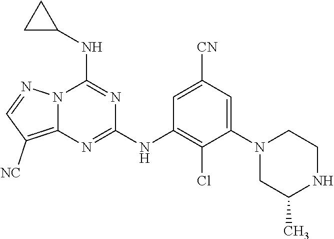 [SMILES: C[C@@H]1CN(c2cc(C#N)cc(Nc3nc(NC4CC4)n4ncc(C#N)c4n3)c2Cl)CCN1]

### Step 1. tert-butyl (R)-4-(3-amino-2-chloro-5-cyanophenyl)-2-methylpiperazine-1-carboxylate

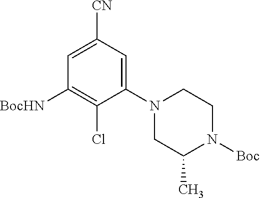 [SMILES: C[C@@H]1CN(c2cc(C#N)cc(NC(=O)OC(C)(C)C)c2Cl)CCN1C(=O)OC(C)(C)C]

A mixture of tert-butyl (3-bromo-2-chloro-5-cyanophenyl)carbamate (500 mg, 1.508 mmol), tert-butyl (R)-2-methylpiperazine-1-carboxylate (362 mg, 1.809 mmol), BINAP (282 mg, 0.452 mmol), Cs₂CO₃ (983 mg, 3.02 mmol) in dioxane (24 mL) was purged with nitrogen and then Pd₂dba₃ (138 mg, 0.151 mmol) was added and purged with nitrogen for 5 min. The mixture was stirred at 105° C. for 16 h. After the brown slurry was cooled to rt, celite was added. The reaction mixture was filtered through celite and washed with EtOAc. The filtrate was concentrated in vacuo. The crude was purified by silica gel column chromatography, eluting with 20% ethyl acetate in hexanes to give tert-butyl (R)-4-(3-((tert-butoxycarbonyl)amino)-2-chloro-5-cyanophenyl)-2-methylpiperazine-1-carboxylate (500 mg, 1.109 mmol, 73.5% yield). LC/MS [M+H]⁺=451.3. ¹H NMR (499 MHz, CHLOROFORM-d) δ ppm 8.41-8.32 (m, 1H), 7.24-7.17 (m, 1H), 7.00-6.92 (m, 1H), 4.55-4.32 (m, 1H), 4.07-3.89 (m, 1H), 3.41-3.29 (m, 1H), 3.25-3.16 (m, 1H), 3.16-3.07 (m, 1H), 2.95-2.83 (m, 1H), 2.79-2.63 (m, 1H), 1.59-1.55 (m, 9H), 1.53-1.49 (m, 9H), 1.43-1.39 (m, 3H).

### Step 2. tert-Butyl (R)-4-(3-amino-2-chloro-5-cyanophenyl)-2-methylpiperazine-1-carboxylate

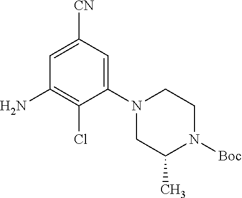 [SMILES: C[C@@H]1CN(c2cc(C#N)cc(N)c2Cl)CCN1C(=O)OC(C)(C)C]

To a mixture of tert-butyl (R)-4-(3-((tert-butoxycarbonyl)amino)-2-chloro-5-cyanophenyl)-2-methylpiperazine-1-carboxylate (500 mg, 1.109 mmol) in CH₂Cl₂ (5 mL) was added TFA (1.623 mL, 21.07 mmol) and the contents stirred at rt for 2 h. The reaction mixture was concentrated, and DCM (5 mL×3) was added, and the contents concentrated to remove all TFA. TEA (0.309 mL, 2.217 mmol) was added, and the contents concentrated. To the residue was added CH₂Cl₂ (5 mL), Boc-anhydride (0.283 mL, 1.220 mmol) and TEA (0.309 mL, 2.217 mmol) and the contents stirred at rt for 2 h. The reaction mixture was diluted with EtOAc and washed with water. The organic layer was dried over MgSO₄, filtered and concentrated. The residue was purified by ISCO, eluting with 20% EtOAc/hexane to afford tert-butyl (R)-4-(3-amino-2-chloro-5-cyanophenyl)-2-methylpiperazine-1-carboxylate (350 mg, 0.998 mmol, 90% yield). LC/MS [M+H]⁺=351.1. ¹H NMR (499 MHz, CHLOROFORM-d) δ ppm 6.85-6.72 (m, 1H), 6.75-6.52 (m, 1H), 4.60-4.26 (m, 3H), 4.08-3.87 (m, 1H), 3.45-3.08 (m, 3H), 2.91-2.45 (m, 2H), 1.53-1.49 (m, 9H), 1.44-1.40 (m, 3H).

### Step 3. tert-Butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2-methylpiperazine-1-carboxylate

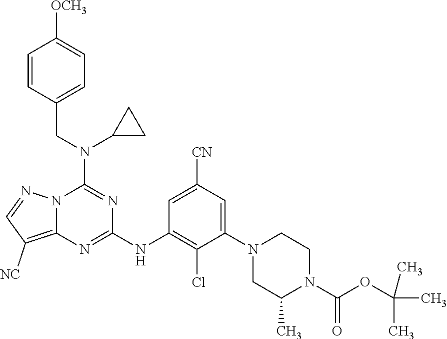 [SMILES: COc1ccc(CN(c2nc(Nc3cc(C#N)cc(N4CCN(C(=O)OC(C)(C)C)[C@H](C)C4)c3Cl)nc3c(C#N)cnn23)C2CC2)cc1]

To a mixture of tert-butyl (R)-4-(3-amino-2-chloro-5-cyanophenyl)-2-methylpiperazine-1-carboxylate (250 mg, 0.713 mmol) in THF (5 mL) was added LiHMDS (1.568 mL, 1.568 mmol) dropwise at −78° C. The contents were stirred at −78° C. for 30 min and then 4-(cyclopropyl(4-methoxybenzyl) amino)-2-(methylsulfonyl)pyrazolo[1,5-a][1,3,5]triazine-8-carbonitrile (284 mg, 0.713 mmol) in 2 mL of THF was added and the contents stirred at −78° C. for 30 min. The reaction was quenched with water and extracted with EtOAc. The organic layer was washed with water, dried over MgSO₄, filtered and concentrated. To the residue was added MeOH. The resulting solid was filtered, washed with MeOH and dried to obtain tert-butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2-methylpiperazine-1-carboxylate (450 mg, 0.672 mmol, 94% yield). LC/MS [M+H]⁺=669.6.

### Step 4. Example 44

A mixture of tert-butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2-methylpiperazine-1-carboxylate (450 mg, 0.672 mmol) in ClCH₂CH₂Cl (5 mL) was added TFA (1.036 mL, 13.45 mmol) and anisole (0.073 mL, 0.672 mmol) and heated at 50° C. for 1.5 h. The reaction mixture was cooled to rt and concentrated. To the residue was added 0.5 mL of 7N NH₃ in MeOH and the solid was filtered and dried to obtain 2-({2-chloro-5-cyano-3-[(3R)-3-methylpiperazin-1-yl]phenyl}amino)-4-(cyclopropylamino) pyrazolo[1,5-a][1,3,5]triazine-8-carbonitrile (200 mg, 0.446 mmol, 66.3% yield). LC/MS [M+H]⁺=449.0. A portion of the crude material (10 mg) was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 13% B, 13-58% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 3.1 mg (46% yield), and its estimated purity by LCMS analysis was 100%. Analytical LC/MS was used to determine the final purity. LC-MS Method A: Purity: 100%; Observed Mass: 449.00; RT: 1.4 min. LC-MS Method B: Purity: 100%; Observed Mass: 449.10; RT: 1.4 min.

### Example 45

### 2-({2-Chloro-5-cyano-3-[(3R)-3,4-dimethylpiperazin-1-yl]phenyl}amino)-4-(cyclopropylamino)pyrazolo[1,5-a][1,3,5]triazine-8-carbonitrile

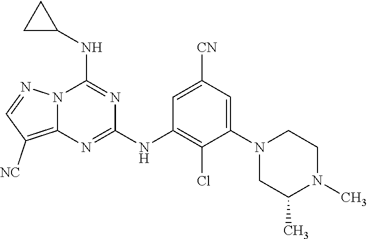 [SMILES: C[C@@H]1CN(c2cc(C#N)cc(Nc3nc(NC4CC4)n4ncc(C#N)c4n3)c2Cl)CCN1C]

A mixture of 2-({2-chloro-5-cyano-3-[(3R)-3-methylpiperazin-1-yl]phenyl}amino)-4-(cyclopropylamino)pyrazolo[1,5-a][1,3,5]triazine-8-carbonitrile (20 mg, 0.045 mmol), acetic acid (0.013 mL, 0.223 mmol) and formaldehyde (4.01 mg, 0.134 mmol) was stirred at rt for 0.5 h, then sodium triacetoxyborohydride (47.2 mg, 0.223 mmol) was added. The mixture was stirred at rt for 2 h. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 22% B, 22-62% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 6.2 mg (30.1% yield), and its estimated purity by LCMS analysis was 100%. Analytical LC/MS was used to determine the final purity. LC-MS Method A: Purity: 100%; Observed Mass: 463.10; RT: 1.78 min. LC-MS Method B: 100%; Observed Mass: 463.20; RT: 1.4 min.

### Example 46

### 2-[(2R)-4-(2-Chloro-5-cyano-3-{[8-cyano-4-(cyclopropylamino)pyrazolo[1,5-a][1,3,5]triazin-2-yl]amino}phenyl)-2-methylpiperazin-1-yl]acetamide

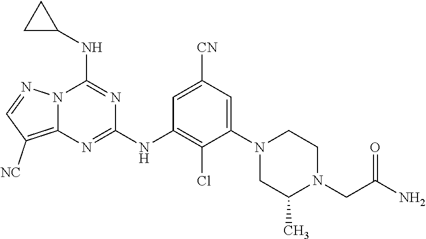 [SMILES: C[C@@H]1CN(c2cc(C#N)cc(Nc3nc(NC4CC4)n4ncc(C#N)c4n3)c2Cl)CCN1CC(N)=O]

This compound was prepared according to the method employed to prepare Example 37. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 23% B, 23-63% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 2.1 mg, and its estimated purity by LCMS analysis was 98%. Analytical LC/MS was used to determine the final purity. LC-MS Method A: Purity: 97.7%; Observed Mass: 506.30; RT: 1.66 min. LC-MS Method B: Purity: 100%; Observed Mass: 506.20; RT: 1.31 min.

### Example 47

### 2-({2-Chloro-5-cyano-3-[(3R)-4-methanesulfonyl-3-methylpiperazin-1-yl]phenyl}amino)-4-(cyclopropylamino)pyrazolo[1,5-a][1,3,5]triazine-8-carbonitrile

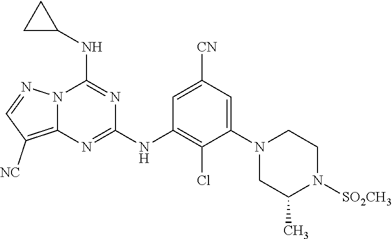 [SMILES: C[C@@H]1CN(c2cc(C#N)cc(Nc3nc(NC4CC4)n4ncc(C#N)c4n3)c2Cl)CCN1S(C)(=O)=O]

To a mixture of 2-({2-chloro-5-cyano-3-[(3R)-3-methylpiperazin-1-yl]phenyl}amino)-4-(cyclopropylamino)pyrazolo[1,5-a][1,3,5]triazine-8-carbonitrile, TFA (10 mg, 0.018 mmol) in DMF (1 mL) was added Hunig's base (9.31 μL, 0.053 mmol) and methanesulfonyl chloride (2.035 mg, 0.018 mmol) and the contents stirred at rt for 15 min. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 28% B, 28-68% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 5.9 mg (63% yield), and its estimated purity by LCMS analysis was 100%. Analytical LC/MS was used to determine the final purity. LC-MS Method A: Purity: 100%; Observed Mass: 527.00; RT: 1.9 min. LC-MS Method B: Purity: 100%; Observed Mass: 527.00; RT: 1.87 min.

### Example 48

### (2R)-2-[(2R)-4-(2-Chloro-5-cyano-3-{[8-cyano-4-(cyclopropylamino)pyrazolo[1,5-a][1,3,5]triazin-2-yl]amino}phenyl)-2-methylpiperazin-1-yl]propanamide

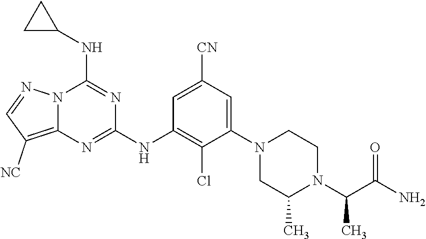 [SMILES: C[C@@H]1CN(c2cc(C#N)cc(Nc3nc(NC4CC4)n4ncc(C#N)c4n3)c2Cl)CCN1[C@H](C)C(N)=O]

A solution of 2-({2-chloro-5-cyano-3-[(3R)-3-methylpiperazin-1-yl]phenyl}amino)-4-(cyclopropylamino)pyrazolo[1,5-a][1,3,5]triazine-8-carbonitrile (10 mg, 0.022 mmol), (S)-1-amino-1-oxopropan-2-yl methanesulfonate (5.59 mg, 0.033 mmol), Hunig's base (7.78 μL, 0.045 mmol) in DMF (1 mL) was stirred at 80° C. for 24 h. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 26% B, 26-66% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 2.8 mg (24.2% yield), and its estimated purity by LCMS analysis was 100%. Analytical LC/MS was used to determine the final purity. LC-MS Method A: Purity: 100%; Observed Mass: 520.30; RT: 1.84 min. LC-MS Method B: 100%; Observed Mass: 520.20; RT: 1.32 min.

### Example 49

### 2-{4-[4-(2-Chloro-5-cyano-3-{[8-cyano-4-(cyclopropylamino)pyrazolo[1,5-a][1,3,5]triazin-2-yl]amino}phenyl)piperazin-1-yl]piperidin-1-yl}acetamide

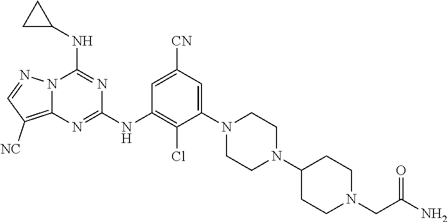 [SMILES: N#Cc1cc(Nc2nc(NC3CC3)n3ncc(C#N)c3n2)c(Cl)c(N2CCN(C3CCN(CC(N)=O)CC3)CC2)c1]

### Step 1. tert-Butyl 4-(4-(2-chloro-5-cyano-3-((8-cyano-4-(cyclopropylamino) pyrazolo[1,5-a][1,3,5]triazin-2-yl)amino)phenyl)piperazin-1-yl)piperidine-1-carboxylate

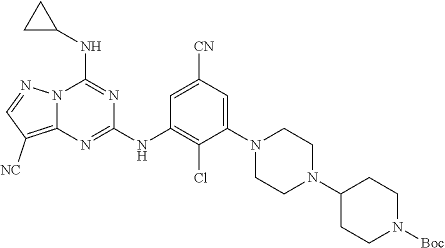 [SMILES: CC(C)(C)OC(=O)N1CCC(N2CCN(c3cc(C#N)cc(Nc4nc(NC5CC5)n5ncc(C#N)c5n4)c3Cl)CC2)CC1]

A mixture of 2-((2-chloro-5-cyano-3-(piperazin-1-yl)phenyl)amino)-4-(cyclopropylamino)pyrazolo[1,5-a][1,3,5]triazine-8-carbonitrile (227 mg, 0.522 mmol), acetic acid (0.060 mL, 1.044 mmol) and 1-Boc-4-piperidone (125 mg, 0.626 mmol) was stirred at rt for 0.5 h, then sodium triacetoxyborohydride (221 mg, 1.044 mmol) was added. The mixture was stirred at rt for 16 h. A sat. aq. NaHCO₃ solution was added to the reaction mixture and the resulting solid was filtered, washed with water and dried to obtain tert-butyl 4-(4-(2-chloro-5-cyano-3-((8-cyano-4-(cyclopropylamino)pyrazolo[1,5-a][1,3,5]triazin-2-yl)amino)phenyl)piperazin-1-yl)piperidine-1-carboxylate (293 mg, 0.453 mmol, 87% yield). [M+H]⁺=619.3.

### Step 2. 2-((2-Chloro-5-cyano-3-(4-(piperidin-4-yl)piperazin-1-yl)phenyl) amino)-4-(cyclopropylamino)pyrazolo[1,5-a][1,3,5]triazine-8-carbonitrile

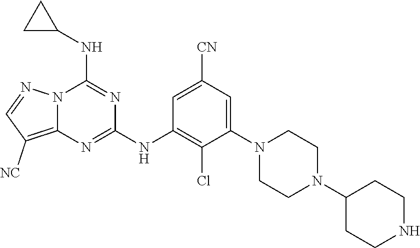 [SMILES: N#Cc1cc(Nc2nc(NC3CC3)n3ncc(C#N)c3n2)c(Cl)c(N2CCN(C3CCNCC3)CC2)c1]

To a mixture of tert-butyl 4-(4-(2-chloro-5-cyano-3-((8-cyano-4-(cyclopropylamino)pyrazolo[1,5-a][1,3,5]triazin-2-yl)amino)phenyl)piperazin-1-yl)piperidine-1-carboxylate (292 mg, 0.472 mmol) in CH₂Cl₂ (5 mL) was added TFA (1.092 mL, 14.17 mmol) and the contents stirred at rt for 1 h. The reaction mixture was concentrated to yield 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2 TFA (350 mg, 0.462 mmol, 98% yield) which was used as such for the subsequent step without further purification.

### Step 3. Example 49

To a mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2TFA salt (54 mg, 0.104 mmol) in DMSO (1 mL) was added 2-bromoacetamide (14.38 mg, 0.104 mmol) and K₂CO₃ (28.8 mg, 0.208 mmol) and the contents stirred at rt for 2 h. The crude material was purified via preparative LC/MS employing the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-min hold at 0% B, 0-40% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 18.5 mg (22.10% yield), and its estimated purity by LCMS analysis was 100%. Analytical LC/MS was used to determine the final purity. LC-MS Method B: Purity: 100%; Observed Mass: 575.20; RT: 1.11 min. LC-MS Method A: Purity: 100%; Observed Mass: 575.20; RT: 1.44 min.

### Example 50

### Methyl 4-[4-(2-chloro-5-cyano-3-{[8-cyano-4-(cyclopropylamino)pyrazolo[1,5-a][1,3,5]triazin-2-yl]amino}phenyl)piperazin-1-yl]piperidine-1-carboxylate

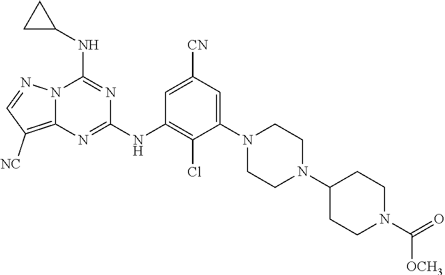 [SMILES: COC(=O)N1CCC(N2CCN(c3cc(C#N)cc(Nc4nc(NC5CC5)n5ncc(C#N)c5n4)c3Cl)CC2)CC1]

To a mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2TFA salt (15 mg, 0.029 mmol) in DMSO (1 mL) was added Hunig's base (10.11 μL, 0.058 mmol) and methyl carbonochloridate (4.10 mg, 0.043 mmol) and the contents stirred at rt for 15 min. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 26% B, 26-66% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 3.7 mg (20.90% yield), and its estimated purity by LCMS analysis was 94%. Analytical LC/MS was used to determine the final purity. LC-MS Method A: Purity: 94.2%; Observed Mass: 576.20; RT: 1.79 min. LC-MS Method B: Purity: 95.7%; Observed Mass: 576.20; RT: 1.36 min.

The following compounds were prepared in a similar procedure as described for Example 50 employing the R^{b} group identified in the following table:

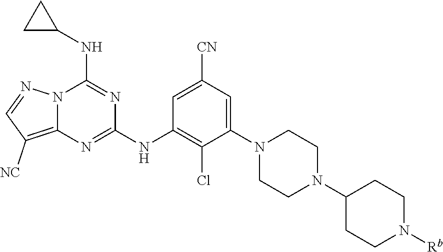

| | | LC/MS [M + H]⁺/ |
|---|---|---|
| | | RT |
| Example # | R^{b} | (Method A) |
| 51 | 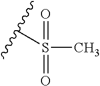 [SMILES: *S(C)(=O)=O] | 596.3 1.75 min |
| 52 | 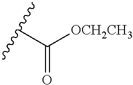 [SMILES: *C(=O)OCC] | 590.2 1.92 min |
| 53 | 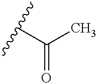 [SMILES: *C(C)=O] | 560.2 1.55 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 54

### 2-[(2-Chloro-5-cyano-3-{4-[1-(2-methoxyethyl)piperidin-4-yl]piperazin-1-yl}phenyl)amino]-4-(cyclopropylamino)pyrazolo[1,5-a][1,3,5]triazine-8-carbonitrile

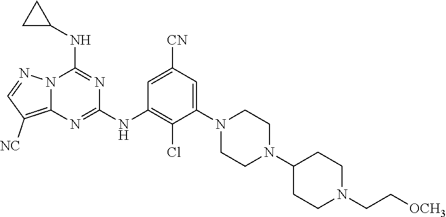 [SMILES: COCCN1CCC(N2CCN(c3cc(C#N)cc(Nc4nc(NC5CC5)n5ncc(C#N)c5n4)c3Cl)CC2)CC1]

A mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2 TFA (15 mg, 0.020 mmol), Et₃N (8.41 μL, 0.060 mmol), 1-bromo-2-methoxyethane (3.07 mg, 0.022 mmol) in DMF (0.5 mL) was heated at 50° C. for 16 h. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 20% B, 20-45% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 4.2 mg (36.3% yield), and its estimated purity by LCMS analysis was 100%. Analytical LC/MS was used to determine the final purity. LC-MS Method A: Purity: 100%; Observed Mass: 576.30; RT: 1.6 min. LC-MS Method B: Purity: 100%; Observed Mass: 576.30; RT: 1.2 min.

### Example 55

### 2-[(2-Chloro-5-cyano-3-{4-[1-(oxetan-3-yl)piperidin-4-yl]piperazin-1-yl}phenyl) amino]-4-(cyclopropylamino)pyrazolo[1,5-a][1,3,5]triazine-8-carbonitrile

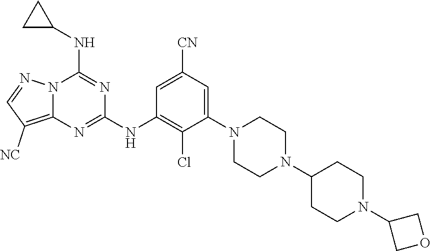 [SMILES: N#Cc1cc(Nc2nc(NC3CC3)n3ncc(C#N)c3n2)c(Cl)c(N2CCN(C3CCN(C4COC4)CC3)CC2)c1]

A mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2 TFA (15 mg, 0.020 mmol), Hunig's base (7.02 μL, 0.040 mmol) acetic acid (2.302 μL, 0.040 mmol) and oxetan-3-one (2.90 mg, 0.040 mmol) was stirred at rt for 0.5 h, then sodium cyanoborohydride (0.040 mL, 0.040 mmol) was added. The mixture was stirred at rt for 16 h. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 4-min hold at 1% B, 1-41% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 9.0 mg (78% yield), and its estimated purity by LCMS analysis was 100%. Analytical LC/MS was used to determine the final purity. LC-MS Method B: Purity: 100%; Observed Mass: 574.20; RT: 1.2 min. LC-MS Method A: Purity: 100%; Observed Mass: 574.30; RT: 1.56 min.

### Example 56

### 2-[(2-Chloro-5-cyano-3-{4-[1-(3-hydroxycyclobutyl)piperidin-4-yl]piperazin-1-yl}phenyl)amino]-4-(cyclopropylamino)pyrazolo[1,5-a][1,3,5]triazine-8-carbonitrile

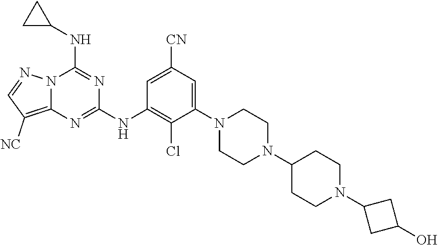 [SMILES: N#Cc1cc(Nc2nc(NC3CC3)n3ncc(C#N)c3n2)c(Cl)c(N2CCN(C3CCN(C4CC(O)C4)CC3)CC2)c1]

A mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2 TFA (15 mg, 0.020 mmol), acetic acid (2.302 μL, 0.040 mmol) and 3-hydroxycyclobutan-1-one (3.46 mg, 0.040 mmol) was stirred at rt for 0.5 h, then sodium cyanoborohydride (0.040 mL, 0.040 mmol) was added. The mixture was stirred at rt for 16 h. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 10% B, 10-50% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 3.2 mg (25.09% yield), and its estimated purity by LCMS analysis was 93%. Analytical LC/MS was used to determine the final purity. LC-MS Method A: 92.7%; Observed Mass: 588.20, 588.20; RT: 1.43, 1.47 min. LC-MS Method B: Purity: 100%; Observed Mass: 588.20, 588.20; RT: 1.21, 1.27 min.

### Example 57

### 2-[(2-Chloro-5-cyano-3-{4-[1-(3-fluoro-2-hydroxypropyl)piperidin-4-yl]piperazin-1-yl}phenyl)amino]-4-(cyclopropylamino)pyrazolo[1,5-a][1,3,5]triazine-8-carbonitrile

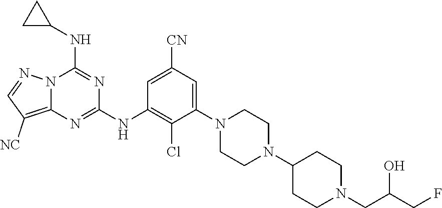 [SMILES: N#Cc1cc(Nc2nc(NC3CC3)n3ncc(C#N)c3n2)c(Cl)c(N2CCN(C3CCN(CC(O)CF)CC3)CC2)c1]

A mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile, 2 TFA salt (20 mg, 0.039 mmol), Hunig's base (0.013 mL, 0.077 mmol), 2-(fluoromethyl)oxirane (4.41 mg, 0.058 mmol) in DMF (1 mL) was heated at 80° C. for 16 h. The crude material was purified via preparative LC/MS employing the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 18% B, 18-58% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 2.7 mg (11.69% yield), and its estimated purity by LCMS analysis was 99%. Analytical LC/MS was used to determine the final purity. LC-MS Method A: Purity: 100%; Observed Mass: 594.20; RT: 1.52 min. LC-MS Method B: Purity: 99.3%; Observed Mass: 594.30, 594.30; RT: 1.19, 1.22 min.

### Example 58

### 2-{4-[4-(2-Chloro-5-cyano-3-{[8-cyano-4-(cyclopropylamino)pyrazolo[1,5-a][1,3,5]triazin-2-yl]amino}phenyl)piperazin-1-yl]piperidin-1-yl}-2-oxoacetamide

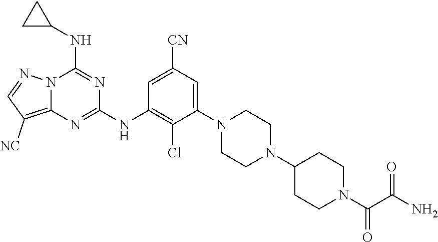 [SMILES: N#Cc1cc(Nc2nc(NC3CC3)n3ncc(C#N)c3n2)c(Cl)c(N2CCN(C3CCN(C(=O)C(N)=O)CC3)CC2)c1]

A mixture of 2-((2-chloro-5-cyano-3-(4-(piperidin-4-yl)piperazin-1-yl)phenyl)amino)-4-(cyclopropylamino)pyrazolo[1,5-a][1,3,5]triazine-8-carbonitrile (20 mg, 0.039 mmol), 2-amino-2-oxoacetic acid (4.13 mg, 0.046 mmol), HATU (17.62 mg, 0.046 mmol), Hunig's base (0.020 mL, 0.116 mmol) in DMF (0.5 mL) was stirred at rt for 2 h. The crude material was purified using preparative LC/MS employing the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 10% B, 10-50% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation The yield of the product was 5.7 mg (25.06% yield), and its estimated purity by LCMS analysis was 100%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 589.30; RT: 1.24 min. Injection 2 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 2 results: Purity: 100%; Observed Mass: 589.20; RT: 1.5 min.

### Example 59

### (2R)-2-{3-[4-(2-Chloro-5-cyano-3-{[8-cyano-4-(cyclopropylamino)pyrazolo[1,5-a][1,3,5]triazin-2-yl]amino}phenyl)piperazin-1-yl]azetidin-1-yl}propanamide

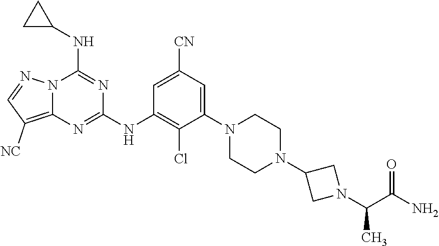 [SMILES: C[C@H](C(N)=O)N1CC(N2CCN(c3cc(C#N)cc(Nc4nc(NC5CC5)n5ncc(C#N)c5n4)c3Cl)CC2)C1]

### Step 1. tert-Butyl 3-(4-(2-chloro-5-cyano-3-((8-cyano-4-(cyclopropylamino) pyrazolo[1,5-a][1,3,5]triazin-2-yl)amino)phenyl)piperazin-1-yl)azetidine-1-carboxylate

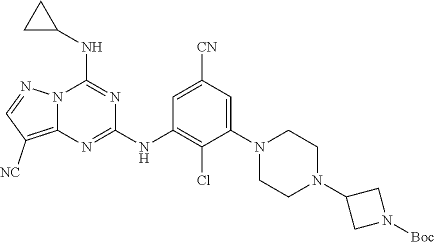 [SMILES: CC(C)(C)OC(=O)N1CC(N2CCN(c3cc(C#N)cc(Nc4nc(NC5CC5)n5ncc(C#N)c5n4)c3Cl)CC2)C1]

A mixture of 2-((2-chloro-5-cyano-3-(piperazin-1-yl)phenyl)amino)-4-(cyclopropylamino)pyrazolo[1,5-a][1,3,5]triazine-8-carbonitrile (150 mg, 0.345 mmol), acetic acid (0.039 mL, 0.690 mmol) and tert-butyl 3-oxoazetidine-1-carboxylate (89 mg, 0.517 mmol) was stirred at rt for 0.5 h, then sodium triacetoxyborohydride (146 mg, 0.690 mmol) was added. The mixture was stirred at rt for 16 h. Water was added, and the resulting solid was filtered and dried to obtain 2-((2-chloro-5-cyano-3-(piperazin-1-yl)phenyl)amino)-4-(cyclopropylamino)pyrazolo[1,5-a][1,3,5]triazine-8-carbonitrile (150 mg, 0.345 mmol). [M+H]⁺=590.7.

### Step 2. 2-((3-(4-(Azetidin-3-yl)piperazin-1-yl)-2-chloro-5-cyanophenyl)amino)-4-(cyclopropylamino)pyrazolo[1,5-a][1,3,5]triazine-8-carbonitrile

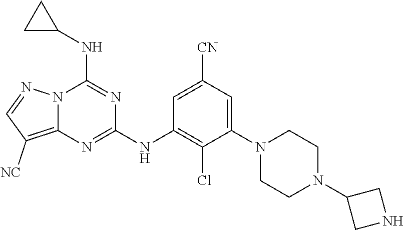 [SMILES: N#Cc1cc(Nc2nc(NC3CC3)n3ncc(C#N)c3n2)c(Cl)c(N2CCN(C3CNC3)CC2)c1]

A mixture of tert-butyl 3-(4-(2-chloro-5-cyano-3-((8-cyano-4-(cyclopropylamino)pyrazolo[1,5-a][1,3,5]triazin-2-yl)amino)phenyl)piperazin-1-yl)azetidine-1-carboxylate (175 mg, 0.297 mmol) in CH₂Cl₂ (2 mL) was added TFA (0.685 mL, 8.90 mmol) and the contents stirred at rt for 2 h. The reaction mixture was concentrated to yield 2-((3-(4-(azetidin-3-yl)piperazin-1-yl)-2-chloro-5-cyanophenyl)amino)-4-(cyclopropylamino)pyrazolo[1,5-a][1,3,5]triazine-8-carbonitrile, 2 TFA (200 mg, 0.279 mmol, 94% yield); [M+H]⁺=490.4 which was used as such for the subsequent step without further purification.

### Step 3. Example 59

A solution of 2-((3-(4-(azetidin-3-yl)piperazin-1-yl)-2-chloro-5-cyanophenyl)amino)-4-(cyclopropylamino)pyrazolo[1,5-a][1,3,5]triazine-8-carbonitrile, 2 TFA salt (15 mg, 0.021 mmol), Hunig's base (10.95 μl, 0.063 mmol), (S)-1-amino-1-oxopropan-2-yl methanesulfonate (5.24 mg, 0.031 mmol) in DMSO (0.5 mL) was stirred at 80° C. for 16 h. The crude material was purified via preparative LC/MS employing the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 18% B, 18-43% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 1.5 mg (9.85% yield), and its estimated purity by LCMS analysis was 93%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 1 results: Purity: 95.1%; Observed Mass: 561.10; RT: 1.32 min. Injection 2 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 2 results: Purity: 92.6%; Observed Mass: 561.20; RT: 1.52 min.

### Example 60

### (2S)-2-(4-{2-Chloro-5-cyano-3-[(8-cyano-4-{[(1R,2S)-2-fluorocyclopropyl]amino}pyrazolo[1,5-a][1,3,5]triazin-2-yl)amino]phenyl}piperazin-1-yl)propanamide

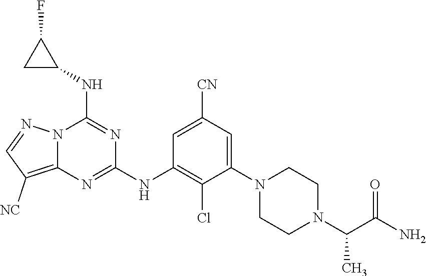 [SMILES: C[C@@H](C(N)=O)N1CCN(c2cc(C#N)cc(Nc3nc(N[C@@H]4C[C@@H]4F)n4ncc(C#N)c4n3)c2Cl)CC1]

### Step 1. 4-(((1 S,2S)-2-Fluorocyclopropyl)amino)-2-(methylthio)pyrazolo[1,5-a][1,3,5]triazine-8-carbonitrile

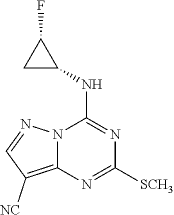 [SMILES: CSc1nc(N[C@@H]2C[C@@H]2F)n2ncc(C#N)c2n1]

A mixture of 4-chloro-2-(methylthio)pyrazolo[1,5-a][1,3,5]triazine-8-carbonitrile (0.5 g, 2.216 mmol) in THF (10 ml) was added (1R,2S)-2-fluorocyclopropan-1-amine, pTSA (0.603 g, 2.437 mmol) and Hunig's base (0.968 mL, 5.54 mmol). The mixture was stirred at rt for 2 h. It was diluted with EtOAc and washed with water. The organic layer was dried over MgSO₄, filtered, and concentrated to obtain a brown solid, which was purified by ISCO, eluting with 30% EtOAc/hexane to obtain a yellow solid. The solid was washed with MeOH and dried to obtain 4-(((1R,2S)-2-fluorocyclopropyl) amino)-2-(methylthio)pyrazolo[1,5-a][1,3,5]triazine-8-carbonitrile (450 mg, 1.703 mmol, 77% yield). [M+H]⁺=265.0.

### Step 2. 4-(((1R,2S)-2-Fluorocyclopropyl)(4-methoxybenzyl)amino)-2-(methylthio)pyrazolo[1,5-a][1,3,5]triazine-8-carbonitrile

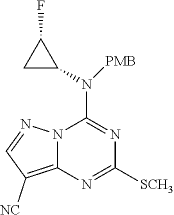

A mixture of 4-(((1R,2S)-2-fluorocyclopropyl)amino)-2-(methylthio) pyrazolo[1,5-a][1,3,5]triazine-8-carbonitrile (340 mg, 1.287 mmol) in THF (2573 μL) was added triphenylphosphine (506 mg, 1.930 mmol), (4-methoxyphenyl)methanol (231 mg, 1.672 mmol) and diisopropyl (E)-diazene-1,2-dicarboxylate (354 μL, 1.801 mmol). It was then stirred at rt for 16 h. Additional amount of (4-methoxyphenyl)methanol (231 mg, 1.672 mmol) and diisopropyl (E)-diazene-1,2-dicarboxylate (354 μL, 1.801 mmol) was added and the contents stirred at rt for another 16 h. The reaction mixture was concentrated, and the residue was purified by ISCO, eluting with 40% EtOAc/hexane to yield 4-(((1R,2S)-2-fluorocyclopropyl)(4-methoxybenzyl) amino)-2-(methylthio)pyrazolo[1,5-a][1,3,5]triazine-8-carbonitrile (310 mg, 0.806 mmol, 62.7% yield). [M+H]⁺=385.0. ¹H NMR (499 MHz, DMSO-d₆) δ 8.77-8.35 (m, 1H), 7.49-7.10 (m, 2H), 7.04-6.65 (m, 2H), 5.80-4.61 (m, 3H), 3.83-3.62 (m, 3H), 2.58-2.54 (m, 3H), 1.59-1.38 (m, 1H), 1.29-1.18 (m, 2H).

### Step 3. 4-(((1R,2S)-2-Fluorocyclopropyl)(4-methoxybenzyl)amino)-2-(methylsulfonyl)pyrazolo[1,5-a][1,3,5]triazine-8-carbonitrile

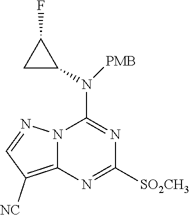

To a mixture of 4-(((1R,2S)-2-fluorocyclopropyl)(4-methoxybenzyl) amino)-2-(methylthio)pyrazolo[1,5-a][1,3,5]triazine-8-carbonitrile (310 mg, 0.806 mmol) in CH₂Cl₂ (10 mL) was added mCPBA (596 mg, 2.419 mmol) in CH₂Cl₂ (10 mL) at 0° C. It was stirred at 0° C. for 1.5 h then 1 h at rt. The solid formed was filtered and to the DCM layer was added sat. Na₂S₂O₃ solution and stirred at rt for 5 min. The organic layer was separated, washed with sat. Na₂S₂O₃ (2×), then with sat. NaHCO₃ (3×) and water. The organic layer was dried over MgSO₄, filtered, and concentrated to yield 4-(((1R,2S)-2-fluorocyclopropyl)(4-methoxybenzyl)amino)-2-(methylsulfonyl)pyrazolo[1,5-a][1,3,5]triazine-8-carbonitrile (300 mg, 0.720 mmol, 89% yield) as a white foam. [M+H]⁺=417.1.

### Step 4. tert-Butyl 4-(2-chloro-5-cyano-3-((8-cyano-4-(((1R,2S)-2-fluorocyclopropyl)(4-methoxybenzyl)amino)pyrazolo[1,5-a][1,3,5]triazin-2-yl)amino)phenyl)piperazine-1-carboxylate

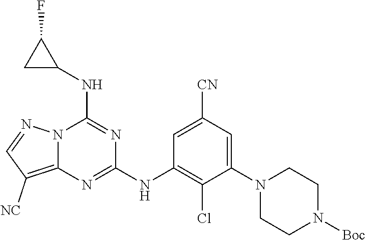 [SMILES: CC(C)(C)OC(=O)N1CCN(c2cc(C#N)cc(Nc3nc(NC4C[C@@H]4F)n4ncc(C#N)c4n3)c2Cl)CC1]

To a mixture of tert-butyl 4-(3-amino-2-chloro-5-cyanophenyl) piperazine-1-carboxylate (81 mg, 0.240 mmol) in tetrahydrofuran (5 mL) was added LiHMDS (0.360 mL, 0.360 mmol) dropwise at 0° C. It was stirred at 0° C. for 30 min. and then 4-(((1R,2S)-2-fluorocyclopropyl)(4-methoxybenzyl) amino)-2-(methylsulfonyl)pyrazolo[1,5-a][1,3,5]triazine-8-carbonitrile (100 mg, 0.240 mmol) in tetrahydrofuran (5 mL) was added and stirred at 0° C. for 15 min. then rt for 30 min. The reaction was quenched with water and extracted with EtOAc. The organic layer was washed with water, dried over MgSO₄, filtered and concentrated. Th residue was purified by ISCO, eluting with 40% EtOAc/hexane to afford tert-butyl 4-(2-chloro-5-cyano-3-((8-cyano-4-(((1R,2S)-2-fluorocyclopropyl)(4-methoxybenzyl)amino)pyrazolo[1,5-a][1,3,5]triazin-2-yl)amino)phenyl)piperazine-1-carboxylate (122 mg, 0.181 mmol, 75% yield). [M+H]⁺=673.2.

### Step 5. 2-((2-Chloro-5-cyano-3-(piperazin-1-yl)phenyl)amino)-4-(((1R,2S)-2-fluorocyclopropyl)amino)pyrazolo[1,5-a][1,3,5]triazine-8-carbonitrile

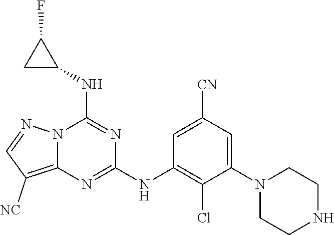 [SMILES: N#Cc1cc(Nc2nc(N[C@@H]3C[C@@H]3F)n3ncc(C#N)c3n2)c(Cl)c(N2CCNCC2)c1]

A mixture of tert-butyl 4-(2-chloro-5-cyano-3-((8-cyano-4-(((1R,2S)-2-fluorocyclopropyl)(4-methoxybenzyl)amino)pyrazolo[1,5-a][1,3,5]triazin-2-yl)amino)phenyl)piperazine-1-carboxylate (122 mg, 0.181 mmol), TFA (0.559 mL, 7.25 mmol) in CH₂Cl₂ (2 mL) was stirred at rt for 1 h. The reaction mixture was concentrated, and the residue was dissolved in ClCH₂CH₂Cl (2 mL) followed by the addition of TFA (0.559 mL, 7.25 mmol), anisole (0.079 mL, 0.725 mmol). The contents were heated at 50° C. for 16 h, and at 65° C. for 2 h and concentrated. To the residue was added 7M ammonia (0.129 mL, 0.906 mmol) in MeOH. The solid was filtered and dried to obtain 2-((2-chloro-5-cyano-3-(piperazin-1-yl)phenyl)amino)-4-(((1R,2S)-2-fluorocyclopropyl)amino) pyrazolo[1,5-a][1,3,5]triazine-8-carbonitrile (80 mg, 0.177 mmol, 97% yield). [M+H]⁺=453.3.

### Step 6. Example 60

A mixture of (R)-1-amino-1-oxopropan-2-yl methanesulfonate (7.38 mg, 0.044 mmol), 2-((2-chloro-5-cyano-3-(piperazin-1-yl)phenyl)amino)-4-(((1R,2S)-2 fluorocyclopropyl)amino)pyrazolo[1,5-a][1,3,5]triazine-8-carbonitrile (10 mg, 0.022 mmol), Hunig's base (3.86 μL, 0.022 mmol) in DMSO (0.5 mL) was heated at 80° C. for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.3 mg (28.3% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 99.3%; Observed Mass: 524.1; RT: 1.55 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 524.1; RT: 1.24 min.

### Example 61

### Methyl 4-{2-chloro-5-cyano-3-[(8-cyano-4-{[(1R,2S)-2-fluorocyclopropyl]amino}pyrazolo[1,5-a][1,3,5]triazin-2-yl)amino]phenyl}piperazine-1-carboxylate

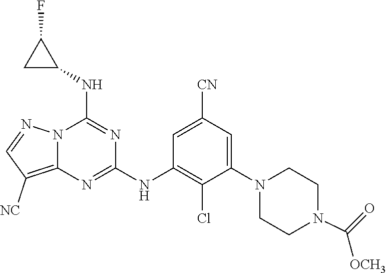 [SMILES: COC(=O)N1CCN(c2cc(C#N)cc(Nc3nc(N[C@@H]4C[C@@H]4F)n4ncc(C#N)c4n3)c2Cl)CC1]

A mixture of 2-((2-chloro-5-cyano-3-(piperazin-1-yl)phenyl)amino)-4-(((1R,2S)-2-fluorocyclopropyl)amino)pyrazolo[1,5-a][1,3,5]triazine-8-carbonitrile (10 mg, 0.022 mmol), Hunig's base (3.86 μL, 0.022 mmol), methyl carbonochloridate (4.17 mg, 0.044 mmol) in DMSO (0.5 mL) was stirred at rt for 10 min. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.7 mg (32.2% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.3%; Observed Mass: 511.1; RT: 1.82 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 99.2%; Observed Mass: 511; RT: 1.82 min.

### Example 62

### 2-{[3-(4-Acetylpiperazin-1-yl)-2-chloro-5-cyanophenyl]amino}-4-{[(1R,2S)-2-fluorocyclopropyl]amino}pyrazolo[1,5-a][1,3,5]triazine-8-carbonitrile

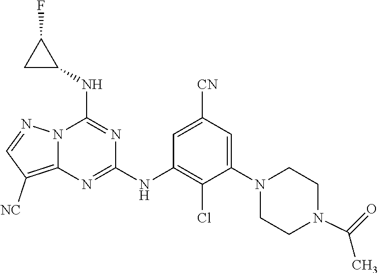 [SMILES: CC(=O)N1CCN(c2cc(C#N)cc(Nc3nc(N[C@@H]4C[C@@H]4F)n4ncc(C#N)c4n3)c2Cl)CC1]

A mixture of 2-((2-chloro-5-cyano-3-(piperazin-1-yl)phenyl)amino)-4-(((1R,2S)-2-fluorocyclopropyl)amino)pyrazolo[1,5-a][1,3,5]triazine-8-carbonitrile (10 mg, 0.022 mmol), Hunig's base (0.012 mL, 0.066 mmol), acetic anhydride (2.71 mg, 0.026 mmol) in DMSO (0.5 mL) was stirred at rt for 10 min. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.8 mg (33.8% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.1%; Observed Mass: 495; RT: 1.57 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 97.3%; Observed Mass: 495; RT: 1.57 min.

### Example 63

### 2-({2-Chloro-5-cyano-3-[4-(oxetan-3-yl)piperazin-1-yl]phenyl}amino)-4-{[(1R,2S)-2-fluorocyclopropyl]amino}pyrazolo[1,5-a][1,3,5]triazine-8-carbonitrile

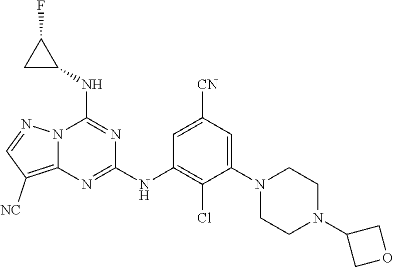 [SMILES: N#Cc1cc(Nc2nc(N[C@@H]3C[C@@H]3F)n3ncc(C#N)c3n2)c(Cl)c(N2CCN(C3COC3)CC2)c1]

A mixture of 2-((2-chloro-5-cyano-3-(piperazin-1-yl)phenyl)amino)-4-(((1R,2S)-2-fluorocyclopropyl)amino)pyrazolo[1,5-a][1,3,5]triazine-8-carbonitrile (10 mg, 0.022 mmol), oxetan-3-one (3.18 mg, 0.044 mmol), acetic acid (3.79 μL, 0.066 mmol) in DMSO (0.5 mL) was stirred at rt for 1 h. It was then added sodium triacetoxyborohydride (14.04 mg, 0.066 mmol) and stirred at rt for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.9 mg (61.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 509.1; RT: 1.62 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 509; RT: 1.25 min.

### Example 64

### 2-({3-[(8aR)-6-Oxo-octahydropyrrolo[1,2-a]piperazin-2-yl]-2-chloro-5-cyanophenyl}amino)-4-{[(1R,2S)-2-fluorocyclopropyl]amino}pyrazolo[1,5-a][1,3,5]triazine-8-carbonitrile

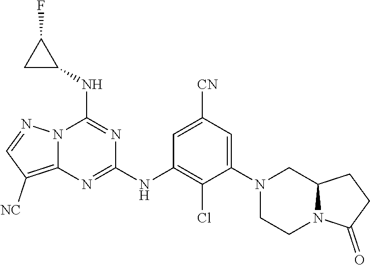 [SMILES: N#Cc1cc(Nc2nc(N[C@@H]3C[C@@H]3F)n3ncc(C#N)c3n2)c(Cl)c(N2CCN3C(=O)CC[C@@H]3C2)c1]

### Step 1. 2-((3-Bromo-2-chloro-5-cyanophenyl)amino)-4-(((1R,2S)-2-fluorocyclopropyl)(4-methoxybenzyl)amino)pyrazolo[1,5-a][1,3,5]triazine-8-carbonitrile

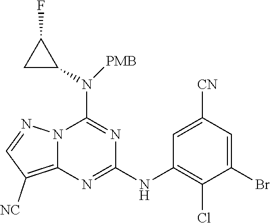

To a mixture of 3-amino-5-bromo-4-chlorobenzonitrile (55.6 mg, 0.240 mmol) in tetrahydrofuran (2 mL) was added LiHMDS (0.480 mL, 0.480 mmol) dropwise at 0° C. It was stirred at 0° C. for 30 min and then 4-(((1R,2S)-2-fluorocyclopropyl)(4-methoxybenzyl)amino)-2-(methylsulfonyl)pyrazolo[1,5-a][1,3,5]triazine-8-carbonitrile (100 mg, 0.240 mmol) in tetrahydrofuran (2 mL) was added and the contents stirred at 0° C. for 15 min. It was then warmed to rt and stirred at rt for 15 min. The reaction was quenched with water and extracted with EtOAc. The organic layer was washed with water, dried over MgSO₄, filtered and concentrated. The residue was purified by ISCO, eluting with 40% EtOAc/hexane to afford 2-((3-bromo-2-chloro-5-cyanophenyl) amino)-4-(((1R,2S)-2-fluorocyclopropyl)(4-methoxybenzyl)amino)pyrazolo[1,5-a][1,3,5]triazine-8-carbonitrile (75 mg, 0.132 mmol, 55.0% yield). LCMS (M+1)=567.0.

### Step 2. 2-((2-Chloro-5-cyano-3-((R)-6-oxohexahydropyrrolo[1,2-a]pyrazin-2(1H)-yl)phenyl)amino)-4-(((1R,2S)-2-fluorocyclopropyl)(4-methoxybenzyl)amino)pyrazolo[1,5-a][1,3,5]triazine-8-carbonitrile

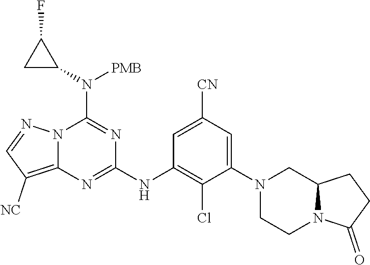

To a mixture of 2-((3-bromo-2-chloro-5-cyanophenyl)amino)-4-(((1R,2S)-2-fluorocyclopropyl)(4-methoxybenzyl)amino)pyrazolo[1,5-a][1,3,5]triazine-8-carbonitrile (25 mg, 0.044 mmol), (R)-hexahydropyrrolo[1,2-a]pyrazin-6(2H)-one, HCl (12.44 mg, 0.070 mmol), in dioxane (10 mL) was added BINAP (8.22 mg, 0.013 mmol), Cs₂CO₃ (86 mg, 0.264 mmol) and the contents purged with nitrogen for 5 min. Then Pd₂(dba)₃ (4.03 mg, 4.40 μmol) was added, and the contents purged with N₂ for another 2 min. The reaction mixture was stirred at 105° C. for 30 h. After the brown slurry was cooled to rt, celite was added. The reaction mixture was filtered through celite. The filtrate was concentrated in vacuo to give a crude mixture of 2-((2-chloro-5-cyano-3-((R)-6-oxohexahydropyrrolo[1,2-a]pyrazin-2(1H)-yl)phenyl)amino)-4-(((1R,2S)-2-fluorocyclopropyl)(4-methoxybenzyl)amino)pyrazolo[1,5-a][1,3,5]triazine-8-carbonitrile (25 mg, 0.040 mmol, 91% yield). LCMS (M+1)=627.5.

### Step 3. Example 64

A mixture of 2-((2-chloro-5-cyano-3-((R)-6-oxohexahydropyrrolo[1,2-a]pyrazin-2(1H)-yl)phenyl)amino)-4-(((1R,2S)-2-fluorocyclopropyl)(4-methoxybenzyl)amino)pyrazolo[1,5-a][1,3,5]triazine-8-carbonitrile (25 mg, 0.040 mmol), TFA (3.07 μL, 0.040 mmol), anisole (4.35 μL, 0.040 mmol) in ClCH₂CH₂Cl (1 mL) was heated at 65° C. for 2 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 7.5 mg (37.1% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 507; RT: 1.54 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 507; RT: 1.59 min.

### Example 65

### 2-({2-Chloro-5-cyano-3-[(2S)-2-methylpiperazin-1-yl]phenyl}amino)-4-(ethylamino)pyrazolo[1,5-a][1,3,5]triazine-8-carbonitrile

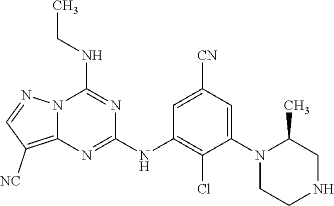 [SMILES: CCNc1nc(Nc2cc(C#N)cc(N3CCNC[C@@H]3C)c2Cl)nc2c(C#N)cnn12]

### Step 1. tert-Butyl (S)-4-(3-((tert-butoxycarbonyl)amino)-2-chloro-5-cyanophenyl)-3-methylpiperazine-1-carboxylate

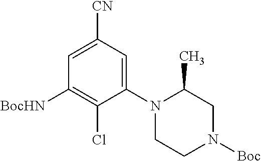 [SMILES: C[C@H]1CN(C(=O)OC(C)(C)C)CCN1c1cc(C#N)cc(NC(=O)OC(C)(C)C)c1Cl]

A mixture of tert-butyl (3-bromo-2-chloro-5-cyanophenyl)carbamate (500 mg, 1.508 mmol), tert-butyl (S)-3-methylpiperazine-1-carboxylate (453 mg, 2.262 mmol), BINAP (282 mg, 0.452 mmol), Cs₂CO₃ (1720 mg, 5.28 mmol) in toluene (15 mL) was purged with nitrogen and then Pd₂(dba)₃ (138 mg, 0.151 mmol) was added and the contents purged with nitrogen for 5 min. The mixture was stirred at 115° C. for 16 h. It was filtered through celite, the filtrate was concentrated and purified by ISCO, eluting with 20% EtOAc/hexane to afford tert-butyl (S)-4-(3-((tert-butoxycarbonyl)amino)-2-chloro-5-cyanophenyl)-3-methylpiperazine-1-carboxylate (0.25 g, 0.554 mmol, 36.8% yield). LCMS (M+1)=451.2. ¹H NMR (499 MHz, DMSO-d₆) δ 8.97-8.80 (m, 1H), 7.85-7.76 (m, 1H), 7.57-7.49 (m, 1H), 3.66-3.57 (m, 1H), 3.55-3.36 (m, 4H), 3.20-3.06 (m, 2H), 1.48-1.46 (m, 9H), 1.45-1.42 (m, 9H), 0.83-0.77 (m, 3H).

### Step 2. tert-Butyl (S)-4-(3-((tert-butoxycarbonyl)(8-cyano-4-(ethyl(4-methoxybenzyl)amino)pyrazolo[1,5-a][1,3,5]triazin-2-yl)amino)-2-chloro-5-cyanophenyl)-3-methylpiperazine-1-carboxylate

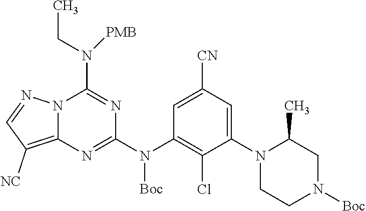

To a mixture of tert-butyl (S)-4-(3-((tert-butoxycarbonyl)amino)-2-chloro-5-cyanophenyl)-3-methylpiperazine-1-carboxylate (140 mg, 0.310 mmol) in tetrahydrofuran (5 mL) was added LiHMDS (0.621 mL, 0.621 mmol) dropwise at 0° C. It was stirred at 0° C. for 30 min and then 4-(ethyl(4-methoxybenzyl)amino)-2-(methylsulfonyl)pyrazolo[1,5-a][1,3,5]triazine-8-carbonitrile (180 mg, 0.466 mmol) in tetrahydrofuran (5 mL) was added and stirred at 0° C. for 15 min, warmed to rt and stirred at rt for 2.5 h. The reaction was quenched with water and extracted with EtOAc. The organic layer was washed with water, dried over MgSO₄, filtered and concentrated. The residue was combined with a 90 mg scale reaction and purified by ISCO, eluting with 40% EtOAc/hexane to afford tert-butyl (S)-4-(3-((tert-butoxycarbonyl)(8-cyano-4-(ethyl(4-methoxybenzyl)amino)pyrazolo[1,5-a][1,3,5]triazin-2-yl)amino)-2-chloro-5-cyanophenyl)-3-methylpiperazine-1-carboxylate (242 mg, 0.320 mmol, 63% yield). LCMS (M+1)=757.9.

### Step 3. Example 65

A mixture of tert-butyl (S)-4-(3-((tert-butoxycarbonyl)(8-cyano-4-(ethyl(4-methoxybenzyl)amino)pyrazolo[1,5-a][1,3,5]triazin-2-yl)amino)-2-chloro-5-cyanophenyl)-3-methylpiperazine-1-carboxylate (242 mg, 0.320 mmol), TFA (0.985 mL, 12.78 mmol) in CH₂Cl₂ (3 mL) was stirred at rt for 1h. The reaction mixture was concentrated, the residue was dissolved in ClCH₂CH₂Cl (3 mL), anisole (0.140 mL, 1.278 mmol) and TFA (0.985 mL, 12.78 mmol) were added, and the contents heated at 50° C. for 1 h. The reaction mixture was cooled to RT and concentrated. To the residue was added 7N NH₃. The resulting solid was filtered, washed with MeOH and dried to obtain 2-({2-chloro-5-cyano-3-[(2S)-2-methylpiperazin-1-yl]phenyl}amino)-4-(ethylamino)pyrazolo[1,5-a][1,3,5]triazine-8-carbonitrile (120 mg, 0.263 mmol, 82% yield). LCMS (M+1)=437.9.

### Example 66

### (2S)-2-[(3S)-4-(2-Chloro-5-cyano-3-{[8-cyano-4-(ethylamino)pyrazolo[1,5-a][1,3,5]triazin-2-yl]amino}phenyl)-3-methylpiperazin-1-yl]propanamide

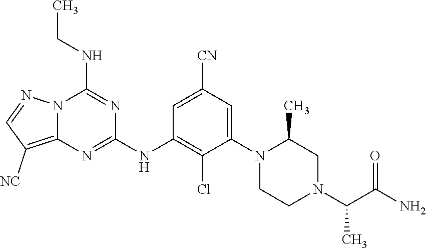 [SMILES: CCNc1nc(Nc2cc(C#N)cc(N3CCN([C@@H](C)C(N)=O)C[C@@H]3C)c2Cl)nc2c(C#N)cnn12]

A solution of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), (R)-1-amino-1-oxopropan-2-yl methanesulfonate (5.74 mg, 0.034 mmol), Hunig's base (0.012 mL, 0.069 mmol) in DMSO (0.5 mL) was stirred at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 10.6 mg (72.7% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.6%; Observed Mass: 508.1; RT: 1.3 min. Injection 4 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 4 results: Purity: 100%; Observed Mass: 508; RT: 1.88 min.

The following compounds were prepared in a similar procedure as described for Example 66 employing the R⁵ group identified in the following table:

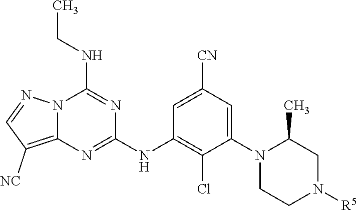

| | | LC/MS [M + H]⁺/ |
|---|---|---|
| Example # | R⁵ | RT |
| 67 | 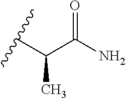 [SMILES: *[C@H](C)C(N)=O] | 508 1.47 min |
| 68 | 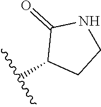 [SMILES: *[C@H]1CCNC1=O] | 520.1 1.68 min |
| 69 | 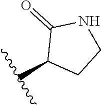 [SMILES: *[C@@H]1CCNC1=O] | 520.1 1.41 min |
| 70 | 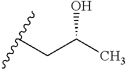 [SMILES: *C[C@@H](C)O] | 495.1 1.81 min |
| 71 | 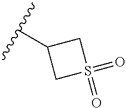 [SMILES: *C1CS(=O)(=O)C1] | 541.0 1.56 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 72

### 2-({2-Chloro-5-cyano-3-[(2S)-2-methyl-4-(oxetan-3-yl)piperazin-1-yl]phenyl}amino)-4-(ethylamino)pyrazolo[1,5-a][1,3,5]triazine-8-carbonitrile

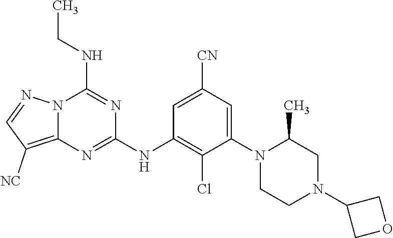 [SMILES: CCNc1nc(Nc2cc(C#N)cc(N3CCN(C4COC4)C[C@@H]3C)c2Cl)nc2c(C#N)cnn12]

A mixture of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), oxetan-3-one (1.649 mg, 0.023 mmol), acetic acid (6.55 μl, 0.114 mmol) in DMSO (0.5 mL) was stirred at rt for 1 h, then sodium triacetoxyborohydride (14.55 mg, 0.069 mmol) was added and stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 11.8 mg (84% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.8%; Observed Mass: 493.1; RT: 1.3 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 493.2; RT: 1.83 min.

### Example 73

### 2-({2-Chloro-5-cyano-3-[(2S)-4-(2-hydroxyethyl)-2-methylpiperazin-1-yl]phenyl}amino)-4-(ethylamino)pyrazolo[1,5-a][1,3,5]triazine-8-carbonitrile

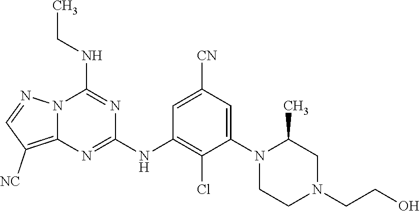 [SMILES: CCNc1nc(Nc2cc(C#N)cc(N3CCN(CCO)C[C@@H]3C)c2Cl)nc2c(C#N)cnn12]

A mixture of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), 1,4-dioxane-2,5-diol (2.75 mg, 0.023 mmol), acetic acid (6.55 μL, 0.114 mmol) in DMSO (0.5 mL) was stirred at rt for 1h. Sodium triacetoxyborohydride (14.55 mg, 0.069 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.5 mg (22.71% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 481.1; RT: 1.62 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 481.1; RT: 1.41 min.

### Example 74

### (S)-2-((2-chloro-5-cyano-3-(4-(1,1-dioxidotetrahydro-2H-thiopyran-4-yl)-2-methylpiperazin-1-yl)phenyl)amino)-4-(ethylamino)pyrazolo[1,5-a][1,3,5]triazine-8-carbonitrile

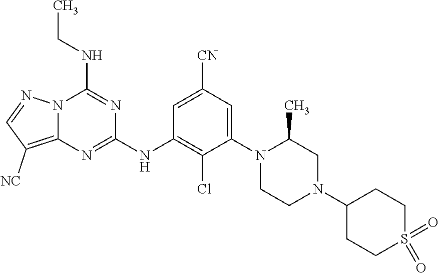 [SMILES: CCNc1nc(Nc2cc(C#N)cc(N3CCN(C4CCS(=O)(=O)CC4)C[C@@H]3C)c2Cl)nc2c(C#N)cnn12]

A mixture of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), tetrahydro-4H-thiopyran-4-one 1,1-dioxide (10.17 mg, 0.069 mmol), acetic acid (6.55 μL, 0.114 mmol) in DMSO (0.5 mL) was stirred at rt for 1h. Sodium triacetoxyborohydride (14.55 mg, 0.069 mmol) was added and the contents stirred at rt for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 7.5 mg (48.0% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 569.1; RT: 1.33 min. Injection 4 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 4 results: Purity: 100%; Observed Mass: 569; RT: 1.96 min.

### Example 75

### Methyl (3S)-4-(2-chloro-5-cyano-3-{[8-cyano-4-(ethylamino)pyrazolo[1,5-a][1,3,5]triazin-2-yl]amino}phenyl)-3-methylpiperazine-1-carboxylate

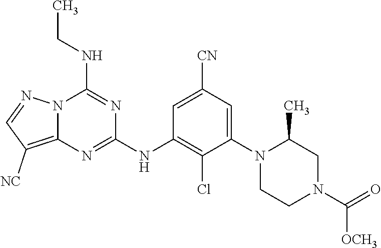 [SMILES: CCNc1nc(Nc2cc(C#N)cc(N3CCN(C(=O)OC)C[C@@H]3C)c2Cl)nc2c(C#N)cnn12]

A solution of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), methyl carbonochloridate (2.163 mg, 0.023 mmol), Hunig's base (0.012 mL, 0.069 mmol) in DMSO (0.5 mL) was stirred at rt for 15 min. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.7 mg (23.33% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.9%; Observed Mass: 494.9; RT: 2.13 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.3%; Observed Mass: 495; RT: 2.13 min.

### Example 76

### 2-({3-[(2S)-4-Acetyl-2-methylpiperazin-1-yl]-2-chloro-5-cyanophenyl}amino)-4-(ethylamino)pyrazolo[1,5-a][1,3,5]triazine-8-carbonitrile

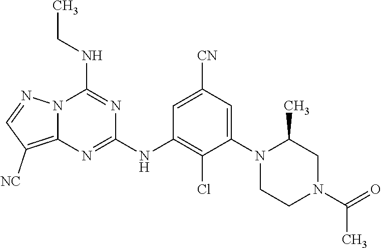 [SMILES: CCNc1nc(Nc2cc(C#N)cc(N3CCN(C(C)=O)C[C@@H]3C)c2Cl)nc2c(C#N)cnn12]

A solution of (S)-2-((2-chloro-5-cyano-3-(2-methylpiperazin-1-yl)phenyl)amino)-4-(ethylamino)pyrazolo[1,5-a][1,3,5]triazine-8-carbonitrile (10 mg, 0.023 mmol), acetic anhydride (2.337 mg, 0.023 mmol), Hunig's base (0.012 mL, 0.069 mmol) in DMSO (0.5 mL) was stirred at rt for 15 min. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.6 mg (23.72% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0-3 min), 100% B (3-3.5 min); Flow: 1 mL/min; Detection: UV (220 nm) and MS (ESI+). Injection 1 results: Purity: 100%; Observed Mass: 478.9; RT: 1.85 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0-3 min), 100% B (3-3.5 min); Flow: 1 mL/min; Detection: UV (220 nm) and MS (ESI+). Injection 2 results: Purity: 100%; Observed Mass: 479; RT: 1.85 min.

### Example 77

### 2-[(3S)-4-(2-Chloro-5-cyano-3-{[8-cyano-4-(cyclopropylamino)pyrazolo[1,5-a][1,3,5]triazin-2-yl]amino}phenyl)-3-methylpiperazin-1-yl]-2-oxoacetamide

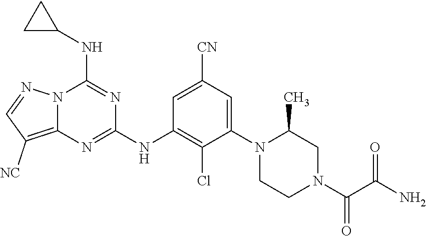 [SMILES: C[C@H]1CN(C(=O)C(N)=O)CCN1c1cc(C#N)cc(Nc2nc(NC3CC3)n3ncc(C#N)c3n2)c1Cl]

A mixture of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (10 mg, 0.022 mmol), 2-amino-2-oxoacetic acid (2.98 mg, 0.033 mmol), HATU (12.71 mg, 0.033 mmol), Hunig's base (0.012 mL, 0.067 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.5 mg (45.2% yield). Analytical Reverse Phase chromatography was used to determine the final purity. Injection 1 conditions: Colum XBridge C18, 2.1 mm×50 mm, 1.7 μm particles; Mobile Phase A: ACN/H₂O (5:95) with 0.05% TFA; Mobile Pha B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.2%; Observed Mass: 520; RT: 1.52 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 96.5%; Observed Mass: 520.1; RT: 1.61 min.

### Example 78

### 2-({3-[(2S)-4-(Azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile

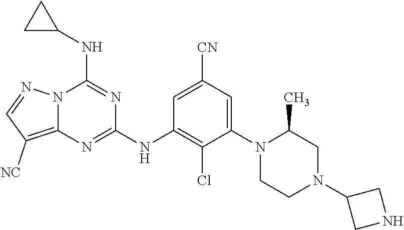 [SMILES: C[C@H]1CN(C2CNC2)CCN1c1cc(C#N)cc(Nc2nc(NC3CC3)n3ncc(C#N)c3n2)c1Cl]

### Step 1. tert-Butyl (S)-3-(4-(2-chloro-5-cyano-3-((8-cyano-4-(cyclopropylamino)pyrazolo[1,5-a][1,3,5]triazin-2-yl)amino)phenyl)-3-methylpiperazin-1-yl)azetidine-1-carboxylate

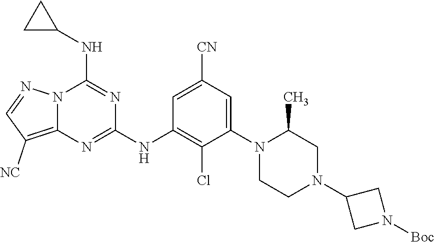 [SMILES: C[C@H]1CN(C2CN(C(=O)OC(C)(C)C)C2)CCN1c1cc(C#N)cc(Nc2nc(NC3CC3)n3ncc(C#N)c3n2)c1Cl]

A mixture of tert-butyl 3-oxoazetidine-1-carboxylate (191 mg, 1.114 mmol), (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (250 mg, 0.557 mmol), acetic acid (0.096 mL, 1.671 mmol) in DMSO (3 mL) was stirred at rt for 1 h, then sodium triacetoxyborohydride (354 mg, 1.671 mmol) was added and stirred at rt for 16 h. Water was added and the solid was filtered, washed with water and dried to obtain tert-butyl (S)-3-(4-(2-chloro-5-cyano-3-((8-cyano-4-(cyclopropylamino)pyrazolo[1,5-a][1,3,5]triazin-2-yl)amino)phenyl)-3-methylpiperazin-1-yl)azetidine-1-carboxylate (332 mg, 0.550 mmol, 99% yield). LCMS (M+1)=604.5.

### Step 2. Example 78

To tert-butyl (S)-3-(4-(2-chloro-5-cyano-3-((8-cyano-4-(cyclopropyl amino)pyrazolo[1,5-a][1,3,5]triazin-2-yl)amino)phenyl)-3-methylpiperazin-1-yl)azetidine-1-carboxylate (332 mg, 0.550 mmol), in CH₂Cl₂ (5 mL) was added TFA (1.694 mL, 21.98 mmol) and the contents stirred at rt for 1 h. The reaction mixture was concentrated and to the residue was added 7N NH₃ and the solid was filtered and dried to obtain (S)-2-((3-(4-(azetidin-3-yl)-2-methylpiperazin-1-yl)-2-chloro-5-cyanophenyl)amino)-4-(cyclopropylamino)pyrazolo[1,5-a][1,3,5]triazine-8-carbonitrile (214 mg, 0.416 mmol, 76% yield). LCMS (M+1)=504.4. The crude material (15 mg) was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 9.8 mg (79% yield), its estimated purity by Reverse Phase analysis was 98%, Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98%; Observed Mass: 504.2; RT: 1.31 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 504.1; RT: 1.38 min.

### Example 79

### (2S)-2-{3-[(3S)-4-(2-Chloro-5-cyano-3-{[8-cyano-4-(cyclopropylamino)pyrazolo[1,5-a][1,3,5]triazin-2-yl]amino}phenyl)-3-methylpiperazin-1-yl]azetidin-1-yl}propanamide

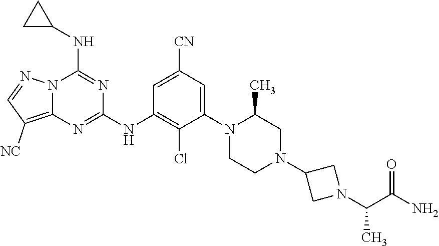 [SMILES: C[C@@H](C(N)=O)N1CC(N2CCN(c3cc(C#N)cc(Nc4nc(NC5CC5)n5ncc(C#N)c5n4)c3Cl)[C@@H](C)C2)C1]

To a solution of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), in DMSO (0.5 mL) was added (R)-1-amino-1-oxopropan-2-yl methanesulfonate (4.98 mg, 0.030 mmol) and Hunig's base (10.40 μL, 0.060 mmol) and the contents stirred at 80° C. for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.5 mg (47.8% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 99.1%; Observed Mass: 575.2; RT: 1.58 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 99.1%; Observed Mass: 575.2; RT: 1.33 min.

The following compounds were prepared in a similar procedure as described for Example 79 employing the R^{b} group identified in the following table:

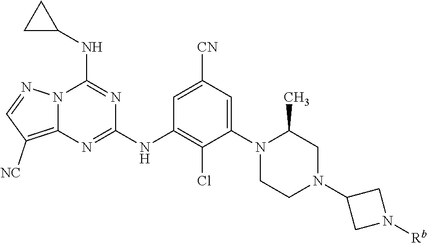

| | | LC/MS [M + H]⁺/ |
|---|---|---|
| Example # | R^{b} | RT |
| 80 | 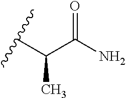 [SMILES: *[C@H](C)C(N)=O] | 575.2 1.43 min |
| 81 | 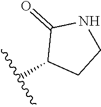 [SMILES: *[C@H]1CCNC1=O] | 587.2 1.44 min |
| 82 | 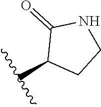 [SMILES: *[C@@H]1CCNC1=O] | 587.2 1.32 min |
| 83 | 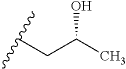 [SMILES: *C[C@@H](C)O] | 562.3 1.37 min |
| 84 | 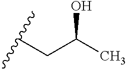 [SMILES: *C[C@H](C)O] | 562.1 1.37 min |
| 85 | 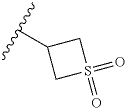 [SMILES: *C1CS(=O)(=O)C1] | 608.1 1.77 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 86

### Methyl 3-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]azetidine-1-carboxylate

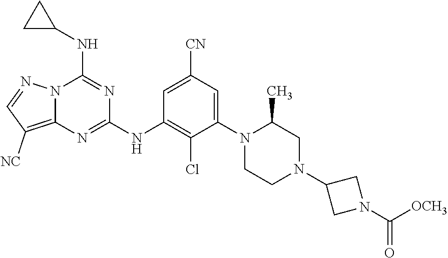 [SMILES: COC(=O)N1CC(N2CCN(c3cc(C#N)cc(Nc4nc(NC5CC5)n5ncc(C#N)c5n4)c3Cl)[C@@H](C)C2)C1]

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (15 mg, 0.030 mmol), methyl carbonochloridate (2.81 mg, 0.030 mmol), Hunig's base (5.20 μL, 0.030 mmol) in DMSO (0.5 mL) was stirred at rt for 10 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 4.7 mg (27.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.6%; Observed Mass: 562.2; RT: 1.93 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.8%; Observed Mass: 562.1; RT: 1.4 min.

### Example 87

### 2-({2-Chloro-5-cyano-3-[(2S)-4-(1-methanesulfonylazetidin-3-yl)-2-methylpiperazin-1-yl]phenyl}amino)-4-(cyclopropylamino)pyrazolo[1,5-a][1,3,5]triazine-8-carbonitrile

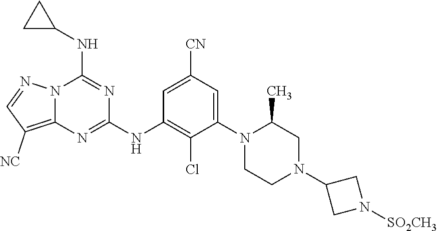 [SMILES: C[C@H]1CN(C2CN(S(C)(=O)=O)C2)CCN1c1cc(C#N)cc(Nc2nc(NC3CC3)n3ncc(C#N)c3n2)c1Cl]

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), methanesulfonyl chloride (2.273 mg, 0.020 mmol), Hunig's base (3.47 μL, 0.020 mmol) in DMSO (0.5 mL) was stirred at rt for 10 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 4.1 mg (32.3% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 91.1%; Observed Mass: 582.2; RT: 1.86 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 91.8%; Observed Mass: 582.1; RT: 1.44 min.

### Example 88

### 2-({2-Chloro-5-cyano-3-[(2S)-4-[1-(2-methoxyethyl)azetidin-3-yl]-2-methylpiperazin-1-yl]phenyl}amino)-4-(cyclopropylamino)pyrazolo[1,5-a][1,3,5]triazine-8-carbonitrile

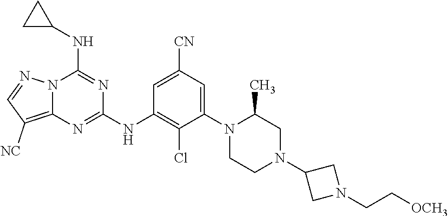 [SMILES: COCCN1CC(N2CCN(c3cc(C#N)cc(Nc4nc(NC5CC5)n5ncc(C#N)c5n4)c3Cl)[C@@H](C)C2)C1]

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), 1-bromo-2-methoxyethane (3.31 mg, 0.024 mmol), Hunig's base (10.40 μL, 0.060 mmol) in DMSO (0.5 mL) was stirred at 50° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 8.6 mg (61.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.8%; Observed Mass: 562.2; RT: 1.38 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.3%; Observed Mass: 562.2; RT: 1.6 min.

### Example 89

### 2-({2-Chloro-5-cyano-3-[(2S)-4-[1-(2-hydroxyethyl)azetidin-3-yl]-2-methylpiperazin-1-yl]phenyl}amino)-4-(cyclopropylamino)pyrazolo[1,5-a][1,3,5]triazine-8-carbonitrile

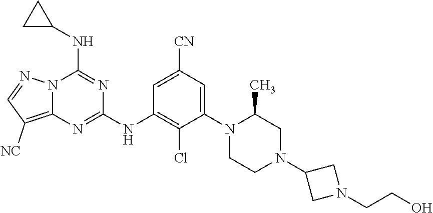 [SMILES: C[C@H]1CN(C2CN(CCO)C2)CCN1c1cc(C#N)cc(Nc2nc(NC3CC3)n3ncc(C#N)c3n2)c1Cl]

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), 1,4-dioxane-2,5-diol (4.77 mg, 0.040 mmol), acetic acid (3.41 μL, 0.060 mmol) in DMSO (0.5 mL) was stirred at rt for 1 h, then sodium triacetoxyborohydride (12.62 mg, 0.060 mmol) was added and the contents stirred at rt for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.9 mg (35.6% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 99.4%; Observed Mass: 548.1; RT: 1.33 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 548.1; RT: 1.5 min.

### Example 90

### 2-({2-Chloro-5-cyano-3-[(2S)-2-methyl-4-[1-(oxetan-3-yl)azetidin-3-yl]piperazin-1-yl]phenyl}amino)-4-(cyclopropylamino)pyrazolo[1,5-a][1,3,5]triazine-8-carbonitrile

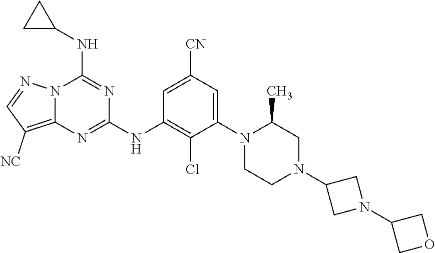 [SMILES: C[C@H]1CN(C2CN(C3COC3)C2)CCN1c1cc(C#N)cc(Nc2nc(NC3CC3)n3ncc(C#N)c3n2)c1Cl]

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), oxetan-3-one (2.86 mg, 0.040 mmol), acetic acid (3.41 μL, 0.060 mmol) in DMSO (0.5 mL) was stirred at rt for 1 h, then sodium triacetoxyborohydride (12.62 mg, 0.060 mmol) was added and the contents stirred at rt for 16 h. The crude material was purified via preparative reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative Reverse Phase employing the following conditions: Column: Xbridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.5 mg (25.3% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.5%; Observed Mass: 560.2; RT: 1.37 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.5%; Observed Mass: 560.2; RT: 1.76 min.

### Example 91

### 2-({2-Chloro-5-cyano-3-[(2S)-2-methyl-4-{1-[(2S)-3,3,3-trifluoro-2-hydroxy-2-methylpropanoyl]azetidin-3-yl}piperazin-1-yl]phenyl}amino)-4-(cyclopropylamino)pyrazolo[1,5-a][1,3,5]triazine-8-carbonitrile

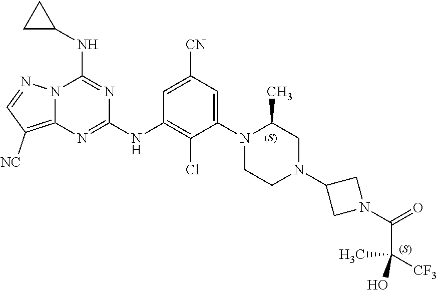

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), (S)-3,3,3-trifluoro-2-hydroxy-2-methylpropanoic acid (4.70 mg, 0.030 mmol), HATU (11.32 mg, 0.030 mmol), Hunig's base (10.40 μL, 0.060 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 8.2 mg (61.6% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96%; Observed Mass: 644.2; RT: 1.86 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 644.1; RT: 1.49 min.

### Example 92

### 2-({2-Chloro-5-cyano-3-[(2S)-2-methyl-4-{1-[(2R)-3,3,3-trifluoro-2-hydroxy-2-methylpropanoyl]azetidin-3-yl}piperazin-1-yl]phenyl}amino)-4-(cyclopropylamino)pyrazolo[1,5-a][1,3,5]triazine-8-carbonitrile

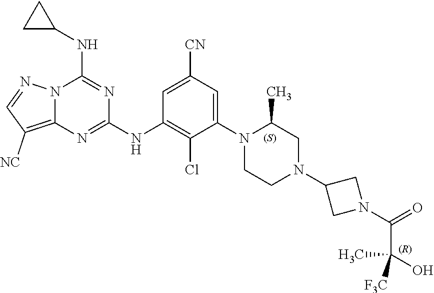

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), (R)-3,3,3-trifluoro-2-hydroxy-2-methylpropanoic acid (4.70 mg, 0.030 mmol), Hunig's base (10.40 μL, 0.060 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 8 mg (60.9% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.3%; Observed Mass: 644.2; RT: 1.86 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 644.1; RT: 1.49 min.

### Example 93

### 2-{3-[(3S)-4-(2Chloro-5-cyano-3-{[8-cyano-4-(cyclopropylamino)pyrazolo[1,5-a][1,3,5]triazin-2-yl]amino}phenyl)-3-methylpiperazin-1-yl]azetidin-1-yl}-2-oxoacetamide

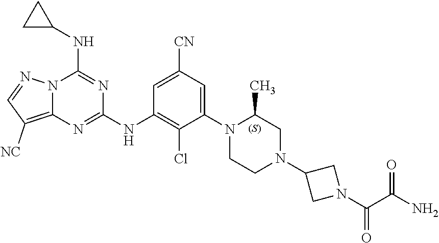

A mixture of 2-({3-[(2S)-4-(azetidin-3-yl)-2-methylpiperazin-1-yl]-2-chloro-5-cyanophenyl}amino)-4-(cyclopropylamino)pyrazolo[1,5-a][1,3,5]triazine-8-carbonitrile (10 mg, 0.020 mmol), 2-amino-2-oxoacetic acid (2.65 mg, 0.030 mmol), Hunig's base (10.40 μL, 0.060 mmol), HATU (11.32 mg, 0.030 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 8.1 mg (69.9% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.4%; Observed Mass: 575.2; RT: 1.58 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 99%; Observed Mass: 575.2; RT: 1.31 min.

### Example 94

### 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile

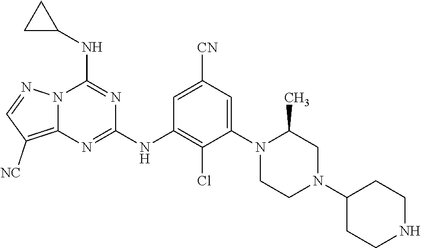 [SMILES: C[C@H]1CN(C2CCNCC2)CCN1c1cc(C#N)cc(Nc2nc(NC3CC3)n3ncc(C#N)c3n2)c1Cl]

### Step 1. tert-Butyl (S)-4-(4-(2-chloro-5-cyano-3-((8-cyano-4-(cyclopropylamino) pyrazolo[1,5-a][1,3,5]triazin-2-yl)amino)phenyl)-3-methylpiperazin-1-yl)piperidine-1-carboxylate

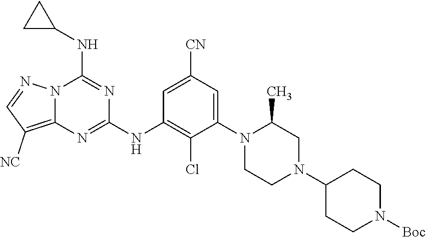 [SMILES: C[C@H]1CN(C2CCN(C(=O)OC(C)(C)C)CC2)CCN1c1cc(C#N)cc(Nc2nc(NC3CC3)n3ncc(C#N)c3n2)c1Cl]

A mixture of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (250 mg, 0.557 mmol), tert-butyl 4-oxopiperidine-1-carboxylate (222 mg, 1.114 mmol), acetic acid (0.096 mL, 1.671 mmol) in DMSO (3 mL) was stirred at rt for 1 h, then sodium triacetoxyborohydride (354 mg, 1.671 mmol) was added and the contents stirred at rt for 16 h. The reaction mixture was diluted with water and the solid was filtered, washed with water and dried to obtain tert-butyl (S)-4-(4-(2-chloro-5-cyano-3-((8-cyano-4-(cyclopropylamino)pyrazolo[1,5-a][1,3,5]triazin-2-yl)amino)phenyl)-3-methylpiperazin-1-yl)piperidine-1-carboxylate (320 mg, 0.506 mmol, 91% yield). LCMS (M+1)=632.6.

### Step 2. Example 94

A mixture of tert-butyl (S)-4-(4-(2-chloro-5-cyano-3-((8-cyano-4-(cyclopropylamino)pyrazolo[1,5-a][1,3,5]triazin-2-yl)amino)phenyl)-3-methylpiperazin-1-yl)piperidine-1-carboxylate (320 mg, 0.506 mmol), TFA (1.560 mL, 20.25 mmol) in CH₂Cl₂ (3 mL) was stirred at rt for 1 h. The reaction mixture was concentrated and to the residue was added 7N NH₃. The resulting solid was filtered to obtain 2-({2-chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (200 mg, 0.376 mmol, 74.3% yield). LCMS (M+1)=532.3. The crude material (10 mg) was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.2 mg (38.1% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 532.3; RT: 1.2 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 532.3; RT: 1.44 min.

### Example 95

### (2S)-2-{4-[(3S)-4-(2-Chloro-5-cyano-3-{[8-cyano-4-(cyclopropylamino)pyrazolo[1,5-a][1,3,5]triazin-2-yl]amino}phenyl)-3-methylpiperazin-1-yl]piperidin-1-yl}propanamide

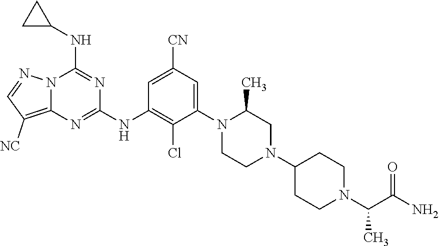 [SMILES: C[C@@H](C(N)=O)N1CCC(N2CCN(c3cc(C#N)cc(Nc4nc(NC5CC5)n5ncc(C#N)c5n4)c3Cl)[C@@H](C)C2)CC1]

A solution of 2-({2-chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), (R)-1-amino-1-oxopropan-2-yl methanesulfonate (4.71 mg, 0.028 mmol), Hunig's base (9.85 μL, 0.056 mmol) in DMSO (0.5 mL) was stirred at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 7.9 mg (66.3% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.1%; Observed Mass: 603.2; RT: 1.71 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 95.6%; Observed Mass: 603.2; RT: 1.28 min.

The following compounds were prepared in a similar procedure as described for Example 95 employing the R^{b} group identified in the following table:

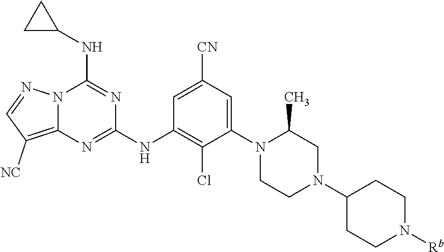

| | | LC/MS [M + H]⁺/ |
|---|---|---|
| Example # | R^{b} | RT |
| 96 | 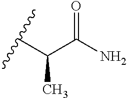 [SMILES: *[C@H](C)C(N)=O] | 603.2 1.27 min |
| 97 | 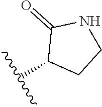 [SMILES: *[C@H]1CCNC1=O] | 615.2 1.61 min |
| 98 | 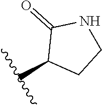 [SMILES: *[C@@H]1CCNC1=O] | 615.2 1.61 min |
| 99 | 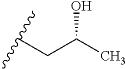 [SMILES: *C[C@@H](C)O] | 590.2 1.53 min |
| 100 | 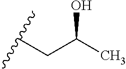 [SMILES: *C[C@H](C)O] | 590.2 1.61 min |
| 101 | 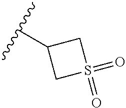 [SMILES: *C1CS(=O)(=O)C1] | 636.1 1.79 min |

### LC/MS Conditions:

Column: Waters XBridge 018, 2.1 mm×50 mm, 1.7 μm particles; Mobile Phase A: 5:95 acetonitrile-water with 10 mM ammonium acetate; Mobile Phase B: 95:5 acetonitrile-water with 10 mM ammonium acetate; Temperature: 50 OC; Gradient: 0% B to 100% B over 3 min, then a 0.50 min hold at 100% B; Flow: 1 mL/min; Detection: MS and UV (220 nm).

### Example 102

### 2-({2-Chloro-5-cyano-3-[(2S)-4-[1-(2-methoxyethyl)piperidin-4-yl]-2-methylpiperazin-1-yl]phenyl}amino)-4-(cyclopropylamino)pyrazolo[1,5-a][1,3,5]triazine-8-carbonitrile

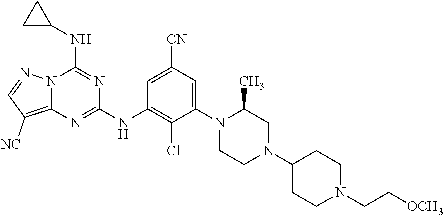 [SMILES: COCCN1CCC(N2CCN(c3cc(C#N)cc(Nc4nc(NC5CC5)n5ncc(C#N)c5n4)c3Cl)[C@@H](C)C2)CC1]

A solution of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), 1-bromo-2-methoxyethane (3.13 mg, 0.023 mmol), Hunig's base (9.85 μL, 0.056 mmol) in DMSO (0.5 mL) was stirred at 50° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.7 mg (58.5% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.8%; Observed Mass: 590.2; RT: 1.6 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.1%; Observed Mass: 590.2; RT: 1.19 min.

### Example 103

### Methyl 4-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]piperidine-1-carboxylate

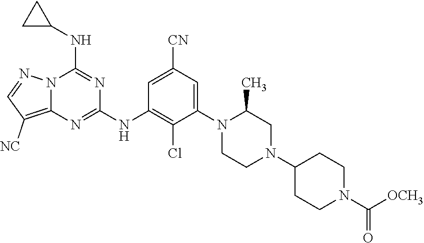 [SMILES: COC(=O)N1CCC(N2CCN(c3cc(C#N)cc(Nc4nc(NC5CC5)n5ncc(C#N)c5n4)c3Cl)[C@@H](C)C2)CC1]

A mixture of (2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (15 mg, 0.028 mmol), methyl carbonochloridate (4.00 mg, 0.042 mmol), Hunig's base (0.015 mL, 0.085 mmol) in DMSO (0.5 mL) was stirred at rt for 10 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 4.3 mg (24.58% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.1%; Observed Mass: 590.1; RT: 1.63 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 97.5%; Observed Mass: 590.1; RT: 2.02 min.

### Example 104

### 2-({3-[(2S)-4-(1-Acetylpiperidin-4-yl)-2-methylpiperazin-1-yl]-2-chloro-5-cyanophenyl}amino)-4-(cyclopropylamino)pyrazolo[1,5-a][1,3,5]triazine-8-carbonitrile

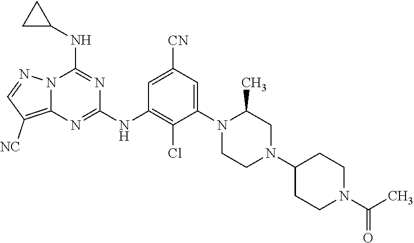 [SMILES: CC(=O)N1CCC(N2CCN(c3cc(C#N)cc(Nc4nc(NC5CC5)n5ncc(C#N)c5n4)c3Cl)[C@@H](C)C2)CC1]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), acetic anhydride (2.88 mg, 0.028 mmol), Hunig's base (9.85 μL, 0.056 mmol) in DMSO (0.5 mL) was stirred at rt for 10 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.4 mg (46.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 92.8%; Observed Mass: 574.2; RT: 1.68 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 97%; Observed Mass: 574.2; RT: 1.34 min.

### Example 105

### 2-({2-Chloro-5-cyano-3-[(2S)-2-methyl-4-{1-[(2S)-3,3,3-trifluoro-2-hydroxy-2-methylpropanoyl]piperidin-4-yl}piperazin-1-yl]phenyl}amino)-4-(cyclopropylamino)pyrazolo[1,5-a][1,3,5]triazine-8-carbonitrile

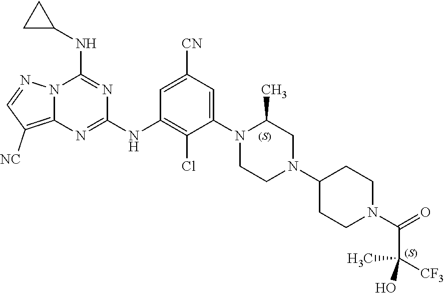

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), (S)-3,3,3-trifluoro-2-hydroxy-2-methylpropanoic acid (4.46 mg, 0.028 mmol), Hunig's base (9.85 μL, 0.056 mmol), HATU (10.72 mg, 0.028 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 9.8 mg (76% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.6%; Observed Mass: 672.2; RT: 1.54 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.5%; Observed Mass: 672.2; RT: 2.03 min.

### Example 106

### 2-({2-Chloro-5-cyano-3-[(2S)-2-methyl-4-{1-[(2S)-3,3,3-trifluoro-2-hydroxy-2-methylpropanoyl]piperidin-4-yl}piperazin-1-yl]phenyl}amino)-4-(cyclopropylamino)pyrazolo[1,5-a][1,3,5]triazine-8-carbonitrile

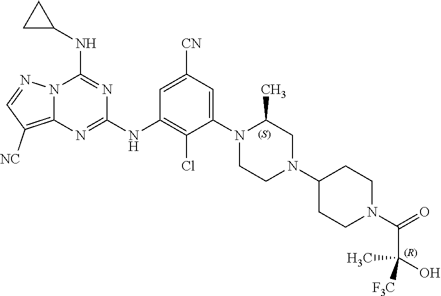

A mixture 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), (R)-3,3,3-trifluoro-2-hydroxy-2-methylpropanoic acid (4.46 mg, 0.028 mmol), Hunig's base (9.85 μL, 0.056 mmol), HATU (10.72 mg, 0.028 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 7 mg (54.7% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.8%; Observed Mass: 672.2; RT: 1.56 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 672.2; RT: 2.02 min.

### Example 107

### 2-{4-[(3S)-4-(2-Chloro-5-cyano-3-{[8-cyano-4-(cyclopropylamino)pyrazolo[1,5-a][1,3,5]triazin-2-yl]amino}phenyl)-3-methylpiperazin-1-yl]piperidin-1-yl}-2-oxoacetamide

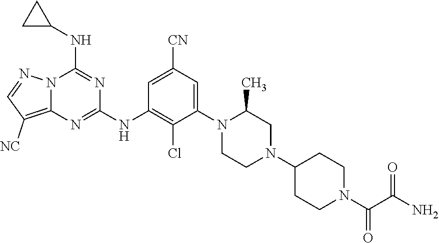 [SMILES: C[C@H]1CN(C2CCN(C(=O)C(N)=O)CC2)CCN1c1cc(C#N)cc(Nc2nc(NC3CC3)n3ncc(C#N)c3n2)c1Cl]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), HATU (10.72 mg, 0.028 mmol), Hunig's base (9.85 μL, 0.056 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.7 mg (58.6% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 99.2%; Observed Mass: 603.1; RT: 1.6 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 603.1; RT: 1.24 min.

### Example 108

### 2-({2-Chloro-5-cyano-3-[(2S)-4-[1-(2-hydroxyethyl)piperidin-4-yl]-2-methylpiperazin-1-yl]phenyl}amino)-4-(cyclopropylamino)pyrazolo[1,5-a][1,3,5]triazine-8-carbonitrile

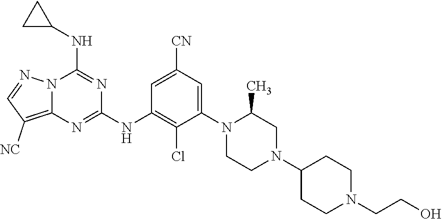 [SMILES: C[C@H]1CN(C2CCN(CCO)CC2)CCN1c1cc(C#N)cc(Nc2nc(NC3CC3)n3ncc(C#N)c3n2)c1Cl]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), 1,4-dioxane-2,5-diol (2.257 mg, 0.019 mmol), acetic acid (3.23 μL, 0.056 mmol) in DMSO (0.5 mL) was stirred at rt for 1 h, then sodium triacetoxyborohydride (11.95 mg, 0.056 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.6 mg (21.85% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 91%; Observed Mass: 576.2; RT: 1.56 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 97%; Observed Mass: 576.2; RT: 1.28 min. ¹H NMR (500 MHz, DMSO-d₆) δ 8.50-8.24 (m, 1H), 8.24-8.01 (m, 1H), 7.62-7.21 (m, 1H), 3.27-3.01 (m, 1H), 2.98-2.70 (m, 4H), 2.70-2.58 (m, 2H), 2.39-2.29 (m, 2H), 2.29-2.05 (m, 2H), 2.02-1.87 (m, 2H), 1.86-1.81 (m, 2H), 1.75-1.62 (m, 2H), 1.47-1.30 (m, 2H), 0.88-0.79 (m, 3H), 0.79-0.66 (m, 4H), two protons missing due to water suppression. LCMS (M+1)=576.3.

### Example 109

### 2-({2-Chloro-5-cyano-3-[(2S)-2-methyl-4-[1-(oxetan-3-yl)piperidin-4-yl]piperazin-1-yl]phenyl}amino)-4-(cyclopropylamino)pyrazolo[1,5-a][1,3,5]triazine-8-carbonitrile

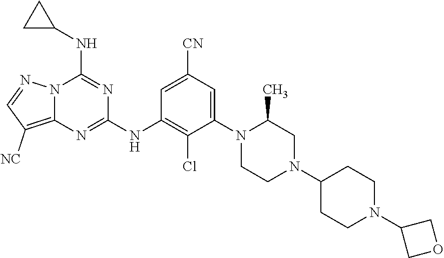 [SMILES: C[C@H]1CN(C2CCN(C3COC3)CC2)CCN1c1cc(C#N)cc(Nc2nc(NC3CC3)n3ncc(C#N)c3n2)c1Cl]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), oxetan-3-one (2.71 mg, 0.038 mmol), acetic acid (3.23 μL, 0.056 mmol) in DMSO (0.5 mL) was stirred at rt for 1 h, then sodium triacetoxyborohydride (11.95 mg, 0.056 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.8 mg (24.47% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.6%; Observed Mass: 588.2; RT: 1.23 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 588.2; RT: 1.77 min.

### Example 110

### (S)-2-((2-chloro-5-cyano-3-(4-(1-(1,1-dioxidotetrahydro-2H-thiopyran-4-yl)piperidin-4-yl)-2-methylpiperazin-1-yl)phenyl)amino)-4-(cyclopropylamino)pyrazolo[1,5-a][1,3,5]triazine-8-carbonitrile

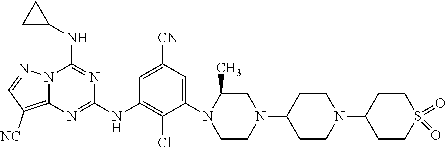 [SMILES: C[C@H]1CN(C2CCN(C3CCS(=O)(=O)CC3)CC2)CCN1c1cc(C#N)cc(Nc2nc(NC3CC3)n3ncc(C#N)c3n2)c1Cl]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.019 mmol), tetrahydro-4H-thiopyran-4-one 1,1-dioxide (5.57 mg, 0.038 mmol), acetic acid (3.23 μL, 0.056 mmol) in DMSO (0.5 mL) was stirred at rt for 1 h, then sodium cyanoborohydride (3.54 mg, 0.056 mmol) was added and stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 9.2 mg (73.7% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 664.2; RT: 1.73 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 664.1; RT: 1.2 min.

### Example 111

### 2-((2-chloro-5-cyano-3-((2S)-4-(2,6-dimethyl-1,1-dioxidotetrahydro-2H-thiopyran-4-yl)-2-methylpiperazin-1-yl)phenyl)amino)-4-(cyclopropylamino)pyrazolo[1,5-a][1,3,5]triazine-8-carbonitrile

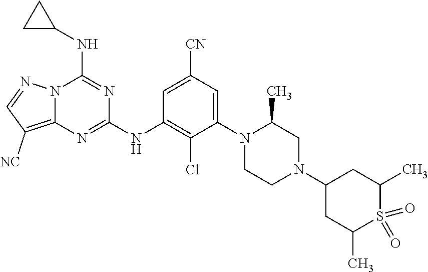 [SMILES: CC1CC(N2CCN(c3cc(C#N)cc(Nc4nc(NC5CC5)n5ncc(C#N)c5n4)c3Cl)[C@@H](C)C2)CC(C)S1(=O)=O]

A mixture of 2-({2-Chloro-5-cyano-3-[(2S)-2-methyl-4-(piperidin-4-yl)piperazin-1-yl]phenyl}amino)-4-(cyclopropylamino)pyrazolo[1,5-a][1,3,5]triazine-8-carbonitrile (10 mg, 0.022 mmol), acetic acid (6.38 μL, 0.111 mmol), 2,6-dimethyltetrahydro-4H-thiopyran-4-one 1,1-dioxide (5.89 mg, 0.033 mmol) in DMSO (0.5 mL) was stirred at rt for 1 h. Sodium triacetoxyborohydride (14.16 mg, 0.067 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product (as a mixture of diastereomers) were combined and dried via centrifugal evaporation. The recovery of the product was 7.2 mg (51.5% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.1%; Observed Mass: 609.2; RT: 2.14 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 97.1%; Observed Mass: 609.1; RT: 1.49 min.

### Example 112

### 2-({2-Chloro-5-cyano-3-[(2S)-2-methyl-4-[(2S)-3,3,3-trifluoro-2-hydroxy-2-methylpropanoyl]piperazin-1-yl]phenyl}amino)-4-(cyclopropylamino)pyrazolo[1,5-a][1,3,5]triazine-8-carbonitrile

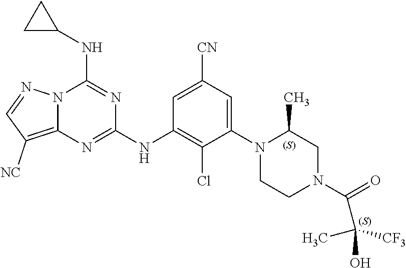

A mixture of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (10 mg, 0.022 mmol), (S)-3,3,3-trifluoro-2-hydroxy-2-methylpropanoic acid (5.28 mg, 0.033 mmol) HATU (12.71 mg, 0.033 mmol), Hunig's base (0.012 mL, 0.067 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 9.4 mg (69.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.9%; Observed Mass: 588.9; RT: 2.01 min. Injection 4 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 4 results: Purity: 100%; Observed Mass: 589.1; RT: 1.99 min.

### Example 113

### 2-({2-Chloro-5-cyano-3-[(2S)-2-methyl-4-[(2R)-3,3,3-trifluoro-2-hydroxy-2-methylpropanoyl]piperazin-1-yl]phenyl}amino)-4-(cyclopropylamino)pyrazolo[1,5-a][1,3,5]triazine-8-carbonitrile

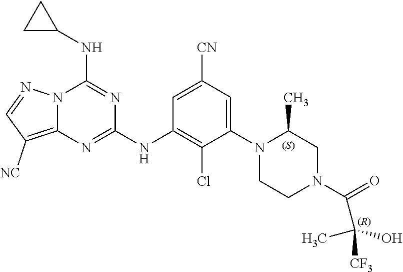

A mixture of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (10 mg, 0.022 mmol), (R)-3,3,3-trifluoro-2-hydroxy-2-methylpropanoic acid (5.28 mg, 0.033 mmol), HATU (12.71 mg, 0.033 mmol), Hunig's base (0.012 mL, 0.067 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.7 mg, (48.5% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95%; Observed Mass: 589.1; RT: 2.01 min. Injection 3 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 3 results: Purity: 96.2%; Observed Mass: 589.2; RT: 1.92 min.

### Example 114

### (2S)-2-[(3S)-4-(2-Chloro-5-cyano-3-{[8-cyano-4-(cyclopropylamino)pyrazolo [1,5-a][1,3,5]triazin-2-yl]amino}phenyl)-3-ethylpiperazin-1-yl]propanamide

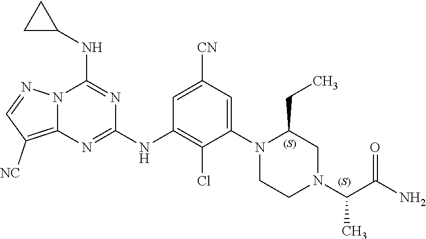

### Step 1. tert-butyl (S)-4-(3-bromo-2-chloro-5-cyanophenyl)-3-ethylpiperazine-1-carboxylate

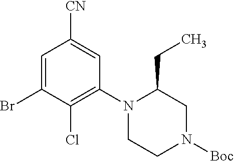 [SMILES: CC[C@H]1CN(C(=O)OC(C)(C)C)CCN1c1cc(C#N)cc(Br)c1Cl]

To a stirred solution of 3,5-dibromo-4-chlorobenzonitrile (2 g, 6.77 mmol) in toluene (20 mL). tert-butyl (S)-3-ethylpiperazine-1-carboxylate (1.596 g, 7.45 mmol), (oxybis(2,1-phenylene))bis(diphenylphosphane) (0.438 g, 0.813 mmol) and cesium carbonate (6.62 g, 20.31 mmol) were added at room temperature under N₂ atm. After the contents are degassed for 10 min., Pd₂(dba)₃ (0.248 g, 0.271 mmol) was added followed by degassing for another 10 min. The reaction mixture was heated to 95° C. for 3d. After cooling to rt, the reaction mixture was filtered through a pad of celite and washed with ethyl acetate, The combined organic layer was washed with water and brine, dried over anhydrous sodium sulfate, filtered, and concentrated, purified by ISCO using silica (60-120) mesh, column size 120 g, ethyl acetate in hexane as eluant. The product was eluted at 20% ethyl acetate in hexane to get tert-butyl (S)-4-(3-bromo-2-chloro-5-cyanophenyl)-3-ethylpiperazine-1-carboxylate (1.7 g, 3.96 mmol, 58.6% yield) as a white solid. LCMS (M+1)=428.0.

### Step 2. 2-Amino-4-(cyclopropyl(4-methoxybenzyl)amino)pyrazolo[1,5-a][1,3,5]triazine-8-carbonitrile

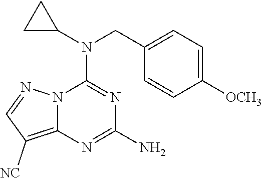 [SMILES: COc1ccc(CN(c2nc(N)nc3c(C#N)cnn23)C2CC2)cc1]

A mixture of 4-(cyclopropyl(4-methoxybenzyl)amino)-2-(methylsulfonyl) pyrazolo[1,5-a][1,3,5]triazine-8-carbonitrile (2 g, 5.02 mmol) in 0.4 M ammonia (37.6 mL, 15.06 mmol)/dioxane was heated at 80° C. for 2 h. It was then cooled to rt and concentrated. To the residue was added MeOH and the solid was filtered and dried to obtain 2-amino-4-(cyclopropyl(4-methoxybenzyl) amino)pyrazolo[1,5-a][1,3,5]triazine-8-carbonitrile (1.5 g, 4.47 mmol, 89% yield). LCMS (M+1)=336.0. Step 3. tert-Butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-ethylpiperazine-1-carboxylate

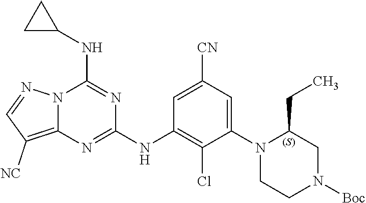

A mixture of 2-amino-4-(cyclopropyl(4-methoxybenzyl)amino) pyrazolo[1,5-a][1,3,5]triazine-8-carbonitrile (200 mg, 0.596 mmol), tert-butyl (S)-4-(3-bromo-2-chloro-5-cyanophenyl)-3-ethylpiperazine-1-carboxylate (426 mg, 0.686 mmol), Cs₂CO₃ (583 mg, 1.789 mmol), (9,9-dimethyl-9H-xanthene-4,5-diyl)bis(diphenylphosphane) (34.5 mg, 0.060 mmol), Pd₂(dba)₃ (27.3 mg, 0.030 mmol) in dioxane (5 mL) was purged with N₂ and then heated at 90° C. for 16 h. The reaction mixture was cooled to RT and filtered through celite. The filtrate was concentrated and purified by ISCO, eluting with 40% EtOAc/hexane to afford tert-butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-ethylpiperazine-1-carboxylate (240 mg, 0.351 mmol, 58.9% yield). LCMS (M+1)=683.6.

### Step 4. Example 114

A solution of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), (R)-1-amino-1-oxopropan-2-yl methanesulfonate (5.42 mg, 0.032 mmol), Hunig's base (0.011 mL, 0.065 mmol) in DMSO (0.5 mL) was stirred at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 7 mg (57.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 94.6%; Observed Mass: 534.1; RT: 1.4 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 534.1; RT: 1.85 min.

### Example 115

### (2R)-2-[(3S)-4-(2-Chloro-5-cyano-3-{[8-cyano-4-(cyclopropylamino)pyrazolo[1,5-a][1,3,5]triazin-2-yl]amino}phenyl)-3-ethylpiperazin-1-yl]propanamide

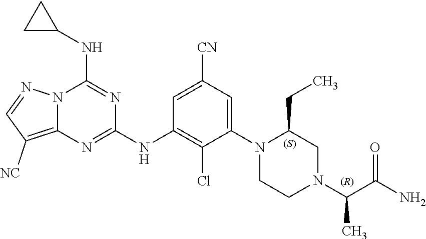

A solution of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), (S)-1-amino-1-oxopropan-2-yl methanesulfonate (5.42 mg, 0.032 mmol), Hunig's base (0.011 mL, 0.065 mmol) in DMSO (0.5 mL) was stirred at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative Reverse Phase employing the following conditions: Column: Xbridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.9 mg (46.8% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 91.5%; Observed Mass: 534.2; RT: 1.82 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 534.1; RT: 1.39 min.

### Example 116

### (S)-2-((2-chloro-5-cyano-3-(4-(1,1-dioxidothietan-3-yl)-2-ethylpiperazin-1-yl)phenyl)amino)-4-(cyclopropylamino)pyrazolo[1,5-a][1,3,5]triazine-8-carbonitrile

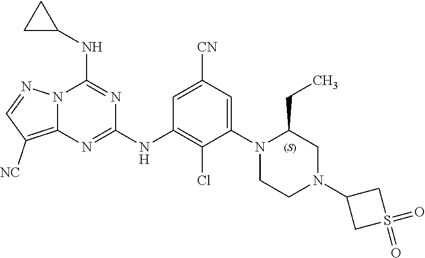

A mixture of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), 3-bromothietane 1,1-dioxide (6.00 mg, 0.032 mmol), Hunig's base (0.011 mL, 0.065 mmol) in DMSO (0.5 mL) was heated at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.9 mg (44.4% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0-3 min), 100% B (3-3.5 min); Flow: 1 mL/min; Detection: UV (220 nm) and MS (ESI+). Injection 1 results: Purity: 92.2%; Observed Mass: 567.1; RT: 1.68 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0-3 min), 100% B (3-3.5 min); Flow: 1 mL/min; Detection: UV (220 nm) and MS (ESI+). Injection 2 results: Purity: 100%; Observed Mass: 567.2; RT: 1.95 min.

### Example 117

### 2-({2-Chloro-5-cyano-3-[(2S)-2-ethyl-4-(oxetan-3-yl)piperazin-1-yl]phenyl}amino)-4-(cyclopropylamino)pyrazolo[1,5-a][1,3,5]triazine-8-carbonitrile

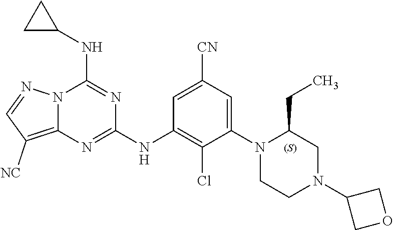

A mixture of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), acetic acid (6.18 μL, 0.108 mmol), oxetan-3-one (2.335 mg, 0.032 mmol) in DMSO (0.5 mL) was stirred at rt for 1 h, then sodium triacetoxyborohydride (13.73 mg, 0.065 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.5 mg (31.2% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 519.1; RT: 1.96 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 519.2; RT: 1.45 min.

### Example 118

### (S)-2-((2-chloro-5-cyano-3-(4-(1,1-dioxidotetrahydro-2H-thiopyran-4-yl)-2-ethylpiperazin-1-yl)phenyl)amino)-4-(cyclopropylamino)pyrazolo[1,5-a][1,3,5]triazine-8-carbonitrile

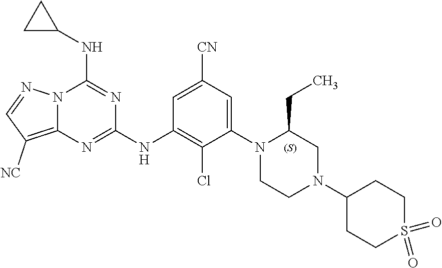

A mixture of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), acetic acid (6.18 μL, 0.108 mmol), tetrahydro-4H-thiopyran-4-one 1,1-dioxide (4.80 mg, 0.032 mmol) in DMSO (0.5 mL) was stirred at rt for 1 h. Sodium triacetoxyborohydride (13.73 mg, 0.065 mmol) was added and contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5.3 mg (34.6% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 595.1; RT: 1.96 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 595.2; RT: 1.44 min.

### Example 119

### 2-({3-[(2S)-4-Acetyl-2-ethylpiperazin-1-yl]-2-chloro-5-cyanophenyl}amino)-4-(cyclopropylamino)pyrazolo[1,5-a][1,3,5]triazine-8-carbonitrile

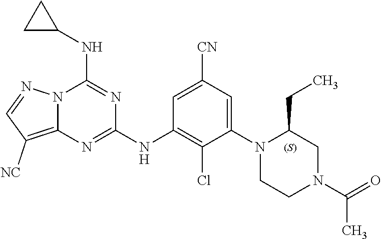

A mixture of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), acetic anhydride (4.41 mg, 0.043 mmol), Hunig's base (0.011 mL, 0.065 mmol) in DMSO (0.5 mL) was stirred at rt for 15 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative Reverse Phase employing the following conditions: Column: Xbridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 4.8 mg (44.0% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 505; RT: 1.85 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 505.2; RT: 1.82 min.

### Example 120

### Methyl (3S)-4-(2-chloro-5-cyano-3-{[8-cyano-4-(cyclopropylamino)pyrazolo[1,5-a][1,3,5]triazin-2-yl]amino}phenyl)-3-ethylpiperazine-1-carboxylate

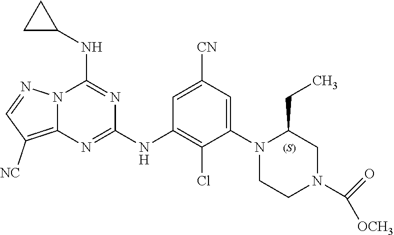

A mixture of (S)-2-((2-chloro-5-cyano-3-(2-ethylpiperazin-1-yl)phenyl)amino)-4-(cyclopropylamino)pyrazolo[1,5-a][1,3,5]triazine-8-carbonitrile (10 mg, 0.022 mmol), methyl carbonochloridate (4.08 mg, 0.043 mmol), Hunig's base (0.011 mL, 0.065 mmol) in DMSO (0.5 mL) was stirred at rt for 15 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 5 mg (40.1% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 90.3%; Observed Mass: 521.1; RT: 2.12 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 94.5%; Observed Mass: 521.2; RT: 2.01 min.

### Example 121

### (2S)-2-[(3S)-4-(2-Chloro-5-cyano-3-{[8-cyano-4-(methylamino)pyrazolo[1,5-a][1,3,5]triazin-2-yl]amino}phenyl)-3-methylpiperazin-1-yl]propanamide

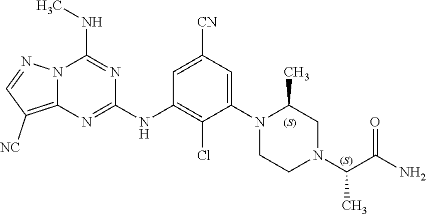

### Step 1. 4-((4-methoxybenzyl)(methyl)amino)-2-(methylthio)pyrazolo[1,5-a][1,3,5]triazine-8-carbonitrile

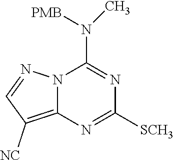

A mixture of 4-chloro-2-(methylthio)pyrazolo[1,5-a][1,3,5]triazine-8-carbonitrile (2.099 g, 9.3 mmol) in THF (30 ml) was added 1-(4-methoxyphenyl)-N-methylmethanamine (1.547 g, 10.23 mmol) and Hunig's base (1.949 mL, 11.16 mmol). The mixture was stirred at rt for 2 h. It was then diluted with EtOAc and washed with water. The organic layer was dried over MgSO₄, filtered, and concentrated to obtain a brown solid, which was then purified by ISCO, eluting with 30% EtOAc/hexane to obtain 2.7 g of the product as a solid. LCMS (M+1)=341.2.

### Step 2. 4-((4-Methoxybenzyl)(methyl)amino)-2-(methylsulfonyl)pyrazolo[1,5-a][1,3,5]triazine-8-carbonitrile

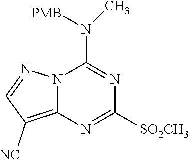

To a solution of 4-((4-methoxybenzyl)(methyl)amino)-2-(methylthio) pyrazolo[1,5-a][1,3,5]triazine-8-carbonitrile (2.3 g, 6.76 mmol) in DCM (50 mL) was added mCPBA (4.54 g, 20.27 mmol) dissolved in DCM (2 mL), at 0° C., over a period of 30 min. The reaction mixture was allowed to warm to room temperature and was stirred for 1 h. The resulting suspension was diluted with DCM (20 mL), washed with 5% Na₂S₂O₃ (2×20 mL) and 5% aq. NaHCO₃ (1×20 mL). The resulting solution was dried (Na₂SO₄), filtered and concentrated to dryness and used as such for the subsequent step without further purification. LCMS (M+1)=341.2.

### Step 3. tert-Butyl (S)-4-(3-((tert-butoxycarbonyl)(8-cyano-4-((4-methoxybenzyl)(methyl)amino)pyrazolo[1,5-a][1,3,5]triazin-2-yl)amino)-2-chloro-5-cyanophenyl)-3-methylpiperazine-1-carboxylate

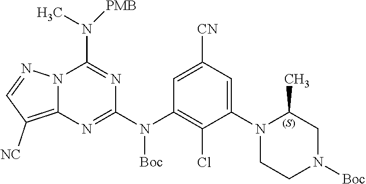

To a mixture of tert-butyl (S)-4-(3-((tert-butoxycarbonyl)amino)-2-chloro-5-cyanophenyl)-3-methylpiperazine-1-carboxylate (250 mg, 0.554 mmol) in tetrahydrofuran (5 mL) was added LiHMDS (1.109 mL, 1.109 mmol) dropwise at 0° C. It was stirred at 0° C. for 30 min and then 4-((4-methoxybenzyl)(methyl)amino)-2-(methylsulfonyl)pyrazolo[1,5-a][1,3,5]triazine-8 carbonitrile (310 mg, 0.832 mmol) in tetrahydrofuran (5 mL) was added and the contents stirred at 0° C. for 15 min, warmed to rt and stirred at rt for 2.5 h. The reaction was quenched with water, extracted with EtOAc. The organic layer was washed with water, dried over MgSO₄, filtered and concentrated. The residue was purified by ISCO, eluting with 35% EtOAc/hexane to afford tert-butyl (S)-4-(3-((tert-butoxycarbonyl)(8-cyano-4-((4-methoxybenzyl)(methyl)amino)pyrazolo[1,5-a][1,3,5]triazin-2-yl)amino)-2-chloro-5-cyanophenyl)-3-methylpiperazine-1-carboxylate (380 mg, 0.511 mmol, 92% yield).

### Step 4. (S)-2-((2-Chloro-5-cyano-3-(2-methylpiperazin-1-yl)phenyl)amino)-4-(methylamino)pyrazolo[1,5-a][1,3,5]triazine-8-carbonitrile

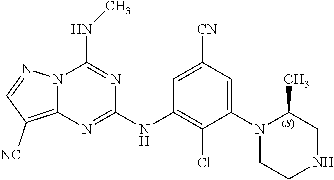

A mixture of tert-butyl (S)-4-(3-((tert-butoxycarbonyl)(8-cyano-4-((4-methoxybenzyl)(methyl)amino)pyrazolo[1,5-a][1,3,5]triazin-2-yl)amino)-2-chloro-5-cyanophenyl)-3-methylpiperazine-1-carboxylate (380 mg, 0.511 mmol), TFA (1.576 mL, 20.45 mmol) in CH₂Cl₂ (2 mL) was stirred at rt for 2 h. The reaction mixture was concentrated, the residue dissolved in ClCH₂CH₂Cl (2 mL) followed by the addition of TFA (1.576 mL, 20.45 mmol) and anisole (0.223 mL, 2.045 mmol). The contents were heated at 50° C. for 1 h. It was then concentrated and to the residue was added 7 N NH₃ in MeOH and the solid was filtered and dried to obtain (S)-2-((2-chloro-5-cyano-3-(2-methylpiperazin-1-yl)phenyl)amino)-4-(methylamino)pyrazolo[1,5-a][1,3,5]triazine-8-carbonitrile (180 mg, 0.426 mmol, 83% yield).

### Step 5. Example 121

A solution of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), (R)-1-amino-1-oxopropan-2-yl methanesulfonate (5.93 mg, 0.035 mmol), Hunig's base (0.012 mL, 0.071 mmol) in DMSO (0.5 mL) was stirred at 80° C. for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.9 mg (56.6% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.9%; Observed Mass: 494.2; RT: 1.61 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 494.1; RT: 1.18 min.

The following compounds were prepared in a similar procedure as described for Example 121 employing the R⁵ group identified in following table:

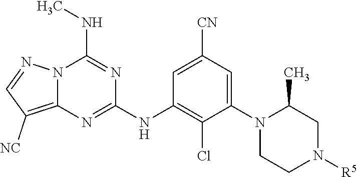

| | | LC/MS [M + H]⁺/ |
|---|---|---|
| Example # | R⁵ | RT |
| 122 | 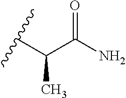 [SMILES: *[C@H](C)C(N)=O] | 494.1 1.62 min |
| 123 | 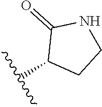 [SMILES: *[C@H]1CCNC1=O] | 506.0 1.55 min |
| 124 | 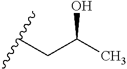 [SMILES: *C[C@H](C)O] | 481.0 1.31 min |
| 125 | 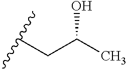 [SMILES: *C[C@@H](C)O] | 481.1 1.25 min |
| 126 | 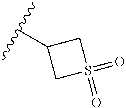 [SMILES: *C1CS(=O)(=O)C1] | 527.0 1.72 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

### Example 127

### Methyl (3S)-4-(2-chloro-5-cyano-3-{[8-cyano-4-(methylamino)pyrazolo[1,5-a][1,3,5]triazin-2-yl]amino}phenyl)-3-methylpiperazine-1-carboxylate

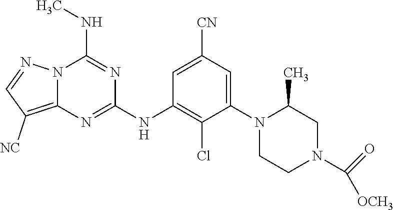 [SMILES: CNc1nc(Nc2cc(C#N)cc(N3CCN(C(=O)OC)C[C@@H]3C)c2Cl)nc2c(C#N)cnn12]

A mixture of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), methyl carbonochloridate (4.47 mg, 0.047 mmol), Hunig's base (0.012 mL, 0.071 mmol) in DMSO (0.5 mL) was stirred at rt for 15 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 3.4 mg (28.9% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.7%; Observed Mass: 481.1; RT: 1.84 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.4%; Observed Mass: 481.1; RT: 1.91 min.

### Example 128

### 2-({3-[(2S)-4-Acetyl-2-methylpiperazin-1-yl]-2-chloro-5-cyanophenyl}amino)-4-(methylamino)pyrazolo[1,5-a][1,3,5]triazine-8-carbonitrile

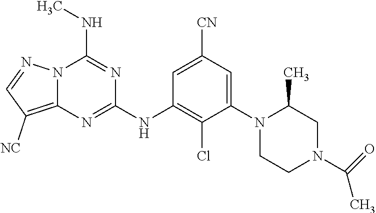 [SMILES: CNc1nc(Nc2cc(C#N)cc(N3CCN(C(C)=O)C[C@@H]3C)c2Cl)nc2c(C#N)cnn12]

A mixture of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), Hunig's base (0.012 mL, 0.071 mmol), acetic anhydride (4.83 mg, 0.047 mmol) in DMSO (0.5 mL) was stirred at rt for 15 min. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 2.6 mg (23.27% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.4%; Observed Mass: 465.1; RT: 1.62 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 98.8%; Observed Mass: 465; RT: 1.55 min.

### Example 129

### 2-({2-Chloro-5-cyano-3-[(2S)-2-methyl-4-(oxetan-3-yl)piperazin-1-yl]phenyl}amino)-4-(methylamino)pyrazolo[1,5-a][1,3,5]triazine-8-carbonitrile

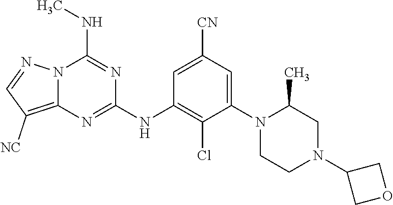 [SMILES: CNc1nc(Nc2cc(C#N)cc(N3CCN(C4COC4)C[C@@H]3C)c2Cl)nc2c(C#N)cnn12]

A mixture of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), oxetan-3-one (2.56 mg, 0.035 mmol), acetic acid (6.77 μL, 0.118 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 4.5 mg (32.1% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 479; RT: 1.69 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 479.2; RT: 1.26 min.

### Example 130

### 2-({2-Chloro-5-cyano-3-[(2S)-4-(2-hydroxyethyl)-2-methylpiperazin-1-yl]phenyl}amino)-4-(methylamino)pyrazolo[1,5-a][1,3,5]triazine-8-carbonitrile

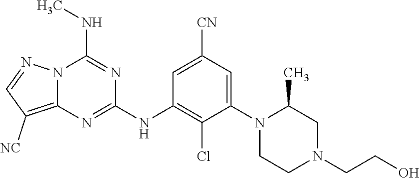 [SMILES: CNc1nc(Nc2cc(C#N)cc(N3CCN(CCO)C[C@@H]3C)c2Cl)nc2c(C#N)cnn12]

A mixture of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), 1,4-dioxane-2,5-diol (5.68 mg, 0.047 mmol), acetic acid (4.06 μL, 0.071 mmol) in DMSO (0.5 mL) was stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 1.2 mg (10.87% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 467; RT: 1.27 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI 10+). Injection 2 results: Purity: 100%; Observed Mass: 467; RT: 1.5 min.

### Example 131

### (S)-2-((2-chloro-5-cyano-3-(4-(1,1-dioxidotetrahydro-2H-thiopyran-4-yl)-2-methylpiperazin-1-yl)phenyl)amino)-4-(methylamino)pyrazolo[1,5-a][1,3,5]triazine-8-carbonitrile

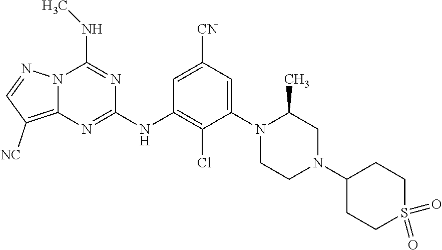 [SMILES: CNc1nc(Nc2cc(C#N)cc(N3CCN(C4CCS(=O)(=O)CC4)C[C@@H]3C)c2Cl)nc2c(C#N)cnn12]

A mixture of (S)-2-((2-chloro-5-cyano-3-(2-methylpiperazin-1-yl)phenyl)amino)-4-(methylamino)pyrazolo[1,5-a][1,3,5]triazine-8-carbonitrile (10 mg, 0.024 mmol), acetic acid (6.77 μL, 0.118 mmol), tetrahydro-4H-thiopyran-4-one 1,1-dioxide (5.26 mg, 0.035 mmol) in DMSO (0.5 mL) was stirred at rt for 1 h. Sodium triacetoxyborohydride (15.04 mg, 0.071 mmol) was added and the contents stirred at rt for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 6.4 mg (38.5% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.1%; Observed Mass: 555; RT: 1.3 min. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 95.7%; Observed Mass: 555; RT: 1.71 min.

### Example 132

### 2-{[2-chloro-5-cyano-3-(4-methanesulfonylpiperazin-1-yl)phenyl]amino}-4-(ethylamino)pyrazolo[1,5-a][1,3,5]triazine-8-carbonitrile

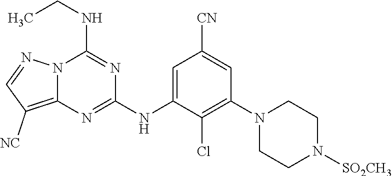 [SMILES: CCNc1nc(Nc2cc(C#N)cc(N3CCN(S(C)(=O)=O)CC3)c2Cl)nc2c(C#N)cnn12]

### Step 1. 4-(ethyl(4-methoxybenzyl)amino)-2-(methylthio)pyrazolo[1,5-a][1,3,5]triazine-8-carbonitrile

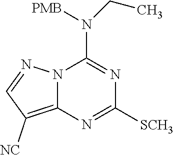

A mixture of 4-chloro-2-(methylthio)pyrazolo[1,5-a][1,3,5]triazine-8-carbonitrile (0.32 g, 1.418 mmol) in THF (10 ml) was added N-(4-methoxybenzyl)ethanamine (0.258 g, 1.560 mmol) and Hunig's base (0.297 mL, 1.702 mmol). The mixture was stirred at rt for 2 h, diluted with EtOAc and washed with water. The organic layer was dried over MgSO₄, filtered, and concentrated to afford a brown solid, which was then purified by ISCO, eluting with 30-50% EtOAc/hexane to obtain 400 mg of 4-(ethyl(4-methoxybenzyl)amino)-2-(methylthio)pyrazolo[1,5-a][1,3,5]triazine-8-carbonitrile. LC/MS [M+H]⁺=355.1.

### Step 2. 4-(ethyl(4-methoxybenzyl)amino)-2-(methylsulfonyl)pyrazolo[1,5-a][1,3,5]triazine-8-carbonitrile

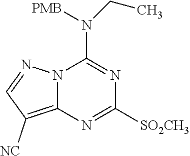

To a solution of 4-(ethyl(4-methoxybenzyl)amino)-2-(methylthio) pyrazolo[1,5-a][1,3,5]triazine-8-carbonitrile (1.7 g, 4.80 mmol) in DCM (50 mL) was added mCPBA (3.22 g, 14.39 mmol) dissolved in DCM (2 mL), at 0° C., over 30 min. The reaction mixture was allowed to warm to room temperature and was stirred for 1 h. The resulting suspension was diluted with DCM (20 mL) and washed with 5% Na₂S₂O₃ (2×20 mL), 5% aq. NaHCO₃ (1×20 mL), dried (Na₂SO₄), filtered and concentrated to give 1.7 g of 4-(ethyl(4-methoxybenzyl)amino)-2-(methylsulfonyl)pyrazolo[1,5-a][1,3,5]triazine-8-carbonitrile which was used as such for the subsequent step without further purification. LC/MS [M+H]⁺=387.1.

### Step 3. tert-butyl 4-(2-chloro-5-cyano-3-((8-cyano-4-(ethyl(4-methoxybenzyl) amino)pyrazolo[1,5-a][1,3,5]triazin-2-yl)amino)phenyl)piperazine-1-carboxylate

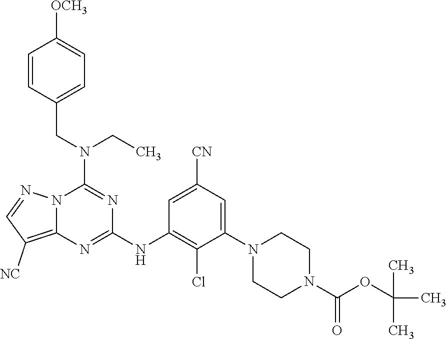 [SMILES: CCN(Cc1ccc(OC)cc1)c1nc(Nc2cc(C#N)cc(N3CCN(C(=O)OC(C)(C)C)CC3)c2Cl)nc2c(C#N)cnn12]

To a solution of tert-butyl 4-(3-amino-2-chloro-5-cyanophenyl) piperazine-1-carboxylate (337 mg, 1 mmol) in THF (10 mL) at 0° C. was added potassium tert-butoxide (2.20 mL, 2.200 mmol) dropwise. After 30 min, a solution of 4-(ethyl(4-methoxybenzyl)amino)-2-(methylsulfonyl)pyrazolo[1,5-a][1,3,5]triazine-8-carbonitrile (386 mg, 1.000 mmol) in THF (6 mL) was added dropwise. After stirring for 10 h, the reaction was quenched with 1 N HCl, extracted with EtOAc, washed with brine, dried over MgSO₄, filtered, concentrated and purified by flash chromatography eluting with % 30-60 EtOAc in hexanes to give 400 mg of tert-butyl 4-(2-chloro-5-cyano-3-((8-cyano-4-(ethyl(4-methoxybenzyl)amino)pyrazolo[1,5-a][1,3,5]triazin-2-yl)amino)phenyl)piperazine-1-carboxylate as a solid. LC/MS [M+H]⁺=643.6.

### Step 4. 2-{[2-chloro-5-cyano-3-(piperazin-1-yl)phenyl]amino}-4-(ethylamino)pyrazolo[1,5-a][1,3,5]triazine-8-carbonitrile

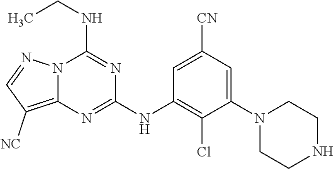 [SMILES: CCNc1nc(Nc2cc(C#N)cc(N3CCNCC3)c2Cl)nc2c(C#N)cnn12]

To a solution of tert-butyl 4-(2-chloro-5-cyano-3-((8-cyano-4-(ethyl(4-methoxybenzyl)amino)pyrazolo[1,5-a][1,3,5]triazin-2-yl)amino)phenyl) piperazine-1-carboxylate (450 mg, 0.700 mmol) in 3 ml of DCM was added 2 mL of TFA and the contents stirred for 0.5 h. The reaction mixture was concentrated and to the residue was added DCE (4665 μL) followed by anisole (306 μL, 2.80 mmol) and TFA (2156 μL, 28.0 mmol). The resulting solution was heated at 50° C. for 1 h. The reaction mixture was cooled to rt, the solvent was removed in vacuo and the material azeotroped with DCM (3×) to remove the excess TFA. 2 mL of 7N NH₃ in MeOH was added. The resulting solid was filtered and washed with MeOH to give -{[2-chloro-5-cyano-3-(piperazin-1-yl)phenyl]amino}-4-(ethylamino)pyrazolo[1,5-a][1,3,5]triazine-8-carbonitrile (270 mg, 0.638 mmol, 91% yield) as white solid. LC/MS [M+H]⁺=423.3.

### Step 5. Example 132

Example 132 was prepared from 2-{[2-chloro-5-cyano-3-(piperazin-1-yl)phenyl]amino}-4-(ethylamino)pyrazolo[1,5-a][1,3,5]triazine-8-carbonitrile in a similar procedure as described for Example 13. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 96.4%; Observed Mass: 501.00; RT: 1.76 min. Injection 2 conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 2 results: Purity: 95.6%; Observed Mass: 501.10; RT: 1.72 min.

### Example 133

### 2-{[3-(4-acetylpiperazin-1-yl)-2-chloro-5-cyanophenyl]amino}-4-(ethylamino)pyrazolo[1,5-a][1,3,5]triazine-8-carbonitrile

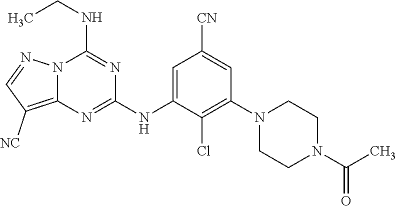 [SMILES: CCNc1nc(Nc2cc(C#N)cc(N3CCN(C(C)=O)CC3)c2Cl)nc2c(C#N)cnn12]

Example 133 was prepared from 2-{[2-chloro-5-cyano-3-(piperazin-1-yl)phenyl]amino}-4-(ethylamino)pyrazolo[1,5-a][1,3,5]triazine-8-carbonitrile in a similar procedure as described for Example 13. Analytical LC/MS was used to determine the final purity. Injection conditions: Column: Waters XBridge C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 3 min, then a 0.50 min hold at 100% B; Flow: 1 mL/min; Detection: MS and UV (220 nm). Injection 1 results: Purity: 99.2%; Observed Mass: 465.10; RT: 1.58 min.

The following compounds were prepared from 2-{[2-chloro-5-cyano-3-(piperazin-1-yl)phenyl]amino}-4-(ethylamino)pyrazolo[1,5-a][1,3,5]triazine-8-carbonitrile in a similar procedure as described for Example 1 employing the R⁵ group identified in the table below:

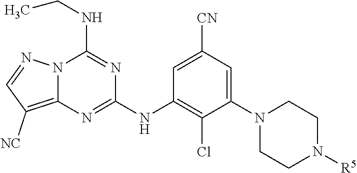

| | | LC/MS [M + H]⁺/ |
|---|---|---|
| Example # | R⁵ | RT |
| 134 | 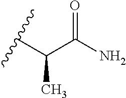 [SMILES: *[C@H](C)C(N)=O] | 494.2 1.6 min |
| 135 | 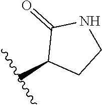 [SMILES: *[C@@H]1CCNC1=O] | 506.1 1.55 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

The following compounds were prepared from 2-{[2-chloro-5-cyano-3-(piperazin-1-yl)phenyl]amino}-4-(ethylamino)pyrazolo[1,5-a][1,3,5]triazine-8-carbonitrile in a similar procedure as described for Example 27 employing the R⁵ group identified in the table below:

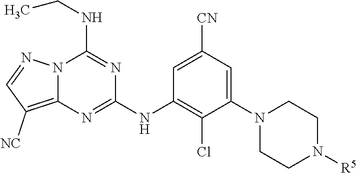

| | | LC/MS [M + H]⁺/ |
|---|---|---|
| Example # | R⁵ | RT |
| 136 | 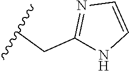 [SMILES: *Cc1ncc[nH]1] | 503.2 1.5 min |
| 137 | 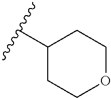 [SMILES: *C1CCOCC1] | 507.3 1.86 min |
| 138 | 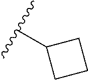 [SMILES: *C1CCC1] | 477.2 1.52 min |

### Example 139

### 2-[(2-chloro-5-cyano-3-{4-[(2S)-2-hydroxypropyl]piperazin-1-yl}phenyl)amino]-4-(cyclobutylamino)pyrazolo[1,5-a][1,3,5]triazine-8-carbonitrile

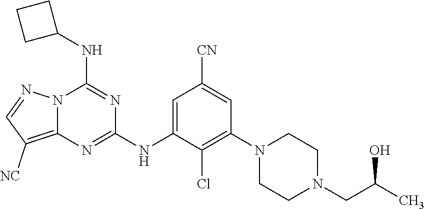 [SMILES: C[C@H](O)CN1CCN(c2cc(C#N)cc(Nc3nc(NC4CCC4)n4ncc(C#N)c4n3)c2Cl)CC1]

### Step 1. 4-(cyclobutyl(4-methoxybenzyl)amino)-2-(methylthio)pyrazolo[1,5-a][1,3,5]triazine-8-carbonitrile

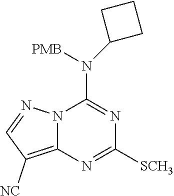

To a mixture of 4-chloro-2-(methylthio)pyrazolo[1,5-a][1,3,5]triazine-8-carbonitrile (293 mg, 1.3 mmol) in THF (30 mL) was added N-(4-methoxybenzyl)cyclobutanamine (249 mg, 1.300 mmol) and Hunig's base (0.272 mL, 1.560 mmol). The mixture was stirred at rt for 2 h. It was then diluted with EtOAc and washed with water. The organic layer was dried over MgSO₄, filtered, and concentrated to afford a brown solid, which was purified by ISCO, eluting with 30% EtOAc/hexane to obtain 440 mg of 4-(cyclobutyl(4-methoxybenzyl)amino)-2-(methylthio)pyrazolo[1,5-a][1,3,5]triazine-8-carbonitrile. LC/MS [M+H]⁺=381.3.

### Step 2. 4-(cyclobutyl(4-methoxybenzyl)amino)-2-(methylsulfonyl)pyrazolo[1,5-a][1,3,5]triazine-8-carbonitrile

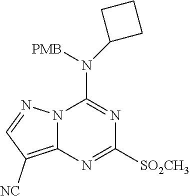

To a solution of 4-(cyclobutyl(4-methoxybenzyl)amino)-2-(methylthio) pyrazolo[1,5-a][1,3,5]triazine-8-carbonitrile (360 mg, 0.946 mmol) in DCM (20 mL) was added mCPBA (636 mg, 2.84 mmol) dissolved in 2 mL of DCM at 0° C. over 30 min. The reaction mixture was allowed to warm to rt and was stirred for 1 h. The resulting suspension was diluted with DCM (20 mL) and washed with 5% Na₂S₂O₃ (2×20 mL) and 5% aq. NaHCO₃ (1×20 mL). The resulting solution was dried (Na₂SO₄), filtered and concentrated to give 375 mg of 4-(cyclobutyl(4-methoxybenzyl)amino)-2-(methylsulfonyl)pyrazolo[1,5-a][1,3,5]triazine-8-carbonitrile which was used as such for the subsequent step without further purification. LC/MS [M+H]⁺=413.3

### Step 3. tert-butyl 4-(2-chloro-5-cyano-3-((8-cyano-4-(cyclobutyl(4-methoxybenzyl)amino)pyrazolo[1,5-a][1,3,5]triazin-2-yl)amino)phenyl)piperazine-1-carboxylate

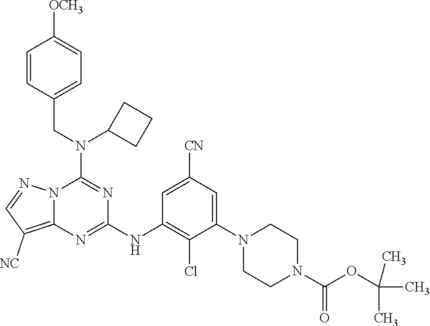 [SMILES: COc1ccc(CN(c2nc(Nc3cc(C#N)cc(N4CCN(C(=O)OC(C)(C)C)CC4)c3Cl)nc3c(C#N)cnn23)C2CCC2)cc1]

To a solution of tert-butyl 4-(3-amino-2-chloro-5-cyanophenyl) piperazine-1-carboxylate (337 mg, 1 mmol) in THF (10 mL) at 0° C. was added potassium tert-butoxide (2.200 mL, 2.200 mmol) dropwise. After stirring for 30 min, a solution of 4-(cyclobutyl(4-methoxybenzyl)amino)-2-(methylsulfonyl) pyrazolo[1,5-a][1,3,5]triazine-8-carbonitrile (412 mg, 1.000 mmol) in THF (6 mL) was added dropwise. After stirring for 10 min, the reaction was quenched with 1 N HCl, extracted with EtOAc, washed with brine, dried over MgSO₄, filtered, concentrated and purified by flash chromatography eluting with 30-60% ethyl acetate in hexanes to give tert-butyl 4-(2-chloro-5-cyano-3-((8-cyano-4-(cyclobutyl(4-methoxybenzyl)amino)pyrazolo[1,5-a][1,3,5]triazin-2-yl)amino)phenyl)piperazine-1-carboxylate (400 mg, 0.598 mmol, 59.8% yield). LC/MS [M+H]⁺=669.7.

### Step 4. 2-((2-chloro-5-cyano-3-(piperazin-1-yl)phenyl)amino)-4-(cyclobutylamino)pyrazolo[1,5-a][1,3,5]triazine-8-carbonitrile

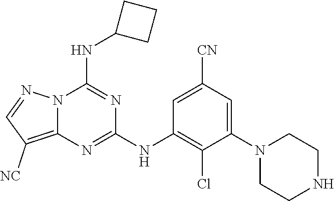 [SMILES: N#Cc1cc(Nc2nc(NC3CCC3)n3ncc(C#N)c3n2)c(Cl)c(N2CCNCC2)c1]

To a solution tert-butyl 4-(2-chloro-5-cyano-3-((8-cyano-4-(cyclobutyl(4-methoxybenzyl)amino)pyrazolo[1,5-a][1,3,5]triazin-2-yl)amino)phenyl) piperazine-1-carboxylate (400 mg, 0.598 mmol) in 0.3 mL of DCM was added 0.2 mL of TFA and the contents stirred at rt for 0.5 h. After concentration, DCE (3985 μL) was added followed by addition of TFA (1842 μL, 23.91 mmol) and anisole (261 μL, 2.391 mmol) and the reaction mixture was heated at 50° C. for 1 h. The reaction mixture was cooled to rt, concentrated and the residue azeotroped with DCM (3×) to remove the excess TFA. 0.2 mL of 7N NH₃ in MeOH was added. The resulting solid was filtered and washed with MeOH to give 2-((2-chloro-5-cyano-3-(piperazin-1-yl)phenyl)amino)-4-(cyclobutylamino)pyrazolo[1,5-a][1,3,5]triazine-8-carbonitrile (250 mg, 0.557 mmol, 93% yield) as white solid. LC/MS [M+H]⁺=449.4

### Step 5. Example 139

Example 139 was prepared from 2-((2-chloro-5-cyano-3-(piperazin-1-yl)phenyl)amino)-4-(cyclobutylamino)pyrazolo[1,5-a][1,3,5]triazine-8-carbonitrile in a similar procedure as described for Example 1. Analytical LC/MS was used to determine the final purity. Injection conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection results: Purity: 98.1%; Observed Mass: 507.20; RT: 1.86 min.

### Example 140

### 2-{[2-chloro-5-cyano-3-(piperazin-1-yl)phenyl]amino}-4-{[(1 S,2S)-2-methylcyclopropyl]amino}pyrazolo[1,5-a][1,3,5]triazine-8-carbonitrile

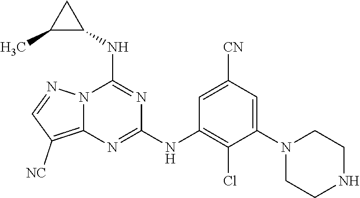 [SMILES: C[C@H]1C[C@@H]1Nc1nc(Nc2cc(C#N)cc(N3CCNCC3)c2Cl)nc2c(C#N)cnn12]

### Step 1. 4-((4-methoxybenzyl)((1 S,2S)-2-methylcyclopropyl)amino)-2-(methylthio)pyrazolo[1,5-a][1,3,5]triazine-8-carbonitrile

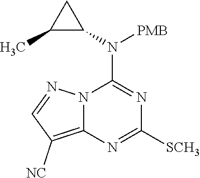

A mixture of 4-chloro-2-(methylthio)pyrazolo[1,5-a][1,3,5]triazine-8-carbonitrile (0.5 g, 2.216 mmol) in THF (100 ml) was added (1S,2S)—N-(4-methoxybenzyl)-2-methylcyclopropan-1-amine (0.424 g, 2.216 mmol) and Hunig's base (0.774 mL, 4.43 mmol). The mixture was stirred at rt for 2 h. It was diluted with EtOAc and washed with water. The organic layer was dried over MgSO₄, filtered, concentrated and to the residue was added 4 mL of cold MeOH and stirred for 30 min. The white precipitate was collected by filtering washing with cold MeOH to provide 0.6 g 4-((4-methoxybenzyl) ((1 S,2S)-2-methylcyclopropyl)amino)-2-(methylthio)pyrazolo[1,5-a][1,3,5]triazine-8-carbonitrile. LC/MS [M+H]⁺=381.3.

### Step 2. 4-((4-methoxybenzyl)((1 S,2S)-2-methylcyclopropyl)amino)-2-(methylsulfonyl)pyrazolo[1,5-a][1,3,5]triazine-8-carbonitrile

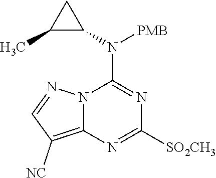

To a solution of 4-((4-methoxybenzyl)((1 S,2S)-2-methylcyclopropyl) amino)-2-(methylthio)pyrazolo[1,5-a][1,3,5]triazine-8-carbonitrile (500 mg, 1.314 mmol) in DCM (10 mL) was added mCPBA (884 mg, 3.94 mmol) dissolved in 2 mL of DCM at 0° C. over 30 min. The reaction mixture was allowed to warm to rt and was stirred for 1 h. The resulting suspension was diluted with DCM (20 mL) and washed with 5% Na₂S₂O₃ (2×20 mL) and 5% aq. NaHCO₃ (1×20 mL). The resulting solution was dried (Na₂SO₄), filtered and concentrated to dryness. The crude was purified by silica gel column chromatography, eluting with 20-50% ethyl acetate in hexanes to give 501 mg of 4-((4-methoxybenzyl)((1 S,2S)-2-methylcyclopropyl)amino)-2-(methyl sulfonyl)pyrazolo[1,5-a][1,3,5]triazine-8-carbonitrile. LC/MS [M+H]⁺=412.9.

### Step 3. tert-butyl 4-(2-chloro-5-cyano-3-((8-cyano-4-((4-methoxybenzyl)((1 S,2S)-2-methylcyclopropyl)amino)pyrazolo[1,5-a][1,3,5]triazin-2-yl)amino)phenyl)piperazine-1-carboxylate

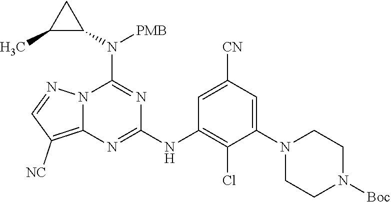

To a solution of tert-butyl 4-(3-amino-2-chloro-5-cyanophenyl) piperazine-1-carboxylate (305 mg, 0.906 mmol)) in THF (50 mL) at 0° C. was added potassium tert-butoxide (1.992 mL, 1.992 mmol) (1.980 mL, 1.980 mmol) dropwise. After stirring for 30 min, a solution of 4-((4-methoxybenzyl) ((1 S,2S)-2-methylcyclopropyl)amino)-2-(methylsulfonyl)pyrazolo[1,5-a][1,3,5]triazine-8-carbonitrile (374 mg, 0.906 mmol) in THF (6 mL) was added dropwise. After stirring for 10 min, the reaction was quenched with 1N HCl, extracted with EtOAc, washed with brine, dried over MgSO₄, filtered, concentrated and purified by flash chromatography eluting with 20-50% ethyl acetate in hexanes to give 410 mg tert-butyl 4-(2-chloro-5-cyano-3-((8-cyano-4-((4-methoxybenzyl)((1 S,2S)-2-methylcyclopropyl)amino)pyrazolo[1,5-a][1,3,5]triazin-2-yl)amino)phenyl)piperazine-1-carboxylate. LC/MS [M+H]⁺=669.6.

### Step 4. Example 140

To a solution tert-butyl 4-(2-chloro-5-cyano-3-((8-cyano-4-((4-methoxybenzyl)((1 S,2S)-2-methylcyclopropyl)amino)pyrazolo[1,5-a][1,3,5]triazin-2-yl)amino)phenyl)piperazine-1-carboxylate (400 mg, 0.598 mmol) in 3 mL of DCM was added 2 mL of TFA and the contents stirred for 0.5 h. After concentration, the residue was dissolved in DCE (3985 μl) followed by addition of TFA (1842 μL, 23.91 mmol) and anisole (261 μL, 2.391 mmol) and the contents heated at 50° C. for 1 h. The solvent was removed in vacuo and the material azeotroped with DCM (3×) to remove the excess TFA. 2 mL of 7N NH₃ in MeOH was added. The resulting solid was filtered and washed with MeOH to give 2-((2-chloro-5-cyano-3-(piperazin-1-yl)phenyl)amino)-4-(((1S,2S)-2-methylcyclopropyl)amino)pyrazolo[1,5-a][1,3,5]triazine-8-carbonitrile (220 mg, 0.490 mmol, 82% yield) as white solid. LC/MS [M+H]⁺=449.5.

### Example 141

### methyl 4-{2-chloro-5-cyano-3-[(8-cyano-4-{[(1 S,2S)-2-methylcyclopropyl]amino}pyrazolo[1,5-a][1,3,5]triazin-2-yl)amino]phenyl}piperazine-1-carboxylate

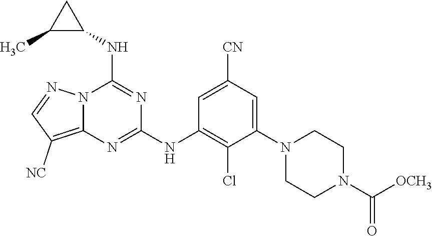 [SMILES: COC(=O)N1CCN(c2cc(C#N)cc(Nc3nc(N[C@H]4C[C@@H]4C)n4ncc(C#N)c4n3)c2Cl)CC1]

Example 141 was prepared from 2-((2-chloro-5-cyano-3-(piperazin-1-yl)phenyl)amino)-4-(((1S,2S)-2-methylcyclopropyl)amino)pyrazolo[1,5-a][1,3,5]triazine-8-carbonitrile in a similar procedure as described for Example 13. Analytical LC/MS was used to determine the final purity. Injection 1 conditions: Column: Waters XBridge C18, 2.1 mm×50 mm, 1.7 μm particles, Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 507.20; RT: 2.09 min.

The following compounds were prepared from 2-((2-chloro-5-cyano-3-(piperazin-1-yl)phenyl)amino)-4-(((1 S,2S)-2-methylcyclopropyl)amino) pyrazolo[1,5-a][1,3,5]triazine-8-carbonitrile in a similar procedure as described for Example 13 employing the R⁵ group identified in the table below:

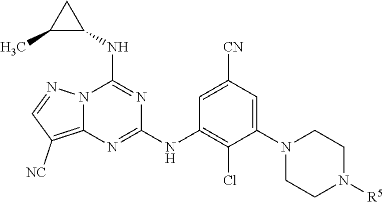

| | | LC/MS [M + H]⁺/ |
|---|---|---|
| Example # | R⁵ | RT |
| 142 | 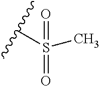 [SMILES: *S(C)(=O)=O] | 527.2 1.88 min |
| 143 | 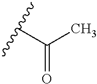 [SMILES: *C(C)=O] | 491.2 1.75 min |

### LC/MS Method:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

The following compounds were prepared from 2-((2-chloro-5-cyano-3-(piperazin-1-yl)phenyl)amino)-4-(((1 S,2S)-2-methylcyclopropyl)amino) pyrazolo[1,5-a][1,3,5]triazine-8-carbonitrile in a similar procedure as described for Example 1 employing the R⁵ group identified in the table below:

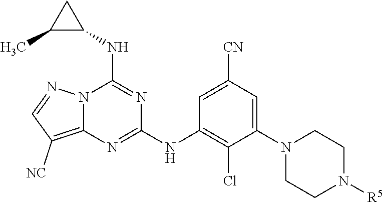

| | | LC/MS [M + H]⁺/ |
|---|---|---|
| Example # | R⁵ | RT |
| 144 | 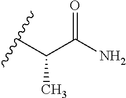 [SMILES: *[C@@H](C)C(N)=O] | 520.2 1.81 min |
| 145 | 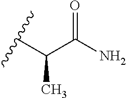 [SMILES: *[C@H](C)C(N)=O] | 520.2 1.8 min |
| 146 | 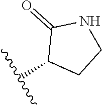 [SMILES: *[C@H]1CCNC1=O] | 532.3 1.74 min |
| 147 | 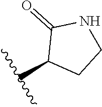 [SMILES: *[C@@H]1CCNC1=O] | 532.3 1.73 min |
| 148 | 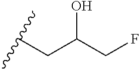 [SMILES: *CC(O)CF] | 525.2 1.91 min |

### LC/MS Conditions:

Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm).

The following compounds were prepared from 2-((2-chloro-5-cyano-3-(piperazin-1-yl)phenyl)amino)-4-(((1 S,2S)-2-methylcyclopropyl)amino) pyrazolo[1,5-a][1,3,5]triazine-8-carbonitrile in a similar procedure as described for Example 27 employing the R⁵ group identified in the table below:

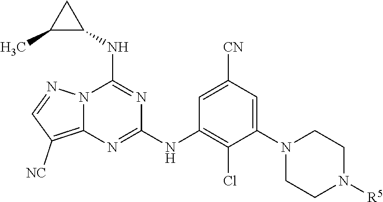

| | | LC/MS [M + H]⁺/ |
|---|---|---|
| Example # | R⁵ | RT |
| 149 | 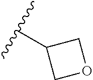 [SMILES: *C1COC1] | 505.2 1.83 min |
| 150 | 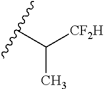 [SMILES: *C(C)C(F)F] | 527.2 2.37 min |

### Example 151

### 2-(4-{2-chloro-5-cyano-3-[(8-cyano-4-{[(1 S,2S)-2-methylcyclopropyl]amino}pyrazolo[1,5-a][1,3,5]triazin-2-yl)amino]phenyl}piperazin-1-yl)acetamide

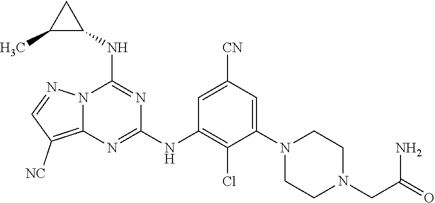 [SMILES: C[C@H]1C[C@@H]1Nc1nc(Nc2cc(C#N)cc(N3CCN(CC(N)=O)CC3)c2Cl)nc2c(C#N)cnn12]

To a solution of 2-((2-chloro-5-cyano-3-(piperazin-1-yl)phenyl)amino)-4-(((1 S,2S)-2-methylcyclopropyl)amino)pyrazolo[1,5-a][1,3,5]triazine-8-carbonitrile (8.98 mg, 0.02 mmol) in DMF (1 mL) was added K₂CO₃ (5.53 mg, 0.040 mmol) followed by 2-bromoacetamide (2.76 mg, 0.020 mmol) at rt and stirred for 2 h. After filtration, the material was purified by reverse phase column chromatography to give 10 mg 2-(4-{2-chloro-5-cyano-3-[(8-cyano-4-{[(1 S,2S)-2-methylcyclopropyl]amino}pyrazolo[1,5-a][1,3,5]triazin-2-yl)amino]phenyl}piperazin-1-yl)acetamide. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 98.8%; Observed Mass: 506.20; RT: 1.74 min.

### Example 152

### 2-({2-chloro-5-cyano-3-[4-(2-methoxyethyl)piperazin-1-yl]phenyl}amino)-4-{[(1 S,2S)-2-methylcyclopropyl]amino}pyrazolo[1,5-a][1,3,5]triazine-8-carbonitrile

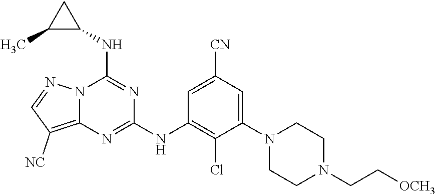 [SMILES: COCCN1CCN(c2cc(C#N)cc(Nc3nc(N[C@H]4C[C@@H]4C)n4ncc(C#N)c4n3)c2Cl)CC1]

A solution of 2-((2-chloro-5-cyano-3-(piperazin-1-yl)phenyl)amino)-4-(((1 S,2S)-2-methylcyclopropyl)amino)pyrazolo[1,5-a][1,3,5]triazine-8-carbonitrile (8.98 mg, 0.02 mmol), 1-bromo-2-methoxyethane (4.17 mg, 0.030 mmol) and Hunig's base (6.99 μL, 0.040 mmol) in DMSO (1 mL) was stirred at 50° C. for 48 h. The material was submitted by reverse phase column purification to give 2-((2-chloro-5-cyano-3-(4-(2-methoxyethyl)piperazin-1-yl)phenyl)amino)-4-(((1 S,2S)-2-methylcyclopropyl)amino)pyrazolo [1,5-a][1,3,5]triazine-8-carbonitrile (8.8 mg, 0.017 mmol, 87% yield). Injection conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection results: Purity: 100%; Observed Mass: 507.10; RT: 2 min.

### Example 153

### 2-({3-[(8aR)-3-oxo-hexahydro-1H-[1,3]oxazolo[3,4-a]piperazin-7-yl]-2-chloro-5-cyanophenyl}amino)-4-(cyclopropylamino)pyrazolo[1,5-a][1,3,5]triazine-8-carbonitrile

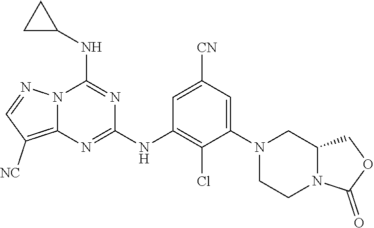 [SMILES: N#Cc1cc(Nc2nc(NC3CC3)n3ncc(C#N)c3n2)c(Cl)c(N2CCN3C(=O)OC[C@H]3C2)c1]

### Step 1. 2-((3-bromo-2-chloro-5-cyanophenyl)amino)-4-(cyclopropyl(4-methoxybenzyl)amino)pyrazolo[1,5-a][1,3,5]triazine-8-carbonitrile

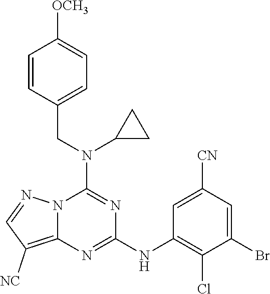 [SMILES: COc1ccc(CN(c2nc(Nc3cc(C#N)cc(Br)c3Cl)nc3c(C#N)cnn23)C2CC2)cc1]

To a solution of 3-amino-5-bromo-4-chlorobenzonitrile (2.8 g, 12.10 mmol) in THF (100 mL) was added LiHMDS (24.80 mL, 24.80 mmol) dropwise at −20° C. It was stirred at −20° C. for 30 min and then 4-(cyclopropyl(4-methoxybenzyl)amino)-2-(methylsulfonyl)pyrazolo[1,5-a][1,3,5]triazine-8-carbonitrile (5.06 g, 12.70 mmol) in THF (100 mL) was added and stirred at 0° C. for 30 min. The reaction was quenched with water, neutralized with 1 N HCl and extracted with EtOAc. The organic layer was washed with water, dried over MgSO₄, filtered and concentrated. To the residue was added MeOH and the solid was filtered to yield 2-((3-bromo-2-chloro-5-cyanophenyl)amino)-4-(cyclopropyl(4-methoxybenzyl)amino) pyrazolo[1,5-a][1,3,5]triazine-8-carbonitrile (4.6 g, 8.37 mmol, 69.2% yield) as an off white solid. LC/MS [M+H]⁺=549. ¹H NMR (499 MHz, DMSO-d₆) δ 9.96 (s, 1H), 8.44 (s, 1H), 8.28 (s, 1H), 8.16 (d, J=1.7 Hz, 1H), 7.15 (br d, J=8.5 Hz, 2H), 6.85 (d, J=8.6 Hz, 2H), 5.32-5.04 (m, 2H), 3.72 (s, 3H), 2.97-2.75 (m, 1H), 0.93-0.66 (m, 4H).

### Step 2. Example 153

A mixture of 2-((3-bromo-2-chloro-5-cyanophenyl)amino)-4-(cyclopropyl(4 methoxybenzyl)amino)pyrazolo[1,5-a][1,3,5]triazine-8-carbonitrile (20 mg, 0.036 mmol), (R)-hexahydro-3H-oxazolo[3,4-a]pyrazin-3-one, HCl (10.40 mg, 0.058 mmol), Pd₂(dba)₃ (3.33 mg, 3.64 μmol), BINAP (6.80 mg, 10.91 μmol), Cs₂CO₃ (71.1 mg, 0.218 mmol) in dioxane (2 mL) was purged with nitrogen and then stirred at 105° C. for 16 h. The reaction mixture was cooled to rt, and celite was added. The reaction mixture was filtered through celite. The filtrate was concentrated in vacuo. To the residue was added 0.5 mL of TFA and 1 mL of DCE followed by anisole (0.016 mL, 0.146 mmol). The resulting solution was stirred for 1 h at 50° C. After concentration, the crude material was purified via preparative LC/MS employing the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 18% B, 18-58% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product 2-({3-[(8aR)-3-oxo-hexahydro-1H-[1,3]oxazolo[3,4-a]piperazin-7-yl]-2-chloro-5-cyanophenyl}amino)-4-(cyclopropylamino)pyrazolo[1,5-a][1,3,5]triazine-8-carbonitrile was 4.2 mg, and its estimated purity by LCMS analysis was 99%. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 99%; Observed Mass: 491.00; RT: 1.7 min. ¹H NMR (500 MHz, DMSO-d₆) δ 8.40 (s, 1H), 8.17 (s, 1H), 7.42 (s, 1H), 4.43 (br t, J=7.8 Hz, 1H), 4.14-3.95 (m, 2H), 3.72 (br. d, J=11.6 Hz, 1H), 3.58-3.35 (m, 2H), 3.29-3.14 (m, 2H), 2.91-2.66 (m, 2H), 0.86-0.66 (m, 4H).

The following compounds were prepared from 2-((3-bromo-2-chloro-5-cyanophenyl)amino)-4-(cyclopropyl(4-methoxybenzyl)amino)pyrazolo[1,5-a][1,3,5]triazine-8-carbonitrile in a similar procedure as described for Example 153 employing the R⁴ group identified in the table below:

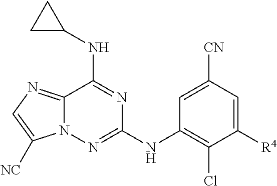

| | | LC/MS [M + H]⁺/ |
|---|---|---|
| Example # | R⁴ | RT |
| 154 | 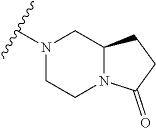 [SMILES: *N1CCN2C(=O)CC[C@@H]2C1] | 489.1 1.71 min |
| 155 | 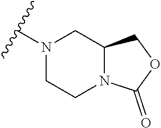 [SMILES: *N1CCN2C(=O)OC[C@@H]2C1] | 491.1 1.72 min |
| 156 | 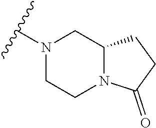 [SMILES: *N1CCN2C(=O)CC[C@H]2C1] | 489.1 1.65 min |
| 157 | 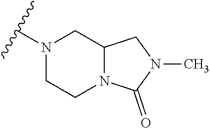 [SMILES: *N1CCN2C(=O)N(C)CC2C1] | 504.1 1.67 min |

The following compounds were prepared from 2-((3-bromo-2-chloro-5-cyanophenyl)amino)-4-(cyclopropyl(4-methoxybenzyl)amino)pyrazolo[1,5-a][1,3,5]triazine-8-carbonitrile in a similar procedure as described for Example 153 then followed by another similar procedure as described for Example 1 or Example 13 or Example 27 employing the R¹⁰ group identified in the table below:

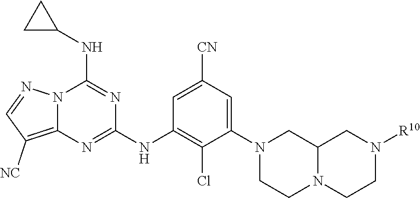

| | | LC/MS [M + H]⁺/ |
|---|---|---|
| Example # | R¹⁰ | RT |
| 158 | 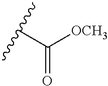 [SMILES: *C(=O)OC] | 548.2 1.8 min |
| 159 | 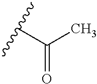 [SMILES: *C(C)=O] | 532.1 1.53 min |
| 160 | 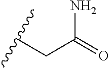 [SMILES: *CC(N)=O] | 547.2 1.46 min |
| 161 | 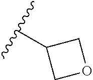 [SMILES: *C1COC1] | 546.2 1.56 min |
| 162 | 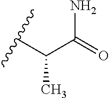 [SMILES: *[C@@H](C)C(N)=O] | 561.2 1.51 min |
| 163 | 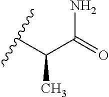 [SMILES: *[C@H](C)C(N)=O] | 561.2 1.51 min |
| 164 | 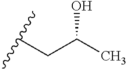 [SMILES: *C[C@@H](C)O] | 548.1 1.53 min |
| 165 | 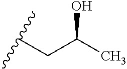 [SMILES: *C[C@H](C)O] | 548.2 1.55 min |
| 166 | 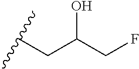 [SMILES: *CC(O)CF] | 566.2 1.58 min |

The following compounds were prepared from 4-(cyclopropyl(4-methoxybenzyl)amino)-2-(methylsulfonyl)pyrazolo[1,5-a][1,3,5]triazine-8-carbonitrile and tert-butyl 3-(4-(3-amino-2-chloro-5-cyanophenyl)piperazin-1-yl)pyrrolidine-1-carboxylate instead of (tert-Butyl 4-(3-amino-2-chloro-5-cyanophenyl)piperazine-1-carboxylate) in a similar procedure as described for Example 1 or Example 13 or Example 27 employing the R^{b} group identified in the table below:

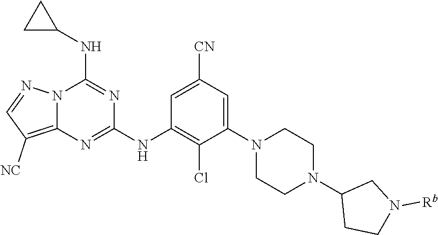

| | | LC/MS [M + H]⁺/ |
|---|---|---|
| Example # | R^{b} | RT |
| 167 | 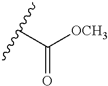 [SMILES: *C(=O)OC] | 562.2 1.94 min |
| 168 | 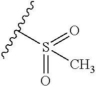 [SMILES: *S(C)(=O)=O] | 582.1 1.86 min |
| 169 | 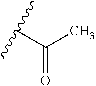 [SMILES: *C(C)=O] | 546.2 1.66 min |
| 170 | 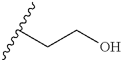 [SMILES: *CCO] | 548.1 1.43 min |
| 171 | 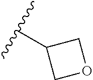 [SMILES: *C1COC1] | 560.2 1.58 min |
| 172 | 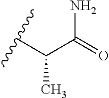 [SMILES: *[C@@H](C)C(N)=O] | 575.2 1.57 min |
| 173 | 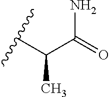 [SMILES: *[C@H](C)C(N)=O] | 575.2 1.57 min |
| 174 | 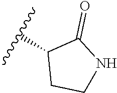 [SMILES: *[C@H]1CCNC1=O] | 587.3 1.55 min |
| 175 | 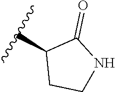 [SMILES: *[C@@H]1CCNC1=O] | 587.2 1.52 min |
| 176 | 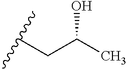 [SMILES: *C[C@@H](C)O] | 562.2 1.54 min |
| 177 | 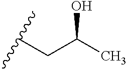 [SMILES: *C[C@H](C)O] | 562.2 1.55 min |
| 178 | 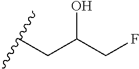 [SMILES: *CC(O)CF] | 580.2 1.62 min |
| 179 | 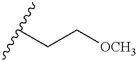 [SMILES: *CCOC] | 562.3 1.7 min |

The following compounds were prepared from 2-({2-chloro-5-cyano-3-[(2S)-2-methyl piperazin-1-yl]phenyl}amino)-4-(cyclopropylamino)pyrazolo[1,5-a][1,3,5]triazine-8-carbonitrile in a similar procedure as described for Example 1 or Example 27 employing the R⁵ group identified in the table below:

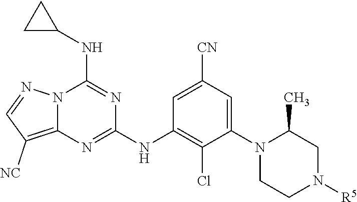

| | | LC/MS [M + H]⁺/ |
|---|---|---|
| Example # | R⁵ | RT |
| 180 | 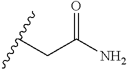 [SMILES: *CC(N)=O] | 506 1.66 min |
| 181 | 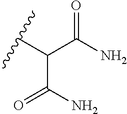 [SMILES: *C(C(N)=O)C(N)=O] | 549.2 1.49 min |
| 182 | 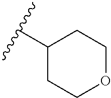 [SMILES: *C1CCOCC1] | 533.2 1.92 min |
| 183 | 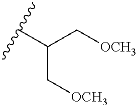 [SMILES: *C(COC)COC] | 551.2 2.05 min |

### Example 184 and Example 185

### 2-({2-chloro-5-cyano-3-[(2S)-2-methyl-4-[(3R)-oxolan-3-yl]piperazin-1-yl]phenyl}amino)-4-(cyclopropylamino)pyrazolo[1,5-a][1,3,5]triazine-8-carbonitrile and 2-({2-chloro-5-cyano-3-[(2S)-2-methyl-4-[(3S)-oxolan-3-yl]piperazin-1-yl]phenyl}amino)-4-(cyclopropylamino)pyrazolo[1,5-a][1,3,5]triazine-8-carbonitrile

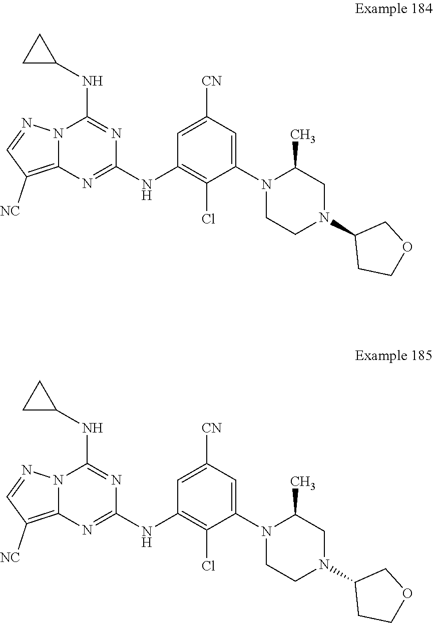 [SMILES: C[C@H]1CN([C@@H]2CCOC2)CCN1c1cc(C#N)cc(Nc2nc(NC3CC3)n3ncc(C#N)c3n2)c1Cl ; C[C@H]1CN([C@H]2CCOC2)CCN1c1cc(C#N)cc(Nc2nc(NC3CC3)n3ncc(C#N)c3n2)c1Cl]

A solution of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (17.96 mg, 0.04 mmol), dihydrofuran-3(2H)-one (10.33 mg, 0.120 mmol)) and acetic acid (1.145 μL, 0.020 mmol) in DMF (1 mL) was stirred at rt for 2 h. Sodium triacetoxyborohydride (25.4 mg, 0.120 mmol) was added at rt and the contents stirred for 16 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative SFC employing the following conditions: Column: Chiral AD-H, 30 mm×250 mm, 5-μm particles; Flow Rate: 100 mL/min; Column Temperature: 50° C. Fraction collection was triggered by UV (220 nm). Fractions containing the desired product were combined and dried via centrifugal evaporation to give 2-({2-chloro-5-cyano-3-[(2S)-2-methyl-4-[(3R)-oxolan-3-yl]piperazin-1-yl]phenyl}amino)-4-(cyclopropylamino)pyrazolo[1,5-a][1,3,5]triazine-8-carbonitrile (2.7 mg, 5.09 μmol, 12.73% yield) as Example 184 and 2-({2-chloro-5-cyano-3-[(2S)-2-methyl-4-[(3S)-oxolan-3-yl]piperazin-1-yl]phenyl}amino)-4-(cyclopropylamino)pyrazolo[1,5-a][1,3,5]triazine-8-carbonitrile (2.8 mg, 5.39 μmol, 13.49% yield) as Example 185. The stereochemistry on the carbon atom anchoring the tetrahydrofuran group to the piperazine nitrogen in Example 184 and Example 185 was tentatively assigned as shown. Example 184 analytical data: Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.9%; Observed Mass: 519.2; RT: 1.39 min.

Example 185 analytical data: Analytical Reverse Phase chromatography was used to determine the final purity. Injection conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 519.2; RT: 1.92 min.

### Example 186 and Example 187

### (2R)-3-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]-2-hydroxypropanamide and (2S)-3-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]-2-hydroxypropanamide

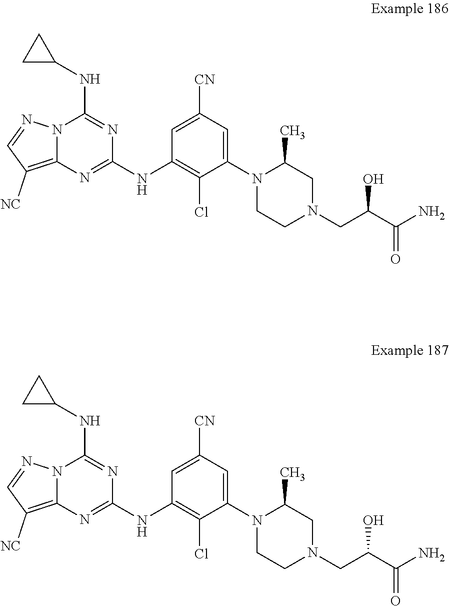 [SMILES: C[C@H]1CN(C[C@@H](O)C(N)=O)CCN1c1cc(C#N)cc(Nc2nc(NC3CC3)n3ncc(C#N)c3n2)c1Cl ; C[C@H]1CN(C[C@H](O)C(N)=O)CCN1c1cc(C#N)cc(Nc2nc(NC3CC3)n3ncc(C#N)c3n2)c1Cl]

To solution of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (9.88 mg, 0.022 mmol) in DMF (1 mL) was added K₂CO₃ (6.08 mg, 0.044 mmol) followed by oxirane-2-carboxamide (1.916 mg, 0.022 mmol) at rt and the contents were stirred at rt for 2 h. After filtration, the crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The material was further purified via preparative SFC employing the following conditions: Column: Chiral OJ-H, 30 mm×250 mm, 5-μm particles; Flow Rate: 100 mL/min; Column Temperature: 50° C. Fraction collection was triggered by UV (220 nm). Fractions containing the desired product were combined and dried via centrifugal evaporation to give 4.33 mg of (2R)-3-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]-2-hydroxypropanamide as Example 186 and 3.7 mg of (2S)-3-[(3S)-4-(2-chloro-5-cyano-3-{[8-cyano-4-(cyclopropylamino)pyrazolo[1,5-a][1,3,5]triazin-2-yl]amino}phenyl)-3-methylpiperazin-1-yl]-2-hydroxypropanamide as Example 187. The stereochemistry of the hydroxy group in Example 186 and Example 187 was tentatively assigned as shown.

Example 186 analytical data: Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 100%; Observed Mass: 536.2; RT: 1.54 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 536.2; RT: 1.29 min.
Example 187 analytical data: Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 98.9%; Observed Mass: 536.2; RT: 1.54 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 536.2; RT: 1.3 min.

### Example 188 and Example 189

### 2-({2-chloro-5-cyano-3-[(2S)-4-[(2R)-3-fluoro-2-hydroxypropyl]-2-methylpiperazin-1-yl]phenyl}amino)-4-(cyclopropylamino)pyrazolo[1,5-a][1,3,5]triazine-8-carbonitrile and 2-({2-chloro-5-cyano-3-[(2S)-4-[(2S)-3-fluoro-2-hydroxypropyl]-2-methylpiperazin-1-yl]phenyl}amino)-4-(cyclopropylamino)pyrazolo[1,5-a][1,3,5]triazine-8-carbonitrile

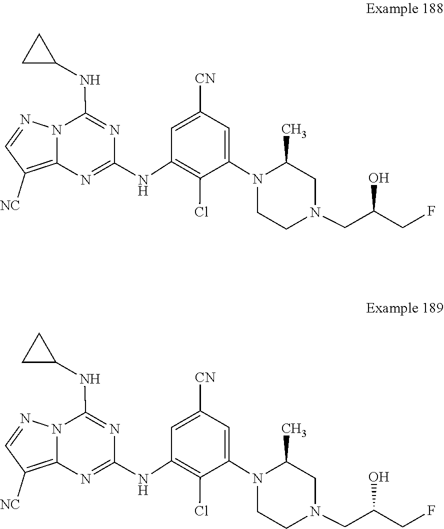 [SMILES: C[C@H]1CN(C[C@@H](O)CF)CCN1c1cc(C#N)cc(Nc2nc(NC3CC3)n3ncc(C#N)c3n2)c1Cl ; C[C@H]1CN(C[C@H](O)CF)CCN1c1cc(C#N)cc(Nc2nc(NC3CC3)n3ncc(C#N)c3n2)c1Cl]

A diastereomeric mixture of 2-((2-chloro-5-cyano-3-((2S)-4-(3-fluoro-2-hydroxypropyl)-2-methylpiperazin-1-yl)phenyl)amino)-4-(cyclopropylamino)pyrazolo[1,5-a][1,3,5]triazine-8-carbonitrile (20 mg, 0.038 mmol) was purified via preparative SFC chromatography employing the following conditions: Column: Chiralpak ADH, 30 mm×250 mm, 5 μm particles; Flow Rate: 100 mL/min; Column Temperature: 50° C. Fraction collection was triggered by UV (220 nm). Fractions containing the desired product were combined and dried via centrifugal evaporation to give 2-({2-chloro-5-cyano-3-[(2S)-4-[(2R)-3-fluoro-2-hydroxypropyl]-2-methylpiperazin-1-yl]phenyl}amino)-4-(cyclopropylamino)pyrazolo[1,5-a][1,3,5]triazine-8-carbonitrile (3.9 mg, 7.43 μmol, 19.50% yield) as Example 188 and 2-({2-chloro-5-cyano-3-[(2S)-4-[(2S)-3-fluoro-2-hydroxypropyl]-2-methylpiperazin-1-yl]phenyl}amino)-4-(cyclopropylamino)pyrazolo[1,5-a][1,3,5]triazine-8-carbonitrile (4.1 mg, 7.81 μmol, 20.50% yield) as Example 189. The stereochemistry of the hydroxy group in Example 188 and Example 189 was tentatively assigned as shown.

Example 188 analytical data: Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 525.2; RT: 1.87 min. Analytical SFC was used to determine the % ee. Conditions: Column: Chiral AD, 4.6 mm×100 mm, 5 μm particles; Mobile Phase A: CO₂; Mobile Phase B: MeOH with 0.1% DEA; Temperature: 50° C.; Isocratic elution at 35% B over 10 min; Flow: 2 mL/min; Detection: UV (220 nm). Injection 2 results: Purity: 99% ee; RT: 3.6 min.
Example 189 analytical data: Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 525.1; RT: 1.87 min. Analytical SFC was used to determine the % ee. Conditions: Column: Chiral AD, 4.6 mm×100 mm, 5 μm particles; Mobile Phase A: C₀₂; Mobile Phase B: MeOH with 0.1% DEA; Temperature: 50° C.; Isocratic elution at 35% B over 10 min; Flow: 2 mL/min; Detection: UV (220 nm). Injection 2 results: Purity: 99% ee; RT: 4 min.

### Example 190

### 2-({3-[(2S)-4-(1-acetylpyrrolidin-3-yl)-2-methylpiperazin-1-yl]-2-chloro-5-cyanophenyl}amino)-4-(cyclopropylamino)pyrazolo[1,5-a][1,3,5]triazine-8-carbonitrile

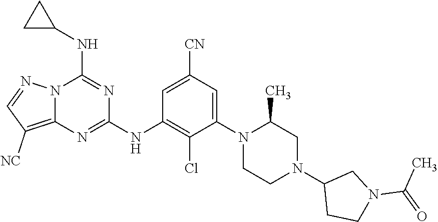 [SMILES: CC(=O)N1CCC(N2CCN(c3cc(C#N)cc(Nc4nc(NC5CC5)n5ncc(C#N)c5n4)c3Cl)[C@@H](C)C2)C1]

### Step 1. tert-butyl 3-((S)-4-(2-chloro-5-cyano-3-((8-cyano-4-(cyclopropylamino)pyrazolo[1,5-a][1,3,5]triazin-2-yl)amino)phenyl)-3-methylpiperazin-1-yl)pyrrolidine-1-carboxylate

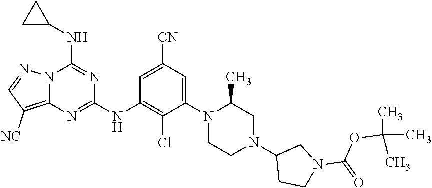 [SMILES: C[C@H]1CN(C2CCN(C(=O)OC(C)(C)C)C2)CCN1c1cc(C#N)cc(Nc2nc(NC3CC3)n3ncc(C#N)c3n2)c1Cl]

A solution of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (180 mg, 0.4 mmol), tert-butyl 3-oxopyrrolidine-1-carboxylate (222 mg, 1.200 mmol) and acetic acid (0.011 mL, 0.200 mmol) in DMF (2 mL) was stirred for 2 h at rt. Sodium triacetoxyborohydride (254 mg, 1.200 mmol) was added and the contents stirred for 16 h at RT. The reaction was diluted with water. The precipitate was collected washed with water and dried to give 201 mg of tert-butyl 3-((S)-4-(2-chloro-5-cyano-3-((8-cyano-4-(cyclopropylamino) pyrazolo[1,5-a][1,3,5]triazin-2-yl)amino)phenyl)-3-methylpiperazin-1-yl)pyrrolidine-1-carboxylate. LC/MS [M+H]⁺=618.3.

### Step 2. 2-((2-chloro-5-cyano-3-((2S)-2-methyl-4-(pyrrolidin-3-yl)piperazin-1-yl)phenyl)amino)-4-(cyclopropylamino)pyrazolo[1,5-a][1,3,5]triazine-8-carbonitrile

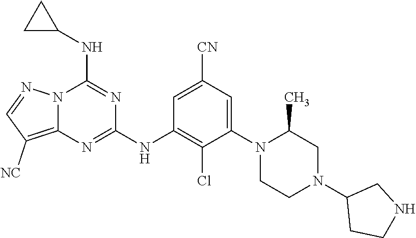 [SMILES: C[C@H]1CN(C2CCNC2)CCN1c1cc(C#N)cc(Nc2nc(NC3CC3)n3ncc(C#N)c3n2)c1Cl]

To a solution tert-butyl 3-((S)-4-(2-chloro-5-cyano-3-((8-cyano-4-(cyclopropylamino)pyrazolo[1,5-a][1,3,5]triazin-2-yl)amino)phenyl)-3-methylpiperazin-1-yl)pyrrolidine-1-carboxylate (350 mg, 0.566 mmol) in 3 mL of DCM was added 2 mL of TFA and the contents stirred at rt for 0.5 h. The reaction mixture was concentrated to give 2-((2-chloro-5-cyano-3-((2S)-2-methyl-4-(pyrrolidin-3-yl)piperazin-1-yl)phenyl)amino)-4-(cyclopropylamino)pyrazolo[1,5-a][1,3,5]triazine-8-carbonitrile (180 mg, 0.347 mmol, 61.4% yield) as white solid. LC/MS [M+H]⁺=518.4.

### Step 3. Example 190

To a solution of 2-((2-chloro-5-cyano-3-((2S)-2-methyl-4-(pyrrolidin-3-yl)piperazin-1 yl)phenyl)amino)-4-(cyclopropylamino)pyrazolo[1,5-a][1,3,5]triazine-8-carbonitrile (31.1 mg, 0.06 mmol) and Hunig's base (0.031 mL, 0.180 mmol) in DMF (1 mL) was added acetic anhydride (6.13 mg, 0.060 mmol). After stirring for 0.5 h, the reaction mixture was concentrated and purified by reverse phase column chromatography to give 13.8 mg of 2-({3-[(2S)-4-(1-acetylpyrrolidin-3-yl)-2-methylpiperazin-1-yl]-2-chloro-5-cyanophenyl}amino)-4-(cyclopropylamino)pyrazolo[1,5-a][1,3,5]triazine-8-carbonitrile as a mixture of diastereomers. Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 91.5%; Observed Mass: 560.2; RT: 1.64 min.

### Example 191 and Example 192

### 2-({3-[(2S)-4-[(3R)-1-acetylpyrrolidin-3-yl]-2-methylpiperazin-1-yl]-2-chloro-5-cyanophenyl}amino)-4-(cyclopropylamino)pyrazolo[1,5-a][1,3,5]triazine-8-carbonitrile and 2-({3-[(2S)-4-[(3S)-1-acetylpyrrolidin-3-yl]-2-methylpiperazin-1-yl]-2-chloro-5-cyanophenyl}amino)-4-(cyclopropylamino)pyrazolo[1,5-a][1,3,5]triazine-8-carbonitrile

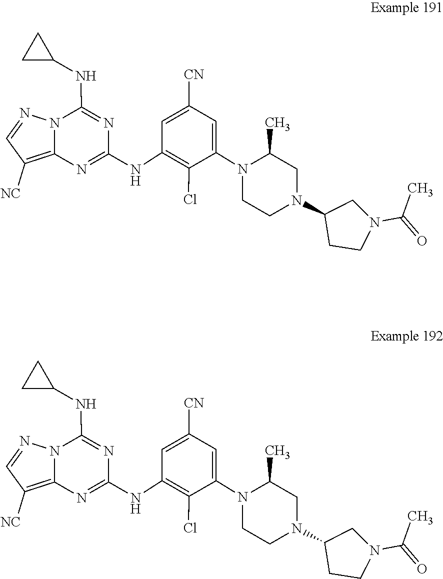 [SMILES: CC(=O)N1CC[C@@H](N2CCN(c3cc(C#N)cc(Nc4nc(NC5CC5)n5ncc(C#N)c5n4)c3Cl)[C@@H](C)C2)C1 ; CC(=O)N1CC[C@H](N2CCN(c3cc(C#N)cc(Nc4nc(NC5CC5)n5ncc(C#N)c5n4)c3Cl)[C@@H](C)C2)C1]

A diastereomer mixture of 2-({3-[(2S)-4-(1-acetylpyrrolidin-3-yl)-2-methylpiperazin-1-yl]-2-chloro-5-cyanophenyl}amino)-4-(cyclopropylamino) pyrazolo[1,5-a][1,3,5]triazine-8-carbonitrile (13.8 mg) was purified via preparative SFC employing the following conditions: Column: Chiralcel OJ-H, 30 mm×250 mm, 5 μm particles; Flow Rate: 100 mL/min; Column Temperature: 50° C. Fraction collection was triggered by UV (220 nm).

Fractions containing the desired product were combined and dried via centrifugal evaporation to give 3.2 mg 2-({3-[(2S)-4-[(3R)-1-acetylpyrrolidin-3-yl]-2-methylpiperazin-1-yl]-2-chloro-5-cyanophenyl}amino)-4-(cyclopropyl amino)pyrazolo[1,5-a][1,3,5]triazine-8-carbonitrile as Example 190 and 3.3 mg of 2-({3-[(2S)-4-[(3S)-1-acetylpyrrolidin-3-yl]-2-methylpiperazin-1-yl]-2-chloro-5-cyanophenyl}amino)-4-(cyclopropylamino)pyrazolo[1,5-a][1,3,5]triazine-8-carbonitrile as Example 191. The stereochemistry on the carbon atom anchoring the pyrrolidine group to the piperazine nitrogen in Example 191 and Example 192 was tentatively assigned as shown.

Example 191 analytical data: Analytical Reverse Phase chromatography was used to determine the final purity. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 560.2; RT: 1.75 min. Analytical SFC was used to determine the % ee. Conditions: Column: Chiral OJ, 4.6 mm×100 mm, 5 μm particles; Mobile Phase A: C₀₂; Mobile Phase B: MeOH with 0.1% DEA; Temperature: 50° C.; Isocratic elution at 30% B over 10 min; Flow: 2 mL/min; Detection: UV (220 nm). Injection 2 results: Purity: 99% ee; RT: 2.3 min.
Example 192 analytical data: Analytical Reverse Phase chromatography was used to determine the final purity. Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 100%; Observed Mass: 560.2; RT: 1.77 min. Analytical SFC was used to determine the % ee. Conditions: Column: Chiral OJ, 4.6 mm×100 mm, 5 μm particles; Mobile Phase A: C₀₂; Mobile Phase B: MeOH with 0.1% DEA; Temperature: 50° C.; Isocratic elution at 30% B over 10 min; Flow: 2 mL/min; Detection: UV (220 nm). Injection 2 results: Purity: 99% ee; RT: 2.8 min.

The following compounds were prepared from 2-((2-chloro-5-cyano-3-((2S)-2-methyl-4-(pyrrolidin-3-yl)piperazin-1-yl)phenyl)amino)-4-(cyclopropylamino)pyrazolo[1,5-a][1,3,5]triazine-8-carbonitrile in a similar procedure as described for Example 191 and Example 192 employing the R⁵ group identified in the table below:

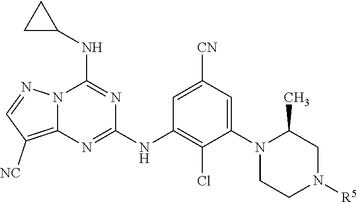

| | | LC/MS [M + H]⁺/ |
|---|---|---|
| Example # | R⁵ | RT |
| 193 | 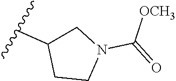 [SMILES: *C1CCN(C(=O)OC)C1] | 576.1 1.98 min |
| 194 | 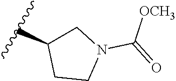 [SMILES: *[C@@H]1CCN(C(=O)OC)C1] | 576.3 2.01 min |
| 195 | 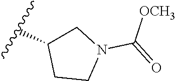 [SMILES: *[C@H]1CCN(C(=O)OC)C1] | 576.2 2.02 min |

### Example 196

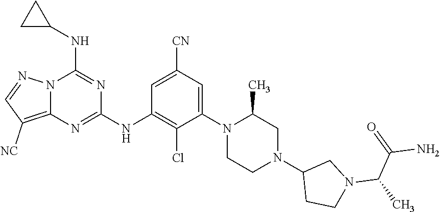 [SMILES: C[C@@H](C(N)=O)N1CCC(N2CCN(c3cc(C#N)cc(Nc4nc(NC5CC5)n5ncc(C#N)c5n4)c3Cl)[C@@H](C)C2)C1]

Example 196 as a mixture of diastereomers (at the carbon anchoring the pyrrolidine group to the piperazine nitrogen) was prepared from 2-((2-chloro-5-cyano-3-((2S)-2-methyl-4-(pyrrolidin-3-yl)piperazin-1-yl)phenyl) amino)-4-(cyclopropylamino)pyrazolo[1,5-a][1,3,5]triazine-8-carbonitrile in a similar procedure as described for Example 1. Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.8%; Observed Mass: 589.2; RT: 1.67 min.

### Example 197

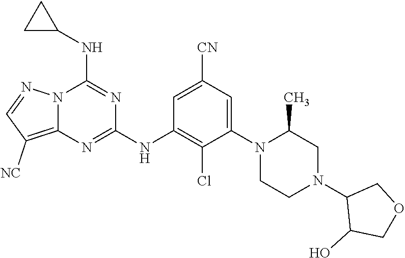 [SMILES: C[C@H]1CN(C2COCC2O)CCN1c1cc(C#N)cc(Nc2nc(NC3CC3)n3ncc(C#N)c3n2)c1Cl]

Example 197 as a mixture of diastereomers was prepared from 2-((2-chloro-5-cyano-3-((2S)-2-methyl-4-(pyrrolidin-3-yl)piperazin-1-yl)phenyl) amino)-4-(cyclopropylamino)pyrazolo[1,5-a][1,3,5]triazine-8-carbonitrile in a similar procedure as described for Example 1. Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 97.2%; Observed Mass: 535.1; RT: 1.58 min.

### Example 198

### 2-({3-[(2S)-4-[2-(azetidin-1-yl)acetyl]-2-methylpiperazin-1-yl]-2-chloro-5-cyanophenyl}amino)-4-(cyclopropylamino)pyrazolo[1,5-a][1,3,5]triazine-8-carbonitrile

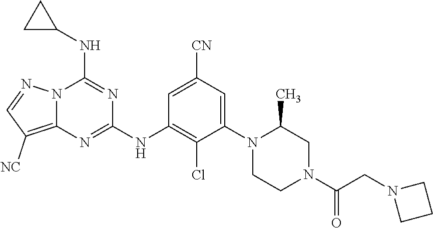 [SMILES: C[C@H]1CN(C(=O)CN2CCC2)CCN1c1cc(C#N)cc(Nc2nc(NC3CC3)n3ncc(C#N)c3n2)c1Cl]

A mixture of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (0.012 mL, 0.067 mmol), HATU (12.71 mg, 0.033 mmol) and 2-(azetidin-1-yl)acetic acid (3.85 mg, 0.033 mmol) in DMF (0.5 mL) was stirred at rt for 2 h. The reaction mixture was concentrated and purified by reverse phase chromatography to afford 2-({3-[(2S)-4-[2-(azetidin-1-yl)acetyl]-2-methylpiperazin-1-yl]-2-chloro-5-cyanophenyl}amino)-4-(cyclopropylamino) pyrazolo[1,5-a][1,3,5]triazine-8-carbonitrile (3.9 mg, 6.84 μmol, 30.7% yield). Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 95.8%; Observed Mass: 546; RT: 1.59 min.

The following compounds were prepared from 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile in a similar procedure as described for Example 198 employing the R⁵ group identified in the table below:

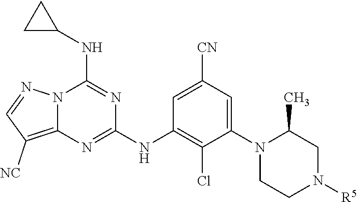

| | | LC/MS [M + H]⁺/ |
|---|---|---|
| Example # | R⁵ | RT |
| 199 | 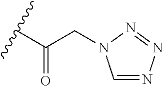 [SMILES: *C(=O)Cn1cnnn1] | 559.1 1.68 min. |
| 200 | 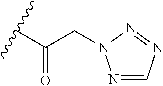 [SMILES: *C(=O)Cn1ncnn1] | 558.9 1.88 min. |
| 201 | 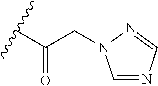 [SMILES: *C(=O)Cn1cncn1] | 558.1 1.64 min. |
| 202 | 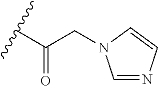 [SMILES: *C(=O)Cn1ccnc1] | 557.2 1.62 min. |
| 203 | 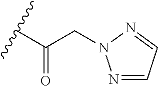 [SMILES: *C(=O)Cn1nccn1] | 558.1 1.86 min. |

### Example 204

### (2R)-2-[(2S)-4-(2-chloro-5-cyano-3-{[8-cyano-4-(ethylamino)pyrazolo[1,5-a][1,3,5]triazin-2-yl]amino}phenyl)-2-methylpiperazin-1-yl]propanamide

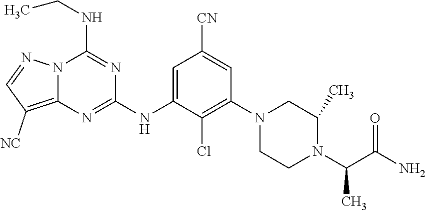 [SMILES: CCNc1nc(Nc2cc(C#N)cc(N3CCN([C@H](C)C(N)=O)[C@@H](C)C3)c2Cl)nc2c(C#N)cnn12]

Example 204 was prepared in a similar procedure as described for Example 1. Analytical LC/MS was used to determine the final purity. Injection 1 conditions: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). Injection 1 results: Purity: 100%; Observed Mass: 508.30; RT: 1.75 min.

### Example 205

### 2-[(2-chloro-5-cyano-3-{4-[(2R)-2-hydroxypropyl]piperazin-1-yl}phenyl)amino]-4-(ethylamino)pyrazolo[1,5-a][1,3,5]triazine-8-carbonitrile

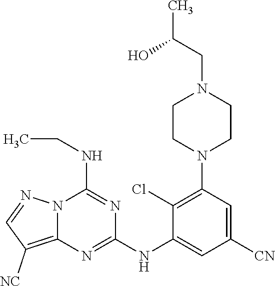 [SMILES: CCNc1nc(Nc2cc(C#N)cc(N3CCN(C[C@@H](C)O)CC3)c2Cl)nc2c(C#N)cnn12]

### Step 1. 2-{[2-chloro-5-cyano-3-(piperazin-1-yl)phenyl]amino}-4-{ethyl[(4-methoxyphenyl)methyl]amino}pyrazolo[1,5-a][1,3,5]triazine-8-carbonitrile

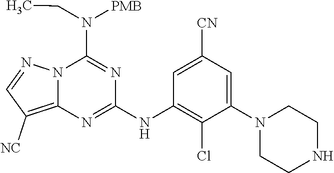

Tert-butyl 4-{2-chloro-5-cyano-3-[(8-cyano-4-{ethyl[(4-methoxyphenyl) methyl]amino}pyrazolo[1,5-a][1,3,5]triazin-2-yl)amino]phenyl}piperazine-1-carboxylate (0.3 g, 0.466 mmol) was dissolved in CH₂Cl₂ (5.83 mL) at rt. TFA (0.90 mL, 11.66 mmol) was added dropwise and the mixture was stirred at rt for 60 min. It was concentrated, evaporated with DCM (3×) and triturated with Et₂O. The Et₂₀ layer was decanted, the residue was air-dried to give 2-{[2-chloro-5-cyano-3-(piperazin-1-yl)phenyl]amino}-4-{ethyl[(4-methoxyphenyl) methyl]amino}pyrazolo[1,5-a][1,3,5]triazine-8-carbonitrile (0.30 g, 98%) as a TFA salt after drying. Used as such for the subsequent step. LC/MS [M+H]⁺=543.2.

### Step 2. Example 205

To a vial charged with 2-{[2-chloro-5-cyano-3-(piperazin-1-yl)phenyl]amino}-4-{ethyl[(4-methoxyphenyl)methyl]amino}pyrazolo[1,5-a][1,3,5]triazine-8-carbonitrile (0.05 g, 0.092 mmol) in acetonitrile (0.307 mL) was added triethylamine (0.19 mL, 0.92 mmol) and R(+)-propylene oxide (0.032 mL, 0.46 mmol). The reaction mixture was heated at 85° C. for 1 h. The reaction mixture was cooled to rt and partitioned between ethyl acetate and water. The organics were dried over anhydrous MgSO₄, filtered, and concentrated in vacuo. The above residue was dissolved in DCM (1 mL) and anisole (0.030 mL, 0.28 mmol) was added followed by TFA (1 mL). The reaction was heated at 40° C. for 3 h. The solvents were concentrated and dissolved in MeOH and filtered. The crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 10% B, 10-63% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 10.4 mg, and its estimated purity by LCMS analysis was 98%. Analytical LC/MS was used to determine the final purity. LC-MS Method A: Purity: 97.9%; Observed Mass: 481.20; RT: 1.62 min. LC-MS Method B: Column: Purity: 98%; Observed Mass: 481.20; RT: 1.23 min. ¹H NMR (500 MHz, DMSO-d₆) δ 9.56 (s, 1H), 9.05 (m, 1H), 8.41 (s, 1H), 7.91 (d, J=1.8 Hz, 1H), 7.40 (d, J=1.5 Hz, 1H), 3.81 (br d, J=5.8 Hz, 1H), 3.04 (br s, 2H), 2.55 (DMSO), 2.65-2.57 (m, 2H), 2.37-2.20 (m, 2H), 1.18 (s, 2H), 1.08 (d, J=6.1 Hz, 2H) ppm (H₂O suppressed at 3.33 ppm).

### Example 206

### 2-({2-chloro-5-cyano-3-[4-(oxetan-3-yl)piperazin-1-yl]phenyl}amino)-4-(ethylamino)pyrazolo[1,5-a][1,3,5]triazine-8-carbonitrile

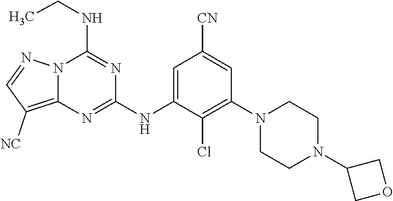 [SMILES: CCNc1nc(Nc2cc(C#N)cc(N3CCN(C4COC4)CC3)c2Cl)nc2c(C#N)cnn12]

To a solution of 2-{[2-chloro-5-cyano-3-(piperazin-1-yl)phenyl]amino}-4-{ethyl[(4-methoxyphenyl)methyl]amino}pyrazolo[1,5-a][1,3,5]triazine-8-carbonitrile (40 mg, 0.074 mmol) in methanol (1.5 mL) was added trimethyl orthoformate (0.5 mL, 4.52 mmol), oxetan-3-one (0.043 mL, 0.74 mmol) and acetic acid (0.017 mL, 0.30 mmol). The mixture was stirred at rt for 45 min. Sodium cyanoborohydride (1M in THF, 0.59 mL, 0.59 mmol) was added and the mixture was stirred at rt for 2 days. It was partitioned between EtOAc and dilute aq. NaHCO₃ solution. The organic layer was washed with brine, and the solvents were evaporated. To the residue was added DCE (1 mL), anisole (0.024 mL, 0.22 mmol), followed by TFA (0.5 mL). The resulting mixture was heated at 50° C. for 3 h. It was cooled, concentrated, dissolved in MeOH, filtered. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 12% B, 12-52% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 9.7 mg, and its estimated purity by LCMS analysis was 95%. Analytical LC/MS was used to determine the final purity. LC-MS Method A: Purity: 94.7%; Observed Mass: 479.10; RT: 1.8 min. LC-MS Method B: Purity: 99.2%; Observed Mass: 479.20; RT: 1.33 min. ¹H NMR (500 MHz, DMSO-d₆) δ 9.57 (s, 1H), 9.05 (m, 1H), 8.40 (s, 1H), 7.92 (s, 1H), 7.43 (s, 1H), 4.57 (t, J=6.3 Hz, 2H), 4.51-4.39 (m, 1H), 3.07 (br s, 2H), 2.55 (DMSO), 2.48-2.39 (m, 2H), 1.26-1.08 (m, 3H) ppm (H₂O suppressed at 3.33 ppm).

### Example 207

### 2-({2-chloro-5-cyano-3-[4-(1-methylazetidin-3-yl)piperazin-1-yl]phenyl}amino)-4-(ethylamino)pyrazolo[1,5-a][1,3,5]triazine-8-carbonitrile

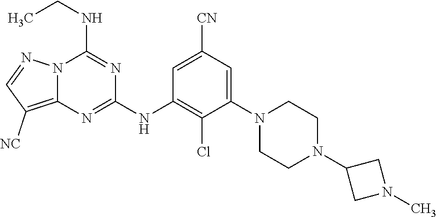 [SMILES: CCNc1nc(Nc2cc(C#N)cc(N3CCN(C4CN(C)C4)CC3)c2Cl)nc2c(C#N)cnn12]

### Step 1. tert-butyl 3-(4-{2-chloro-5-cyano-3-[(8-cyano-4-{cyclopropyl[(4-methoxyphenyl)methyl]amino}pyrazolo[1,5-a][1,3,5]triazin-2-yl)amino]phenyl}piperazin-1-yl)azetidine-1-carboxylate

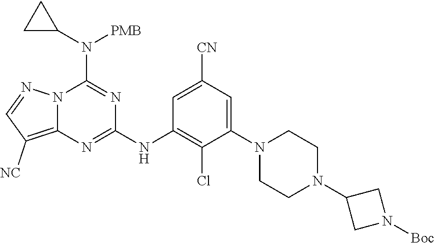

A solution of 2-{[2-chloro-5-cyano-3-(piperazin-1-yl)phenyl]amino}-4-{cyclopropyl[(4-methoxyphenyl)methyl]amino}pyrazolo[1,5-a][1,3,5]triazine-8-carbonitrile (95 mg, 0.55 mmol) and acetic acid (0.063 mL, 1.11 mmol) in DCE (2 mL) was stirred at rt for 1 h. Sodium triacetoxyborohydride (146 mg, 0.69 mmol) was added and the reaction mixture was stirred at room temperature for 20 h. The reaction mixture was diluted with EtOAc, washed with sat'd NaHCO₃, brine, dried over MgSO₄, filtered and concentrated to give crude tert-butyl 3-(4-{2-chloro-5-cyano-3-[(8-cyano-4-{cyclopropyl[(4-methoxyphenyl)methyl]amino}pyrazolo[1,5-a][1,3,5]triazin-2-yl)amino]phenyl}piperazin-1-yl)azetidine-1-carboxylate (228 mg) as tan solids containing a small amount of unreacted tert-butyl 3-oxoazetidine-1-carboxylate. Used as it in the next step. LC/MS [M+H]⁺=710.7.

### Step 2. 2-({3-[4-(azetidin-3-yl)piperazin-1-yl]-2-chloro-5-cyanophenyl}amino)-4-(cyclopropylamino)pyrazolo[1,5-a][1,3,5]triazine-8-carbonitrile

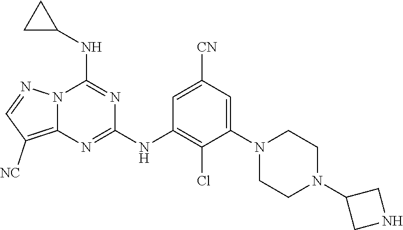 [SMILES: N#Cc1cc(Nc2nc(NC3CC3)n3ncc(C#N)c3n2)c(Cl)c(N2CCN(C3CNC3)CC2)c1]

The above crude material (228 mg, 0.32 mmol) was stirred in CH₂Cl₂ (3 mL) and TFA (3 mL) was added. The reaction was stirred at rt for 1 h. The mixture was concentrated and dried under vacuum. To the crude was added ClCH₂CH₂Cl (3 mL), anisole (0.11 mL, 0.96 mmol) followed by TFA (1.24 mL, 16.05 mmol). The reaction was stirred at 50° C. for 2 h. It was concentrated, evaporated with DCM (3×), dried under vacuum, and triturated with Et₂₀ (3×). The solids were dried and used directly for the next step. A small aliquot was subjected to preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 8% B, 8-48% B over 23 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 4.8 mg, and its estimated purity by LCMS analysis was 92%. Analytical LC/MS was used to determine the final purity. LC-MS Method A: Purity: 92.2%; Observed Mass: 490.30; RT: 1.43 min. LC-MS Method B: Purity: 94.8%; Observed Mass: 490.30; RT: 1.29 min. ¹H NMR (500 MHz, DMSO-d₆) δ 8.47-8.33 (m, 2H), 8.09 (m, 1H), 7.41 (d, J=1.7 Hz, 1H), 3.90-3.69 (m, 3H), 3.14-2.79 (m, 3H), 2.55 (DMSO), 0.85-0.78 (m, 2H), 0.78-0.73 (m, 2H) ppm (H₂O suppressed at 3.33 ppm).

### Step 3. Example 207

To a solution of the above solids (25 mg, 0.052 mmol) in MeOH (0.7 mL) and acetic acid (0.015 mL, 0.262 mmol) was added formaldehyde (21.22 mg, 0.26 mmol) followed by sodium triacetoxyborohydride (27.7 mg, 0.13 mmol). The resulting solution was stirred at RT for 2 h. It was partitioned between EtOAc and aq. NaHCO₃. The organic layer was washed with water, concentrated, dissolved in MeOH, and filtered. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 5% B, 5-45% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 4.9 mg, and its estimated purity by LCMS analysis was 93%. Analytical LC/MS was used to determine the final purity. LC-MS Method A: Purity: 93.7%; Observed Mass: 492.20; RT: 1.38 min. LC-MS Method B: Purity: 93%; Observed Mass: 492.30; RT: 1.29 min. ¹H NMR (500 MHz, DMSO-d₆) δ 9.58 (s, 1H), 9.06 (br t, J=5.8 Hz, 1H), 8.40 (s, 1H), 7.93 (d, J=1.5 Hz, 1H), 7.44 (d, J=1.5 Hz, 1H), 4.68-3.78 (m, 4H), 3.10 (br s, 2H), 2.87 (s, 1H), 2.99-2.78 (m, 1H), 2.70-2.57 (m, 2H), 2.55 (DMSO), 1.23-1.15 (m, 3H) ppm (H₂O suppressed at 3.33 ppm).

### Example 208

### 2-[(2-Chloro-5-cyano-3-{3,6-diazabicyclo[3.1.1]heptan-3-yl}phenyl)amino]-4-(ethylamino)pyrazolo[1,5-a][1,3,5]triazine-8-carbonitrile

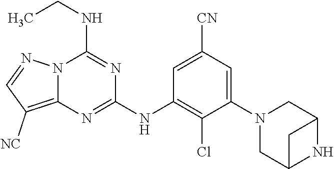 [SMILES: CCNc1nc(Nc2cc(C#N)cc(N3CC4CC(C3)N4)c2Cl)nc2c(C#N)cnn12]

### Step 1. Tert-butyl 3-(3-{bis[(tert-butoxy)carbonyl]amino}-2-chloro-5-cyanophenyl)-3,6-diazabicyclo[3.1.1]heptane-6-carboxylate

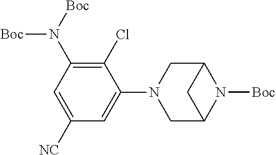 [SMILES: CC(C)(C)OC(=O)N(C(=O)OC(C)(C)C)c1cc(C#N)cc(N2CC3CC(C2)N3C(=O)OC(C)(C)C)c1Cl]

A stirred solution of tert-butyl N-(3-bromo-2-chloro-5-cyanophenyl)-N-[(tert-butoxy)carbonyl]carbamate (646 mg, 1.50 mmol) in dioxane (10 mL) was treated with tert-butyl 3,6-diazabicyclo[3.1.1]heptane-6-carboxylate (356 mg, 1.80 mmol), followed by Pd₂(dba)₃ (206 mg, 0.22 mmol), BINAP (168 mg, 0.27 mmol) and Cs₂CO₃ (1024 mg, 3.14 mmol). The reaction was purged with nitrogen for 3 min, then capped and stirred at 105° C. for 16 h. The reaction was diluted with ethyl acetate, the solid was removed by filtration, and the filtrate was concentrated. The crude was purified by ISCO silica column (40 g), eluted with ethyl acetate: hexanes using a 0-100% gradient. The desired fractions were concentrated to yield tert-butyl 3-(3-{bis[(tert-butoxy)carbonyl]amino}-2-chloro-5-cyanophenyl)-3,6-diazabicyclo[3.1.1]heptane-6-carboxylate (660 mg, 1.20 mmol, 80% yield) as light brown film. LC/MS [M+Na]⁺=571, RT=2.00 min (Method C).

### Step 2. Tert-butyl 3-(3-amino-2-chloro-5-cyanophenyl)-3,6-diazabicyclo[3.1.1]heptane-6-carboxylate

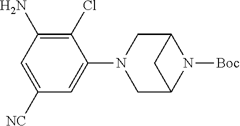 [SMILES: CC(C)(C)OC(=O)N1C2CC1CN(c1cc(C#N)cc(N)c1Cl)C2]

The above product of Step 1 (660 mg, 1.202 mmol) was stirred in DCM (10 mL) and TFA (8 mL) was added. The mixture was stirred at rt for 3 h. It was concentrated, evaporated with DCM (2×), then triturated with Et₂O to give tert-butyl 3-(3-amino-2-chloro-5-cyanophenyl)-3,6-diazabicyclo[3.1.1]heptane-6-carboxylate as TFA salts as light tan solids (0.45 g). To a solution of 3-amino-5-(3,6-diazabicyclo[3.1.1]heptan-3-yl)-4-chlorobenzonitrile (345 mg, 0.72 mmol) in DCM (5 mL) at 0° C. was added TEA (0.30 mL, 2.17 mmol) and then Boc₂O (0.17 mL, 0.72 mmol) in DCM (1 mL) dropwise and the reaction mixture became clear and stirred at RT for 15-20 min. The reaction mixture was concentrated, and the residue was purified by ISCO (hexanes/EtOAc) to give tert-butyl 3-(3-amino-2-chloro-5-cyanophenyl)-3,6-diazabicyclo[3.1.1]heptane-6-carboxylate (180 mg, 0.52 mmol, 71.3% yield) as a tan film. LC/MS [M+H]⁺=293.1, RT=1.59 min (Method C). ¹H NMR (500 MHz, CHLOROFORM-d) δ 6.93-6.85 (m, 1H), 6.80-6.70 (m, 1H), 4.38-4.29 (m, 2H), 4.27-4.17 (m, 2H), 3.81-3.70 (m, 2H), 3.59-3.46 (m, 1H), 2.64-2.51 (m, 1H), 1.90-1.84 (m, 2H), 1.52-1.47 (m, 9H) ppm.

### Step 3. Tert-butyl 3-{2-chloro-5-cyano-3-[(8-cyano-4-{ethyl[(4-methoxyphenyl)methyl]amino}pyrazolo[1,5-a][1,3,5]triazin-2-yl)amino]phenyl}-3,6-diazabicyclo[3.1.1]heptane-6-carboxylate

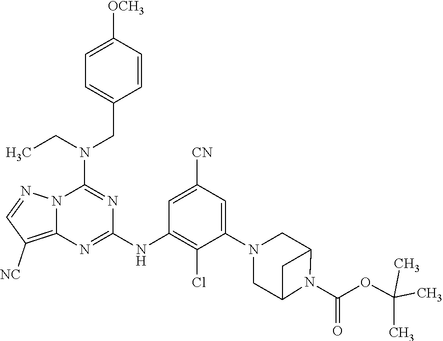 [SMILES: CCN(Cc1ccc(OC)cc1)c1nc(Nc2cc(C#N)cc(N3CC4CC(C3)N4C(=O)OC(C)(C)C)c2Cl)nc2c(C#N)cnn12]

To a solution of tert-butyl 3-(3-amino-2-chloro-5-cyanophenyl)-3,6-diazabicyclo[3.1.1]heptane-6-carboxylate (170 mg, 0.49 mmol) in THF (5 mL) at 0° C. was added potassium tert-butoxide (1.07 mL, 1.07 mmol) dropwise. After stirring 30 min at 0° C., to the solution was added a solution of 4-(ethyl(4-methoxybenzyl)amino)-2-(methylsulfonyl)pyrazolo[1,5-a][1,3,5]triazine-8-carbonitrile (188 mg, 0.487 mmol) in THF (5 mL) dropwise. After stirring for 1 h at 0° C., the reaction was quenched with sat'd NH₄Cl, extracted with EtOAc, washed with brine, dried over MgSO₄, filtered, concentrated. It was purified by flash chromatography eluting with ethyl acetate in hexanes to give the desired product as a light brown film (180 mg, 56%). LC/MS [M+H]⁺=655.3, RT=2.04 min (Method C).

### Step 4. Example 208

Tert-butyl 3-(2-chloro-5-cyano-3-((8-cyano-4-(ethyl(4-methoxybenzyl) amino)pyrazolo[1,5-a][1,3,5]triazin-2-yl)amino)phenyl)-3,6-diazabicyclo[3.1.1]heptane-6-carboxylate (180 mg, 0.275 mmol) was stirred in DCE (2 mL) at rt, anisole (0.150 mL, 1.374 mmol) was added followed by TFA (1.1 mL, 14 mmol). The reaction was stirred at 50° C. for 2 h. LCMS showed completion of the reaction. The reaction mixture was concentrated and azeotroped with DCM (3×). 7N NH₃ in MeOH (1 mL) was added and the contents concentrated. The residue was triturated with Et₂₀ (2×), and ether layer was decanted to give light brown solids. Used directly in the next step after drying. A small amount of the crude material was purified via preparative LC/MS employing the following conditions: Column: XBridge C18, 200 mm×19 mm, 5-μm particles; Mobile Phase A: 5:95 acetonitrile: water with ammonium acetate; Mobile Phase B: 95:5 acetonitrile: water with ammonium acetate; Gradient: a 0-min hold at 9% B, 9-49% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 2.8 mg, and its estimated purity by LCMS analysis was 99%.

Analytical LC/MS was used to determine the final purity. LC-MS Method A: Purity: 100%; Observed Mass: 435.20; RT: 1.31 min. LC-MS Method B: Purity: 99.1%; Observed Mass: 435.10; RT: 1.27 min. ¹H NMR (500 MHz, DMSO-d₆) δ 8.41 (s, 1H), 7.86 (d, J=1.5 Hz, 1H), 7.64 (d, J=0.9 Hz, 1H), 2.55 (DMSO), 2.49-2.41 (m, 1H), 1.97-1.89 (br s, 3H), 1.18 (t, J=7.2 Hz, 3H) ppm (H₂O suppressed at 3.33 ppm).

### Example 209

### Methyl 3-(2-chloro-5-cyano-3-{[8-cyano-4-(ethylamino)pyrazolo[1,5-a][1,3,5]triazin-2-yl]amino}phenyl)-3,6-diazabicyclo[3.1.1]heptane-6-carboxylate

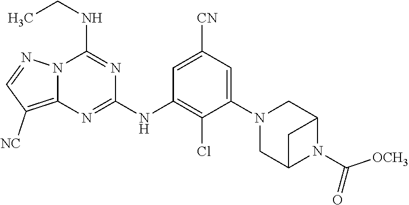 [SMILES: CCNc1nc(Nc2cc(C#N)cc(N3CC4CC(C3)N4C(=O)OC)c2Cl)nc2c(C#N)cnn12]

To the amine from Step 4. Example 208 (17 mg, 0.039 mmol) was added CH₂Cl₂ (1 mL). DIEA (0.015 mL, 0.086 mmol) was added followed by the addition of methyl carbonochloridate (5.91 mg, 0.063 mmol). The reaction was stirred at rt. Water was added to the reaction mixture and the contents concentrated. The residue was dissolved in DMF and filtered. The crude material was purified via preparative LC/MS employing the following conditions: Column: XBridge C18, 200 mm×19 mm, 5-μm particles; Mobile Phase A: 5:95 acetonitrile: water with 0.05% trifluoroacetic acid; Mobile Phase B: 95:5 acetonitrile: water with 0.05% trifluoroacetic acid; Gradient: a 0-min hold at 28% B, 28-68% B over 20 min, then a 0-min hold at 100% B; Flow Rate: 20 mL/min; Column Temperature: 25° C. Fraction collection was triggered by MS and UV signals. Fractions containing the desired product were combined and dried via centrifugal evaporation. The yield of the product was 3.9 mg, and its estimated purity by LCMS analysis was 95%. Analytical LC/MS was used to determine the final purity. LC-MS Method A: Purity: 97.4%; Observed Mass: 493.10; RT: 1.98 min. LC-MS Method B: Purity: 94.7%; Observed Mass: 493.20; RT: 1.99 min. ¹H NMR (500 MHz, DMSO-d₆) δ 9.58 (s, 1H), 9.04 (br. t, J=6.0 Hz, 1H), 8.40 (s, 1H), 7.89 (d, J=1.2 Hz, 1H), 7.65 (d, J=1.5 Hz, 1H), 4.22 (br. d, J=5.8 Hz, 2H), 3.83-3.67 (m, 2H), 3.60 (s, 2H), 3.03-2.78 (m, 1H), 2.55 (DMSO), 1.73 (d, J=8.2 Hz, 1H), 1.27-1.00 (m, 3H) ppm (H₂O suppressed at 3.33 ppm).

### Example 210

### 2-{[2-chloro-5-(difluoromethoxy)-3-[4-(oxetan-3-yl)piperazin-1-yl]phenyl]amino}-4-(cyclopropylamino)pyrazolo[1,5-a][1,3,5]triazine-8-carbonitrile

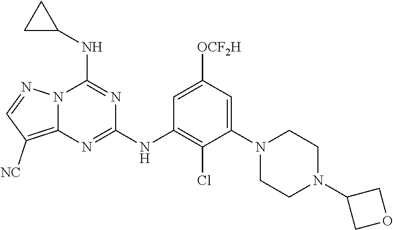

### Step 1. tert-butyl N-[3-bromo-2-chloro-5-(difluoromethoxy)phenyl]carbamate

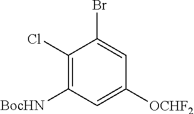 [SMILES: CC(C)(C)OC(=O)Nc1cc(OC(F)F)cc(Br)c1Cl]

To a solution of 1,3-dibromo-2-chloro-5-(difluoromethoxy)benzene (1.55 g, 4.61 mmol) in dioxane (30.7 mL) are added tert-butyl carbamate (0.43 g, 3.69 mmol), palladium(II) acetate (0.103 g, 0.461 mmol), xantphos (0.40 g, 0.69 mmol) and cesium carbonate (6.01 g, 18.43 mmol) at rt. The mixture was degassed with N₂ for 5 min. It was capped and heated to 105° C. After 3 h, it was cooled to rt, diluted with EtOAc, filtered through celite, concentrated, and purified by ISCO (80 g SiO₂, 0 to 40% EtOAc-hexane gradient elution). The fractions were concentrated and dried under vacuum to give white solids, tert-butyl (3-bromo-2-chloro-5-(difluoromethoxy)phenyl)carbamate (960 mg, 55.9% yield). LC/MS [M+H]⁺=611.4, RT=1.99 min (Method C). ¹H NMR (400 MHz, CHLOROFORM-d) δ 8.12 (d, J=2.6 Hz, 1H), 7.23-7.16 (m, 1H), 7.13 (d, J=2.7 Hz, 1H), 6.85-6.28 (m, 1H), 1.66-1.44 (m, 9H) ppm.

### Step 2. 3-bromo-2-chloro-5-(difluoromethoxy)aniline

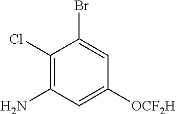

The product from Step 1 (960 mg) was stirred in DCM (5 mL) and TFA (4 mL) at rt for 1 h. The reaction mixture was concentrated, evaporated with DCM (3×), dissolved in EtOAc, and washed with sat'd NaHCO₃, brine, dried over MgSO₄, filtered, and concentrated to give white solids (590 mg, 84%). LC/MS [M+H]⁺=274.0, 276.0, RT=1.59 min (Method C).

### Step 3. 2-{[3-bromo-2-chloro-5-(difluoromethoxy)phenyl]amino}-4-{cyclopropyl[(4-methoxyphenyl)methyl]amino}pyrazolo[1,5-a][1,3,5]triazine-8-carbonitrile

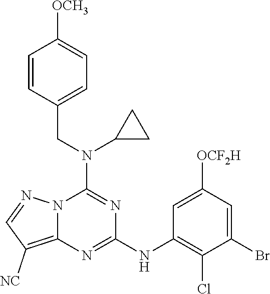

To a mixture of 3-bromo-2-chloro-5-(difluoromethoxy)aniline (0.59 g, 2.165 mmol) in THF (15 mL) was added LiHMDS (5.41 mL, 5.41 mmol) dropwise at −78° C. It was stirred at −78° C. for 30 min and then 4-{cyclopropyl[(4-methoxyphenyl)methyl]amino}-2-methanesulfonylpyrazolo[1,5-a][1,3,5]triazine-8-carbonitrile (0.86 g, 2.17 mmol) in THF (3 mL) was added and stirred at −78° C. for 2 h. The reaction was quenched with water, extracted with EtOAc. The organic layer was washed with water, brine, dried over MgSO₄, filtered and concentrated. The residue was washed with MeOH, and the solids were collected to give 2-((3-bromo-2-chloro-5-(difluoromethoxy)phenyl)amino)-4-(cyclopropyl(4-methoxybenzyl)amino)pyrazolo[1,5-a][1,3,5]triazine-8-carbonitrile (0.99 g, 1.68 mmol, 77% yield) as an off-white solid. LC/MS [M+H]⁺=590.3, 592.2, RT=2.09 min (Method C).

### Step 4. Example 210

A stirred solution of 2-((3-bromo-2-chloro-5-(difluoromethoxy)phenyl) amino)-4-(cyclopropyl(4-methoxybenzyl)amino)pyrazolo[1,5-a][1,3,5]triazine-8-carbonitrile (35 mg, 0.059 mmol) in dioxane (2 mL) was treated with 1-(oxetan-3-yl)piperazine (13.48 mg, 0.095 mmol), followed by Pd₂(dba)₃ (8.14 mg, 8.89 μmol), BINAP (6.64 mg, 10.66 μmol) and Cs₂CO₃ (57.9 mg, 0.178 mmol), purged with N₂ for 5 min, then stirred at 105° C. overnight. The reaction mixture was cooled to rt, diluted with EA. The solid was removed by filtration, the filtrate was concentrated. The crude residue was purified by ISCO silica column (12 g), eluting with EtOAc:Hexane 0-100% gradient over 12 min. The desired fractions were concentrated. The residue was dried under vacuum and DCE (1.5 mL) was added followed by anisole (0.021 mL, 0.19 mmol) and TFA (0.45 mL). The reaction mixture was heated with stirring at 45° C. for 30 min. LCMS showed completion of the reaction. The reaction mixture was concentrated, dissolved in DMF/MeOH, and filtered. The crude material was purified via preparative LC/MS employing the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-min hold at 30% B, 30-70% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 6.2 mg, and its estimated purity by LCMS analysis was 94%. LC-MS Method A: Purity: 93.9%; Observed Mass: 532.10; RT: 1.83 min. LC-MS Method B: Purity: 95.6%; Observed Mass: 532.20; RT: 1.44 min. ¹H NMR (500 MHz, DMSO-d₆) δ 9.20 (s, 1H), 9.09 (br. d, J=3.7 Hz, 1H), 8.34 (s, 1H), 7.57 (d, J=1.8 Hz, 1H), 7.38-6.97 (m, 2H), 6.76 (d, J=2.1 Hz, 1H), 4.61-4.52 (m, 2H), 4.48 (br. t, J=6.1 Hz, 2H), 3.69 (br. d, J=17.7 Hz, 4H), 3.17 (s, 1H), 3.03 (br. s, 4H), 2.46 (br. s, 4H), 0.84-0.64 (m, 4H). (H₂O suppressed at 3.33 ppm).

### Example 211

### (2-{[2-chloro-5-(difluoromethoxy)-3-{4-[(1R)-1-(5-oxo-4,5-dihydro-1,3,4-oxadiazol-2-yl)ethyl]piperazin-1-yl}phenyl]amino}-4-(cyclopropylamino)pyrazolo[1,5-a][1,3,5]triazine-8-carbonitrile

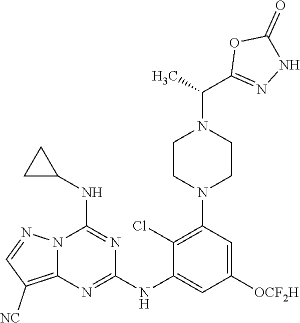

### Step 1. 2-{[2-chloro-5-(difluoromethoxy)-3-(piperazin-1-yl)phenyl]amino}-4-{cyclopropyl[(4-methoxyphenyl)methyl]amino}pyrazolo[1,5-a][1,3,5]triazine-8-carbonitrile

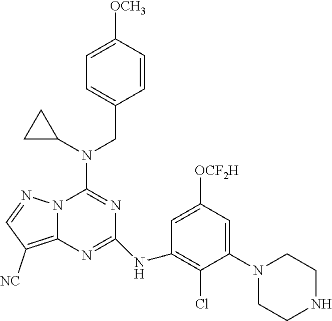

A stirred solution of 2-((3-bromo-2-chloro-5-(difluoromethoxy)phenyl) amino)-4-(cyclopropyl(4-methoxybenzyl)amino)pyrazolo[1,5-a][1,3,5]triazine-8-carbonitrile (350 mg, 0.59 mmol) in dioxane (8 mL) was treated with tert-butyl piperazine-1-carboxylate (221 mg, 1.19 mmol), followed by Pd₂(dba)₃ (81 mg, 0.089 mmol), BINAP (73.8 mg, 0.118 mmol) and Cs₂CO₃ (676 mg, 2.07 mmol), purged with N₂ for 5 min, then stirred at 105° C. overnight. It was diluted with EtOAc, the solid was removed by filtration, and the filtrate was concentrated. The crude residue was purified by ISCO silica column (12 g), eluting with 0-100% EtOAc in hexanes. The desired fractions were collected and concentrated to yield tert-butyl 4-(2-chloro-3-((8-cyano-4-(cyclopropyl(4-methoxybenzyl)amino)pyrazolo[1,5-a][1,3,5]triazin-2-yl)amino)-5-(difluoromethoxy)phenyl)piperazine-1-carboxylate. LC/MS [M+H]⁺=696.5, RT=2.13 min (Method C). The residue was dried under vacuum and DCM (2 mL) and TFA (2 mL) were added. The reaction was stirred at rt for 1 h. It was concentrated, evaporated with DCM (3×), dried under vacuum to give crude 2-{[2-chloro-5-(difluoromethoxy)-3-(piperazin-1-yl)phenyl]amino}-4-{cyclopropyl [(4-methoxyphenyl)methyl]amino}pyrazolo[1,5-a][1,3,5]triazine-8-carbonitrile (210 mg, 50%). Used as such for the subsequent step. LC/MS [M+H]⁺=596.5, RT=1.52 min (Method C).

### Step 2. methyl (2R)-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanoate

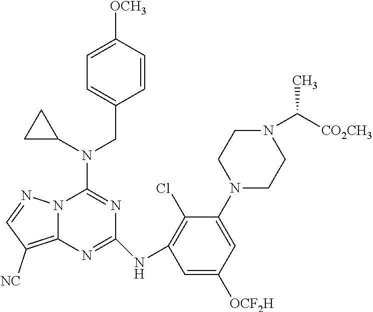

To a suspension of 2-((2-chloro-5-(difluoromethoxy)-3-(piperazin-1-yl)phenyl)amino)-4-(cyclopropyl(4-methoxybenzyl)amino)pyrazolo[1,5-a][1,3,5]triazine-8-carbonitrile (170 mg, 0.29 mmol) and methyl (S)-2-((methylsulfonyl)oxy)propanoate (104 mg, 0.570 mmol) in acetonitrile (3.6 mL) was added Hunig's base (187 μL, 1.07 mmol). The reaction was heated at 85° C. for 16 h, cooled to RT and concentrated. The residue was purified via ISCO (24 g column; Hexane/EtOAc; 0 to 100% gradient) to give methyl (2R)-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanoate (100 mg, 51.4%). LC/MS [M+H]⁺=682.5, RT=1.59 min (Method C).

### Step 3. (2R)-2-(4-{2-chloro-3-[(8-cyano-4-{cyclopropyl[(4-methoxyphenyl) methyl]amino}pyrazolo[1,5-a][1,3,5]triazin-2-yl)amino]-5-(difluoromethoxy) phenyl}piperazin-1-yl)propanoic acid

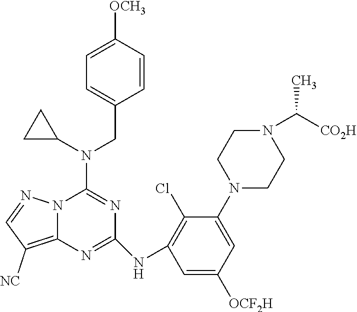

The product from Step 2 (100 mg, 0.15 mmol) was dissolved in THF/MeOH/H₂O (0.5 mL/0.5 mL/0.5 mL) at rt and LiOH (7 mg, 0.29 mmol) was added. It was stirred for 16 h. Additional LiOH (8 mg, 2 equiv.) was added. It was stirred for 2 more days. The reaction was concentrated and dried under vacuum to give crude (2R)-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanoic acid (98 mg), which was used as such for the next step. LC/MS [M+H]⁺=669.2, RT=1.57 min (Method C).

### Step 4. (2R)—N′-[(tert-butoxy)carbonyl]-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanehydrazide

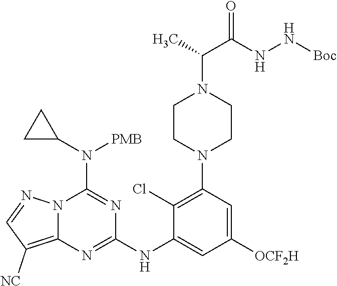

To a solution of (2R)-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanoic acid (100 mg, 0.15 mmol) in dichloromethane (1.5 mL) was added EDC (43.0 mg, 0.23 mmol) and HOBT (34.4 mg, 0.23 mmol). After 5 min, tert-butyl hydrazinecarboxylate (21.76 mg, 0.17 mmol) and triethylamine (31.3 μl, 0.23 mmol) were added. The reaction was stirred for 18 h. The reaction was diluted with DCM, transferred to a separating funnel and washed with sat. sodium bicarbonate followed by brine. The organic layer was separated and dried over sodium sulfate. The solvent was evaporated, and the residue was purified via ISCO (24 g column; CH₂Cl₂/MeOH; 0 to 10% gradient) to give (2R)—N′-[(tert-butoxy)carbonyl]-2-(4-{2-chloro-3-[(8-cyano-4-{cyclopropyl[(4-methoxyphenyl)methyl]amino}pyrazolo[1,5-a][1,3,5]triazin-2-yl)amino]-5-(difluoromethoxy)phenyl}piperazin-1-yl)propanehydrazide (68 mg). LC/MS [M+H]⁺=782.7, RT=1.66 min (Method C).

### Step 5. Example 211

The crude product from Step 4 (68 mg) was stirred in DCM (1 mL) and TFA (1 mL) for 1 h. The reaction mixture was concentrated, evaporated with DCM (3×) and dried under vacuum. It was dissolved in THF (1 mL), CDI (44.2 mg, 0.27 mmol) and triethylamine (0.063 mL, 0.454 mmol) were added, and the contents were stirred at rt. The reaction mixture was diluted with DCM and washed with sat. sodium bicarbonate followed by brine. The organic layers were dried over sodium sulfate and concentrated. The residue was dissolved in DCM (2 mL), anisole (0.030 mL, 0.27 mmol) was added followed by TFA (1 mL). The reaction was heated at 48° C. for 3 h. It was cooled, and concentrated. The residue was dissolved in DMSO and filtered. The crude material was purified via preparative LC/MS employing the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-min hold at 16% B, 16-56% B over 20 min, then a 0-min hold at 100% B; Flow Rate: 20 mL/min; Column Temperature: 25° C. Fraction collection was triggered by MS signals. Fractions containing the desired product were combined and dried via centrifugal evaporation. The yield of the product was 2.0 mg, and its estimated purity by LCMS analysis was 100%. Analytical LC/MS was used to determine the final purity. LC-MS Method B: Purity: 100%; Observed Mass: 588.20; RT: 1.48 min. LC-MS Method A: Purity: 100%; Observed Mass: 588.20; RT: 1.85 min. ¹H NMR (500 MHz, DMSO-d₆) δ 12.37 (br s, 1H), 9.25 (s, 1H), 9.17-9.03 (m, 1H), 8.38 (s, 1H), 7.57 (br d, J=1.8 Hz, 1H), 7.23 (t, J=75 Hz, 1H), 6.77 (br d, J=2.4 Hz, 1H), 3.90 (m, 1H), 3.72-3.44 (m, 2H), 3.05-2.92 (m, 3H), 2.84-2.76 (m, 1H), 2.73-2.63 (m, 1H), 2.55 (DMSO), 1.37 (br d, J=7.0 Hz, 3H), 0.92-0.42 (m, 4H) (H₂O suppressed at 3.33 ppm).

### Example 212

### 2-({5-cyano-2-methoxy-3-[4-(oxetan-3-yl)piperazin-1-yl]phenyl}amino)-4-(cyclopropylamino)pyrazolo[1,5-a][1,3,5]triazine-8-carbonitrile

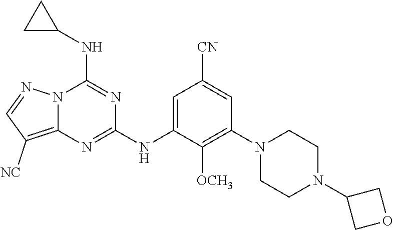 [SMILES: COc1c(Nc2nc(NC3CC3)n3ncc(C#N)c3n2)cc(C#N)cc1N1CCN(C2COC2)CC1]

### Step 1. 3-bromo-4-methoxy-5-[4-(oxetan-3-yl)piperazin-1-yl]benzonitrile

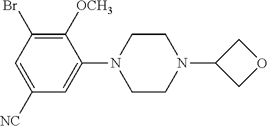 [SMILES: COc1c(Br)cc(C#N)cc1N1CCN(C2COC2)CC1]

3,5-Dibromo-4-methoxybenzonitrile (1.0 g, 3.44 mmol), 1-(oxetan-3-yl)piperazine (0.440 g, 3.09 mmol), palladium(II) acetate (0.077 g, 0.34 mmol), xantphos (0.30 g, 0.52 mmol) and cesium carbonate (3.36 g, 10.31 mmol) were suspended in dioxane (20 mL) at room temperature. The reaction was degassed with N₂ for 10 min. It was capped and heated to 105° C. After 1 day, it was cooled to rt, diluted with EtOAc, filtered through celite and the filtrate was concentrated. It was purified by ISCO, concentrated and dried under vacuum to give 3-bromo-4-methoxy-5-(4-(oxetan-3-yl)piperazin-1-yl)benzonitrile as white solids (350 mg, 28.9% yield). LC/MS [M+H]⁺=354.2, RT=1.18 min (Method C). ¹H NMR (400 MHz, CHLOROFORM-d) δ 7.51 (d, J=2.0 Hz, 1H), 7.12 (d, J=2.0 Hz, 1H), 4.82-4.51 (m, 4H), 3.89 (s, 3H), 3.75-3.49 (m, 1H), 3.32-3.03 (m, 4H), 2.71-2.40 (m, 4H) ppm.

### Step 2. Example 212

A mixture of 2-amino-4-(cyclopropyl(4-methoxybenzyl)amino)pyrazolo [1,5-a][1,3,5]triazine-8-carbonitrile (60 mg, 0.179 mmol), 3-bromo-4-chloro-5-(4-(oxetan-3-yl)piperazin-1-yl)benzonitrile (63.8 mg, 0.179 mmol), xantphos (10.35 mg, 0.018 mmol) and cesium carbonate (175 mg, 0.537 mmol) in dioxane (2 mL) was purged with nitrogen for 10 min, then Pd₂(dba)₃ (8.19 mg, 8.95 μmol) was added followed by purging with nitrogen for another 10 min, and the resulting mixture was capped and stirred at 90° C. for 16 h. After the brown slurry was cooled to rt, celite was added and diluted with ethyl acetate. The reaction mixture was filtered through celite washing with ethyl acetate. The filtrate was concentrated in vacuo to give a crude, which was dissolved in dichloroethane and TFA and anisole (0.020 mL, 0.179 mmol) was added. The reaction was stirred at 50° C. for 2 h. It was cooled and concentrated. The residue was dissolved in DMSO/HOAc filtered and submitted for purification and analysis. The recovery of the product was 5.1 mg, its estimated purity by reverse phase analysis was 100%. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. Analytical Reverse Phase chromatography was used to determine the final purity. LC-MS Method A: Purity: 100%; Observed Mass: 487.2; RT: 1.73 min. LC-MS Method B: Purity: 100%; Observed Mass: 487.1; RT: 1.3 min. ¹H NMR (500 MHz, DMSO-d₆) δ 9.28 (br s, 1H), 8.99 (s, 1H), 8.41 (s, 2H), 7.19 (s, 1H), 4.84-4.50 (m, 4H), 3.88 (s, 3H), 3.55-2.81 (m, 4H), 2.55 (DMSO), 1.03-0.64 (m, 4H) ppm (H₂O suppressed at 3.33 ppm).

### Example 213

### 2-{[2-chloro-5-(difluoromethoxy)-3-[(2S)-2-methylpiperazin-1-yl]phenyl]amino}-4-(cyclopropylamino)pyrazolo[1,5-a][1,3,5]triazine-8-carbonitrile

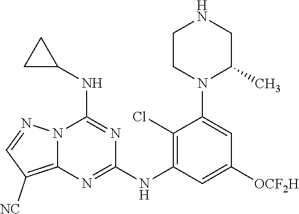

### Step 1. tert-butyl (3S)-4-[3-bromo-2-chloro-5-(difluoromethoxy)phenyl]-3-methylpiperazine-1-carboxylate

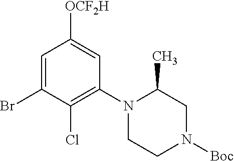

To a stirred solution of 1,3-dibromo-2-chloro-5-(difluoromethoxy) benzene (1.35 g, 4.01 mmol) in toluene (12 mL). (Oxydi-2,1-phenylene) bis(diphenylphosphine) (0.324 g, 0.602 mmol) and Pd₂(dba)₃ (0.184 g, 0.20 mmol) were added at room temperature under N₂. After the contents were degassed for 10 min, cesium carbonate (3.92 g, 12.04 mmol) was added followed by degassing for another 5 min. The resulting reaction mixture was heated to 95° C. for 16 h. After cooling to rt, the reaction mixture was filtered through a pad of celite and washed with ethyl acetate, and concentrated, purified by ISCO using silica (60-120) column (40 g) and eluting with ethyl acetate in hexanes. The desired fractions were concentrated to afford tert-butyl (3S)-4-[3-bromo-2-chloro-5-(difluoromethoxy)phenyl]-3-methylpiperazine-1-carboxylate (0.75 g, 1.646 mmol, 41.0% yield) as a white solid. LC/MS [M+H]⁺=457.3, RT=2.09 min (Method C).

### Step 2. Example 213

A mixture of 2-amino-4-(cyclopropyl(4-methoxybenzyl)amino)pyrazolo [1,5-a][1,3,5]triazine-8-carbonitrile (412 mg, 1.229 mmol), (3S)-4-[3-bromo-2-chloro-5-(difluoromethoxy)phenyl]-3-methylpiperazine-1-carboxylate (560 mg, 1.229 mmol), xantphos (213 mg, 0.369 mmol) and cesium carbonate (1201 mg, 3.69 mmol) in dioxane (10 mL) was purged with nitrogen for 10 min, then Pd₂(dba)₃ (113 mg, 0.12 mmol) was added followed by purging with nitrogen for another 10 min, finally the resulting mixture was stirred at 90° C. for 16 h. After the brown slurry was cooled to rt, celite was added followed by ethyl acetate. The reaction mixture was filtered through Celite and washed with ethyl acetate. The filtrate was concentrated in vacuo. LC/MS [M+H]⁺=710.7, 712.6, RT=2.15 min (Method C). To the crude product was added anisole (0.403 mL, 3.69 mmol), TFA (4.73 mL, 61.4 mmol) and DCE (9 mL), and the contents heated at 50° C. for 2 h. The reaction mixture was concentrated, and to the residue was added ammonia in MeOH (7N). The solid was triturated with EtOAc, and the EtOAc layer was concentrated, and triturated with DCM. The solid was collected and used as such for the next step. A small amount was submitted for purification and analysis. The recovery of the product was 7.9 mg, its estimated purity by Reverse Phase analysis was 98.6%. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. Analytical Reverse Phase chromatography was used to determine the final purity. LC-MS Method A: Purity: 98.6%; Observed Mass: 490.1; RT: 1.51 min. LC-MS Method B: Purity: 100%; Observed Mass: 490; RT: 1.5 min. ¹H NMR (500 MHz, DMSO-d₆) δ 9.28 (s, 1H), 9.17 (br. s, 1H), 8.40 (s, 1H), 7.73 (d, J=2.5 Hz, 1H), 7.48-7.09 (m, 1H), 6.93 (d, J=2.6 Hz, 1H), 3.19 (br. d, J=14.3 Hz, 3H), 2.92 (br. s, 2H), 2.55 (DMSO), 0.94 (d, J=6.3 Hz, 3H), 0.87-0.63 (m, 4H) ppm (H₂O suppressed at 3.33 ppm).

### Example 214

### 2-{[5-cyano-2-(difluoromethoxy)-3-[4-(oxetan-3-yl)piperazin-1-yl]phenyl]amino}-4-(cyclopropylamino)pyrazolo[1,5-a][1,3,5]triazine-8-carbonitrile

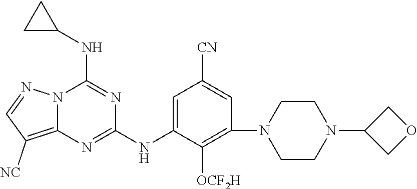

### Step 1. 3-bromo-4-(difluoromethoxy)-5-[4-(oxetan-3-yl)piperazin-1-yl]benzonitrile

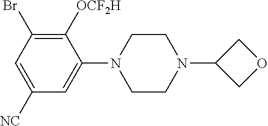

To 3,5-dibromo-4-(difluoromethoxy)benzonitrile (0.072 g, 0.220 mmol), 1-(oxetan-3-yl)piperazine (0.025 g, 0.176 mmol) in toluene (2 mL) was added palladium(II) acetate (4.94 mg, 0.022 mmol), xantphos (0.019 g, 0.033 mmol) and cesium carbonate (0.215 g, 0.66 mmol) at room temperature. It was degassed with N₂ for 3 min, capped and heated to 105° C. for 16 h. The mixture was cooled to room temperature, diluted with EtOAc, filtered through celite and concentrated. The residue was purified by ISCO (80 g SiO₂, 0 to 40% EtOAc-hexane gradient elution) to give 3-bromo-4-(difluoromethoxy)-5-[4-(oxetan-3-yl)piperazin-1-yl]benzonitrile (43 mg) as tan solids. LC/MS [M+H]⁺=390.2, RT=1.01 min (Method C).

### Step 2. Example 214

A mixture of 2-amino-4-(cyclopropyl(4-methoxybenzyl)amino)pyrazolo [1,5-a][1,3,5]triazine-8-carbonitrile (43.2 mg, 0.129 mmol), 3-bromo-4-(difluoromethoxy)-5-(4-(oxetan-3-yl)piperazin-1-yl)benzonitrile (50 mg, 0.129 mmol), xantphos (11.18 mg, 0.019 mmol) and cesium carbonate (126 mg, 0.386 mmol) in dioxane (2 mL) was purged with nitrogen for 10 min, then Pd₂(dba)₃ (5.90 mg, 6.44 μmol) was added followed by purging with nitrogen for another 10 min. The resulting mixture was capped and stirred at 90° C. for 16 h. After the brown slurry was cooled to rt, celite was added followed by ethyl acetate. The reaction mixture was filtered through celite and washed with ethyl acetate. The filtrate was concentrated in vacuo and dried. Anisole (0.014 mL, 0.129 mmol) was added followed by DCE (2 mL) and TFA (1.5 mL), After heating at 50° C. for 2 h, the reaction was cooled, concentrated, and purified by reverse phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The recovery of the product was 8.9 mg. LC-MS Method A: Purity: 95.4%; Observed Mass: 523.2; RT: 1.69 min. LC-MS Method B: Purity: 97.2%; Observed Mass: 523.1; RT: 1.33 min. ¹H NMR (500 MHz, DMSO-d₆) δ 9.27 (br d, J=1.6 Hz, 1H), 9.19 (s, 1H), 8.42 (s, 1H), 8.27 (s, 1H), 7.34 (d, J=1.4 Hz, 1H), 7.24-6.78 (m, 1H), 4.64-4.53 (m, 2H), 4.47 (t, J=6.0 Hz, 2H), 3.06 (br s, 3H), 2.55 (DMSO), 2.43 (br d, J=2.1 Hz, 3H), 0.91-0.69 (m, 4H) ppm (H₂O suppressed at 3.33 ppm)

The following compounds in Table were prepared according to similar procedures as described for Examples 1 and 205 to 214:

| | | LC/MS | |
|---|---|---|---|
| | | [M + H]⁺/ RT | |
| Ex # | Structure | (Method) | ¹H NMR (500 MHz, DMSO-d₆) |
| 215 | 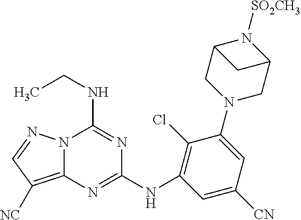 [SMILES: CCNc1nc(Nc2cc(C#N)cc(N3CC4CC(C3)N4S(C)(=O)=O)c2Cl)nc2c(C#N)cnn12] | 513.2 1.8 min (A) | δ 9.66-9.55 (m, 1H), 9.10-9.01 (m, 1H), 8.41 (s, 1H), 7.91 (d, J = 1.5 Hz, 1H), 7.71 (d, J = 1.2 Hz, 1H), 4.41 (br d, J = 6.1 Hz, 2H), 3.83-3.66 (m, 4H), 3.11 (s, 3H), 3.01-2.88 (m, 1H), 2.55 (DMSO), 2.13 (d, J = 8.2 Hz, 1H), 1.19 (t, J = 7.2 Hz, 3H) ppm (H₂O suppressed at 3.33 ppm) |
| 216 | 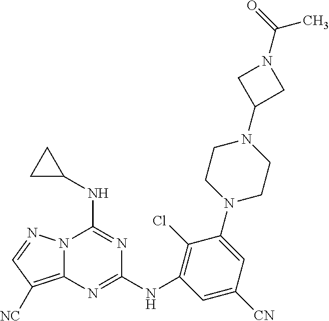 [SMILES: CC(=O)N1CC(N2CCN(c3cc(C#N)cc(Nc4nc(NC5CC5)n5ncc(C#N)c5n4)c3Cl)CC2)C1] | 532.30 1.67 min (A) | δ 9.56 (s, 1H), 9.23 (br d, J = 2.6 Hz, 1H), 8.41 (s, 1H), 8.12 (s, 1H), 7.45 (s, 1H), 4.35-3.73 (m, 3H), 3.26-2.66 (m, 5H), 2.55 (DMSO), 1.78 (s, 3H), 1.17 (t, J = 7.3 Hz, 4H), 0.81 (br s, 2H), 0.78-0.73 (m, 2H) ppm (H₂O suppressed at 3.33 ppm) |
| 217 | 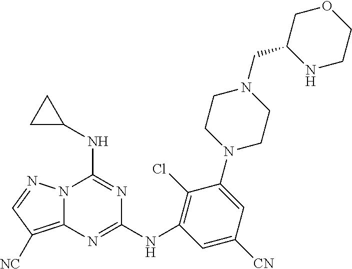 [SMILES: N#Cc1cc(Nc2nc(NC3CC3)n3ncc(C#N)c3n2)c(Cl)c(N2CCN(C[C@@H]3COCCN3)CC2)c1] | 534.2 1.52 min (A) | δ 8.41 (s, 1H), 8.17-8.08 (m, 1H), 7.39 (s, 1H), 3.05 (br s, 4H), 2.55 (DMSO), 2.11 (s, 2H), 1.88 (s, 4H), 1.27-1.21 (m, 7H), 0.88-0.66 (m, 4H) ppm (H₂O suppressed at 3.33 ppm) |
| 218 | 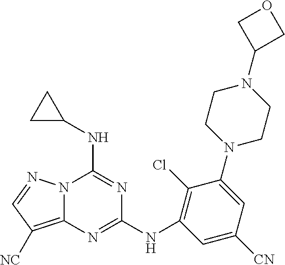 [SMILES: N#Cc1cc(Nc2nc(NC3CC3)n3ncc(C#N)c3n2)c(Cl)c(N2CCN(C3COC3)CC2)c1] | 491.2 1.7 min (A) | δ 9.52 (br s, 1H), 9.20 (br s, 1H), 8.40 (br d, J = 3.1 Hz, 1H), 8.20- 7.99 (m, 1H), 7.40 (br d, J = 2.4 Hz, 1H), 4.57 (br d, J = 4.3 Hz, 2H), 4.48 (br d, J = 4.6 Hz, 2H), 3.38- 3.37 (m, 1H), 3.54-3.37 (m, 4H), 3.07 (br s, 3H), 2.60-2.54 (m, 2H), 2.55 (DMSO), 0.97-0.57 (m, 4H) ppm (H₂O suppressed at 3.33 ppm) |
| 219 | 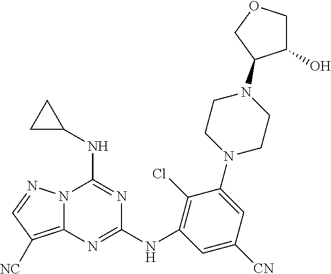 [SMILES: N#Cc1cc(Nc2nc(NC3CC3)n3ncc(C#N)c3n2)c(Cl)c(N2CCN([C@H]3COC[C@@H]3O)CC2)c1] | 521.2 1.45 min (A) | δ 8.38 (br s, 1H), 8.12 (br s, 1H), 7.37 (br s, 1H), 4.21 (br s, 1H), 3.94 (m, 1H), 3.89-3.75 (m, 1H), 3.63-3.35 (m, 5H), 3.09-2.92 (m, 4H), 2.55 (DMSO), 2.75 (m, 3H), 0.99-0.66 (m, 4H) ppm (H₂O suppressed at 3.33 ppm) |
| 220 | 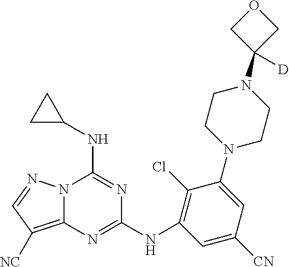 | 492.2 1.24 min (B) | δ 8.52-8.36 (m, 1H), 8.09 (br s, 1H), 7.41 (br s, 1H), 4.56 (br d, J = 5.8 Hz, 2H), 4.52-4.40 (m, 2H), 3.98-3.80 (m, 1H), 3.42 (br s, 2H), 3.07 (br s, 3H), 2.55 (DMSO), 0.92-0.70 (m, 4H) ppm (H₂O suppressed at 3.33 ppm) |
| 221 | 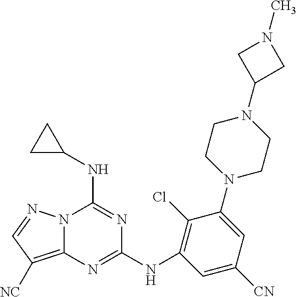 [SMILES: CN1CC(N2CCN(c3cc(C#N)cc(Nc4nc(NC5CC5)n5ncc(C#N)c5n4)c3Cl)CC2)C1] | 504.3 1.29 min (B) | δ 9.55 (br s, 1H), 9.30-9.13 (m, 1H), 8.40 (br s, 1H), 8.09 (br s, 1H), 7.47-7.38 (m, 1H), 4.48- 3.81 (m, 5H), 3.48 (m, 3H), 3.09 (br s, 3H), 2.87 (br s, 4H), 2.55 (DMSO), 0.91-0.59 (m, 4H) ppm (H₂O suppressed at 3.33 ppm) |
| 222 | 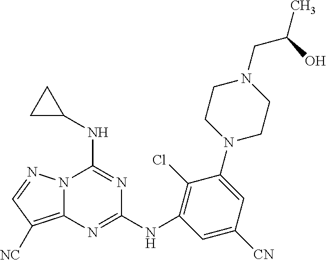 [SMILES: C[C@@H](O)CN1CCN(c2cc(C#N)cc(Nc3nc(NC4CC4)n4ncc(C#N)c4n3)c2Cl)CC1] | 493.1 1.59 min (A) | δ 8.42 (s, 1H), 8.21 (s, 1H), 8.09 (s, 1H), 7.39 (s, 1H), 3.87-3.77 (m, 1H), 3.04 (br s, 4H), 2.68- 2.58 (m, 3H), 2.55 (DMSO), 2.38- 2.30 (m, 1H), 2.30-2.17 (m, 1H), 1.08 (d, J = 6.1 Hz, 3H), 0.87-0.79 (m, 2H), 0.79-0.73 (m, 2H) ppm (H₂O suppressed at 3.33 ppm) |
| 223 | 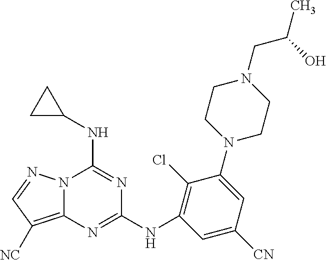 [SMILES: C[C@H](O)CN1CCN(c2cc(C#N)cc(Nc3nc(NC4CC4)n4ncc(C#N)c4n3)c2Cl)CC1] | 493.1 1.59 min (A) | δ 9.60 (s, 1H), 9.25 (br s, 1H), 8.42 (s, 1H), 8.18 (s, 1H), 7.53-7.46 (m, 1H), 4.28-4.08 (m, 1H), 3.57- 3.36 (m, 1H), 3.34-3.18 (m, 1H), 3.15-2.99 (m, 1H), 2.90-2.78 (m, 1H), 2.55 (DMSO), 1.15 (br d, J = 6.1 Hz, 5H), 0.87-0.72 (m, 4H) ppm (H₂O suppressed at 3.33 ppm) |
| 224 | 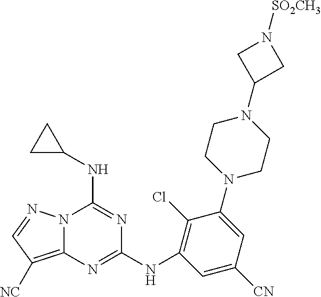 [SMILES: CS(=O)(=O)N1CC(N2CCN(c3cc(C#N)cc(Nc4nc(NC5CC5)n5ncc(C#N)c5n4)c3Cl)CC2)C1] | 568.2 1.73 min (A) | δ 9.58 (s, 1H), 9.23 (br d, J = 2.7 Hz, 1H), 8.41 (s, 1H), 8.13 (s, 1H), 7.46 (s, 1H), 3.99 (br d, J = 6.1 Hz, 3H), 3.37-3.12 (m, 1H), 3.05 (s, 1H), 3.01-2.98 (m, 1H), 3.10- 2.79 (m, 1H), 2.55 (DMSO), 0.89- 0.68 (m, 4H) ppm (H₂O suppressed at 3.33 ppm) |
| 225 | 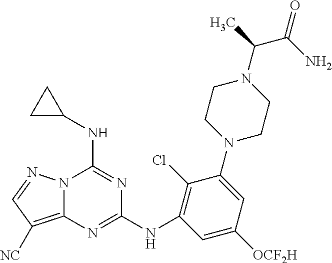 | 547.2 1.87 min (A) | δ 9.45-9.20 (m, 1H), 9.20-9.05 (m, 1H), 8.38 (s, 1H), 7.73-7.49 (m, 1H), 7.45-7.00 (m, 1H), 6.86- 6.66 (m, 1H), 3.69-3.45 (m, 1H), 3.26-3.03 (m, 1H), 3.03-2.77 (m, 1H), 2.55 (DMSO), 1.39-1.11 (m, 3H), 0.90-0.57 (m, 4H) ppm (H₂O suppressed at 3.33 ppm) |
| 226 | 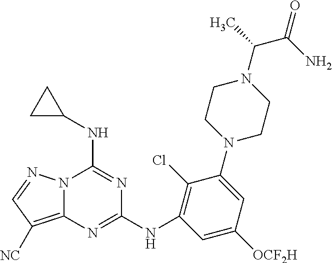 | 547.2 1.87 min (A) | δ 9.42-9.24 (m, 1H), 9.20-9.07 (m, 1H), 8.40 (s, 1H), 7.60 (br s, 2H), 7.26 (s, 3H), 6.92-6.69 (m, 1H), 3.65-2.83 (m, 2H), 2.55 (DMSO), 1.68-1.11 (m, 3H), 0.95- 0.33 (m, 4H) ppm (H₂O suppressed at 3.33 ppm) |
| 227 | 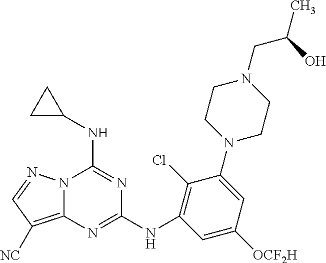 | 534.1 1.92 min (A) | δ 9.38-9.19 (m, 1H), 8.39 (s, 1H), 7.61 (br d, J = 1.8 Hz, 1H), 7.47- 7.00 (m, 1H), 6.82 (br d, J = 2.1 Hz, 1H), 4.14-3.93 (m, 1H), 3.61- 3.13 (m, 1H), 3.02-2.83 (m, 1H), 2.55 (DMSO), 1.13 (br d, J = 5.8 Hz, 3H), 0.85-0.68 (m, 4H) ppm (H₂O suppressed at 3.33 ppm) |
| 228 | 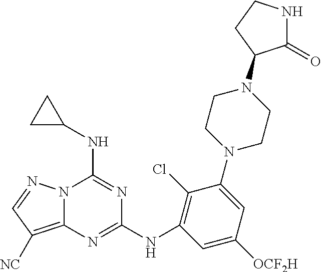 | 559.1 1.42 min (B) | δ 9.25 (s, 1H), 9.18-9.09 (m, 1H), 8.50-8.28 (m, 1H), 7.97-7.75 (m, 1H), 7.57 (d, J = 2.4 Hz, 1H), 7.23 (t, J = 75 Hz, 1H), 6.78 (d, J = 2.1 Hz, 1H), 3.68-3.37 (m, 1H), 3.34-2.72 (m, 6H), 2.55 (DMSO), 2.26-2.11 (m, 1H), 2.11-1.95 (m, 1H), 0.85- 0.69 (m, 4H) ppm (H₂O suppressed at 3.33 ppm) |
| 229 | 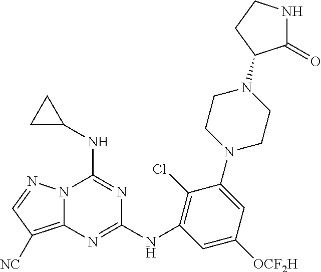 | 559.1 1.71 min (A) | δ 9.27 (s, 1H), 9.19-9.12 (m, 1H), 8.50-8.32 (m, 1H), 7.57 (br s, 1H), 7.24 (t, J = 75 Hz, 1H), 6.79 (br d, J = 2.1 Hz, 1H), 3.40-2.70 (m, 4H), 2.55 (DMSO), 2.27-2.15 (m, 1H), 2.15-1.97 (m, 1H), 0.91- 0.45 (m, 4H) ppm (H₂O suppressed at 3.33 ppm) |
| 230 | 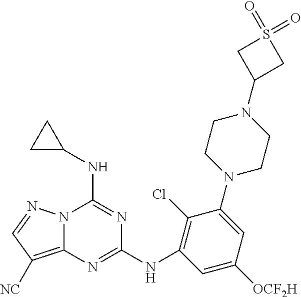 | 580.1 1.57 min (B) | δ 9.28 (br s, 1H), 9.21-9.07 (m, 1H), 8.40 (s, 1H), 7.56 (br s, 1H), 7.46-7.04 (m, 1H), 6.79 (br d, J = 2.1 Hz, 1H), 4.39-4.23 (m, 2H), 4.16 (br dd, J = 14.2, 6.0 Hz, 2H), 3.57-3.39 (m, 2H), 3.36-3.04 (m, 3H), 2.57 (br d, J = 11.3 Hz, 2H), 0.93-0.68 (m, 4H) ppm (H₂O suppressed at 3.33 ppm) |
| 231 | 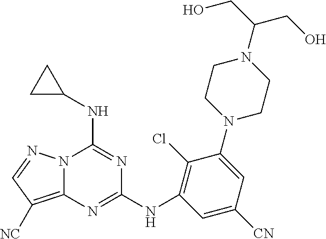 [SMILES: N#Cc1cc(Nc2nc(NC3CC3)n3ncc(C#N)c3n2)c(Cl)c(N2CCN(C(CO)CO)CC2)c1] | 509 1.42 min (A) | δ 9.47 (br s, 1H), 9.28-9.04 (m, 1H), 8.38 (s, 1H), 8.08 (br s, 1H), 7.35 (s, 1H), 3.86-3.45 (m, 3H), 3.01 (br s, 3H), 2.90-2.64 (m, 4H), 2.55 (DMSO), 0.93-0.55 (m, 4H) ppm (H₂O suppressed at 3.33 ppm |
| 232 | 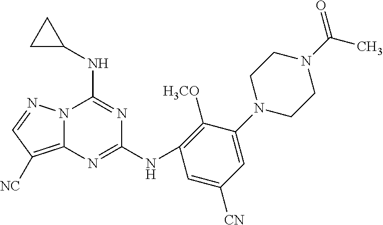 [SMILES: COc1c(Nc2nc(NC3CC3)n3ncc(C#N)c3n2)cc(C#N)cc1N1CCN(C(C)=O)CC1] | 473.1 1.61 min (B) | δ 9.41-9.17 (m, 1H), 8.98 (s, 1H), 8.41 (s, 1H), 8.42 (br s, 1H), 7.14 (d, J = 1.8 Hz, 1H), 3.91 (s, 3H), 3.63 (br s, 2H), 3.27-2.87 (m, 5H), 2.55 (DMSO), 2.05 (s, 3H), 1.35-1.12 (m, 1H), 0.96-0.50 (m, 4H) ppm (H₂O suppressed at 3.33 ppm |
| 233 | 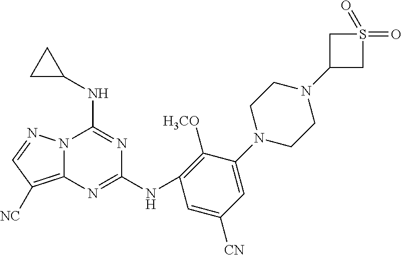 [SMILES: COc1c(Nc2nc(NC3CC3)n3ncc(C#N)c3n2)cc(C#N)cc1N1CCN(C2CS(=O)(=O)C2)CC1] | 535 1.89 min (A) | δ 8.39 (s, 2H), 7.13 (d, J = 0.9 Hz, 1H), 4.33-4.20 (m, 2H), 4.20- 4.03 (m, 2H), 3.87 (s, 2H), 3.38- 2.85 (m, 5H), 2.58 (br s, 3H), 2.55 (DMSO), 0.93-0.67 (m, 4H) ppm (H₂O suppressed at 3.33 ppm) |
| 234 | 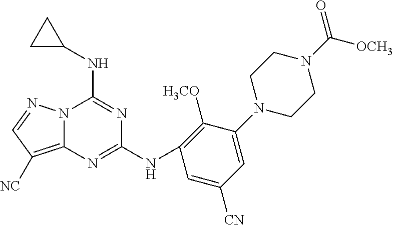 [SMILES: COC(=O)N1CCN(c2cc(C#N)cc(Nc3nc(NC4CC4)n4ncc(C#N)c4n3)c2OC)CC1] | 489.2 1.91 min (A) | δ 9.27-9.19 (m, 1H), 8.94 (s, 1H), 8.42 (br s, 1H), 8.37 (s, 1H), 7.17- 7.11 (m, 1H), 3.89 (s, 2H), 3.70- 3.49 (m, 5H), 3.01 (br s, 4H), 2.94- 2.82 (m, 1H), 0.88-0.73 (m, 4H) ppm (H₂O suppressed at 3.33 ppm) |
| 235 | 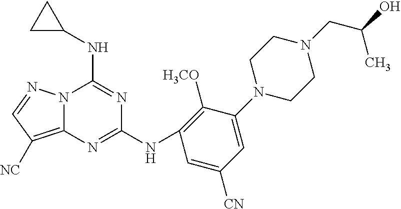 [SMILES: COc1c(Nc2nc(NC3CC3)n3ncc(C#N)c3n2)cc(C#N)cc1N1CCN(C[C@H](C)O)CC1] | 489.1 1.32 min (B) | δ 9.10-8.64 (m, 1H), 8.43-8.37 (m, 1H), 8.37-8.33 (m, 1H), 7.33- 6.89 (m, 1H), 3.86 (s, 1H), 3.77- 3.67 (m, 2H), 3.04 (br s, 3H), 2.93- 2.85 (m, 1H), 2.64-2.57 (m, 3H), 2.55 (DMSO), 2.35-2.27 (m, 1H), 2.27-2.19 (m, 1H), 1.06 (d, J = 6.1 Hz, 3H), 0.88-0.75 (m, 4H) ppm (H₂O suppressed at 3.33 ppm) |
| 236 | 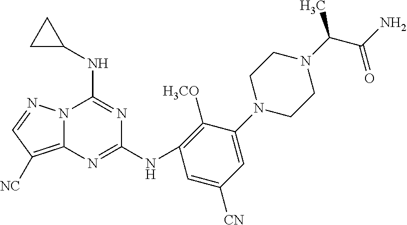 [SMILES: COc1c(Nc2nc(NC3CC3)n3ncc(C#N)c3n2)cc(C#N)cc1N1CCN([C@@H](C)C(N)=O)CC1] | 502.1 1.7 min (A) | δ 9.33-9.21 (m, 1H), 9.00-8.86 (m, 1H), 8.41 (s, 1H), 8.39-8.34 (m, 1H), 7.27 (br d, J = 1.9 Hz, 1H), 7.12 (s, 1H), 7.01 (br d, J = 0.9 Hz, 1H), 3.88 (s, 3H), 3.19-2.83 (m, 4H), 2.76-2.58 (m, 3H), 2.55 (DMSO), 1.14 (d, J = 6.9 Hz, 3H), 0.83 (br s, 4H) ppm (H₂O suppressed at 3.33 ppm |
| 237 | 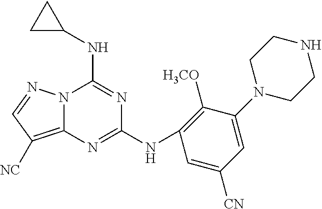 [SMILES: COc1c(Nc2nc(NC3CC3)n3ncc(C#N)c3n2)cc(C#N)cc1N1CCNCC1] | 431.1 0.97 min (A) | δ 8.70 (br d, J = 3.1 Hz, 1H), 8.24 (s, 1H), 7.26-7.23 (m, 2H), 2.93 (br dd, J = 6.4, 2.5 Hz, 1H), 1.25- 0.33 (m, 4H) ppm (H₂O suppressed at 3.33 ppm) |
| 238 | 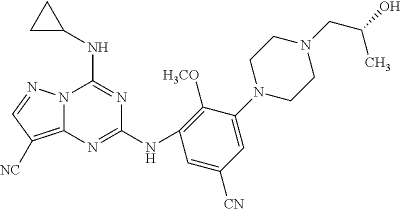 [SMILES: COc1c(Nc2nc(NC3CC3)n3ncc(C#N)c3n2)cc(C#N)cc1N1CCN(C[C@@H](C)O)CC1] | 489.1 1.64 min (A) | δ 8.94 (s, 1H), 8.54-8.04 (m, 2H), 7.12 (s, 1H), 3.88 (d, J = 8.5 Hz, 5H), 3.21-2.63 (m, 5H), 2.55 (DMSO), 2.44-2.22 (m, 2H), 1.07 (d, J = 6.1 Hz, 3H), 0.82 (br s, 4H) ppm (H₂O suppressed at 3.33 ppm) |
| 239 | 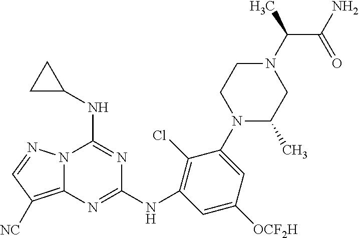 | 561.1 1.49 min (B) | δ 9.25 (s, 1H), 9.17 (br d, J = 3.1 Hz, 1H), 8.39 (s, 1H), 7.74 (br s, 1H), 7.27 (s, 1H), 7.26 (t, J = 75Hz, 1H), 7.20-7.16 (m, 1H), 7.09- 7.04 (m, 1H), 7.03-6.89 (m, 1H), 3.01-2.86 (m, 1H), 2.55 (DMSO), 1.54-1.29 (m, 3H), 0.91 (br d, J = 4.9 Hz, 2H), 0.82-0.69 (m, 4H) ppm (H₂O suppressed at 3.33 ppm) |
| 240 | 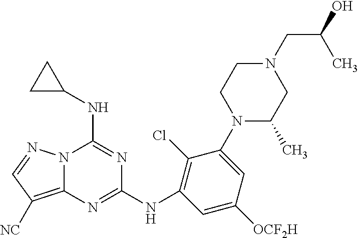 | 548.1 1.99 min (A) | δ 8.39 (s, 1H), 7.71-7.54 (m, 1H), 7.24 (t, J = 75 Hz, 1H), 6.99-6.70 (m, 1H), 3.93-3.70 (m, 1H), 3.11 (br d, J = 2.2 Hz, 1H), 2.98-2.85 (m, 1H), 2.78 (br d, J = 10.9 Hz, 1H), 2.71-2.60 (m, 2H), 2.55 (DMSO), 2.47 (m, 1H), 2.35-2.25 (m, 1H), 2.20 (br dd, J = 12.3, 5.3 Hz, 2H), 1.08 (d, J = 6.2 Hz, 3H), 0.88 (br d, J = 6.1 Hz, 3H), 0.82- 0.67 (m, 4H) ppm (H₂O suppressed at 3.33 ppm |
| 241 | 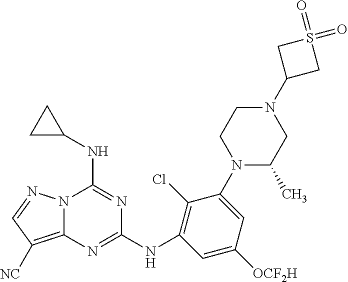 | 594 1.99 min (A) | δ 9.21 (s, 1H), 8.38 (s, 1H), 7.63 (d, J = 2.2 Hz, 1H), 7.24 (t, J = 75 Hz, 1H), 6.88 (br d, J = 2.0 Hz, 1H), 4.27 (br d, J = 8.5 Hz, 2H), 4.17- 4.00 (m, 2H), 3.42-3.08 (m, 2H), 2.95-2.58 (m, 3H), 2.47-2.36 (m, 1H), 2.21-2.05 (m, 1H), 0.88 (d, J = 6.2 Hz, 3H), 0.81-0.75 (m, 2H), 0.72 (br d, J = 6.2 Hz, 2H) ppm (H₂O suppressed at 3.33 ppm |
| 242 | 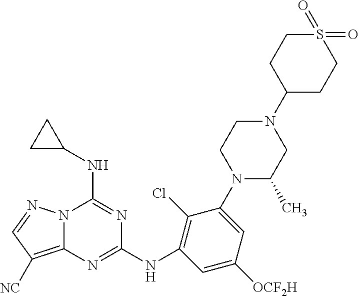 | 622.1 1.49 min (B) | δ 9.39 (m, 1H), 8.37 (s, 1H), 7.63 (d, J = 2.5 Hz, 1H), 7.26 (t, J = 75 Hz, 1H), 6.94-6.74 (m, 1H), 3.26- 3.05 (m, 3H), 3.00 (s, 1H), 2.97- 2.84 (m, 1H), 2.97-2.74 (m, 1H), 2.73-2.60 (m, 3H), 2.60-2.56 (m, 1H), 2.55 (DMSO), 2.36-2.19 (m, 1H), 2.15-1.98 (m, 4H), 0.89 (d, J = 6.1 Hz, 3H), 0.82-0.77 (m, 2H), 0.76-0.69 (m, 2H) ppm (H₂O suppressed at 3.33 ppm) |
| 243 | 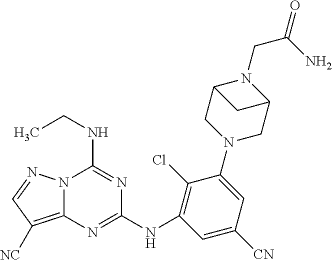 [SMILES: CCNc1nc(Nc2cc(C#N)cc(N3CC4CC(C3)N4CC(N)=O)c2Cl)nc2c(C#N)cnn12] | 492.1 1.57 min (A) | δ 9.64 (s, 1H), 9.19-9.01 (m, 1H), 8.41 (s, 1H), 8.09-7.92 (m, 1H), 7.90-7.28 (m, 2H), 4.59-4.17 (m, 1H), 4.12-3.68 (m, 2H), 2.93 (br d, J = 7.3 Hz, 1H), 2.55 (DMSO), 2.37-2.03 (m, 1H), 1.18 (m, 3H) ppm (H₂O suppressed at 3.33 ppm) |
| 244 | 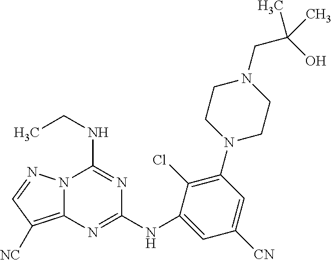 [SMILES: CCNc1nc(Nc2cc(C#N)cc(N3CCN(CC(C)(C)O)CC3)c2Cl)nc2c(C#N)cnn12] | 495.2 2.01 min (A) | δ 9.60 (s, 1H), 9.07 (br t, J = 5.3 Hz, 1H), 8.47-8.34 (m, 1H), 7.99 (br d, J = 0.6 Hz, 1H), 7.48 (br s, 1H), 3.05-2.86 (m, 1H), 2.55 (DMSO), 1.31-1.14 (m, 9H) ppm (H₂O suppressed at 3.33 ppm) |
| 245 | 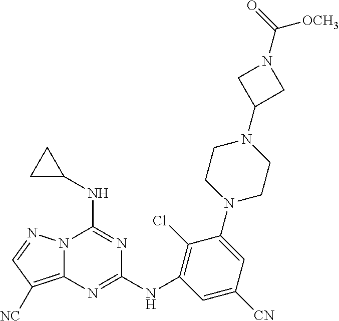 [SMILES: COC(=O)N1CC(N2CCN(c3cc(C#N)cc(Nc4nc(NC5CC5)n5ncc(C#N)c5n4)c3Cl)CC2)C1] | 548.30 1.9 min (A) | δ 9.51 (s, 1H), 9.20 (br d, J = 2.1 Hz, 1H), 8.39 (s, 1H), 8.10 (s, 1H), 7.38 (d, J = 1.5 Hz, 1H), 4.10-3.87 (m, 2H), 3.86-3.72 (m, 1H), 3.56 (s, 1H), 3.27-3.13 (m, 1H), 3.14- 2.90 (m, 3H), 2.88-2.72 (m, 1H), 2.55 (DMSO), 0.87-0.67 (m, 4H) ppm (H₂O suppressed at 3.33 ppm) |
| 246 | 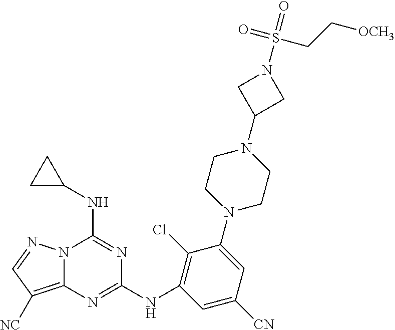 [SMILES: COCCS(=O)(=O)N1CC(N2CCN(c3cc(C#N)cc(Nc4nc(NC5CC5)n5ncc(C#N)c5n4)c3Cl)CC2)C1] | 612.3 1.91 min (A) | δ 8.41 (s, 1H), 8.10 (s, 1H), 7.38- 7.29 (m, 1H), 7.41 (s, 1H), 3.95- 3.78 (m, 4H), 3.69 (t, J = 6.1 Hz, 2H), 3.52-3.38 (m, 1H), 3.34- 2.78 (m, 1H), 2.55 (DMSO), 0.91- 0.69 (m, 4H) ppm (H₂O suppressed at 3.33 ppm) |
| 247 | 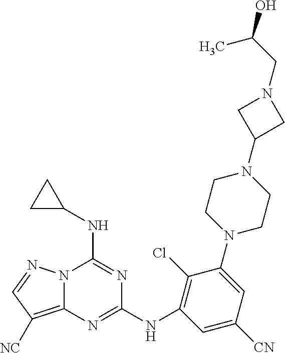 [SMILES: C[C@@H](O)CN1CC(N2CCN(c3cc(C#N)cc(Nc4nc(NC5CC5)n5ncc(C#N)c5n4)c3Cl)CC2)C1] | 548.1 1.5 min (A) | NA |
| 248 | 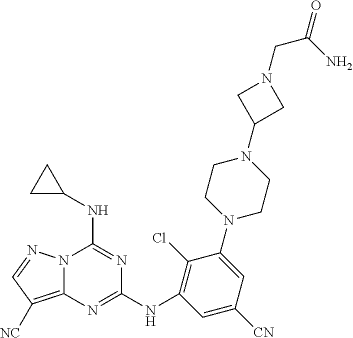 [SMILES: N#Cc1cc(Nc2nc(NC3CC3)n3ncc(C#N)c3n2)c(Cl)c(N2CCN(C3CN(CC(N)=O)C3)CC2)c1] | 547.2 1.46 min (A) | δ 8.41 (s, 1H), 8.09 (d, J = 0.6 Hz, 1H), 7.39 (d, J = 1.2 Hz, 1H), 7.15 (br s, 1H), 7.06 (br s, 1H), 3.54 (br s, 1H), 3.13-2.90 (m, 5H), 2.89- 2.77 (m, 1H), 2.55 (DMSO), 2.48- 2.35 (m, 3H), 0.90-0.63 (m, 4H) pp (H₂O suppressed at 3.33 ppm) |
| 249 | 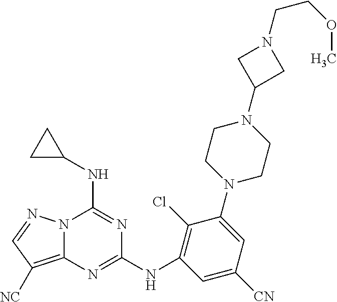 [SMILES: COCCN1CC(N2CCN(c3cc(C#N)cc(Nc4nc(NC5CC5)n5ncc(C#N)c5n4)c3Cl)CC2)C1] | 548.3 1.51 min (A) | δ 8.40 (s, 1H), 8.09 (s, 1H), 7.38 (d, J = 1.8 Hz, 1H), 3.33 (br t, J = 5.3 Hz, 1H), 3.23 (s, 2H), 3.03 (br s, 4H), 2.89-2.76 (m, 1H), 2.69 (br s, 2H), 2.55 (DMSO), 2.43 (br s, 3H), 0.86-0.67 (m, 4H) ppm (H₂O suppressed at 3.33 ppm) |
| 250 | 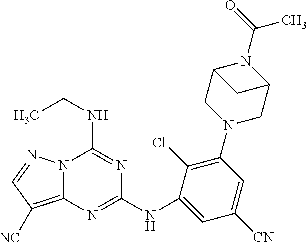 [SMILES: CCNc1nc(Nc2cc(C#N)cc(N3CC4CC(C3)N4C(C)=O)c2Cl)nc2c(C#N)cnn12] | 477.1 1.63 min (A) | δ 9.70-9.47 (m, 1H), 9.20-8.92 (m, 1H), 8.41 (s, 1H), 7.90 (s, 1H), 7.74-7.53 (m, 1H), 4.51 (m, 1H), 4.33 (m, 1H), 3.71 (br d, J = 10.1 Hz, 1H), 3.61 (br d, J = 10.4 Hz, 1H), 3.50-3.47 (m, 1H), 2.55 (DMSO), 1.94 (d, J = 8.2 Hz, 1H), 1.89 (s, 3H), 1.18 (t, J = 7.2 Hz, 3H) ppm (H₂O suppressed at 3.33 ppm) |
| 251 | 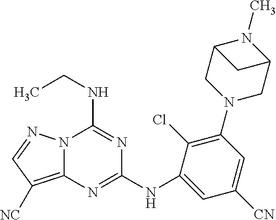 [SMILES: CCNc1nc(Nc2cc(C#N)cc(N3CC4CC(C3)N4C)c2Cl)nc2c(C#N)cnn12] | 449.0 1.33 min (A) | δ 9.65 (br s, 1H), 9.26-8.91 (m, 1H), 8.41 (s, 1H), 8.14-7.81 (m, 1H), 7.71 (s, 1H), 4.42 (br s, 1H), 4.36-4.21 (m, 1H), 3.86 (s, 4H), 3.02-2.74 (m, 1H), 2.67 (s, 1H), 2.55 (DMSO), 2.27 (br d, J = 10.4 Hz, 1H), 1.30-1.08 (m, 4H) ppm (H₂O suppressed at 3.33 ppm) |
| 252 | 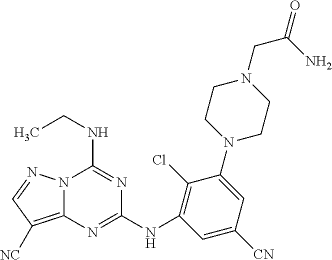 [SMILES: CCNc1nc(Nc2cc(C#N)cc(N3CCN(CC(N)=O)CC3)c2Cl)nc2c(C#N)cnn12] | 535.30 1.45 min (A) | δ 7.91 (d, J = 1.5 Hz, 1H), 7.41 (d, J = 1.2 Hz, 1H), 7.16-7.07 (m, 1H), 7.07-6.95 (m, 1H), 3.50 (br s, 1H), 3.00 (m, 5H), 2.55 (DMSO), 2.48-2.38 (m, 2H), 1.18 (t, J = 7.0 Hz, 3H) ppm (H₂O suppressed at 3.33 ppm) |
| 253 | 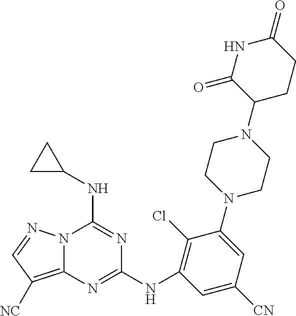 [SMILES: N#Cc1cc(Nc2nc(NC3CC3)n3ncc(C#N)c3n2)c(Cl)c(N2CCN(C3CCC(=O)NC3=O)CC2)c1] | 546.2 1.61 min (A) | NA |
| 254 | 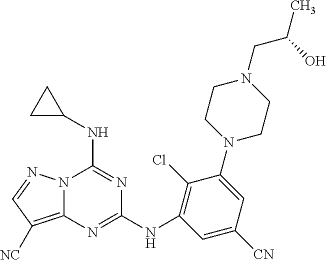 [SMILES: C[C@H](O)CN1CCN(c2cc(C#N)cc(Nc3nc(NC4CC4)n4ncc(C#N)c4n3)c2Cl)CC1] | 534.2 1.85 min (A) | δ 8.41 (s, 1H), 7.62 (br s, 1H), 7.49- 7.01 (m, 2H), 6.83 (br d, J = 1.8 Hz, 1H), 4.25-3.96 (m, 1H), 2.93 (br d, J = 1.2 Hz, 1H), 2.55 (DMSO), 1.14 (br d, J = 6.1 Hz, 3H), 0.92- 0.56 (m, 4H) ppm (H₂O suppressed at 3.33 ppm) |
| 255 | 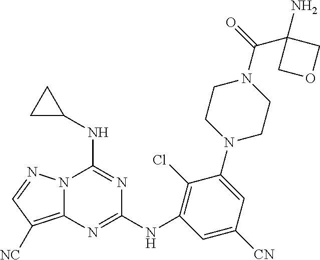 [SMILES: N#Cc1cc(Nc2nc(NC3CC3)n3ncc(C#N)c3n2)c(Cl)c(N2CCN(C(=O)C3(N)COC3)CC2)c1] | 534.2 1.50 min (A) | δ 9.55 (s, 1H), 9.22 (br d, J = 4.9 Hz, 1H), 8.41 (s, 1H), 8.14 (s, 1H), 7.41 (s, 1H), 4.90 (br d, J = 6.4 Hz, 2H), 4.41 (br d, J = 6.1 Hz, 2H), 3.52-3.26 (m, 1H), 3.17-2.73 (m, 4H), 2.55 (DMSO), 1.01-0.17 (m, 4H) ppm (H₂O suppressed at 3.33 ppm) |
| 256 | 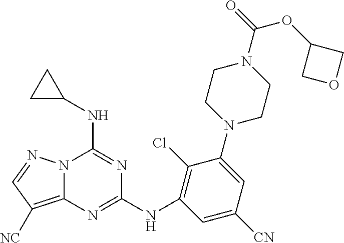 [SMILES: N#Cc1cc(Nc2nc(NC3CC3)n3ncc(C#N)c3n2)c(Cl)c(N2CCN(C(=O)OC3COC3)CC2)c1] | 535.2, 1.78 min (A) | δ 9.55 (s, 1H), 9.21 (br d, J = 2.7 Hz, 1H), 8.40 (s, 1H), 8.14-8.04 (m, 1H), 8.14 (s, 1H), 7.42 (s, 1H), 5.48-5.18 (m, 1H), 4.79 (t, J = 6.9 Hz, 2H), 4.63-4.30 (m, 2H), 3.67- 3.37 (m, 7H), 3.17-2.87 (m, 5H), 2.55 (DMSO), 0.93-0.58 (m, 4H) ppm (H₂O suppressed at 3.33 ppm) |
| 257 | 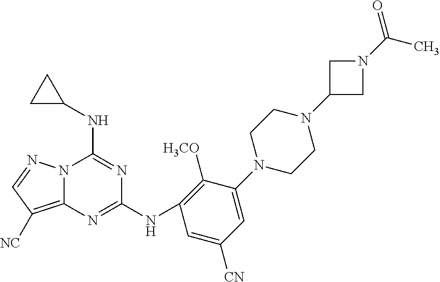 [SMILES: COc1c(Nc2nc(NC3CC3)n3ncc(C#N)c3n2)cc(C#N)cc1N1CCN(C2CN(C(C)=O)C2)CC1] | 528.2 1.27 min (B) | δ 9.34-9.22 (m, 1H), 9.08-8.92 (m, 1H), 8.49-8.43 (m, 1H), 8.41- 8.23 (m, 1H), 7.24-7.19 (m, 1H), 4.42-4.19 (m, 2H), 4.02 (br s, 2H), 3.88 (s, 3H), 3.42-2.81 (m, 3H), 1.80 (s, 3H), 0.93-0.79 (m, 4H) ppm (H₂O suppressed at 3.33 ppm) |
| 258 | 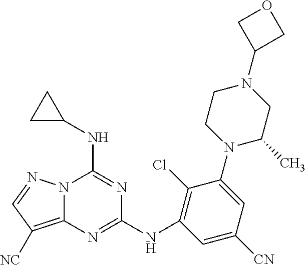 [SMILES: C[C@H]1CN(C2COC2)CCN1c1cc(C#N)cc(Nc2nc(NC3CC3)n3ncc(C#N)c3n2)c1Cl] | 546.1 1.45 min (B) | δ 8.39 (s, 1H), 7.64 (d, J = 2.1 Hz, 1H), 7.47-7.06 (m, 1H), 6.90 (br s, 1H), 4.56 (q, J = 6.5 Hz, 2H), 4.48 (t, J = 6.0 Hz, 1H), 4.44 (t, J = 6.0 Hz, 1H), 3.21-3.09 (m, 1H), 2.98- 2.85 (m, 1H), 2.77-2.65 (m, 1H), 2.65-2.59 (m, 1H), 2.38-2.25 (m, 1H), 2.55 (DMSO), 2.10-1.97 (m, 1H), 0.89 (br d, J = 6.1 Hz, 3H), 0.78 (br d, J = 3.7 Hz, 2H), 0.72 (br s, 2H) ppm (H₂O suppressed at 3.33 ppm) |
| 259 | 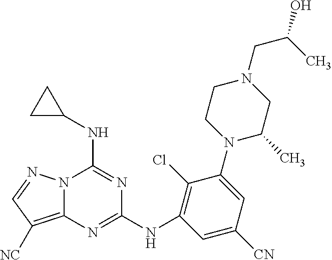 [SMILES: C[C@@H](O)CN1CCN(c2cc(C#N)cc(Nc3nc(NC4CC4)n4ncc(C#N)c4n3)c2Cl)[C@@H](C)C1] | 548.1 2 min (A) | δ 9.28 (s, 1H), 9.18 (br d, J = 3.7 Hz, 1H), 7.88-7.66 (m, 1H), 7.46- 7.09 (m, 1H), 6.98 (br s, 1H), 4.39- 3.95 (m, 1H), 3.54-2.78 (m, 3H), 2.55 (DMSO), 1.27-1.08 (m, 3H), 0.98-0.85 (m, 3H), 0.83-0.69 (m, 4H) ppm (H₂O suppressed at 3.33 ppm) |

### LC/MS Conditions:

### Method A:

Column: Waters XBridge 018, 2.1 mm×50 mm, 1.7 μm particles; Mobile Phase A: 5:95 acetonitrile-water with 10 mM ammonium acetate; Mobile Phase B: 95:5 acetonitrile-water with 10 mM ammonium acetate; Temperature: 50 OC; Gradient: 0% B to 100% B over 3 min, then a 0.50 min hold at 100% B; Flow: 1 mL/min; Detection: MS and UV (220 nm).

### Method B:

Column: Waters XBridge 018, 2.1 mm×50 mm, 1.7 μm particles; Mobile Phase A: 5:95 acetonitrile-water with 0.1% trifluoroacetic acid; Mobile Phase B: 95:5 acetonitrile-water with 0.1% trifluoroacetic acid; Temperature: 50 OC; Gradient: 0% B to 100% B over 3 min, then a 0.50 min hold at 100% B; Flow: 1 mL/min; Detection: MS and UV (220 nm).

### Method C:

Column: Waters ACQUITY UPLCO BEH C18, 2.1 mm×50 mm, 1.7 μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0% B to 100% B over 2 min, then a 0.50 min hold at 100% B; Flow: 1 mL/min; Detection: MS and UV (220 nm).

### Example 260

### 2-{[3-(4-acetylpiperazin-1-yl)-2-chloro-5-cyanophenyl]amino}-4-[(2,2-difluoroethyl)amino]pyrazolo[1,5-a][1,3,5]triazine-8-carbonitrile

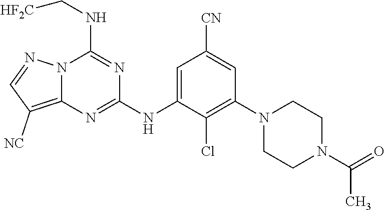

### Step 1. 2-amino-4-((2,2-difluoroethyl)amino)pyrazolo[1,5-a][1,3,5]triazine-8-carbonitrile

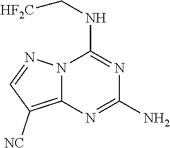

Prepared analogously to 2-amino-4-{cyclopropyl[(4-methoxyphenyl) methyl]amino}pyrazolo[1,5-a][1,3,5]triazine-8-carbonitrile using 2,2-difluoroethan-1-amine instead of N-(4-methoxybenzyl)cyclopropanamine. LC/MS [M+H]⁺=506.5. ¹H NMR (400 MHz, DMSO-d₆) δ 8.98-8.88 (m, 1H), 8.34-8.29 (m, 1H), 7.50-7.24 (m, 2H), 6.41-6.04 (m, 1H), 3.91-3.75 (m, 2H).

### Step 2. tert-butyl 4-(3-bromo-2-chloro-5-cyanophenyl)piperazine-1-carboxylate

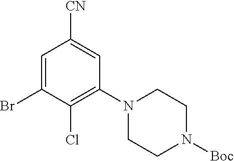 [SMILES: CC(C)(C)OC(=O)N1CCN(c2cc(C#N)cc(Br)c2Cl)CC1]

To a solution of 3,5-dibromo-4-chlorobenzonitrile (1.7 g, 5.76 mmol) and tert-butyl piperazine-1-carboxylate (1.179 g, 6.33 mmol) in toluene (15 mL) was added under nitrogen (oxybis(2,1-phenylene))bis (diphenylphosphane) (0.372 g, 0.691 mmol), Pd₂(dba)₃ (0.211 g, 0.230 mmol) and cesium carbonate (5.63 g, 17.27 mmol) and the contents heated to 95° C. with stirring. After 16 h, the reaction mixture was cooled to rt diluted with ethyl acetate, filtered, and evaporated under reduced pressure. The crude mixture was purified by flash chromatography using a gradient of 0 to 100% ethyl acetate in hexanes to obtain tert-butyl 4-(3-bromo-2-chloro-5-cyanophenyl)piperazine-1-carboxylate (277 mg, 1.15 mmol, 51% Yield) LC/MS [M+H-tBu]⁺=344.1. ¹H NMR (400 MHz, DMSO-d₆) δ 8.06-8.01 (m, 1H), 7.67-7.63 (m, 1H), 3.50-3.46 (m, 4H), 3.01-2.96 (m, 4H), 1.44-1.43 (m, 9H).

### Step 3. tert-butyl 4-(2-chloro-5-cyano-3-((8-cyano-4-((2,2-difluoroethyl) amino)pyrazolo[1,5-a][1,3,5]triazin-2-yl)amino)phenyl)piperazine-1-carboxylate

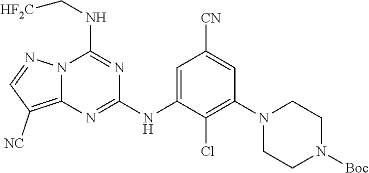

To a solution of 2-amino-4-((2,2-difluoroethyl)amino)pyrazolo[1,5-a][1,3,5]triazine-8-carbonitrile (100 mg, 0.418 mmol), tert-butyl 4-(3-bromo-2-chloro-5-cyanophenyl)piperazine-1-carboxylate (160 mg, 0.398 mmol), and cesium carbonate (324 mg, 0.995 mmol) in dioxane (5 mL) was added under nitrogen Pd₂(dba)₃ (36.5 mg, 0.040 mmol) and xantphos (46.1 mg, 0.080 mmol) and the contents heated at 95° C. with stirring. After 16 h, the reaction is cooled to rt and additional Pd₂(dba)₃ (36.5 mg, 0.040 mmol), xantphos (46.1 mg, 0.080 mmol) and cesium carbonate (324 mg, 0.995 mmol) are added and the contents at 95° C. for 2 days. The reaction mixture is cooled to rt, diluted with ethyl acetate, filtered, and concentrated under reduced pressure. The crude mixture was purified by flash chromatography using a gradient of 0 to 100% ethyl acetate in hexanes to afford tert-butyl 4-(2-chloro-5-cyano-3-((8-cyano-4-((2,2-difluoroethyl)amino)pyrazolo[1,5-a][1,3,5]triazin-2-yl)amino)phenyl)piperazine-1-carboxylate (220 mg, 0.42 mmol, 99% Yield) LC/MS [M+H]⁺=559.5.

### Step 4. 2-((2-chloro-5-cyano-3-(piperazin-1-yl)phenyl)amino)-4-((2,2-difluoroethyl)amino)pyrazolo[1,5-a][1,3,5]triazine-8-carbonitrile

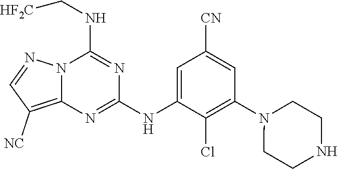

To a solution of tert-butyl 4-(2-chloro-5-cyano-3-((8-cyano-4-((2,2-difluoroethyl)amino)pyrazolo[1,5-a][1,3,5]triazin-2-yl)amino)phenyl)piperazine-1-carboxylate (220 mg, 0.394 mmol) in DCM (5 mL) is added TFA (0.212 mL, 2.76 mmol) and the contents stirred at rt. After 3 h, the reaction was concentrated under reduced pressure and dried under high vacuum to obtain 2-((2-chloro-5-cyano-3-(piperazin-1-yl)phenyl)amino)-4-((2,2-difluoroethyl) amino)pyrazolo[1,5-a][1,3,5]triazine-8-carbonitrile (163 mg, 0.35 mmol, 90% Yield) LC/MS [M+H]⁺=459.4.

### Step 5. Example 260

A solution of 2-((2-chloro-5-cyano-3-(piperazin-1-yl)phenyl)amino)-4-((2,2-difluoroethyl)amino)pyrazolo[1,5-a][1,3,5]triazine-8-carbonitrile (0.036 g, 0.079 mmol), acetic anhydride (8.20 μl, 0.087 mmol) and DIPEA (0.1 mL, 0.573 mmol) in DMF (1 mL) was heated to 80° C. for 5 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. The yield of the product was 12.8 mg and its estimated purity by LCMS analysis was 96%. Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.2%; Observed Mass: 501; RT: 1.61 min. Injection 2 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 2 results: Purity: 96.2%; Observed Mass: 501; RT: 1.62 min. 1H NMR (500 MHz, DMSO-d₆) δ 8.49-8.42 (m, 1H), 7.96-7.87 (m, 1H), 7.46-7.40 (m, 1H), 6.38-6.06 (m, 1H), 3.85-3.69 (m, 2H), 3.66-3.55 (m, 3H), 3.08-2.94 (m, 4H), 2.11-1.97 (m, 3H), 3 protons are missing due to water suppression.

### Example 261

### 2-({3-[(2S)-4-acetyl-2-methylpiperazin-1-yl]-2-chloro-5-cyanophenyl}amino)-4-[(2,2-difluoroethyl)amino]pyrazolo[1,5-a][1,3,5]triazine-8-carbonitrile

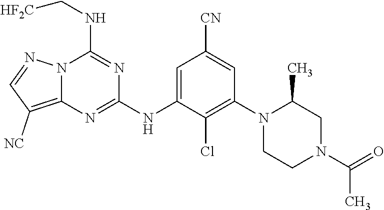

Example 261 was prepared in a similar procedure as described for Example 260. LC/MS [M+H]⁺=515.2. RT=1.63 min. LC/MS condition: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). ¹H NMR (500 MHz, DMSO-d₆) δ 8.52-8.39 (m, 1H), 7.99-7.90 (m, 1H), 7.60-7.51 (m, 1H), 6.35-6.01 (m, 1H), 3.83-3.69 (m, 2H), 3.64-3.54 (m, 1H), 3.19-3.13 (m, 2H), 2.80-2.62 (m, 1H), 2.10-1.99 (m, 3H), 0.93-0.76 (m, 3H) 3 protons are missing due to water suppression.

### Example 262

### (S)-2-((2-chloro-5-cyano-3-(2-methylpiperazin-1-yl)phenyl)amino)-4-((2,2-difluoroethyl)amino)pyrazolo[1,5-a][1,3,5]triazine-8-carbonitrile

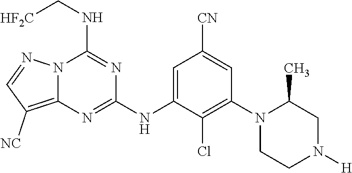

Example 262 prepared in a similar procedure as described for Example 260 Step 3. LC/MS [M+H]⁺=473.1. RT=1.36 min. LC/MS condition: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). ¹H NMR (500 MHz, DMSO-d₆) δ 8.50-8.39 (m, 1H), 7.95-7.88 (m, 1H), 7.54-7.47 (m, 1H), 6.38-6.03 (m, 1H), 3.83-3.68 (m, 2H), 3.14-3.05 (m, 1H), 3.03-2.94 (m, 1H), 2.90-2.77 (m, 2H), 2.62-2.53 (m, 4H), 0.90-0.80 (m, 3H) 2 protons are missing due to water suppression.

### Example 263

### (2S)-2-[(3S)-4-[2-chloro-5-cyano-3-({8-cyano-4-[(2,2-difluoroethyl)amino]pyrazolo[1,5-a][1,3,5]triazin-2-yl}amino)phenyl]-3-methylpiperazin-1-yl]propanamidecarbonitrile

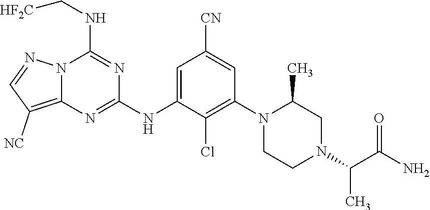

Example 263 was prepared in a similar procedure as described for Example 1 using Example 262 as starting material. LC/MS [M+H]⁺=544.2. RT=1.6 min. LC/MS condition: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). ¹H NMR (500 MHz, DMSO-d₆) δ 9.59-9.49 (m, 1H), 9.17-9.07 (m, 1H), 8.28-8.17 (m, 1H), 7.85-7.74 (m, 1H), 7.54-7.39 (m, 2H), 6.11-5.80 (m, 1H), 3.59-3.45 (m, 2H), 1.32-1.10 (m, 3H), 0.80-0.57 (m, 4H) 8 protons are missing due to water suppression.

### Example 264

### 2-({2-chloro-5-cyano-3-[(2S)-2-methyl-4-(oxetan-3-yl)piperazin-1-yl]phenyl}amino)-4-[(2,2-difluoroethyl)amino]pyrazolo[1,5-a][1,3,5]triazine-8-carbonitrile

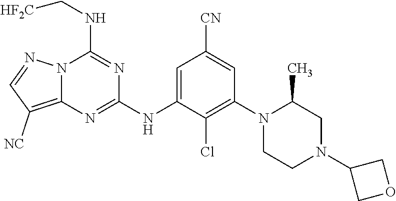

A solution of 2-{[2-chloro-5-cyano-3-(piperazin-1-yl)phenyl]amino}-4-[(2,2-difluoroethyl)amino]pyrazolo[1,5-a][1,3,5]triazine-8-carbonitrile (Example 260) (37.4 mg, 0.079 mmol), oxetan-3-one (28.5 mg, 0.395 mmol), and AcOH (4.52 μL, 0.079 mmol) in DMF (1 mL) was heated to 60° C. for 3 h. To the reaction mixture was added sodium triacetoxyborohydride (0.084 g, 0.395 mmol) and the contents heated a further 2 h. The crude material was purified via preparative Reverse Phase chromatography employing the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation. Analytical Reverse Phase chromatography was used to determine the final purity. Injection 1 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 1 results: Purity: 96.2%; Observed Mass: 473.1; RT: 1.36 min. Injection 3 conditions: Column: XBridge C18, 2.1 mm×50 mm, 1.7 μm particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+). Injection 3 results: Purity: 99.4%; Observed Mass: 473.1; RT: 1.3 min. ¹H NMR (500 MHz, DMSO-d₆) δ 9.78-9.68 (m, 1H), 9.40-9.26 (m, 1H), 8.50-8.42 (m, 1H), 7.96-7.85 (m, 1H), 7.63-7.53 (m, 1H), 6.37-6.02 (m, 1H), 4.64-4.40 (m, 4H), 3.84-3.69 (m, 2H), 2.80-2.68 (m, 1H), 2.66-2.57 (m, 1H), 2.42-2.30 (m, 1H), 2.14-1.99 (m, 1H), 0.96-0.83 (m, 3H) 4 protons are missing due to water suppression.

### Example 265

### 2-({2-chloro-5-cyano-3-[(2R)-4-(oxetan-3-yl)-2-(trifluoromethyl)piperazin-1-yl]phenyl}amino)-4-(cyclopropylamino)pyrazolo[1,5-a][1,3,5]triazine-8-carbonitrile

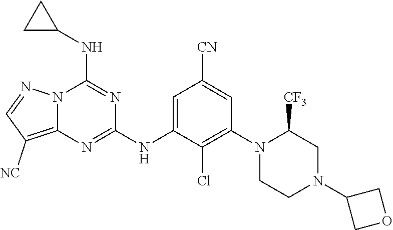 [SMILES: N#Cc1cc(Nc2nc(NC3CC3)n3ncc(C#N)c3n2)c(Cl)c(N2CCN(C3COC3)C[C@@H]2C(F)(F)F)c1]

Example 265 was prepared in a similar procedure as described for Example 264. LC/MS [M+H]⁺=559.2. RT=1.95 min. LC/MS condition: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). ¹H NMR (500 MHz, DMSO-d₆) δ 9.68-9.54 (m, 1H), 9.28-9.14 (m, 1H), 8.44-8.37 (m, 1H), 8.14-8.07 (m, 1H), 7.68-7.62 (m, 1H), 4.65-4.55 (m, 2H), 4.53-4.45 (m, 1H), 4.45-4.34 (m, 2H), 3.77-3.66 (m, 1H), 3.57-3.45 (m, 1H), 3.39-3.30 (m, 1H), 3.06-2.98 (m, 1H), 2.97-2.90 (m, 1H), 2.87-2.79 (m, 1H), 2.78-2.70 (m, 1H), 2.19-2.08 (m, 1H), 0.84-0.68 (m, 4H).

### Example 266

### 2-({2-chloro-5-cyano-3-[(1R,4R)-2,5-diazabicyclo[2.2.1]heptan-2-yl]phenyl}amino)-4-(cyclopropylamino)pyrazolo[1,5-a][1,3,5]triazine-8-carbonitrile

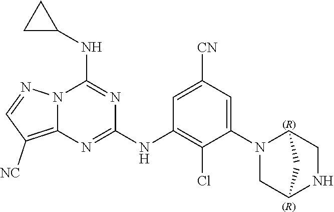

### Step 1. tert-butyl (1R,4R)-5-(3-((tert-butoxycarbonyl)amino)-2-chloro-5-cyanophenyl)-2,5-diazabicyclo[2.2.1]heptane-2-carboxylate

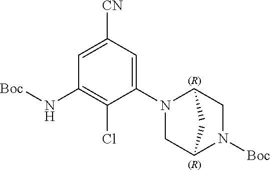

A 250 mL sealed tube was charged tert-butyl (3-bromo-2-chloro-5-cyanophenyl)carbamate (3.0 g, 9.05 mmol), tert-butyl (1R,4R)-2,5-diazabicyclo[2.2.1]heptane-2-carboxylate (2.332 g, 11.76 mmol) and toluene (100 mL). Under nitrogen purging, Pd₂dba₃ (0.828 g, 0.905 mmol), BINAP (1.125 g, 1.809 mmol) and cesium carbonate (7.12 g, 22.62 mmol) were added, nitrogen was bubbled for 15 min and the reaction was heated at 110° C. for 16 h. The reaction mixture was cooled to room temperature and filtered through celite. The filtrate was concentrated in vacuo. The crude obtained was diluted with EtOAc (150 mL) and water (100 mL). The EtOAc layer was separated, washed with brine (100 mL), dried over Na₂SO₄ and concentrated. The residue obtained was purified by Biotage Isolera, eluting with 15% EtOAc/hexane to afford tert-butyl (1R,4R)-5-(3-((tert-butoxycarbonyl)amino)-2-chloro-5-cyanophenyl)-2,5-diazabicyclo[2.2.1]heptane-2-carboxylate (3.8 g, 8.38 mmol, 93% yield) as a pale-yellow solid. LC/MS [M+H]⁺=449.2. ¹H-NMR: (400 MHz, DMSO-d₆): δ 8.82 (s, 1H), 7.42 (s, 1H), 7.21 (s, 1H), 4.50-4.36 (m, 2H), 3.92-3.84 (m, 1H), 3.45-3.36 (m, 1H), 3.13-3.04 (m, 1H), 1.95-1.84 (m, 2H), 1.46 (s, 9H), 1.39 (s, 9H).

### Step 2. tert-butyl (1R,4R)-5-(3-amino-2-chloro-5-cyanophenyl)-2,5-diazabicyclo[2.2.1]heptane-2-carboxylate

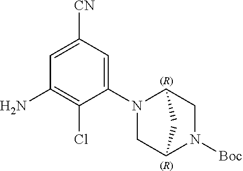

A solution of tert-butyl (1R,4R)-5-(3-((tert-butoxycarbonyl)amino)-2-chloro-5-cyanophenyl)-2,5-diazabicyclo[2.2.1]heptane-2-carboxylate (2.8 g, 6.24 mmol) in DCM (50 mL) was treated with TFA (7.21 mL, 94 mmol) and the contents stirred at rt for 1 h. The reaction mixture was concentrated, and the crude was triturated with Et₂O, and the resulting solid was dried by high vacuum. The solid (2.2 g) was then dissolved in 20 ml of DCM, and treated with triethyl amine (4.47 mL, 31.0 mmol), followed by Boc anhydride (2.030 mL, 8.85 mmol). It was stirred at rt for 2 h and concentrated. The crude obtained was diluted with EtOAc (150 mL) and water (100 mL). The EtOAc layer was separated, washed with brine (100 mL), dried over Na₂SO₄ and concentrated. The residue obtained was purified by Biotage Isolera, eluting with 18% EtOAc/hexane to afford tert-butyl (1R,4R)-5-(3-amino-2-chloro-5-cyanophenyl)-2,5-diazabicyclo[2.2.1]heptane-2-carboxylate (2.1 g, 68.1%). LC/MS [M−H]⁺=349.0. ¹H-NMR: (400 MHz, DMSO-d₆): δ 6.62 (d, J=1.60 Hz, 1H), 6.55 (d, J=2.00 Hz, 1H), 5.74 (brs, 2H), 4.41-4.33 (m, 2H), 3.81 (t, J=10.80 Hz, 1H), 3.45-3.38 (m, 1H), 3.10 (t, J=8.80 Hz, 1H), 1.90-1.81 (m, 2H), 1.41-1.33 (m, 9H).

### Step 3. tert-butyl (1R,4R)-5-(2-chloro-5-cyano-3-((8-cyano-4-(cyclopropyl(4-methoxybenzyl)amino)pyrazolo[1,5-a][1,3,5]triazin-2-yl)amino)phenyl)-2,5-diazabicyclo[2.2.1]heptane-2-carboxylate

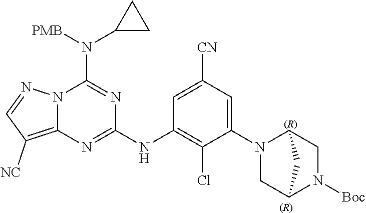

To a mixture of tert-butyl (1R,4R)-5-(3-amino-2-chloro-5-cyanophenyl)-2,5-diazabicyclo[2.2.1]heptane-2-carboxylate (1.4 g, 3.73 mmol) in tetrahydrofuran (50 mL) was added 1 M solution of LiHMDS (7.46 mL, 7.46 mmol) in THF dropwise at −78° C. It was stirred at −78° C. for 30 min and then 4-(cyclopropyl(4-methoxybenzyl)amino)-2-(methylsulfonyl)pyrazolo[1,5-a][1,3,5]triazine-8-carbonitrile (1.502 g, 3.73 mmol) in tetrahydrofuran (15 mL) was added and stirred at −78° C. for 30 min. The reaction was quenched with water and extracted with EtOAc. The organic layer was washed with water, dried over MgSO₄, filtered and concentrated. It was purified by Biotage isolera, eluting with 33% EtOAc/hexane to afford tert-butyl (1R,4R)-5-(2-chloro-5-cyano-3-((8-cyano-4-(cyclopropyl(4-methoxybenzyl)amino) pyrazolo[1,5-a][1,3,5]triazin-2-yl)amino)phenyl)-2,5-diazabicyclo[2.2.1]heptane-2-carboxylate as off white solid. LC/MS [M−H]*=667.2. ¹H-NMR: (400 MHz, DMSO-d₆): δ 9.66 (s, 1H), 8.42 (s, 1H), 7.61 (s, 1H), 7.26 (d, J=1.60 Hz, 1H), 7.21-7.12 (m, 2H), 6.91-6.84 (m, 2H), 5.25-5.05 (m, 2H), 4.52-4.38 (m, 2H), 3.93-3.85 (m, 1H), 3.72 (s, 3H), 3.46-3.41 (m, 1H), 3.13-3.06 (m, 1H), 2.97-2.82 (m, 1H), 1.93-1.83 (m, 2H), 0.89-0.73 (m, 2H).

### Step 4. Example 266

A mixture of tert-butyl (2-chloro-5-cyano-3-((1R,4R)-5-(8-cyano-4-(cyclopropyl(4-methoxybenzyl)amino)pyrazolo[1,5-a][1,3,5]triazin-2-yl)-2,5-diazabicyclo[2.2.1]heptan-2-yl)phenyl)carbamate (1.5 g, 2.248 mmol) in CH₂Cl₂ (30 mL) was added TFA (3.46 mL, 45.0 mmol) and the contents stirred at rt for 1 h and concentrated. To the residue was added DCM and the contents azeotroped three times to yield a light brown solid. The solid 2-((1R,4R)-5-(3-amino-2-chloro-5-cyanophenyl)-2,5-diazabicyclo[2.2.1]heptan-2-yl)-4-(cyclopropyl(4-methoxybenzyl)amino)pyrazolo[1,5-a][1,3,5]triazine-8-carbonitrile (1.3 g, 2.293 mmol) obtained was dissolved in ClCH₂CH₂Cl (30 mL). TFA (7.06 mL, 92 mmol) was added followed by anisole (0.997 mL, 9.17 mmol) and the contents heated at 55° C. for 1 h. The reaction mixture was cooled to RT and concentrated. To the residue was added DCM (2×) and the contents concentrated and dried under high vacuum. To the residue was then added 2 mL of 7 N NH₃ in MeOH. The white ppt. was filtered and washed with MeOH to afford 2-({2-chloro-5-cyano-3-[(1R,4R)-2,5-diazabicyclo[2.2.1]heptan-2-yl]phenyl}amino)-4-(cyclopropylamino)pyrazolo[1,5-a][1,3,5]triazine-8-carbonitrile (750 mg, 66% Yield) as an off-white solid. LC/MS [M+H]⁺=447.0. LCMS injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR: (400 MHz, DMSO-d₆): δ 9.47 (s, 1H), 1.00 (s, 1H), 7.75 (d, J=1.60 Hz, 1H), 7.12 (d, J=1.60 Hz, 1H), 4.34 (s, 1H), 3.86 (dd, J=2.00, 9.00 Hz, 1H), 3.60 (s, 1H), 3.06 (dd, J=3.60, 9.60 Hz, 2H), 2.85-2.91 (m, 2H), 1.77 (dd, J=9.20, Hz, 1H), 1.66 (dd, J=8.80, Hz, 1H), 0.75-0.80 (m, 4H).

### LCMS Conditions:

Method A: Column: Kinetex XB—C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 267

### 2-({2-chloro-5-cyano-3-[(1 S,4S)-2,5-diazabicyclo[2.2.1]heptan-2-yl]phenyl}amino)-4-(cyclopropylamino)pyrazolo[1,5-a][1,3,5]triazine-8-carbonitrile

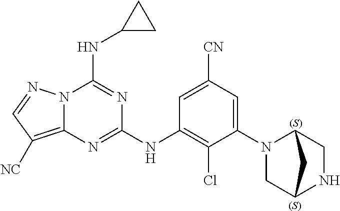

Example 267 was synthesized in a similar fashion as Example 266. LC/MS [M+H]⁺=447.0. LCMS Injection condition: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). ¹H-NMR (400 MHz, DMSO-d₆): δ 9.57 (br s, 1H), 8.41 (s, 1H), 7.82 (s, 1H), 7.21 (d, J=1.60 Hz, 1H), 4.44 (s, 1H), 3.97 (s, 1H), 3.88 (d, J=2.00 Hz, 1H), 3.26-3.22 (m, 3H), 3.07 (d, J=9.60 Hz, 1H), 2.85 (br s, 1H), 1.93 (d, J=10.00 Hz, 1H), 1.78 (d, J=10.00 Hz, 1H), 0.81-0.76 (m, 4H).

### Example 268

### 2-({3-[(1 S,4S)-5-acetyl-2,5-diazabicyclo[2.2.1]heptan-2-yl]-2-chloro-5-cyanophenyl}amino)-4-(cyclopropylamino)pyrazolo[1,5-a][1,3,5]triazine-8-carbonitrile

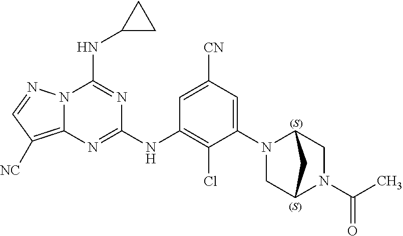

A mixture of 2-({2-chloro-5-cyano-3-[(1 S,4S)-2,5-diazabicyclo[2.2.1]heptan-2-yl]phenyl}amino)-4-(cyclopropylamino)pyrazolo[1,5-a][1,3,5]triazine-8-carbonitrile (30 mg, 0.067 mmol), Hunig's base (0.034 mL, 0.201 mmol) and acetic anhydride (6.85 mg, 0.067 mmol) in DMSO (0.5 mL) was stirred at rt for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({3-[(1 S,4S)-5-acetyl-2,5-diazabicyclo[2.2.1]heptan-2-yl]-2-chloro-5-cyanophenyl}amino)-4-cyclopropylamino)pyrazolo[1,5-a][1,3,5]triazine-8-carbonitrile (9 mg, 0.017 mmol, 25.6% yield) as an off white solid. LC/MS [M+H]⁺=489.0. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.51 (d, J=6.80 Hz, 1H), 9.20 (s, 1H), 8.41 (s, 1H), 7.77 (s, 1H), 7.26 (d, J=1.60 Hz, 1H), 4.73 (s, 0.5H), 4.61 (d, J=5.60 Hz, 1H), 4.51 (s, 1H), 3.96-3.90 (m, 1H), 3.68-3.66 (m, 0.5H), 3.59-3.52 (m, 1H), 3.23 (d, J=9.20 Hz, 0.5H), 3.17 (d, J=5.20 Hz, 1H), 3.12 (d, J=8.80 Hz, 0.5H), 2.84 (br s, 1H), 2.00-1.86 (m, 4H), 0.80-0.74 (m, 4H).

The following compounds were prepared in a similar procedure as described for Example 268 employing the R⁷ group identified in the table below:

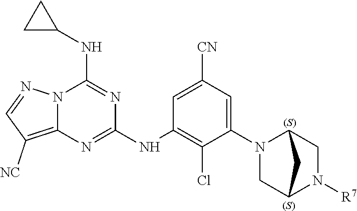

| | | LC/MS [M + H]⁺/ | |
|---|---|---|---|
| Ex # | R⁷ | RT (Method) | ¹H-NMR (400 MHz, DMSO-d₆) |
| 269 | 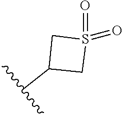 [SMILES: *C1CS(=O)(=O)C1] | 551.2 2.50 min (A) | δ 9.50 (s, 1H), 9.20 (s, 1H), 8.41 (s, 1H), 7.76 (d, J = 1.20 Hz, 1H), 7.15 (d, J = 1.60 Hz, 1H), 4.33-4.21 (m, 3H), 4.04 (d, J = 4.80 Hz, 1H), 4.04-3.92 (m, 2H), 3.72 (d, J = 7.20 Hz, 1H), 3.58 (s, 1H), 3.51-3.48 (m, 1H), 2.84 (s, 3H), 1.88 (d, J = 9.20 Hz, 1H), 1.79 (d, J = 8.80 Hz, 1H), 0.81-0.76 (m, 4H). |
| 270 | 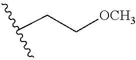 [SMILES: *CCOC] | 503.2 1.49 min (A) | δ 9.47 (s, 1H), 9.18 (s, 1H), 8.40 (s, 1H), 7.73 (d, J = 1.60 Hz, 1H), 7.13 (d, J = 2.00 Hz, 1H), 4.30 (s, 1H), 3.70 (d, J = 2.00 Hz, 1H), 3.55 (s, 1H), 3.49-3.40 (m, 2H), 3.36- |
| | | | 3.24 (m, 1H), 3.22 (s, 3H), 2.93-2.86 (m, |
| | | | 2H), 2.77-2.75 (m, 1H), 2.68-2.63 (m, 2H), |
| | | | 1.84-1.82 (m, 1H), 1.75-1.73 (m, 1H), 0.83- |
| | | | 0.79 (m, 4H). |

### Example 271

### 2-({2-chloro-5-cyano-3-[(1 S,4S)-5-(oxetan-3-yl)-2,5-diazabicyclo[2.2.1]heptan-2-yl]phenyl}amino)-4-(cyclopropylamino)pyrazolo[1,5-a][1,3,5]triazine-8-carbonitrile

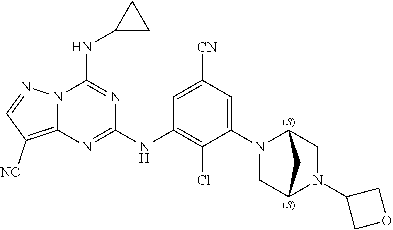

A mixture of 2-({2-chloro-5-cyano-3-[(1 S,4S)-2,5-diazabicyclo[2.2.1]heptan-2-yl]phenyl}amino)-4-(cyclopropylamino)pyrazolo[1,5-a][1,3,5]triazine-8-carbonitrile (40 mg, 0.090 mmol), acetic acid (0.013 mL, 0.230 mmol) and oxetan-3-one (6.45 mg, 0.090 mmol) in DMSO:THF (7:3) (1 mL) was stirred at rt for 2 h. sodium triacetoxyborohydride (56.9 mg, 0.269 mmol) was added and the contents stirred at rt for 16 h. The reaction was quenched with MeOH (0.5 mL) and the contents purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(1 S,4S)-5-(oxetan-3-yl)-2,5-diazabicyclo[2.2.1]heptan-2-yl]phenyl}amino)-4-(cyclopropylamino)pyrazolo[1,5-a][1,3,5]triazine-8-carbonitrile (6.55 mg, 0.013 mmol, 13.97% yield) as an off white solid. LC/MS [M+H]⁺=503.0. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.51 (s, 1H), 9.20 (s, 1H), 8.41 (s, 1H), 7.73 (s, 1H), 7.16 (s, 1H), 4.54 (t, J=6.80 Hz, 2H), 4.45 (s, 2H), 4.35 (t, J=6.00 Hz, 1H), 3.84 (t, J=6.00 Hz, 1H), 3.70 (d, J=2.00 Hz, 1H), 3.49 (s, 1H), 3.09 (d, J=9.60 Hz, 1H), 2.93 (d, J=10.00 Hz, 1H), 2.86 (br s, 1H), 2.80 (d, J=1.60 Hz, 1H), 1.83 (d, J=9.60 Hz, 1H), 1.75 (d, J=8.80 Hz, 1H), 0.81-0.76 (m, 4H).

The following compounds were prepared in a similar procedure as described for Example 271 employing the R⁷ group identified in the table below:

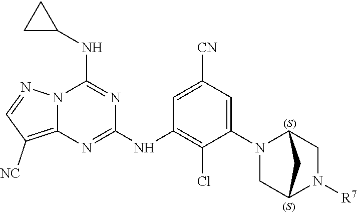

| | | LC/MS [M + H]⁺/ | |
|---|---|---|---|
| Ex # | R⁷ | RT (Method) | ¹H-NMR (400 MHz, DMSO-d₆) |
| 272 | 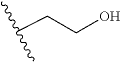 [SMILES: *CCO] | 491.0 1.85 min (B) | δ 9.51 (s, 1H), 9.20 (s, 1H), 8.41 (s, 1H), 7.73 (s, 1H), 7.16 (s, 1H), 4.54 (t, J = 6.80 Hz, 2H), 4.45 (s, 2H), 4.35 (t, J = 6.00 Hz, 1H), 3.84 (t, J = 6.00 Hz, 1H), 3.70 (d, J = 2.00 Hz, 1H), 3.49 (s, 1H), 3.09 (d, J = 9.60 |
| | | | Hz, 1H), 2.93 (d, J = 10.00 Hz, 1H), 2.86 (br |
| | | | s, 1H), 2.80 (d, J = 1.60 Hz, 1H), 1.83 (d, J = |
| | | | 9.60 Hz, 1H), 1.75 (d, J = 8.80 Hz, 1H), |
| | | | 0.81-0.76 (m, 4H). |
| 273 | 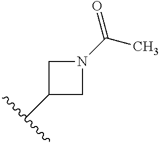 [SMILES: *C1CN(C(C)=O)C1] | 530.0 1.85 min (B) | δ 9.49 (s, 1H), 9.19 (s, 1H), 8.41 (s, 1H), 7.74 (s, 1H), 7.16 (d, J = 0.80 Hz, 1H), 4.36 (br s, 1H), 4.11-4.08 (m, 1H), 3.84-3.48 (m, 2H), 3.53-3.48 (m, 3H), 3.17 (d, J = 5.20 Hz, 2H), 2.88-2.80 (m, 3H), 1.84-1.77 (m, 1H), 1.75-1.71 (m, 1H), 1.72-1.71 (m, 3H), 0.80- 0.75 (m, 4H). |

### Example 274

### 2-({2-chloro-5-cyano-3-[(1 S,4S)-5-[(2R)-2-hydroxypropyl]-2,5-diazabicyclo[2.2.1]heptan-2-yl]phenyl}amino)-4-(cyclopropylamino)pyrazolo[1,5-a][1,3,5]triazine-8-carbonitrile

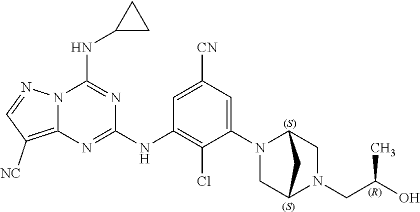

A mixture of 2-({2-chloro-5-cyano-3-[(1 S,4S)-2,5-diazabicyclo[2.2.1]heptan-2-yl]phenyl}amino)-4-(cyclopropylamino)pyrazolo[1,5-a][1,3,5]triazine-8-carbonitrile (40 mg, 0.090 mmol), DIPEA (0.047 mL, 0.269 mmol) and (R)-2-methyloxirane (5.20 mg, 0.090 mmol) was taken in DMSO (1 ml) and stirred at 80° C. for 16 h. The reaction mixture was cooled to rt, MeOH (0.5 mL) was added and the contents purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(1 S,4S)-5-[(2R)-2-hydroxypropyl]-2,5-diazabicyclo[2.2.1]heptan-2-yl]phenyl}amino)-4-(cyclopropylamino)pyrazolo[1,5-a][1,3,5]triazine-8-carbonitrile (5.3 mg, 11.37% yield) as an off white solid. LC/MS [M+H]⁺=505.0. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

The following compounds were prepared in a similar procedure as described for Example 274 employing the R⁵ group identified in the table below:

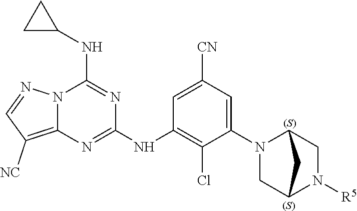

| | | LC/MS [M + H]⁺/ | |
|---|---|---|---|
| Ex # | R⁵ | RT (Method) | ¹H-NMR (400 MHz, DMSO-d₆) |
| 275 | 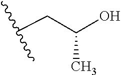 [SMILES: *C[C@@H](C)O] | 505.0 1.88 min (B) | — |
| 276 | 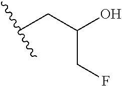 [SMILES: *CC(O)CF] | 523.0 1.90 min (B) | δ 9.49 (s, 1H), 9.20 (s, 1H), 8.40 (s, 1H), 7.71 (d, J = 1.20 Hz, 1H), 7.13 (d, J = 1.60 Hz, 1H), 4.92-4.85 (m, 2H), 4.45-4.42 (m, 0.5H), 4.44- 4.39 (m, 2H), 4.31-4.30 (m, 0.5H), 3.73-3.70 (m, 2H), 3.53 (br s, 1H), 3.30-3.24 (m, 1H), 2.95-2.75 (m, 3H), 1.76 (dd, J = 9.20 Hz, 2H), 0.80-0.74 (m, 4H). |
| 277 | 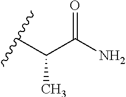 [SMILES: *[C@@H](C)C(N)=O] | 518.0 1.84 min (B) | — |
| 278 | 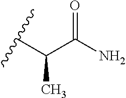 [SMILES: *[C@H](C)C(N)=O] | 518.0 1.84 min (B) | — |
| 279 | 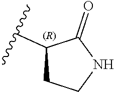 | 530.0 1.84 min (B) | — |
| 280 | 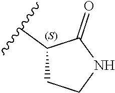 | 530.0 1.84 min (B) | — |

### LCMS Conditions:

Method A: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm).
Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 281

### 2-({2-chloro-5-cyano-3-[8-(2-methoxyethyl)-3,8-diazabicyclo[3.2.1]octan-3-yl]phenyl}amino)-4-(cyclopropylamino)pyrazolo[1,5-a][1,3,5]triazine-8-carbonitrile

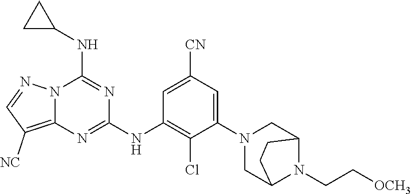 [SMILES: COCCN1C2CCC1CN(c1cc(C#N)cc(Nc3nc(NC4CC4)n4ncc(C#N)c4n3)c1Cl)C2]

### Step 1. tert-butyl 3-(3-((tert-butoxycarbonyl)amino)-2-chloro-5-cyanophenyl)-3,8-diazabicyclo[3.2.1]octane-8-carboxylate

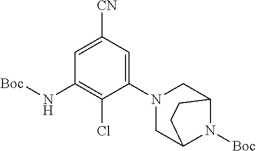 [SMILES: CC(C)(C)OC(=O)Nc1cc(C#N)cc(N2CC3CCC(C2)N3C(=O)OC(C)(C)C)c1Cl]

A 250 mL sealed tube was charged tert-butyl (3-bromo-2-chloro-5-cyanophenyl)carbamate (5 g, 15.08 mmol), tert-butyl 3,8-diazabicyclo[3.2.1]octane-8-carboxylate (4.80 g, 22.62 mmol) and toluene (60 mL). Under nitrogen purging, Pd₂dba₃ (1.381 g, 1.508 mmol), BINAP (0.290 g, 3.02 mmol) and cesium carbonate (14.74 g, 45.2 mmol) was added, and nitrogen was bubbled for 15 min and then reaction was heated at 110° C. for 16 h. The reaction mixture was cooled to room temperature. The reaction mixture was filtered through celite. The filtrate was concentrated in vacuo. The crude residue was diluted with EtOAc (200 mL) and water (100 mL). The EtOAc layer was separated, washed with brine (100 mL), dried over Na₂SO₄ and concentrated. The residue obtained was purified by Biotage Isolera, eluting with 15% EtOAc/hexane to afford tert-butyl 3-(3-((tert-butoxycarbonyl)amino)-2-chloro-5-cyanophenyl)-3,8-diazabicyclo[3.2.1]octane-8-carboxylate (2.5 g, 5.16 mmol, 34.2% yield) as an off white solid. LC/MS [M+H]⁺=463.0. ¹H-NMR (400 MHz, CDCl₃): δ 8.33 (d, J=1.60 Hz, 1H), 7.19 (s, 1H), 7.00 (d, J=1.60 Hz, 1H), 4.41-4.25 (m, 2H), 3.11-3.03 (m, 2H), 3.00-2.92 (m, 2H), 2.17-2.11 (m, 2H), 1.99-1.92 (m, 2H), 1.56 (s, 9H), 1.51 (s, 9H).

### Step 2. tert-butyl 3-(3-amino-2-chloro-5-cyanophenyl)-3,8-diazabicyclo[3.2.1]octane-8-carboxylate

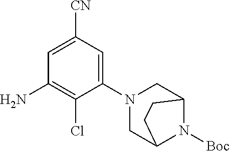 [SMILES: CC(C)(C)OC(=O)N1C2CCC1CN(c1cc(C#N)cc(N)c1Cl)C2]

A solution of tert-butyl 3-(3-((tert-butoxycarbonyl)amino)-2-chloro-5-cyanophenyl)-3,8-diazabicyclo[3.2.1]octane-8-carboxylate (2.5 g, 5.40 mmol) in DCM (40 mL) was treated with trifluoroacetic acid (8.26 mL, 108 mmol) and the contents stirred at rt for 16 h. The reaction mixture was concentrated, and the crude was triturated with Et₂O. The residue obtained was dissolved into 40 mL of DCM, and treated with triethylamine (3.15 mL, 22.60 mmol), followed by Boc-anhydride (0.865 mL, 3.77 mmol). It was stirred at RT for 16 h. The reaction mixture was concentrated, and the crude was purified by Biotage Isolera, eluting with 11% EtOAc/hexane to yield tert-butyl (3-(3,8-diazabicyclo[3.2.1]octan-3-yl)-2-chloro-5-cyanophenyl)carbamate (1.75 g, 4.12 mmol) as an off white solid. LC/MS [M−H]⁺=361.2. ¹H-NMR (400 MHz, DMSO-d₆): δ 6.77 (d, J=1.60 Hz, 1H), 6.70 (d, J=1.60 Hz, 1H), 4.52-4.21 (m, 4H), 3.16-3.08 (m, 2H), 2.96-2.90 (m, 2H), 2.19-2.09 (m, 2H), 2.02-1.90 (m, 2H), 1.52 (s, 9H), 0.92-0.80 (m, 2H).

### Step 3. tert-butyl 3-(2-chloro-5-cyano-3-((8-cyano-4-(cyclopropyl(4-methoxybenzyl)amino)pyrazolo[1,5-a][1,3,5]triazin-2-yl)amino)phenyl)-3,8-diazabicyclo[3.2.1]octane-8-carboxylate

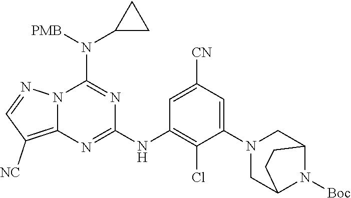

To a mixture of tert-butyl 3-(3-amino-2-chloro-5-cyanophenyl)-3,8-diazabicyclo[3.2.1]octane-8-carboxylate (1.2 g, 3.31 mmol) in tetrahydrofuran (15 mL) was added 1 M solution of LiHMDS (6.61 mL, 6.61 mmol) in THF dropwise at −78° C. It was stirred at −78° C. for 30 min and then 4-(cyclopropyl(4-methoxybenzyl)amino)-2-(methylsulfonyl)pyrazolo[1,5-a][1,3,5]triazine-8-carbonitrile (1.581 g, 3.97 mmol) in tetrahydrofuran (10 mL) was added and stirred at −78° C. for 30 min. The reaction was quenched with water and extracted with EtOAc. The organic layer was washed with water, dried over MgSO₄, filtered and concentrated. It was then purified by Biotage isolera, eluting with 23% EtOAc/hexane to afford tert-butyl 3-(2-chloro-5-cyano-3-((8-cyano-4-(cyclopropyl(4-methoxybenzyl)amino)pyrazolo[1,5-a][1,3,5]triazin-2-yl)amino)phenyl)-3,8-diazabicyclo[3.2.1]octane-8-carboxylate (770 mg, 1.107 mmol, 33.5% yield) as pale yellow fluffy solid. LC/MS [M−H]⁺=681.2. ¹H-NMR (400 MHz, CDCl₃): δ 8.74 (s, 1H), 8.10 (s, 1H), 7.83 (s, 1H), 7.14 (d, J=8.00 Hz, 2H), 7.03 (d, J=4.00 Hz, 1H), 6.86 (d, J=8.00 Hz, 2H), 5.51-5.40 (m, 2H), 4.42-4.28 (m, 2H), 3.80 (s, 3H), 3.15-3.07 (m, 2H), 3.02-2.92 (m, 2H), 2.18-2.13 (m, 2H), 2.00-1.91 (m, 2H), 1.52 (s, 9H), 1.16-1.01 (m, 2H), 0.90-0.85 (m, 2H).

### Step 4. (2-((3-(3,8-diazabicyclo[3.2.1]octan-3-yl)-2-chloro-5-cyanophenyl)amino)-4-(cyclopropylamino)pyrazolo[1,5-a][1,3,5]triazine-8-carbonitrile

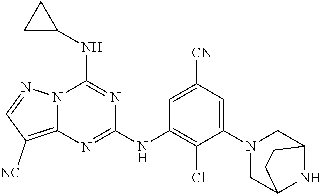 [SMILES: N#Cc1cc(Nc2nc(NC3CC3)n3ncc(C#N)c3n2)c(Cl)c(N2CC3CCC(C2)N3)c1]

To a mixture of tert-butyl 3-(2-chloro-5-cyano-3-((8-cyano-4-(cyclopropyl(4-methoxybenzyl)amino)pyrazolo[1,5-a][1,3,5]triazin-2-yl)amino)phenyl)-3,8-diazabicyclo[3.2.1]octane-8-carboxylate (1.120 g, 1.644 mmol) in DCM (20 mL) was added trifluoroacetic acid (2.52 mL, 32.9 mmol) and the contents stirred at rt for 1 h. The reaction mixture was concentrated, and the residue was azeotroped with DCM (3×) and concentrated to yield a light brown solid. To the solid (1.1 g) dissolved in ClCH₂CH₂Cl (15 mL) was added TFA (4.93 mL, 64.5 mmol) followed by anisole (0.700 mL, 6.45 mmol) and the contents heated at 50° C. for 3 h. The reaction mixture was cooled to rt and concentrated. To the residue was added DCM and the contents concentrated (3×). To the residue was then added 3 mL of 7 N NH₃ in MeOH. A white ppt was formed. It was filtered and washed with MeOH to 2-((3-(3,8-diazabicyclo [3.2.1]octan-3-yl)-2-chloro-5-cyanophenyl)amino)-4-(cyclopropylamino) pyrazolo[1,5-a][1,3,5]triazine-8-carbonitrile (600 mg, 1.232 mmol, 76% yield) as an off-white solid. LC/MS [M+H]⁺=461.2. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.58 (s, 1H), 9.24 (s, 1H), 8.42 (s, 1H), 8.06 (s, 1H), 7.41 (s, 1H), 7.23 (s, 1H), 7.11 (s, 1H), 6.88 (s, 1H), 3.51-3.46 (m, 1H), 3.25-3.21 (m, 1H).

### Step 5. Example 281

A solution of 2-((3-(3,8-diazabicyclo[3.2.1]octan-3-yl)-2-chloro-5-cyanophenyl)amino)-4-(cyclopropylamino)pyrazolo[1,5-a][1,3,5]triazine-8-carbonitrile (20 mg, 0.043 mmol), Hunig's base (0.038 mL, 0.217 mmol) and 1-bromo-2-methoxyethane (12.06 mg, 0.087 mmol) in DMSO (1 mL) was stirred at 85° C. for 16 h. LCMS showed completion of reaction. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[8-(2-methoxyethyl)-3,8-diazabicyclo[3.2.1]octan-3-yl]phenyl}amino)-4-cyclopropylamino)pyrazolo[1,5-a][1,3,5]triazine-8-carbonitrile (10 mg, 0.018 mmol, 41.5% yield) as an off white solid. LC/MS [M+H]⁺=519.2. LCMS Injection condition: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). ¹H-NMR (400 MHz, DMSO-d₆): δ 9.55 (s, 1H), 9.21 (s, 1H), 8.41 (s, 1H), 8.04 (d, J=1.6 Hz, 1H), 7.38 (d, J=2.0 Hz, 1H), 3.47-3.42 (m, 2H), 3.27 (s, 3H), 3.08-3.02 (m, 2H), 2.97-2.91 (m, 2H), 2.87-2.78 (m, 1H), 1.94-1.82 (m, 4H), 0.83-0.72 (m, 4H).

### Example 282

### 2-({2-chloro-5-cyano-3-[8-(oxetan-3-yl)-3,8-diazabicyclo[3.2.1]octan-3-yl]phenyl}amino)-4-(cyclopropylamino)pyrazolo[1,5-a][1,3,5]triazine-8-carbonitrile

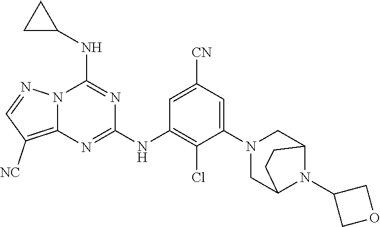 [SMILES: N#Cc1cc(Nc2nc(NC3CC3)n3ncc(C#N)c3n2)c(Cl)c(N2CC3CCC(C2)N3C2COC2)c1]

To a mixture of 2-((3-(3,8-diazabicyclo[3.2.1]octan-3-yl)-2-chloro-5-cyanophenyl)amino)-4-(cyclopropylamino)pyrazolo[1,5-a][1,3,5]triazine-8-carbonitrile (30 mg, 0.065 mmol), acetic acid (0.016 mL, 0.195 mmol) was added oxetan-3-one (7 mg, 0.098 mmol) in DMSO:THF (7:3) (1 mL) and the contents stirred at rt for 2 h. Sodium triacetoxyborohydride (41.4 mg, 0.195 mmol) was added and the contents stirred at rt for 16 h. The reaction was quenched using MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[8-(oxetan-3-yl)-3,8-diazabicyclo [3.2.1]octan-3-yl]phenyl}amino)-4-(cyclopropylamino)pyrazolo[1,5-a][1,3,5]triazine-8-carbonitrile (20.71 mg, 0.040 mmol, 60.9% yield) as an off white solid. LC/MS [M+H]⁺=505.2. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.58 (s, 1H), 9.24 (s, 1H), 8.42 (s, 1H), 8.06 (s, 1H), 7.41 (s, 1H), 7.23 (s, 1H), 7.11 (s, 1H), 3.46 (s, 1H), 3.21 (m, 1H), 3.13-3.09 (m, 2H), 3.03-2.98 (m, 2H), 2.86-2.81 (m, 2H), 1.94-1.88 (m, 3H), 1.78-1.75 (m, 1H), δ 1.36 (d, J=Hz, 1H), 1.15 (d, J=6.40 Hz, 3H), 0.80-0.74 (m, 4H).

### Example 283

### 2-({3-[(3R,5S)-4-acetyl-3,5-dimethylpiperazin-1-yl]-2-chloro-5-cyanophenyl}amino)-4-(cyclopropylamino)pyrazolo[1,5-a][1,3,5]triazine-8-carbonitrile

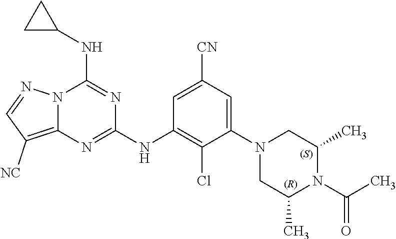

### Step 1. tert-butyl (2R,6S)-4-(3-((tert-butoxycarbonyl)amino)-2-chloro-5-cyanophenyl)-2,6-dimethylpiperazine-1-carboxylate

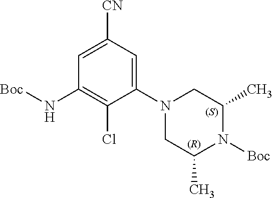

A 250 mL sealed tube was charged with tert-butyl (3-bromo-2-chloro-5-cyanophenyl)carbamate (2 g, 6.03 mmol), tert-butyl (2R,6S)-2,6-dimethyl piperazine-1-carboxylate (1.680 g, 7.84 mmol) and toluene (50 mL). Under nitrogen purging, Pd₂dba₃ (0.552 g, 0.603 mmol), BINAP (0.116 g, 1.206 mmol) and cesium carbonate (4.91 g, 15.08 mmol) was added, nitrogen was bubbled for 5 min and then reaction mixture was heated at 110° C. for 16 h. The reaction mixture was cooled to rt and filtered over celite. The filtrate was concentrated in vacuo. The crude obtained was diluted with EtOAc (150 mL) and water (100 mL). The EtOAc layer was separated, washed with brine (100 mL), dried over Na₂SO₄ and concentrated. The residue obtained was purified by Biotage Isolera, eluting with 10% EtOAc/hexane to afford tert-butyl (2S,6R)-4-(3-((tert-butoxycarbonyl)amino)-2-chloro-5-cyanophenyl)-2,6-dimethylpiperazine-1-carboxylate (640 mg, 1.313 mmol, 21.77% yield) as an off white solid. LC/MS [M−H]⁺=463.2.

### Step 2. tert-butyl (2R,6S)-4-(3-amino-2-chloro-5-cyanophenyl)-2,6-dimethylpiperazine-1-carboxylate

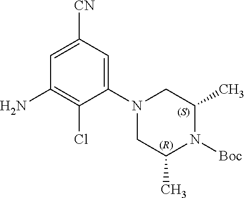

A solution of tert-butyl (2S,6R)-4-(3-((tert-butoxycarbonyl)amino)-2-chloro-5-cyanophenyl)-2,6-dimethylpiperazine-1-carboxylate (640 mg, 1.376 mmol) in DCM (40 mL) was treated with trifluoroacetic acid (2.106 mL, 27.5 mmol) and the contents stirred at rt for 16 h. The reaction mixture was concentrated, and dried under high vacuum. The yellow solid obtained was then dissolved into 30 mL of DCM, and treated with triethylamine (1.424 mL, 10.22 mmol), followed by di-tert-butyl dicarbonate (0.391 mL, 1.703 mmol). It was then stirred for 16 h at rt. The reaction mixture was concentrated. The crude obtained was diluted with EtOAc (75 mL) and water (50 mL). The EtOAc layer was separated, washed with brine (100 mL), dried over Na₂SO₄ and concentrated. The residue obtained was purified by Biotage Isolera, eluting with 20% EtOAc/hexane to yield tert-butyl (2S,6R)-4-(3-amino-2-chloro-5-cyanophenyl)-2,6-dimethylpiperazine-1-carboxylate (200 mg, 28.4% yield) as off white solid. LC/MS [M−H]⁺=309.0.

### Step 3. tert-butyl (2R,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate

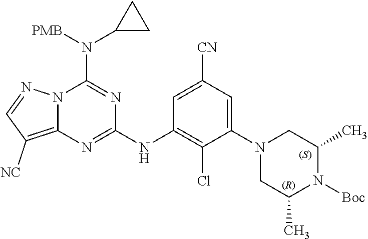

To a mixture of tert-butyl (2S,6R)-4-(3-amino-2-chloro-5-cyanophenyl)-2,6-dimethylpiperazine-1-carboxylate (200 mg, 0.548 mmol) in tetrahydrofuran (10 mL) was added 1 M solution of LiHMDS (1.096 mL, 1.096 mmol) in THF dropwise at −78° C. It was stirred at −78° C. for 30 min and then 4-(cyclopropyl (4-methoxybenzyl)amino)-2-(methylsulfonyl)pyrazolo[1,5-a][1,3,5]triazine-8-carbonitrile (262 mg, 0.658 mmol) dissolved in THF (3 mL) was added and the contents stirred at −78° C. for 30 min. The reaction was quenched with water and extracted with EtOAc. The organic layer was washed with water, dried over MgSO₄, filtered and concentrated. It was then purified by Biotage isolera, eluting with 22% EtOAc/hexane to afford tert-butyl (2R,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (200 mg, 0.288 mmol, 52.5% yield) as an off white solid. LC/MS [M−H]⁺=681.2.

### Step 4. 2-((2-chloro-5-cyano-3-((3R,5S)-3,5-dimethylpiperazin-1-yl)phenyl)amino)-4-(cyclopropylamino)pyrazolo[1,5-a][1,3,5]triazine-8-carbonitrile

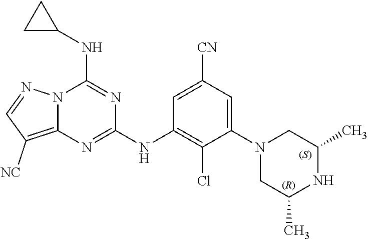

A mixture of tert-butyl (2R,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (920 mg, 1.347 mmol) in DCM (10 mL) was added TFA (2.061 mL, 26.9 mmol) and the contents stirred at rt for 1 h. The reaction mixture was concentrated, the residue was azeotroped with DCM (3×) to yield a light brown solid. The solid (920 mg) was dissolved in ClCH₂CH₂Cl (10 mL). Trifluoroacetic acid (4.04 mL, 52.8 mmol) and anisole (0.574 mL, 5.28 mmol) were added, and the contents heated at 55° C. for 1 h. The reaction mixture was cooled to rt and concentrated. To the residue was added DCM and the contents azeotroped (3×). To the residue was then added 2 mL of 7 N NH₃ in MeOH. The white ppt was filtered and washed with MeOH to afford 2-((2-chloro-5-cyano-3-((3R,5S)-3,5-dimethylpiperazin-1-yl)phenyl)amino)-4-(cyclopropylamino)pyrazolo[1,5-a][1,3,5]triazine-8-carbonitrile (500 mg, 0.997 mmol, 76% yield) as an off-white solid. LC/MS [M+H]⁺=463.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). ¹H-NMR: (400 MHz, DMSO-d₆) δ 9.58 (br s, 1H), 9.23 (br s, 1H), 8.42 (s, 1H), 8.04 (d, J=1.25 Hz, 1H), 7.38 (d, J=1.13 Hz, 1H), 3.25-3.16 (m, 2H), 3.11-2.90 (m, 2H), 2.88-2.79 (m, 1H), 2.39-2.27 (m, 2H), 1.10-0.93 (m, 6H), 0.87-0.72 (m, 4H).

### Step 5. Example 283

A mixture of 2-((2-chloro-5-cyano-3-((3R,5S)-3,5-dimethylpiperazin-1-yl)phenyl)amino)-4-(cyclopropylamino)pyrazolo[1,5-a][1,3,5]triazine-8-carbonitrile (25 mg, 0.054 mmol), DIPEA (0.028 mL, 0.162 mmol) and acetic anhydride (8.27 mg, 0.081 mmol) in DMSO (0.5 mL) was stirred at rt for 16 h. The reaction mixture was quenched with MeOH (1 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({3-[(3R,5S)-4-acetyl-3,5-dimethylpiperazin-1-yl]-2-chloro-5-cyanophenyl}amino)-4-(cyclopropylamino)pyrazolo[1,5-a][1,3,5]triazine-8-carbonitrile (6 mg, 0.012 mmol, 21.68% yield) as an off white solid. LC/MS [M+H]⁺=505.4. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 284

### 2-({2-chloro-5-cyano-3-[(3R,5S)-4-(2-hydroxyethyl)-3,5-dimethylpiperazin-1-yl]phenyl}amino)-4-(cyclopropylamino)pyrazolo[1,5-a][1,3,5]triazine-8-carbonitrile

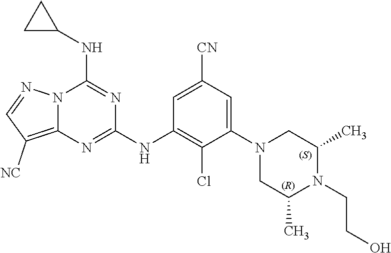

A mixture of 2-((2-chloro-5-cyano-3-((3R,5S)-3,5-dimethylpiperazin-1-yl)phenyl)amino)-4-(cyclopropylamino)pyrazolo[1,5-a][1,3,5]triazine-8-carbonitrile (50 mg, 0.108 mmol), acetic acid (25.9 mg, 0.432 mmol) and 1,4-dioxane-2,5-diol (38.9 mg, 0.324 mmol) in DMSO:THF (7:3) (1 mL) was stirred at rt for 2 h. Sodium triacetoxy borohydride (68.7 mg, 0.324 mmol) was added and the contents stirred at rt for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(3R,5S)-4-(2-hydroxyethyl)-3,5-dimethylpiperazin-1-yl]phenyl}amino)-4-(cyclopropylamino)pyrazolo[1,5-a][1,3,5]triazine-8-carbonitrile (1.67 mg, 3.21 μmol, 2.98% yield) as an off white solid. LC/MS [M+H]⁺=507.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm).

### Example 285

### 2-({2-chloro-5-cyano-3-[(3R,5S)-4-[(2R)-2-hydroxypropyl]-3,5-dimethylpiperazin-1-yl]phenyl}amino)-4-(cyclopropylamino)pyrazolo[1,5-a][1,3,5]triazine-8-carbonitrile

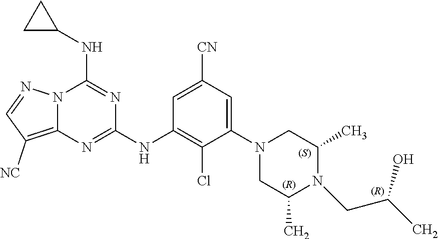

A mixture of 2-((2-chloro-5-cyano-3-((3R,5S)-3,5-dimethylpiperazin-1-yl)phenyl)amino)-4-(cyclopropylamino)pyrazolo[1,5-a][1,3,5]triazine-8-carbonitrile (20 mg, 0.043 mmol), DIPEA (0.023 mL, 0.130 mmol) and (R)-2-methyloxirane (3.76 mg, 0.065 mmol) was taken in DMSO (0.5 mL) and stirred at 80° C. for 16 h. The reaction mixture was cooled to rt, quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(3R,5S)-4-[(2R)-2-hydroxypropyl]-3,5-dimethylpiperazin-1-yl]phenyl}amino)-4-(cyclopropylamino)pyrazolo[1,5-a][1,3,5]triazine-8-carbonitrile (7.4 mg, 0.014 mmol, 32.3% yield) as an off white solid. LC/MS [M+H]⁺=521.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 286

### Methyl (2R,6R)-4-(2-chloro-5-cyano-3-{[8-cyano-4-(cyclopropylamino)pyrazolo[1,5-a][1,3,5]triazin-2-yl]amino}phenyl)-2,6-dimethylpiperazine-1-carboxylate

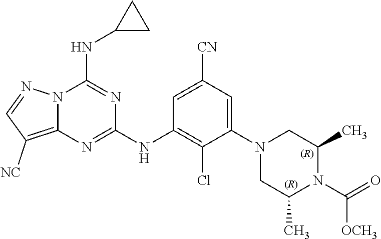

### Step 1. tert-butyl (2R,6R)-4-(3-((tert-butoxycarbonyl)amino)-2-chloro-5-cyanophenyl)-2,6-dimethylpiperazine-1-carboxylate

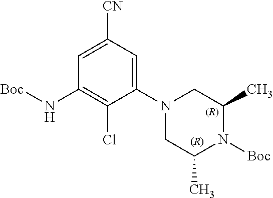

A 250 mL sealed tube was charged tert-butyl (3-bromo-2-chloro-5-cyanophenyl)carbamate (2.5 g, 7.54 mmol), tert-butyl (2R,6R)-2,6-dimethylpiperazine-1-carboxylate (2.100 g, 9.80 mmol), Pd₂dba₃ (0.690 g, 0.754 mmol), BINAP (0.939 g, 1.508 mmol) and cesium carbonate (6.17 g, 19.60 mmol) and toluene (100 mL). Nitrogen was bubbled into the reaction mixture for 15 min and the reaction was heated at 110° C. for 16 h. The reaction mixture was cooled to rt and filtered over celite. The filtrate was concentrated in vacuo. The crude was purified by Biotage Isolera, eluting with 7% EtOAc/hexane to yield tert-butyl (2R,6R)-4-(3-((tert-butoxycarbonyl) amino)-2-chloro-5-cyanophenyl)-2,6-dimethylpiperazine-1-carboxylate (3 g, 84% yield) as a pale-yellow solid. LC/MS [M−H]⁺=463.2.

### Step 2. Tert-butyl (2R,6R)-4-(3-amino-2-chloro-5-cyanophenyl)-2,6-dimethylpiperazine-1-carboxylate

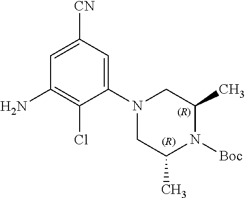

A solution of tert-butyl (2R,6R)-4-(3-((tert-butoxycarbonyl)amino)-2-chloro-5-cyanophenyl)-2,6-dimethylpiperazine-1-carboxylate (3.0 g, 6.45 mmol) in DCM (40 mL) was treated with TFA (7.45 mL, 97 mmol) and the contents stirred at rt for 1 h. The reaction mixture was concentrated, the crude was triturated with Et₂O, and the resulting solid was dried under high vacuum. The solid was then dissolved into 30 ml of DCM, and treated with triethyl amine (4.20 mL, 29.1 mmol), followed by Boc anhydride (1.907 mL, 8.31 mmol). It was stirred at RT for 16 h. The reaction mixture was concentrated, and the crude was purified by Biotage Isolera, eluting with 15% EtOAc/hexane to yield tert-butyl (2R,6R)-4-(3-amino-2-chloro-5-cyanophenyl)-2,6-dimethylpiperazine-1-carboxylate (1.5 g, 46.9% yield) as off white solid. LC/MS [M+H]⁺=365.2.

### Step 3. tert-butyl (2R,6R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate

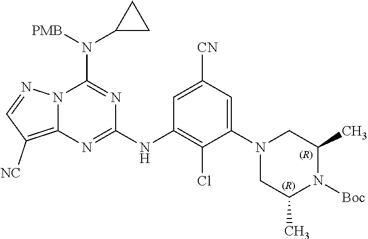

To a mixture of tert-butyl (2R,6R)-4-(3-amino-2-chloro-5-cyanophenyl)-2,6-dimethylpiperazine-1-carboxylate (1.1 g, 3.01 mmol) in tetrahydrofuran (50 mL) was added 1 M solution of LiHMDS (6.03 mL, 6.03 mmol) in THF dropwise at −78° C. It was stirred at −78° C. for 30 min and then 4-(cyclopropyl(4-methoxybenzyl)amino)-2-(methylsulfonyl)pyrazolo[1,5-a][1,3,5]triazine-8-carbonitrile (1.213 g, 3.01 mmol) dissolved in THF was added and stirred at −78° C. for 30 min. The reaction was quenched with water, extracted with EtOAc. The organic layer was washed with water, dried over MgSO₄, filtered and concentrated. It was then purified by Biotage isolera, eluting with 50% EtOAc/hexane to yield tert-butyl (2R,6R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (2.1 g, 69.2% yield) as off white solid. LC/MS [M−H]⁺=681.2.

### Step 4. 2-((2-chloro-5-cyano-3-((3R,5R)-3,5-dimethylpiperazin-1-yl)phenyl)amino)-4-(cyclopropylamino)pyrazolo[1,5-a][1,3,5]triazine-8-carbonitrile

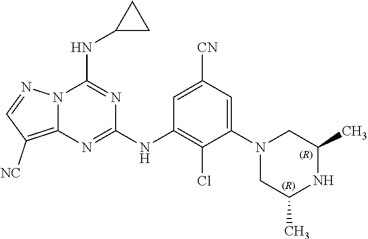

To a solution of tert-butyl (2R,6R)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (1.250 g, 1.830 mmol) in CH₂Cl₂ (20 mL) was added TFA (2.82 mL, 36.6 mmol) and the contents stirred at rt for 2 h. The reaction mixture was concentrated, and the residue was dissolved in DCM and azeotroped three times to yield a light brown solid. The solid was dissolved in ClCH₂CH₂Cl (20 mL), TFA (4.85 mL, 63.0 mmol) was added followed by anisole (0.746 mL, 6.86 mmol) and the contents heated at 50° C. for 1 h. The reaction mixture was cooled to rt and concentrated. The residue was added DCM and the contents concentrated (3×). To the residue was then added 2 mL of 7 N NH₃ in MeOH. The white ppt. was filtered and washed with MeOH to yield 2-((2-chloro-5-cyano-3-((3R,5R)-3,5-dimethylpiperazin-1-yl)phenyl)amino)-4-(cyclopropylamino) pyrazolo[1,5-a][1,3,5]triazine-8-carbonitrile (720 mg) as an off-white solid. LC/MS [M+H]⁺=463.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm).

### Step 5. Example 286

A mixture of 2-((2-chloro-5-cyano-3-((3R,5R)-3,5-dimethylpiperazin-1-yl)phenyl)amino)-4-(cyclopropylamino)pyrazolo[1,5-a][1,3,5]triazine-8-carbonitrile (30 mg, 0.065 mmol), DIPEA (0.034 ml, 0.194 mmol) and methyl carbonochloridate (9.19 mg, 0.097 mmol) in DMSO (1 mL) was stirred at rt for 16 h. The reaction mixture was quenched with MeOH (1 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave methyl (2R,6R)-4-(2-chloro-5-cyano-3-{[8-cyano-4-(cyclopropylamino)pyrazolo[1,5-a][1,3,5]triazin-2-yl]amino}phenyl)-2,6-dimethylpiperazine-1-carboxylate (6.5 mg, 0.012 mmol, 18.96% yield) as an off white solid. LC/MS [M+H]⁺=521.4. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.58 (s, 1H), 9.22 (s, 1H), 8.41 (s, 1H), 7.95 (d, J=1.20 Hz, 1H), 7.41 (d, J=2.00 Hz, 1H), 4.07 (dd, J=3.60, 6.60 Hz, 2H), 3.78 (dd, J=3.20, Hz, 2H), 3.64 (s, 3H), 3.00 (dd, J=3.60, 12.00 Hz, 2H), 2.85 (d, J=4.00 Hz, 1H), 1.28 (d, J=6.80 Hz, 6H), 0.81-0.76 (m, 4H).

The following compound was prepared in a similar procedure as described for Example 286 employing the R⁴ group identified in the table below:

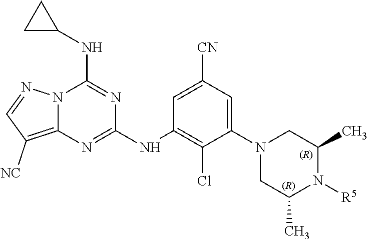

| | | LC/MS [M + H]⁺/ | |
|---|---|---|---|
| Ex # | R⁵ | RT (Method) | ¹H-NMR (400 MHz, DMSO-d₆) |
| 287 | 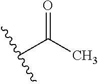 [SMILES: *C(C)=O] | 505.2 2.46 min (B) | δ 9.60 (s, 1H), 9.23 (s, 1H), 8.42 (s, 1H), 7.89 (s, 1H), 7.41 (d, J = 2.00 Hz, 1H), 4.07 (m, 2H), 4.04 (dd, J =, Hz, 2H), 3.13-3.12 (m, 2H), 2.68-2.67 (m, 1H), 2.05 (s, 1H), 1.26-1.24 (m, 7H), 0.81-0.76 (m, 4H). |
| 288 | 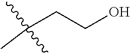 [SMILES: *CCO] | 507.2 (A) | |
| 289 | 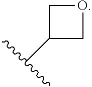 [SMILES: *C1COC1] | 519.2 1.76 min (A) | |

### LCMS Conditions:

Method A: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 290

### 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile

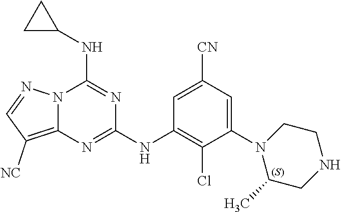

### Step 1. Tert-butyl (S)-4-(3-((tert-butoxycarbonyl)amino)-2-chloro-5-cyanophenyl)-3-methylpiperazine-1-carboxylate

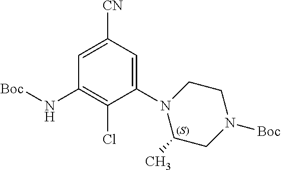

A 500 mL 3 neck RB flask was charged with tert-butyl (3-bromo-2-chloro-5-cyanophenyl)carbamate (5 g, 15.08 mmol), tert-butyl (S)-3-methylpiperazine-1-carboxylate (4.53 g, 22.62 mmol), toluene (75 mL). Pd₂(dba)₃ (1.381 g, 1.508 mmol), BINAP (1.878 g, 3.02 mmol) and Cs₂CO₃ (14.74 g, 45.2 mmol) under N₂ purging. Nitrogen bubbling was continued for 15 min and the reaction was heated at 110° C. for 16 h under nitrogen atmosphere. The reaction mixture was cooled to room temperature. The reaction mixture was filtered over celite. The filtrate was concentrated in vacuo. The crude obtained was purified by Biotage Isolera, eluting with 15% EtOAc/hexane to afford tert-butyl (S)-4-(3-((tert-butoxycarbonyl)amino)-2-chloro-5-cyanophenyl)-3-methylpiperazine-1-carboxylate (2.5 g, 4.82 mmol, 32.0% yield) as a pale-yellow solid. LC/MS [M+H]⁺=451.3. ¹H-NMR (400 MHz, DMSO-d₆): δ 8.90 (s, 1H), 7.79 (d, J=1.60 Hz, 1H), 7.51 (d, J=1.20 Hz, 1H), 3.63-3.59 (m, 1H), 3.47-3.41 (m, 3H), 3.15-3.12 (m, 2H), 2.65-2.61 (m, 1H), 1.47 (s, 9H), 1.41 (s, 9H), 1.80 (d, J=8.00 Hz, 3H).

### Step 2. Tert-butyl (S)-4-(3-amino-2-chloro-5-cyanophenyl)-3-methylpiperazine-1-carboxylate

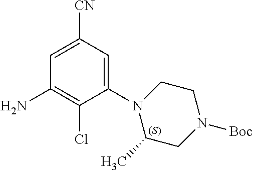

A solution of tert-butyl (S)-4-(3-((tert-butoxycarbonyl)amino)-2-chloro-5-cyanophenyl)-3-methylpiperazine-1-carboxylate (2.8 g, 6.21 mmol) in DCM (30 mL) was treated with trifluoroacetic acid (8.08 mL, 106 mmol) at rt for 1 h. The reaction mixture was concentrated, and the crude was triturated with Et₂O, and the resulting solid was dried under high vacuum. The solid was then dissolved in 30 ml of DCM, and treated with triethylamine (6.42 mL, 46.1 mmol), followed by Boc-anhydride (1.764 mL, 7.68 mmol). It was stirred at rt for 16 h. The reaction mixture was concentrated, and the crude was purified by Biotage Isolera, eluting with 16% EtOAc/hexane to yield tert-butyl (S)-4-(3-amino-2-chloro-5-cyanophenyl)-3-methylpiperazine-1-carboxylate (1.35 g, 2.57 mmol, 33.4% yield) as a pale-yellow gummy solid. LC/MS [M−H]⁺=351.2. ¹H-NMR (400 MHz, CDCl₃): δ 6.81 (d, J=2.00 Hz, 1H), 6.74 (d, J=2.00 Hz, 1H), 4.36 (br s, 1H), 3.75 (br s, 1H), 3.65-3.56 (m, 2H), 3.39-3.37 (m, 1H), 3.22-3.14 (m, 2H), 1.52 (s, 9H), 0.93 (s, 3H).

### Step 3. tert-butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate

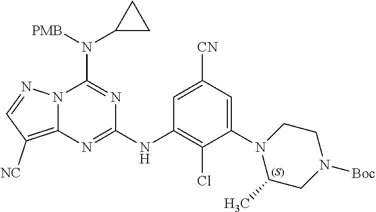

To a mixture of tert-butyl (S)-4-(3-amino-2-chloro-5-cyanophenyl)-3-methylpiperazine-1-carboxylate (1.35 g, 3.85 mmol) in tetrahydrofuran (20 mL) was added 1 M solution of Lithium bis(trimethylsilyl)amide solution (7.70 mL, 7.70 mmol) in THF dropwise at −78° C. The reaction mixture was stirred at −78° C. for 30 min and 4-(cyclopropyl(4-methoxybenzyl)amino)-2-(methylsulfonyl)pyrazolo[1,5-a][1,3,5]triazine-8-carbonitrile (1.533 g, 3.85 mmol) in tetrahydrofuran (20 mL) was added and stirred at −78° C. for 30 min. The reaction was quenched with water, stirred for 10 min at rt and extracted with EtOAc. The organic layer was washed with water, dried over MgSO₄, filtered and concentrated. It was then purified by Biotage isolera, eluting with 30% EtOAc/hexane to afford tert-butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate (1.8 g, 2.62 mmol, 68.0% yield) as off white solid. LC/MS [M−H]⁺=667.2. ¹H-NMR (400 MHz, CDCl₃): δ 9.76 (s, 1H), 8.42 (s, 1H), 7.94 (br s, 1H), 7.53 (s, 1H), 7.16 (d, J=8.40 Hz, 2H), 6.86 (d, J=8.80 Hz, 2H), 5.14-5.11 (m, 2H), 3.72 (s, 3H), 3.55-3.50 (m, 2H), 3.18-3.14 (m, 2H), 2.88 (br s, 1H), 2.62-2.55 (m, 2H), 1.43 (s, 9H), 0.85-0.77 (m, 7H).

### Step 4. Example 290

A mixture of tert-butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate (1.8 g, 2.69 mmol) in CH₂Cl₂ (15 mL) was added trifluoroacetic acid (4.12 mL, 53.8 mmol) and the contents stirred at rt for 1 h and concentrated. The residue obtained was dissolved in dry DCM (10 mL) and concentrated for three times to yield a light brown solid. The solid (S)-2-((2-chloro-5-cyano-3-(2-methylpiperazin-1-yl)phenyl)amino)-4-(cyclopropyl(4-methoxybenzyl)amino)pyrazolo[1,5-a][1,3,5]triazine-8-carbonitrile (1.8 g, 2.64 mmol) obtained was dissolved in ClCH₂CH₂Cl (15 mL). Trifluoroacetic acid (8.07 mL, 105 mmol) was added followed by anisole (1.146 mL, 10.54 mmol) and the contents heated at 50° C. for 2 h cooled to rt and concentrated. To the residue was added DCM and the contents concentrated 3 times. To the residue obtained was added 3 mL of 7 N NH₃ in MeOH. The ppt. was filtered and washed with MeOH to afford 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (900 mg, 1.921 mmol, 72.9% yield) as an off-white solid. LC/MS [M+H]⁺=449.1. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 291

### 2-({2-chloro-5-cyano-3-[(2S)-4-(2-methoxyethyl)-2-methylpiperazin-1-yl]phenyl}amino)-4-(cyclopropylamino)pyrazolo[1,5-a][1,3,5]triazine-8-carbonitrile

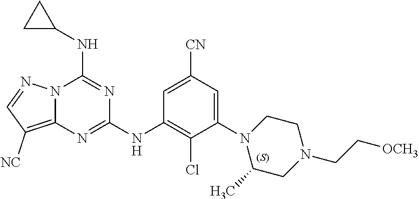

A mixture of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (30 mg, 0.067 mmol), DIPEA (0.012 mL, 0.067 mmol) and 1-bromo-2-methoxyethane (9.29 mg, 0.067 mmol) in DMSO (0.5 mL) was stirred at rt for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(2S)-4-(2-methoxyethyl)-2-methylpiperazin-1-yl]phenyl}amino)-4-(cyclopropylamino)pyrazolo[1,5-a][1,3,5]triazine-8-carbonitrile (10 mg, 0.019 mmol, 29.1% yield) as an off white solid. LC/MS [M+H]⁺=507.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.53 (s, 1H), 9.24 (d, J=3.20 Hz, 1H), 8.42 (s, 1H), 8.15 (d, J=1.60 Hz, 1H), 7.54 (d, J=1.20 Hz, 1H), 3.46-3.49 (m, 3H), 3.33 (s, 3H), 3.15-3.17 (m, 1H), 2.84-2.84 (m, 1H), 2.76-2.83 (m, 1H), 2.65-2.70 (m, 2H), 2.33-2.34 (m, 1H), 0.85-0.87 (m, 3H), 0.85-0.87 (m, 2H), 0.77-0.80 (m, 2H), 0.74-0.76 (m, 2H).

The following compounds were prepared in a similar procedure as described for Example 291 employing the R⁵ group identified in the table below:

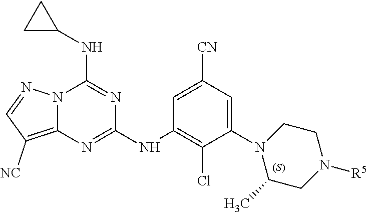

| Ex | | LC/MS [M + H]⁺/ | |
|---|---|---|---|
| # | R⁵ | RT (Method) | ¹H-NMR (400 MHz, DMSO-d₆) |
| 292 | 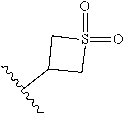 [SMILES: *C1CS(=O)(=O)C1] | 553.2 2.73 min (A) | δ 9.56 (s, 1H), 9.25 (d, J = 3.60 Hz, 1H), 8.43 (s, 1H), 8.17 (s, 1H), 7.58 (s, 1H), 4.31-4.22 (m, 4H), 3.43 (m, 2H), 3.38 (m, 2H), 3.19 (m, 1H), 2.86-2.83 (m, 2H), 2.76-2.74 (m, 2H), 2.28 (m, 1H), 0.89 (d, J = 6.40 Hz, 3H), 0.83-0.74 (m, 4H). |
| 293 | 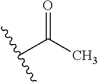 [SMILES: *C(C)=O] | 491.1 2.224 min (B) | — |

### Example 294

### 2-({2-chloro-5-cyano-3-[(2S)-4-(2-hydroxyethyl)-2-methylpiperazin-1-yl]phenyl}amino)-4-(cyclopropylamino)pyrazolo[1,5-a][1,3,5]triazine-8-carbonitrile

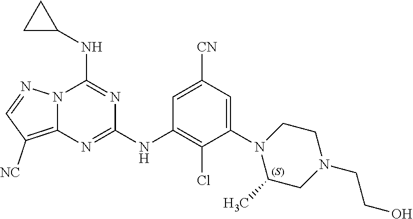

To a stirred solution of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (30 mg, 0.067 mmol in DMSO:THF (7:3) (1 mL) was added acetic acid (0.013 mL, 0.230 mmol) and 1,4-dioxane-2,5-diol (6.45 mg, 0.090 mmol) and the contents stirred at rt for 2 h. Sodium triacetoxyborohydride (42.5 mg, 0.200 mmol) was added and the contents stirred at RT for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(2S)-4-(2-hydroxyethyl)-2-methylpiperazin-1-yl]phenyl}amino)-4-(cyclopropylamino)pyrazolo [1,5-a][1,3,5]triazine-8-carbonitrile (5.5 mg, 10.93 μmol, 16.36% yield) as an off white solid. LC/MS [M+H]⁺=493.2. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.79 (s, 1H), 9.63 (d, J=8.80 Hz, 1H), 9.29 (d, J=2.80 Hz, 1H), 8.44 (s, 1H), 8.30 (s, 1H), 7.69 (d, J=1.60 Hz, 1H), 5.43 (brs, 1H), 3.79 (m, 2H), 3.66-3.63 (m, 4H), 3.28 (m, 2H), 3.22-3.16 (m, 4H), 3.01-2.92 (m, 3H), 1.05 (d, J=6.00 Hz, 3H), 0.90 (d, J=6.00 Hz, 2H).

The following compounds were prepared in a similar procedure as described for Example 294 employing the R⁵ group identified in the table below:

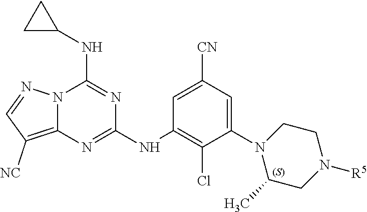

| | | LC/MS [M + H]⁺/ | |
|---|---|---|---|
| Ex # | R⁵ | RT (Method) | ¹H-NMR (400 MHz, DMSO-d₆) |
| 295 | 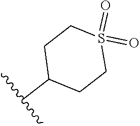 [SMILES: *C1CCS(=O)(=O)CC1] | 581.2 1.698 min (A) | δ 9.84 (d, J = 14.00 Hz, 1H), 9.59 (s, 1H), 9.27 (s, 1H), 8.31 (s, 1H), 8.17 (s, 1H), 7.68 (s, 1H), 3.80 (m, 3H), 3.30-3.22 (m, 9H), 2.94-2.86 (m, 4H), 2.20 (m, 3H), 1.12-1.11 (m, 1H), 0.93 (d, J = 5.20 Hz, 3H), 0.86-0.74 (m, 6H). |
| 296 | 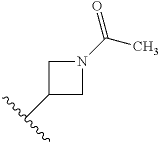 [SMILES: *C1CN(C(C)=O)C1] | 546.2 1.922 min (A) | δ 9.54 (s, 1H), 9.24 (s, 1H), 8.42 (s, 1H), 8.16 (d, J = 1.60 Hz, 1H), 7.55 (s, 1H), 4.10- 4.12 (m, 1H), 3.95-4.00 (m, 1H), 3.84-3.87 (m, 1H), 3.65-3.70 (m, 1H), 3.52-3.54 (m, 1H), 3.19-3.21 (m, 2H), 2.83-2.84 (m, 1H), 2.68-2.69 (m, 1H), 2.41-2.50 (m, 1H), 2.33- 2.34 (m, 1H), 1.77 (d, J = 2.80 Hz, 3H), 0.80-0.90 (m, 3H), 0.74-0.79 (m, 4H). |

### Example 297

### 2-({2-chloro-5-cyano-3-[(2S)-4-[(2R)-2-hydroxypropyl]-2-methylpiperazin-1-yl]phenyl}amino)-4-(cyclopropylamino)pyrazolo[1,5-a][1,3,5]triazine-8-carbonitrile

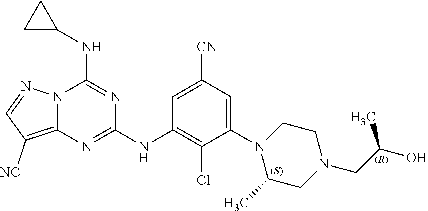

A mixture of 2-({2-chloro-5-cyano-3-[(2S)-2-methylpiperazin-1-yl]phenyl}amino)-4-(cyclopropylamino)pyrazolo[1,5-a][1,3,5]triazine-8-carbonitrile (30 mg, 0.067 mmol), DIPEA (0.035 mL, 0.200 mmol) and (R)-2-methyloxirane (5.82 mg, 0.100 mmol) was taken in DMSO (1 mL) and stirred at 80° C. for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(2S)-4-[(2R)-2-hydroxypropyl]-2-methylpiperazin-1-yl]phenyl}amino)-4-(cyclopropylamino) pyrazolo[1,5-a][1,3,5]triazine-8-carbonitrile (9 mg, 0.017 mmol, 25.2% yield) as an off white solid. LC/MS [M+H]⁺=507.2. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.53 (s, 1H), 9.24 (d, J=3.20 Hz, 1H), 8.42 (s, 1H), 8.15 (s, 1H), 7.52 (s, 1H), 4.33-4.34 (m, 1H), 3.79-3.81 (m, 1H), 3.49-3.52 (m, 1H), 3.15-3.20 (m, 1H), 2.68-2.75 (m, 1H), 2.61-2.67 (m, 2H), 2.24-2.30 (m, 3H), 1.07-1.24 (m, 3H), 0.83-0.89 (m, 3H), 0.78-0.79 (m, 2H), 0.75-0.77 (m, 2H).

The following compounds were prepared in a similar procedure as described for Example 297 employing the R⁵ group identified in the table below:

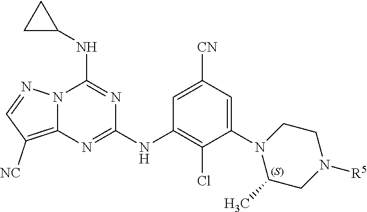

| | | LC/MS [M + H]⁺/ | |
|---|---|---|---|
| Ex # | R⁵ | RT (Method) | ¹H-NMR (400 MHz, DMSO-d₆) |
| 298 | 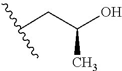 [SMILES: *C[C@H](C)O] | 507.2 1.287 min (A) | δ 9.53 (s, 1H), 9.24 (d, J = 2.80 Hz, 1H), 8.42 (s, 1H), 8.14 (d, J = 1.60 Hz, 1H), 7.53 (d, J = 1.20 Hz, 1H), 4.31 (d, J = 4.00 Hz, 1H), 3.79- 3.81 (m, 1H), 3.50-3.51 (m, 1H), 3.15-3.17 (m, 1H), 2.83-2.85 (m, 1H), 2.76-2.82 (m, |
| | | | 1H), 2.68-2.71 (m, 1H), 2.67-2.68 (m, 1H), |
| | | | 2.33-2.34 (m, 1H), 2.21-2.30 (m, 2H), 1.08 |
| | | | (d, J = 6.40 Hz, 3H), 0.80-0.88 (m, 3H), 0.77- |
| | | | 0.79 (m, 2H), 0.75-0.76 (m, 2H). |
| 299 | 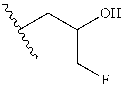 [SMILES: *CC(O)CF] | 525.2 1.315 min (A) | δ 9.53 (s, 1H), 9.24 (d, J = 2.80 Hz, 1H), 8.42 (s, 1H), 8.15 (d, J = 0.80 Hz, 1H), 7.53 (s, 1H), 4.98 (t, J = 3.60 Hz, 1H), 4.41-4.49 (m, 1H), 4.40-4.40 (m, 1H), 4.29-4.39 (m, 1H), 3.92-3.84 (m, 1H), 3.50-3.51 (m, 1H), 3.17- 3.28 (m, 1H), 2.83-2.85 (m, 2H), 2.68-2.69 |
| | | | (m, 2H), 2.43-2.46 (m, 2H), 2.36-2.39 (m, |
| | | | 1H), 0.83-0.89 (m, 3H), 0.75-0.79 (m, 4H). |
| 300 | 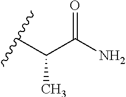 [SMILES: *[C@@H](C)C(N)=O] | 520.0 1.321 min (A) | δ 9.53 (s, 1H), 9.24 (s, 1H), 8.42 (s, 1H), 8.15 (d, J = 1.60 Hz, 1H), 7.52 (d, J = 1.60 Hz, 1H), 7.22 (d, J = 2.80 Hz, 1H), 7.07 (d, J = 2.40 Hz, 1H), 3.50-3.52 (m, 1H), 3.16- 3.19 (m, 1H), 3.02-3.07 (m, 1H), 2.83-2.86 (m, 1H), 2.72-2.77 (m, 2H), 2.68-2.69 (m, 1H), 2.33-2.35 (m, 1H), 1.11-1.13 (m, 3H), |
| | | | 0.82-0.88 (m, 3H), 0.76-0.79 (m, 2H), 0.74- |
| | | | 0.76 (m, 2H). |
| 301 | 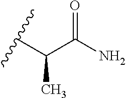 [SMILES: *[C@H](C)C(N)=O] | 520.2 1.319 min (A) | δ 9.53 (s, 1H), 9.24 (s, 1H), 8.42 (s, 1H), 8.15 (d, J = 1.20 Hz, 1H), 7.52 (d, J = 1.60 Hz, 1H), 7.23 (d, J = 2.40 Hz, 1H), 7.06 (d, J = 2.40 Hz, 1H), 3.52-3.53 (m, 1H), 3.16- 3.21 (m, 1H), 3.04-3.07 (m, 1H), 2.78-2.84 (m, 1H), 2.71-2.73 (m, 2H), 2.68-2.70 (m, 2H), 2.31-2.34 (m, 1H), 1.13 (d, J = 6.80 Hz, |
| | | | 3H), 0.81-0.89 (m, 3H), 0.76-0.79 (m, 2H), |
| | | | 0.74-0.76 (m, 2H). |
| 302 | 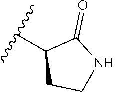 [SMILES: *[C@@H]1CCNC1=O] | 532.0 1.362 min (A) | δ 9.53 (s, 1H), 9.24 (d, J = 3.20 Hz, 1H), 8.42 (s, 1H), 8.15 (d, J = 1.20 Hz, 1H), 7.73 (s, 1H), 7.54 (d, J = 1.60 Hz, 1H), 3.45-3.46 (m, 1H), 3.15-3.18 (m, 3H), 2.95-3.01 (m, 1H), 2.84-2.84 (m, 1H), 2.74-2.76 (m, 1H), 2.68- 2.69 (m, 1H), 2.59-2.62 (m, 3H), 2.13-2.15 (m, 1H), 1.97-1.99 (m, 1H), 0.83-0.87 (m, 3H), 0.77-0.79 (m, 2H), 0.74-0.76 (m, 2H). |
| 303 | 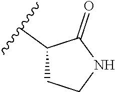 [SMILES: *[C@H]1CCNC1=O] | 532.2 1.624 min (A) | δ 9.52 (s, 1H), 9.24 (s, 1H), 8.42 (s, 1H), 8.15 (d, J = 1.20 Hz, 1H), 7.74 (s, 1H), 7.55 (d, J = 1.60 Hz, 1H), 3.51-3.53 (m, 1H), 3.35- 3.36 (m, 1H), 3.17-3.25 (m, 4H), 2.91-2.93 (m, 1H), 2.84-2.90 (m, 1H), 2.67-2.68 (m, 2H), 2.33-2.34 (m, 1H), 2.13-2.15 (m, 1H), 1.97-1.99 (m, 1H), 0.87 (d, J = 6.00 Hz, 3H), 0.78-0.79 (m, 2H), 0.74-0.77 (m, 2H). |

### LCMS Conditions:

Method A: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 304

### 2-({2-chloro-5-cyano-3-[(3S,5S)-4-(2-methoxyethyl)-3,5-dimethylpiperazin-1-yl]phenyl}amino)-4-(cyclopropylamino)pyrazolo[1,5-a][1,3,5]triazine-8-carbonitrile

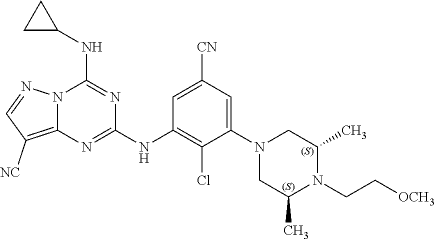

### Step 1. tert-butyl (2S,6S)-4-(3-((tert-butoxycarbonyl)amino)-2-chloro-5-cyanophenyl)-2,6-dimethylpiperazine-1-carboxylate

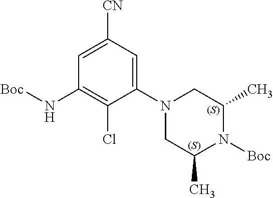

A 250 mL 3 neck RB flask, was charged with tert-butyl (3-bromo-2-chloro-5-cyanophenyl)carbamate (2.5 g, 7.54 mmol), tert-butyl (2S,6S)-2,6-dimethylpiperazine-1-carboxylate (2.100 g, 9.80 mmol), Pd₂dba₃ (0.690 g, 0.754 mmol), BINAP (0.939 g, 1.508 mmol), Cesium carbonate (5.94 g, 18.85 mmol) and toluene (50 mL). Nitrogen was bubbled into the reaction mixture for 10 min and the reaction was heated at 110° C. for 16 h under nitrogen atmosphere. The reaction mixture was cooled to rt, filtered over celite, and washed with EtOAc (50 mL). The filtrate was concentrated in vacuo. The crude was purified by Biotage Isolera, eluting with 16% EtOAc/hexane to afford tert-butyl (2S,6S)-4-(3-((tert-butoxycarbonyl)amino)-2-chloro-5-cyanophenyl)-2,6-dimethylpiperazine-1-carboxylate (2 g, 57% yield) as a pale-yellow solid. LC/MS [M+H]⁺=465.2. ¹H-NMR (400 MHz, DMSO-d₆): δ 8.90 (s, 1H), 7.59 (d, J=1.60 Hz, 1H), 7.36 (d, J=1.60 Hz, 1H), 4.06-3.97 (m, 3H), 3.77-3.72 (m, 2H), 2.95-2.90 (m, 2H), 1.47 (s, 9H), 1.43 (s, 9H), 1.25 (d, J=6.40 Hz, 6H).

### Step 2. tert-butyl (2S,6S)-4-(3-amino-2-chloro-5-cyanophenyl)-2,6-dimethylpiperazine-1-carboxylate

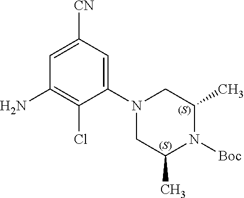

A solution of tert-butyl (2S,6S)-4-(3-((tert-butoxycarbonyl)amino)-2-chloro-5-cyanophenyl)-2,6-dimethylpiperazine-1-carboxylate (2.0 g, 4.30 mmol) in DCM (50 mL) was treated with TFA (4.97 mL, 64.5 mmol) and the contents stirred at rt for 30 min. The reaction mixture was concentrated, triturated with Et₂O and the resulting solid was dried under high vacuum. The solid was dissolved in 20 mL of DCM, and treated with triethyl amine (2.480 mL, 17.19 mmol), followed by Boc anhydride (1.127 mL, 4.91 mmol). The reaction mixture was stirred for 16 h at RT and the crude was purified by Biotage Isolera, eluting with 14% EtOAc/hexane to yield tert-butyl (2S,6S)-4-(3-amino-2-chloro-5-cyanophenyl)-2,6-dimethylpiperazine-1-carboxylate (1.3 g, 68.2% yield) as off white solid. LC/MS [M+H]⁺=365.2. ¹H-NMR (400 MHz, DMSO-d₆): δ 6.75 (d, J=2.00 Hz, 1H), 6.69 (d, J=1.60 Hz, 1H), 5.81 (s, 2H), 4.01-3.94 (m, 2H), 3.65-3.58 (m, 2H), 2.92-2.85 (m, 2H), 1.42 (s, 9H), 1.24 (d, J=6.40 Hz, 6H).

### Step 3. tert-butyl (2S,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate

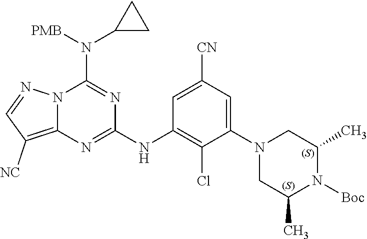

To a mixture of tert-butyl (2S,6S)-4-(3-amino-2-chloro-5-cyanophenyl)-2,6-dimethylpiperazine-1-carboxylate (1.2 g, 3.29 mmol) in tetrahydrofuran (50 mL) was added 1 M solution of LIHMDS (6.58 mL, 6.58 mmol) in THF dropwise at −78° C. It was stirred at −78° C. for 30 min and then 4-(cyclopropyl(4-methoxybenzyl)amino)-2-(methylsulfonyl)pyrazolo[1,5-a][1,3,5]triazine-8-carbonitrile (1.456 g, 3.62 mmol) dissolved in THF was added and stirred at −78° C. for 30 min. The reaction was quenched with water and extracted with EtOAc. The organic layer was washed with water, dried over MgSO₄, filtered and concentrated. It was purified by Biotage isolera, eluting with 33% EtOAc/hexane to afford tert-butyl (2S,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (1.7 g, 74.9% yield) as off white solid. LC/MS [M−H]⁺=681.2.

### Step 4. 2-((2-chloro-5-cyano-3-((3S,5S)-3,5-dimethylpiperazin-1-yl)phenyl)amino)-4-(cyclopropylamino)pyrazolo[1,5-a][1,3,5]triazine-8-carbonitrile

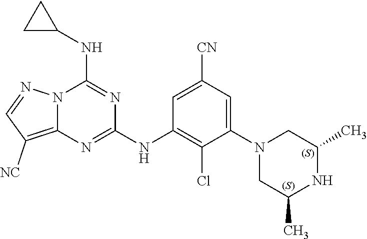

A mixture of tert-butyl (2S,6S)-4-(2-chloro-5-cyano-3-((8-cyano-4-(cyclopropyl(4-methoxybenzyl)amino)pyrazolo[1,5-a][1,3,5]triazin-2-yl)amino)phenyl)-2,6-dimethylpiperazine-1-carboxylate (1.8 g, 2.63 mmol) in CH₂Cl₂ (40 mL) was added TFA (4.06 mL, 52.7 mmol) and the contents stirred at rt for 2 h. The reaction mixture was concentrated, and the residue was dissolved in DCM and concentrated to yield an off white solid (1.4 g) as TFA salt. The solid (0.8 g) was dissolved in ClCH₂CH₂Cl (20 mL) TFA (4.23 mL, 54.9 mmol) was added followed by anisole (0.596 mL, 5.49 mmol) and the contents heated at 55° C. for 2 h. The reaction mixture was cooled to rt, concentrated and the residue azeotroped with DCM (3×). To the residue was added 2.5 mL of 7 N NH₃ in MeOH. The white ppt. formed was filtered and washed with MeOH to afford 2-((2-chloro-5-cyano-3-((3S,5S)-3,5-dimethylpiperazin-1-yl)phenyl)amino)-4-(cyclopropylamino)pyrazolo[1,5-a][1,3,5]triazine-8-carbonitrile (550 mg, 87%) as an off-white solid. LC/MS [M+H]⁺=463.2.

### Step 5. Example 304

A mixture of 2-((2-chloro-5-cyano-3-((3S,5S)-3,5-dimethylpiperazin-1-yl)phenyl)amino)-4-(cyclopropylamino)pyrazolo[1,5-a][1,3,5]triazine-8-carbonitrile (20 mg, 0.043 mmol), DIPEA (0.038 mL, 0.216 mmol) and 1-bromo-2-methoxyethane (9.01 mg, 0.065 mmol) in DMSO (0.5 mL) was stirred at 85° C. for 24 h. The reaction mixture was quenched with MeOH (1 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-((2-chloro-5-cyano-3-((3S,5S)-4-(2-methoxyethyl)-3,5-dimethylpiperazin-1-yl)phenyl)amino)-4-(cyclopropylamino)pyrazolo[1,5-a][1,3,5]triazine-8-carbonitrile (10.5 mg, 0.020 mmol, 45.8% yield) as an off white solid. LC/MS [M+H]⁺=521.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.57 (s, 1H), 9.23 (s, 1H), 8.42 (s, 1H), 8.05 (d, J=1.5 Hz, 1H), 7.36 (d, J=1.9 Hz, 1H), 3.46-3.36 (m, 4H), 3.26 (s, 3H), 3.12-2.96 (m, 4H), 2.87-2.73 (m, 4H), 1.08 (d, J=6.3 Hz, 6H), 0.80 (br d, J=2.4 Hz, 2H), 0.78-0.68 (m, 2H).

### Example 305

### (2S)-2-[(2S,6S)-4-(2-chloro-5-cyano-3-{[8-cyano-4-(cyclopropylamino)pyrazolo[1,5-a][1,3,5]triazin-2-yl]amino}phenyl)-2,6-dimethylpiperazin-1-yl]propanamide

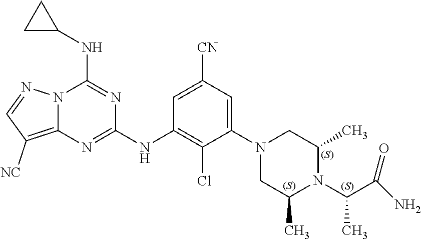

A mixture of 2-((2-chloro-5-cyano-3-((3S,5S)-3,5-dimethylpiperazin-1-yl)phenyl)amino)-4-(cyclopropylamino)pyrazolo[1,5-a][1,3,5]triazine-8-carbonitrile (40 mg, 0.090 mmol), DIPEA (0.094 mL, 0.540 mmol) and (R)-1-amino-1-oxopropan-2-yl methanesulfonate (90 mg, 0.540 mmol) was taken in DMSO (1 mL) and stirred at 80° C. for 16 h. The reaction was quenched by MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave (2S)-2-[(2S,6S)-4-(2-chloro-5-cyano-3-{[8-cyano-4-(cyclopropylamino)pyrazolo[1,5-a][1,3,5]triazin-2-yl]amino}phenyl)-2,6-dimethylpiperazin-1-yl]propenamide (3.0 mg, 5.28 μmol, 4.89% yield) LC/MS [M+H]⁺=534.2. LCMS Injection condition: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm).

### Example 306

### Methyl (3R)-4-(2-chloro-5-cyano-3-{[8-cyano-4-(cyclopropylamino)pyrazolo[1,5-a][1,3,5]triazin-2-yl]amino}phenyl)-3-methylpiperazine-1-carboxylate

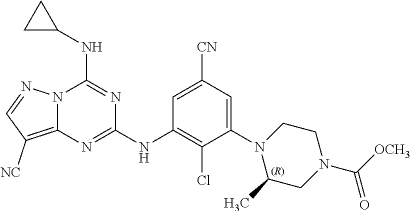

### Step 1. Tert-butyl (R)-4-(3-((tert-butoxycarbonyl)amino)-2-chloro-5-cyanophenyl)-3-methylpiperazine-1-carboxylate

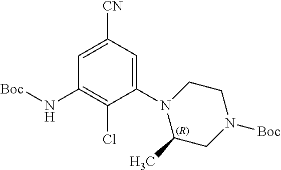

A 250 mL 3 neck RB flask is charged with tert-butyl (3-bromo-2-chloro-5-cyanophenyl)carbamate (2.5 g, 7.54 mmol), tert-butyl (R)-3-methyl piperazine-1-carboxylate (2.265 g, 11.31 mmol) and toluene (50 mL). Nitrogen was bubbled into the mixture for 5 min. Pd₂(dba)₃ (0.690 g, 0.754 mmol), BINAP (0.939 g, 1.508 mmol) and Cs₂CO₃ (7.37 g, 22.62 mmol) was added under N₂ purging. Nitrogen was bubbled for another 5 min and the reaction was heated at 110° C. for 16 h under nitrogen atmosphere. The reaction mixture was cooled to room temperature and filtered over celite. The filtrate was concentrated in vacuo. The crude was purified by Biotage Isolera, eluting with 8% EtOAc/hexane to afford tert-butyl (R)-4-(3-((tert-butoxycarbonyl)amino)-2-chloro-5-cyanophenyl)-3-methylpiperazine-1-carboxylate (1.15 g, 30% yield) as a pale-yellow solid. LC/MS [M+H]⁺=451.2.

### Step 2. tert-butyl (R)-4-(3-amino-2-chloro-5-cyanophenyl)-3-methylpiperazine-1-carboxylate

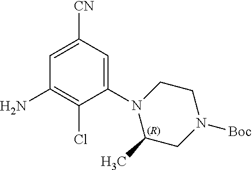

A solution of tert-butyl (R)-4-(3-((tert-butoxycarbonyl)amino)-2-chloro-5-cyanophenyl)-3-methylpiperazine-1-carboxylate (3 g, 6.65 mmol) in DCM (40 mL) was treated with TFA (7.69 mL, 100 mmol) and the contents stirred at rt for 1 h. The reaction mixture was concentrated, the crude was triturated with Et₂O, and the resulting solid was dried under high vacuum. The solid (3 g, 8.22 mmol) was dissolved in 30 mL of DCM, and treated with triethylamine (6.88 mL, 49.3 mmol), followed by di-tert-butyl dicarbonate (1.890 mL, 8.22 mmol). It was stirred for 30 min at rt. The reaction mixture was concentrated, and the crude was purified by Biotage Isolera, eluting with 16% EtOAc/hexane to yield tert-butyl (R)-4-(3-amino-2-chloro-5-cyanophenyl)-3-methylpiperazine-1-carboxylate (1.66 g, 4.38 mmol, 53.2% yield) as pale-yellow gummy solid. LC/MS [M−H]⁺=351.2. ¹H-NMR (400 MHz, CDCl₃): δ 6.81 (d, J=2.00 Hz, 1H), 6.74 (d, J=1.60 Hz, 1H), 4.42-4.29 (m, 2H), 3.80-3.71 (m, 1H), 3.68-3.52 (m, 2H), 3.40-3.32 (m, 1H), 3.27-3.10 (m, 2H), 2.68-2.60 (m, 1H), 1.51 (s, 9H), 0.93 (d, J=6.40 Hz, 3H).

### Step 3. tert-butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate

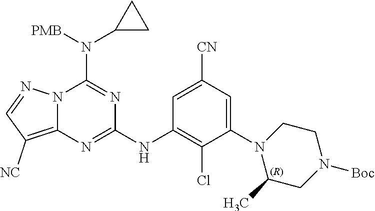

To a mixture of tert-butyl (R)-4-(3-amino-2-chloro-5-cyanophenyl)-3-methylpiperazine-1-carboxylate (1.66 g, 4.73 mmol) in tetrahydrofuran (20 mL) was added 1 M solution of LiHMDS (9.46 mL, 9.46 mmol) in THF dropwise at −78° C. It was stirred at −78° C. for 30 min and then 4-(cyclopropyl (4-methoxybenzyl)amino)-2-(methylsulfonyl)pyrazolo[1,5-a][1,3,5]triazine-8-carbonitrile (1.885 g, 4.73 mmol) in tetrahydrofuran (20 mL) was added and the contents stirred at −78° C. for 30 min. The reaction was quenched with water and extracted with EtOAc. The organic layer was washed with water, dried over MgSO₄, filtered and concentrated. It was then purified by Biotage isolera, eluting with 30% EtOAc/hexane afford tert-butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate (2.6 g, 3.82 mmol, 81% yield) as an off white solid. LC/MS [M−H]⁺=667.6.

### Step 4. (R)-2-((2-chloro-5-cyano-3-(2-methylpiperazin-1-yl)phenyl)amino)-4-(cyclopropylamino)pyrazolo[1,5-a][1,3,5]triazine-8-carbonitrile

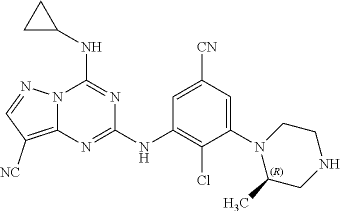

To a mixture of tert-butyl (R)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate (1.6 g, 2.391 mmol) in CH₂Cl₂ (20 mL) was added trifluoroacetic acid (3.66 mL, 47.8 mmol) and the contents stirred at rt for 1 h and concentrated. The residue was dissolved in DCM (3×) and concentrated to yield a light brown solid. The solid obtained was dissolved in ClCH₂CH₂Cl (15 mL). TFA was added (7.7 mL, 94.0 mmol) followed by Anisole (1.018 mL, 9.37 mmol) and the contents heated at 50° C. for 2 h and concentrated. To the residue was added DCM and the contents concentrated (3×). 4 mL of 7 N NH₃ in MeOH and the white ppt was filtered and washed with MeOH to afford (R)-2-((2-chloro-5-cyano-3-(2-methyl piperazin-1-yl)phenyl)amino)-4-(cyclopropylamino)pyrazolo[1,5-a][1,3,5]triazine-8-carbonitrile (750 mg, 1.609 mmol, 68.7% yield) as an off-white solid. LC/MS [M+H]⁺=449.2. ¹H-NMR (400 MHz, DMSO-d₆): δ 9.52 (s, 1H), 8.42 (s, 1H), 8.14 (d, J=1.60 Hz, 1H), 7.48 (d, J=1.60 Hz, 1H), 3.12-3.05 (m, 1H), 2.99-2.93 (m, 1H), 2.88-2.78 (m, 3H), 0.87-0.73 (m, 7H).

### Step 5. Example 306

A mixture of (R)-2-((2-chloro-5-cyano-3-(2-methylpiperazin-1-yl)phenyl)amino)-4-(cyclopropylamino)pyrazolo[1,5-a][1,3,5]triazine-8-carbonitrile (40 mg, 0.089 mmol), Hunig's base (0.047 mL, 0.267 mmol) and methyl carbonochloridate (8.42 mg, 0.089 mmol) in DMSO (0.5 mL) was stirred at rt for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave methyl (3R)-4-(2-chloro-5-cyano-3-{[8-cyano-4-(cyclopropylamino)pyrazolo[1,5-a][1,3,5]triazin-2-yl]amino}phenyl)-3-methylpiperazine-1-carboxylate (12 mg, 0.023 mmol, 25.8% yield) as an off white solid. LC/MS [M+H]⁺=507.1. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.53 (s, 1H), 9.21 (s, 1H), 8.42 (d, J=0.40 Hz, 1H), 7.92 (d, J=1.60 Hz, 1H), 7.41 (d, J=2.00 Hz, 1H), 4.63-4.66 (m, 4H), 3.78-3.75 (m, 1H), 3.32-3.25 (m, 2H), 2.85-2.83 (m, 2H), 2.68-2.67 (m, 1H), 2.65-2.61 (m, 2H), 0.81-0.80 (m, 4H), 0.78-0.75 (m, 1H), 0.68-0.56 (m, 1H).

The following compound were prepared in a similar procedure as described for Example 306 employing the R⁵ group identified in the table below:

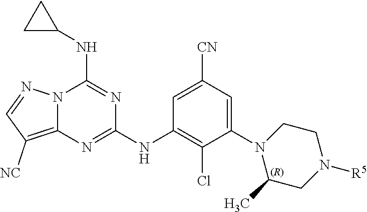

| | | LC/MS [M + H]⁺/ | |
|---|---|---|---|
| Ex # | R⁵ | RT (Method) | ¹H-NMR (400 MHz, DMSO-d₆) |
| 307 | 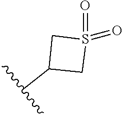 [SMILES: *C1CS(=O)(=O)C1] | 553.2 2.062 min (B) | — |

### Example 308

### 2 2-({2-chloro-5-cyano-3-[(2R)-4-(3-fluoro-2-hydroxypropyl)-2-methylpiperazin-1-yl]phenyl}amino)-4-(cyclopropylamino)pyrazolo[1,5-a][1,3,5]triazine-8-carbonitrile

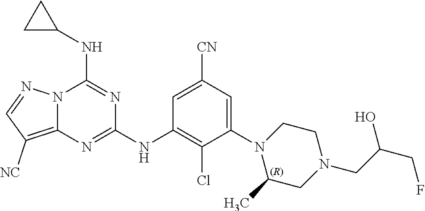

A mixture of (R)-2-((2-chloro-5-cyano-3-(2-methylpiperazin-1-yl)phenyl)amino)-4-(cyclopropylamino)pyrazolo[1,5-a][1,3,5]triazine-8-carbonitrile (30 mg, 0.067 mmol), DIPEA (0.058 mL, 0.334 mmol) and 2-(fluoromethyl)oxirane (5.08 mg, 0.067 mmol) was taken in DMSO (0.5 mL) and stirred at 80° C. for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(2R)-4-(3-fluoro-2-hydroxypropyl)-2-methylpiperazin-1-yl]phenyl}amino)-4-(cyclopropylamino)pyrazolo[1,5-a][1,3,5]triazine-8-carbonitrile (4 mg, 7.41 μmol, 11.09% yield) as an off white solid. LC/MS [M+H]⁺=525.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

The following compounds were prepared in a similar procedure as described for Example 308 employing the R⁵ group identified in the table below:

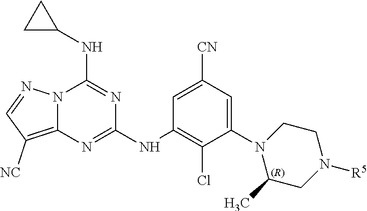

| | | LC/MS [M + H]⁺/ | |
|---|---|---|---|
| Ex # | R⁵ | RT (Method) | ¹H-NMR (400 MHz, DMSO-d₆) |
| 309 | 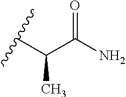 [SMILES: *[C@H](C)C(N)=O] | 520.2 1.876 min (B) | δ 9.55 (s, 1H), 9.25 (d, J = 3.60 Hz, 1H), 8.42 (s, 1H), 8.15 (s, 1H), 7.52 (s, 1H), 7.22 (s, 1H), 7.07 (d, J = 2.40 Hz, 1H), 3.52-3.48 (m, 1H), 3.17 (d, J = 5.20 Hz, 1H), 3.04 (d, J = 6.80 Hz, 1H), 2.85-2.60 (m, 5H), 2.50- 2.34 (m, 1H), 1.12 (d, J = 6.80 Hz, 3H), 0.87 (d, J = 6.00 Hz, 3H), 0.83-0.74 (m, 4H). |
| 310 | 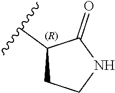 | 532.2 1.883 min (B) | δ 9.54 (s, 1H), 9.25 (d, J = 3.60 Hz, 1H), 8.42 (s, 1H), 8.15 (d, J = 1.60 Hz, 1H), 7.74 (s, 1H), 7.55 (d, J = 1.60 Hz, 1H), 3.48-3.40 (m, 1H), 3.39-3.37 (m, 1H), 3.19-3.07 (m, 4H), 2.93-2.83 (m, 1H), 2.68-2.63 (m, 2H), 2.34-2.29 (m, 1H), 2.17-2.08 (m, 1H), 1.98- 1.90 (m, 1H), 0.87 (d, J = 6.00 Hz, 3H), 0.81-0.73 (m, 4H). |

### LCMS Conditions:

Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 311

### 2-[(2-chloro-5-cyano-3-{4,7-diazaspiro[2.5]octan-7-yl}phenyl)amino]-4-(cyclopropylamino)pyrazolo[1,5-a][1,3,5]triazine-8-carbonitrile

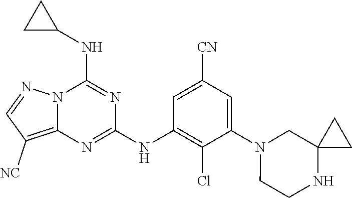 [SMILES: N#Cc1cc(Nc2nc(NC3CC3)n3ncc(C#N)c3n2)c(Cl)c(N2CCNC3(CC3)C2)c1]

### Step 1. tert-butyl 7-(3-((tert-butoxycarbonyl)amino)-2-chloro-5-cyanophenyl)-4,7-diazaspiro[2.5]octane-4-carboxylate

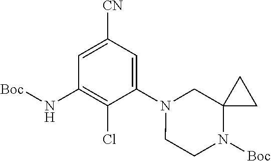 [SMILES: CC(C)(C)OC(=O)Nc1cc(C#N)cc(N2CCN(C(=O)OC(C)(C)C)C3(CC3)C2)c1Cl]

A 250 mL sealed tube was charged tert-butyl (3-bromo-2-chloro-5-cyanophenyl)carbamate (3 g, 9.05 mmol), tert-butyl 4,7-diazaspiro[2.5]octane-4-carboxylate (2.497 g, 11.76 mmol), Pd₂(dba)₃ (0.828 g, 0.905 mmol), BINAP (1.127 g, 1.809 mmol), Cs₂CO₃ (7.37 g, 22.62 mmol) and toluene (75 mL). Nitrogen was bubbled into the reaction mixture for 15 min and the reaction was heated at 110° C. for 16 h. The reaction mixture was cooled to rt and filtered over celite. The filtrate was concentrated in vacuo and the crude was purified by Biotage Isolera, eluting with 12% EtOAc/hexane to afford tert-butyl 7-(3-((tert-butoxycarbonyl)amino)-2-chloro-5-cyanophenyl)-4,7-diazaspiro [2.5]octane-4-carboxylate (2.4 g, 5.18 mmol, 57.3% yield) as pale-yellow solid. LC/MS [M−H]*=461.3. ¹H-NMR (400 MHz, CDCl₃): δ 8.10 (s, 1H), 7.18 (s, 1H), 6.73 (s, 1H), 4.63 (br s, 0.5H), 4.50 (br s, 0.5H), 4.34 (d, J=9.20 Hz, 1H), 3.88-3.83 (m, 1H), 3.69-3.26 (m, 3H), 1.97-1.92 (m, 2H), 1.59 (s, 9H), 1.54 (s, 9H).

### Step 2. tert-butyl 7-(3-amino-2-chloro-5-cyanophenyl)-4,7-diazaspiro[2.5]octane-4-carboxylate

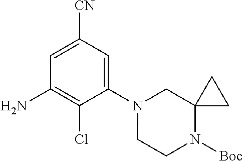 [SMILES: CC(C)(C)OC(=O)N1CCN(c2cc(C#N)cc(N)c2Cl)CC12CC2]

A solution of tert-butyl 7-(3-((tert-butoxycarbonyl)amino)-2-chloro-5-cyanophenyl)-4,7-diazaspiro[2.5]octane-4-carboxylate (2.4 g, 5.18 mmol) in DCM (20 mL) was treated with TFA (3.99 mL, 51.8 mmol) and the contents stirred at rt for 1 h. It was then concentrated, and the crude was triturated with Et₂O, and the resulting solid was dried under high vacuum. The solid was then dissolved into 20 ml of DCM, and treated with TEA (3.61 mL, 25.9 mmol), followed by Boc-anhydride (1.204 mL, 5.18 mmol). It was then stirred for 16 h. The reaction mixture was concentrated, and the crude was purified by Biotage Isolera, eluting with 15% EtOAc/hexane to yield tert-butyl 7-(3-amino-2-chloro-5-cyanophenyl)-4,7-diazaspiro[2.5]octane-4-carboxylate (1.1 g, 3.03 mmol, 58.5% yield) as pale yellow solid. LC/MS [M−H]⁺=363.2.

### Step 3. tert-butyl 7-(2-chloro-5-cyano-3-((8-cyano-4-(cyclopropyl(4-methoxybenzyl)amino)pyrazolo[1,5-a][1,3,5]triazin-2-yl)amino)phenyl)-4,7-diazaspiro[2.5]octane-4-carboxylate

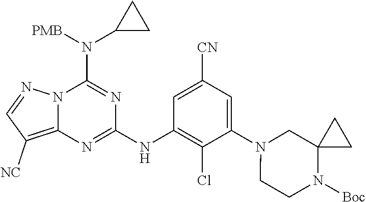

To a mixture of tert-butyl 7-(3-amino-2-chloro-5-cyanophenyl)-4,7-diazaspiro[2.5]octane-4-carboxylate (1.1 g, 3.03 mmol) in tetrahydrofuran (20 mL) was added 1 M solution of LiHMDS (6.06 mL, 6.06 mmol) in THF dropwise at −78° C. It was stirred at −78° C. for 30 min and then 4-(cyclopropyl (4-methoxybenzyl)amino)-2-(methylsulfonyl)pyrazolo[1,5-a][1,3,5]triazine-8-carbonitrile (1.220 g, 3.03 mmol) in tetrahydrofuran (15 mL) was added and stirred at −78° C. for 30 min. The reaction was quenched with water and extracted with EtOAc. The organic layer was washed with water, dried over MgSO₄, filtered and concentrated. It was then purified by Biotage isolera, eluting with 33% EtOAc/hexane to afford tert-butyl 7-(2-chloro-5-cyano-3-((8-cyano-4-(cyclopropyl(4-methoxybenzyl)amino)pyrazolo[1,5-a][1,3,5]triazin-2-yl)amino)phenyl)-4,7-diazaspiro[2.5]octane-4-carboxylate (1.7 g, 2.58 mmol, 81% yield) as pale yellow fluffy solid. LC/MS [M−H]⁺=679.2.

### Step 4. Example 311

A mixture of tert-butyl 7-(2-chloro-5-cyano-3-((8-cyano-4-(cyclopropyl (4-methoxybenzyl)amino)pyrazolo[1,5-a][1,3,5]triazin-2-yl)amino)phenyl)-4,7-diazaspiro[2.5]octane-4-carboxylate (500 mg, 0.734 mmol) in CH₂Cl₂ (20 mL) was added TFA (1.131 mL, 14.68 mmol) and the contents stirred at rt for 1 h and concentrated. The residue was dissolved in DCM and concentrated (3×) to yield a light brown solid. The solid was dissolved in ClCH₂CH₂Cl (5 mL). TFA (1.591 mL, 20.65 mmol) was added followed by anisole (0.224 mL, 2.065 mmol) and the contents heated at 55° C. for 1 h and concentrated. To the residue was added DCM and the contents concentrated (3×). To the residue was added 0.5 mL of 7 N NH₃ in MeOH. The white ppt was filtered and washed with MeOH to afford 2-[(2-chloro-5-cyano-3-{4,7-diazaspiro[2.5]octan-7-yl}phenyl)amino]-4-(cyclopropylamino)pyrazolo[1,5-a][1,3,5]triazine-8-carbonitrile (105 mg) as an off-white solid. LC/MS [M+H]⁺=461.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). ¹H-NMR (400 MHz, DMSO-d₆): δ 9.57 (s, 1H), 9.22 (s, 1H), 8.41 (s, 1H), 8.04 (d, J=1.60 Hz, 1H), 7.34 (d, J=2.00 Hz, 1H), 2.96 (dd, J=3.60, 18.00 Hz, 4H), 2.85 (s, 3H), 0.74-0.81 (m, 4H), 0.54 (dd, J=6.40, Hz, 4H).

### Example 312

### 2-({2-chloro-5-cyano-3-[4-(2-hydroxyethyl)-4,7-diazaspiro[2.5]octan-7-yl]phenyl}amino)-4-(cyclopropylamino)pyrazolo[1,5-a][1,3,5]triazine-8-carbonitrile

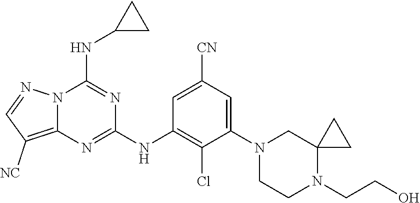 [SMILES: N#Cc1cc(Nc2nc(NC3CC3)n3ncc(C#N)c3n2)c(Cl)c(N2CCN(CCO)C3(CC3)C2)c1]

A mixture of 2-[(2-chloro-5-cyano-3-{4,7-diazaspiro[2.5]octan-7-yl}phenyl)amino]-4-(cyclopropylamino)pyrazolo[1,5-a][1,3,5]triazine-8-carbonitrile (25 mg, 0.054 mmol), acetic acid (13.02 mg, 0.217 mmol) and 1,4-dioxane-2,5-diol (16.29 mg, 0.136 mmol) in DMSO:THF (7:3) (1 mL) was stirred at rt for 2 h. Sodium triacetoxy borohydride (34.5 mg, 0.163 mmol) was added and the contents stirred at rt for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[4-(2-hydroxyethyl)-4,7-diazaspiro[2.5]octan-7-yl]phenyl}amino)-4-(cyclopropylamino)pyrazolo[1,5-a][1,3,5]triazine-8-carbonitrile (4.0 mg, 7.11 μmol, 13.12% yield) as an off white solid. LC/MS [M+H]⁺=505.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm).

### Example 313

### 2-[(3-{4-acetyl-4,7-diazaspiro[2.5]octan-7-yl}-2-chloro-5-cyanophenyl)amino]-4-(cyclopropylamino)pyrazolo[1,5-a][1,3,5]triazine-8-carbonitrile

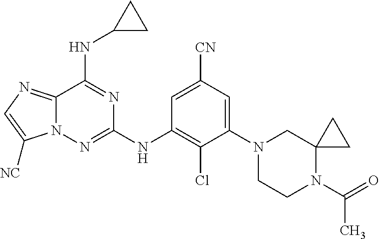 [SMILES: CC(=O)N1CCN(c2cc(C#N)cc(Nc3nc(NC4CC4)c4ncc(C#N)n4n3)c2Cl)CC12CC2]

A mixture of 2-[(2-chloro-5-cyano-3-{4,7-diazaspiro[2.5]octan-7-yl}phenyl)amino]-4-(cyclopropylamino)pyrazolo[1,5-a][1,3,5]triazine-8-carbonitrile (25 mg, 0.054 mmol), Hunig's base (0.047 mL, 0.271 mmol) and acetic anhydride (11.07 mg, 0.108 mmol) in DMSO (0.5 mL) was stirred at rt for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-[(3-{4-acetyl-4,7-diazaspiro[2.5]octan-7-yl}-2-chloro-5-cyanophenyl)amino]-4-(cyclopropylamino)pyrazolo[1,5-a][1,3,5]triazine-8-carbonitrile (5.0 mg, 9.37 μmol, 17.28% yield) as an off white solid. LC/MS [M+H]⁺=503.2. LCMS Injection conditions: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). ¹H-NMR (400 MHz, DMSO-d₆): δ 9.58 (s, 1H), 9.22 (d, J=2.80 Hz, 1H), 8.42 (s, 1H), 8.09 (d, J=1.60 Hz, 1H), 7.42 (d, J=1.20 Hz, 1H), 3.34-3.34 (m, 1H), 2.82-2.85 (m, 3H), 2.67-2.69 (m, 2H), 2.54 (d, J=6.80 Hz, 1H), 2.14-2.33 (m, 3H), 1.04-1.24 (m, 5H), 0.77-0.81 (m, 4H).

The following compounds were prepared in a similar procedure as described for Example 313 employing the R^{a} group identified in the table below:

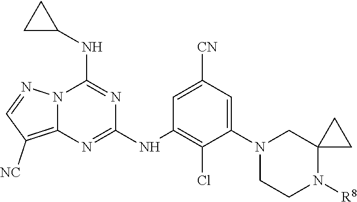

| | | LC/MS [M + H]⁺/ | |
|---|---|---|---|
| Ex # | R⁸ | RT (Method) | ¹H-NMR (400 MHz, DMSO-d₆) |
| 314 | 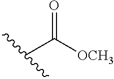 [SMILES: *C(=O)OC] | 519.1 2.55 min (B) | δ 9.57 (s, 1H), 9.22 (s, 1H), 8.41 (s, 1H), 8.08 (s, 1H), 7.42 (d, J = 2.00 Hz, 1H), 3.64 (m, 5H), 3.06 (s, 2H), 2.67-2.68 (m, 3H), 0.92 (d, J = 34.40 Hz, 4H), 0.74-0.79 (m, 4H). |
| 315 | 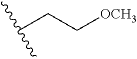 [SMILES: *CCOC] | 519.2 1.985 min (B) | δ 9.53 (brs, 1H), 9.20 (brs, 1H), 8.41 (s, 1H), 8.05 (d, J = 1.6 Hz, 1H), 7.42 (d, J = 1.9 Hz, 1H), 3.38-3.33 (m, 3H), 3.24 (s, 3H), 3.08-3.00 (m, 4H), 2.89-2.79 (m, 5H), 0.84-0.70 (m, 4H), 0.64-0.53 (m, 4H). |

### Example 316

### (2S)-2-[7-(2-chloro-5-cyano-3-{[8-cyano-4-(cyclopropylamino)pyrazolo[1,5-a][1,3,5]triazin-2-yl]amino}phenyl)-4,7-diazaspiro[2.5]octan-4-yl]propanamide

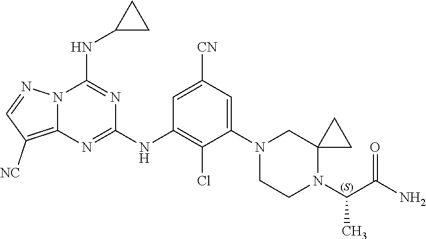

A mixture of 2-[(2-chloro-5-cyano-3-{4,7-diazaspiro[2.5]octan-7-yl}phenyl)amino]-4-(cyclopropylamino)pyrazolo[1,5-a][1,3,5]triazine-8-carbonitrile (50 mg, 0.108 mmol), DIPEA (0.095 mL, 0.542 mmol) and (R)-1-amino-1-oxopropan-2-yl methanesulfonate (91 mg, 0.542 mmol) was taken in DMSO (1 mL) and stirred at 80° C. for 32 h. the reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave (2S)-2-[7-(2-chloro-5-cyano-3-{[8-cyano-4-(cyclopropylamino)pyrazolo[1,5-a][1,3,5]triazin-2-yl]amino}phenyl)-4,7-diazaspiro[2.5]octan-4-yl]propanamide (14 mg, 23.2% yield) as an off white solid. LC/MS [M+H]⁺=532.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H NMR (400 MHz, DMSO-d₆): δ 9.55 (s, 1H), 9.21 (d, J=3.20 Hz, 1H), 8.41 (s, 1H), 8.06 (d, J=1.20 Hz, 1H), 7.43 (d, J=1.60 Hz, 1H), 7.15 (d, J=2.40 Hz, 1H), 6.97 (d, J=2.40 Hz, 1H), 3.54 (q, J=6.80 Hz, 1H), 3.19 (t, J=8.80 Hz, 2H), 2.92-2.97 (m, 3H), 2.82-2.83 (m, 1H), 2.68-2.69 (m, 1H), 1.24 (d, J=7.20 Hz, 3H), 0.89-0.67 (m, 6H), 0.55-0.52 (m, 1H).

The following compounds were prepared in a similar procedure as described for Example 316 employing the R^{a} group identified in the table below:

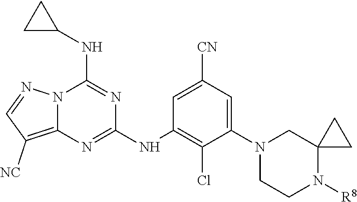

| Ex | | LC/MS [M + H]⁺/ | |
|---|---|---|---|
| # | R⁸ | RT (Method) | ¹H-NMR (400 MHz, DMSO-d₆) |
| 317 | 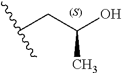 | 519.1 1.93 min (B) | δ 9.55 (s, 1H), 9.21 (s, 1H), 8.41 (s, 1H), 8.05 (d, J = 1.60 Hz, 1H), 7.41 (d, J = 2.00 Hz, 1H), 4.17 (d, J = 3.60 Hz, 1H), 3.57-3.59 (m, 1H), 3.04 (s, 4H), 2.82 (s, 3H), 2.68 (d, J = 1.60 Hz, 2H), 1.24 (m, 4H), 0.74-0.83 (m, 6H), 0.63-0.72 (m, 2H). |
| 318 | 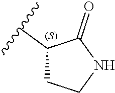 | 544.1 1.91 min (B) | — |

### LCMS Conditions:

Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 319

### Methyl 5-(2-chloro-5-cyano-3-{[8-cyano-4-(cyclopropylamino)pyrazolo[1,5-a][1,3,5]triazin-2-yl]amino}phenyl)-2,5-diazabicyclo[4.1.0]heptane-2-carboxylate

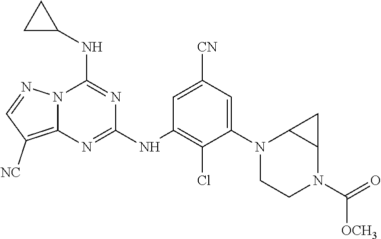 [SMILES: COC(=O)N1CCN(c2cc(C#N)cc(Nc3nc(NC4CC4)n4ncc(C#N)c4n3)c2Cl)C2CC21]

### Step 1. tert-butyl 5-(3-((tert-butoxycarbonyl)amino)-2-chloro-5-cyanophenyl)-2,5-diazabicyclo[4.1.0]heptane-2-carboxylate

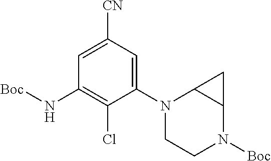 [SMILES: CC(C)(C)OC(=O)Nc1cc(C#N)cc(N2CCN(C(=O)OC(C)(C)C)C3CC32)c1Cl]

A 250-mL sealed tube was charged with tert-butyl (3-bromo-2-chloro-5-cyanophenyl)carbamate (2.5 g, 7.54 mmol), tert-butyl 2,5-diazabicyclo[4.1.0]heptane-2-carboxylate (1.794 g, 9.05 mmol), Pd₂(dba)₃ (0.690 g, 0.754 mmol), BINAP (0.939 g, 1.508 mmol), Cs₂CO₃ (6.14 g, 18.85 mmol) and toluene (40 mL). Nitrogen was bubbled into the reaction mixture for 15 min and the reaction was heated at 110° C. for 20 h. The reaction mixture was cooled to rt and filtered over celite. The filtrate was concentrated in vacuo. The crude obtained was purified by Biotage Isolera, eluting with 32% EtOAc/hexane to afford tert-butyl 5-(3-((tert-butoxycarbonyl)amino)-2-chloro-5-cyanophenyl)-2,5-diazabicyclo[4.1.0]heptane-2-carboxylate (2.6 g, 5.79 mmol, 77% yield) as a pale-yellow solid. LC/MS [M+H]⁺=449.2.

### Step 2. tert-butyl 5-(3-amino-2-chloro-5-cyanophenyl)-2,5-diazabicyclo[4.1.0]heptane-2-carboxylate

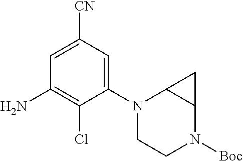 [SMILES: CC(C)(C)OC(=O)N1CCN(c2cc(C#N)cc(N)c2Cl)C2CC21]

A solution of tert-butyl 5-(3-((tert-butoxycarbonyl)amino)-2-chloro-5-cyanophenyl)-2,5-diazabicyclo[4.1.0]heptane-2-carboxylate (2.5 g, 5.57 mmol) in DCM (30 mL) was treated with TFA (4.29 mL, 55.7 mmol) and the contents stirred at rt for 1 h. The reaction mixture was concentrated, and the crude was triturated with Et₂O, and the resulting solid was dried under high vacuum. The solid was then dissolved into 30 mL of DCM, and treated with Et₃N (3.88 mL, 27.8 mmol), followed by Boc-anhydride (1.293 mL, 5.57 mmol). It was then stirred for 16 h. The reaction mixture was concentrated, and the crude was purified by Biotage Isolera, eluting with 18% EtOAc/hexane to yield tert-butyl 5-(3-amino-2-chloro-5-cyanophenyl)-2,5-diazabicyclo[4.1.0]heptane-2-carboxylate (1.35 g, 3.87 mmol, 69.5% yield) as a pale yellow solid. LC/MS [M−H]⁺=349.2.

### Step 3. tert-butyl 5-(2-chloro-5-cyano-3-((8-cyano-4-(cyclopropyl(4-methoxybenzyl)amino)pyrazolo[1,5-a][1,3,5]triazin-2-yl)amino)phenyl)-2,5-diazabicyclo[4.1.0]heptane-2-carboxylate

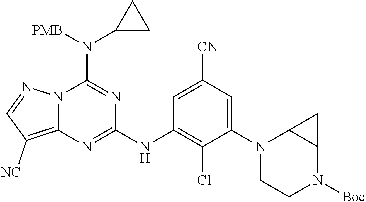

To a mixture of tert-butyl 5-(3-amino-2-chloro-5-cyanophenyl)-2,5-diazabicyclo[4.1.0]heptane-2-carboxylate (1.3 g, 3.73 mmol) in tetrahydrofuran (20 mL) was added 1 M solution of LiHMDS (7.45 mL, 7.45 mmol) in THF dropwise at −78° C. It was stirred at −78° C. for 30 min and then 4-(cyclopropyl(4-methoxybenzyl)amino)-2-(methylsulfonyl)pyrazolo[1,5-a][1,3,5]triazine-8-carbonitrile (1.485 g, 3.73 mmol) in tetrahydrofuran (15 mL) was added and the contents stirred at −78° C. for 30 min. The reaction was quenched with water and extracted with EtOAc. The organic layer was washed with water, dried over MgSO₄, filtered and concentrated. It was purified by Biotage isolera, eluting with 50% EtOAc/hexane to afford tert-butyl 5-(2-chloro-5-cyano-3-((8-cyano-4-(cyclopropyl(4-methoxybenzyl)amino) pyrazolo[1,5-a][1,3,5]triazin-2-yl)amino)phenyl)-2,5-diazabicyclo[4.1.0]heptane-2-carboxylate (2 g, 2.128 mmol, 57.1% yield) as a pale yellow fluffy solid. LC/MS [M−H]⁺=665.2.

### Step 4. 2-[(2-Chloro-5-cyano-3-{2,5-diazabicyclo[4.1.0]heptan-2-yl}phenyl)amino]-4-(cyclopropylamino)pyrazolo[1,5-a][1,3,5]triazine-8-carbonitrile (

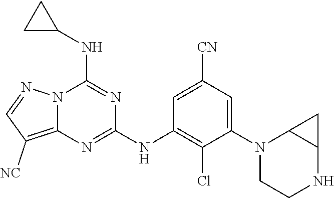 [SMILES: N#Cc1cc(Nc2nc(NC3CC3)n3ncc(C#N)c3n2)c(Cl)c(N2CCNC3CC32)c1]

To a mixture of tert-butyl 5-(2-chloro-5-cyano-3-((8-cyano-4-(cyclopropyl(4-methoxybenzyl)amino)pyrazolo[1,5-a][1,3,5]triazin-2-yl)amino)phenyl)-2,5-diazabicyclo[4.1.0]heptane-2-carboxylate (800 mg, 1.199 mmol) in CH₂Cl₂ (20 mL) was added TFA (1.847 mL, 23.98 mmol) and the contents stirred at rt for 1 h and concentrated. The residue was dissolved in DCM and concentrated three times to yield a light brown solid (800 mg). A portion of the solid obtained was dissolved in ClCH₂CH₂Cl (15 mL) TFA (1.358 mL, 17.64 mmol) was added followed by anisole (0.192 mL, 1.764 mmol) and the contents heated at 50° C. for 1 h and concentrated. To the residue was added DCM and the contents concentrated (3×). To the residue was then added 3 mL of 7 N NH₃ in MeOH. The white ppt was washed with MeOH to afford a solid (140 mg). 40 mg of the solid was further purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-[(2-chloro-5-cyano-3-{2,5-diazabicyclo[4.1.0]heptan-2-yl}phenyl)amino]-4-(cyclopropylamino)pyrazolo[1,5-a][1,3,5]triazine-8-carbonitrile (2.5 mg, 5.47 μmol, 1.240% yield) as an off-white solid. LC/MS [M+H]⁺=447.1. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Step 5. Example 319

A mixture of 2-[(2-chloro-5-cyano-3-{2,5-diazabicyclo[4.1.0]heptan-2-yl}phenyl)amino]-4-(cyclopropylamino)pyrazolo[1,5-a][1,3,5]triazine-8-carbonitrile (60 mg, 0.134 mmol), Hunig's base (0.070 mL, 0.403 mmol), and methyl carbonochloridate (19.03 mg, 0.201 mmol) in DMSO (0.5 mL) was stirred at rt for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave methyl 5-(2-chloro-5-cyano-3-{[8-cyano-4-cyclopropylamino)pyrazolo[1,5-a][1,3,5]triazin-2-yl]amino}phenyl)-2,5-diazabicyclo[4.1.0]heptane-2-carboxylate (8 mg, 0.015 mmol, 11.52% yield) as an off white solid. LC/MS [M+H]⁺=505.2. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

The following compounds were prepared in a similar procedure as described for Example 319 employing the R^{a} group identified in the table below:

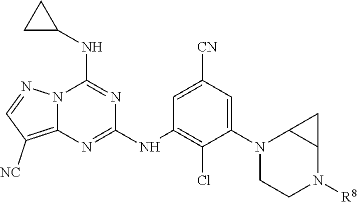

| | | LC/MS [M + H]⁺/ | |
|---|---|---|---|
| Ex # | R⁸ | RT (Method) | ¹H-NMR (400 MHz, DMSO-d₆) |
| 320 | 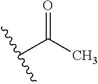 [SMILES: *C(C)=O] | 489.1 2.174 min (B) | δ 9.63 (s, 1H), 9.24 (s, 1H), 8.42 (s, 1H), 8.02 (s, 1H), 7.58 (d, J = 1.60 Hz, 1H), 3.94-3.87 (m, 1H), 3.30-3.20 (m, 2H), 3.10-2.98 (m, 2H), 2.88-2.82 (m, 1H), 0.84-0.73 (m, 5H). |
| 321 | 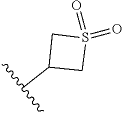 [SMILES: *C1CS(=O)(=O)C1] | 551.1 2.259 min (B) | — |

### Example 322

### 2-((2-chloro-5-cyano-3-(5-(1,1-dioxidotetrahydro-2H-thiopyran-4-yl)-2,5-diazabicyclo[4.1.0]heptan-2-yl)phenyl)amino)-4-(cyclopropylamino)pyrazolo[1,5-a][1,3,5]triazine-8-carbonitrile

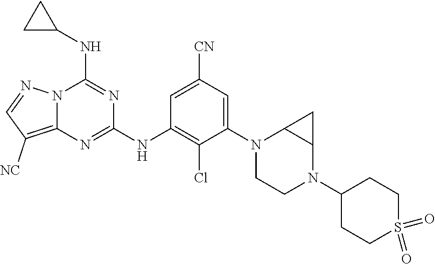 [SMILES: N#Cc1cc(Nc2nc(NC3CC3)n3ncc(C#N)c3n2)c(Cl)c(N2CCN(C3CCS(=O)(=O)CC3)C3CC32)c1]

A mixture of 2-[(2-chloro-5-cyano-3-{2,5-diazabicyclo[4.1.0]heptan-2-yl}phenyl)amino]-4-(cyclopropylamino)pyrazolo[1,5-a][1,3,5]triazine-8-carbonitrile (40 mg, 0.090 mmol), acetic acid (21 mg, 0.358 mmol) and tetrahydro-4H-thiopyran-4-one 1,1-dioxide (39.8 mg, 0.269 mmol) in DMSO:THF (7:3) (0.5 mL) was stirred at RT for 2 h. Sodium cyanoborohydride (56.9 mg, 0.269 mmol) was added and the contents stirred for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave the titled compound (21 mg, 0.035 mmol, 39.5% yield) as an off white solid. LC/MS [M+H]⁺=579.1. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.50 (s, 1H), 9.20 (d, J=3.20 Hz, 1H), 8.41 (s, 1H), 7.92 (s, 1H), 7.39 (d, J=2.00 Hz, 1H), 3.10-3.31 (m, 4H), 2.84-2.88 (m, 2H), 2.69-2.77 (m, 2H), 2.25-2.50 (m, 4H), 0.80-0.81 (m, 4H), 0.68 (q, J=6.40 Hz, 1H), 0.58 (q, J=4.40 Hz, 1H).

The following compounds were prepared in a similar procedure as described for Example 322 employing the R⁸ group identified in the table below:

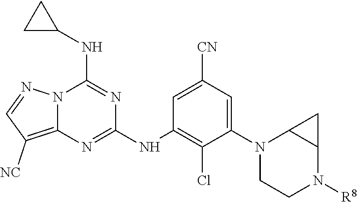

| | | LC/MS [M + H]⁺/ | |
|---|---|---|---|
| Ex # | R⁸ | RT (Method) | ¹H-NMR (400 MHz, DMSO-d₆) |
| 323 | 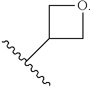 [SMILES: *C1COC1] | 503.1 1.90 min (B) | δ 9.56 (s, 1H), 9.25 (d, J = 2.80 Hz, 1H), 8.42 (s, 1H), 8.18 (d, J = 1.60 Hz, 1H), 7.55 (d, J = 2.00 Hz, 1H), 3.54-3.72 (m, 4H), 3.45-3.49 (m, 3H), 3.16-3.22 (m, 2H), 2.83-2.85 (m, 1H), 2.68-2.72 (m, 1H), 0.77-0.86 (m, 7H). |
| 324 | 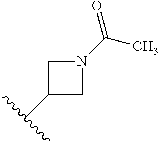 [SMILES: *C1CN(C(C)=O)C1] | 544.2 1.85 min (B) | δ 9.55 (s, 1H), 9.22 (d, J = 2.80 Hz, 1H), 8.42 (s, 1H), 7.93 (s, 1H), 7.42 (s, 1H), 4.09-4.11 (m, 2H), 3.79-3.89 (m, 2H), 3.41-3.46 (m, 3H), 3.24-3.31 (m, 2H), 2.84 (d, J = 5.60 Hz, 2H), 1.78 (s, 3H), 0.69-0.80 (m, 5H), 0.56 (q, J = 6.40 Hz, 1H). |

### Example 325

### 2-[(2-Chloro-5-cyano-3-{5-[(2R)-2-hydroxypropyl]-2,5-diazabicyclo[4.1.0]heptan-2-yl}phenyl)amino]-4-(cyclopropylamino)pyrazolo[1,5-a][1,3,5]triazine-8-carbonitrile

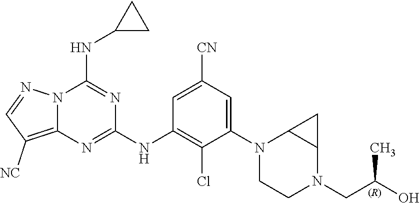

A mixture of 2-[(2-chloro-5-cyano-3-{2,5-diazabicyclo[4.1.0]heptan-2-yl}phenyl)amino]-4-(cyclopropylamino)pyrazolo[1,5-a][1,3,5]triazine-8-carbonitrile (40 mg, 0.090 mmol), DIPEA (0.047 mL, 0.269 mmol) and (R)-2-methyloxirane (10.40 mg, 0.179 mmol) was taken in DMSO (1 mL) and the contents stirred at 80° C. for 16 h. LCMS showed completion of reaction. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-[(2-chloro-5-cyano-3-{5-[(2R)-2-hydroxypropyl]-2,5-diazabicyclo[4.1.0]heptan-2-yl}phenyl)amino]-4-(cyclopropylamino) pyrazolo[1,5-a][1,3,5]triazine-8-carbonitrile (6 mg, 0.011 mmol, 12.42% yield) as an off white solid. LC/MS [M+H]⁺=505.1. LCMS Injection conditions: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

The following compounds were prepared in a similar procedure as described for Example 325 employing the R^{a} group identified in the table below:

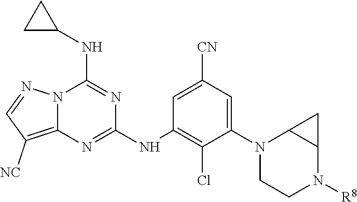

| | | LC/MS [M + H]⁺/ | |
|---|---|---|---|
| Ex # | R⁸ | RT (Method) | ¹H-NMR (400 MHz, DMSO-d₆) |
| 326 | 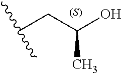 | 505.2 1.25 min (A) | — |
| 327 | 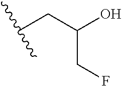 [SMILES: *CC(O)CF] | 523.1 1.92 min (B) | δ 9.53 (s, 1H), 9.22 (s, 1H), 8.42 (s, 1H), 7.92 (s, 1H), 7.40 (s, 1H), 5.04-5.02 (m, 1H), 4.51- 4.31 (m, 3H), 4.10-4.05 (m, 1H), 3.22-3.19 (m, 3H), 2.86-2.74 (m, 4H), 2.63-2.60 (m, 3H), 2.47-2.44 (m, 3H), 0.80-0.75 (m, 5H), 0.66-0.57 (m, 2H). |
| 328 | 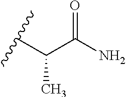 [SMILES: *[C@@H](C)C(N)=O] | 518.2 1.85 min (B) | — |
| 329 | 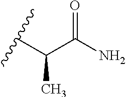 [SMILES: *[C@H](C)C(N)=O] | 518.1 1.86 min (B) | δ 9.50 (s, 1H), 9.19 (s, 1H), 8.41 (d, J = 2.40 Hz, 1H), 7.93 (d, J = 1.60 Hz, 1H), 7.40 (d, J = 1.60 Hz, 1H), 7.27 (d, J = 16.80 Hz, 1H), 7.01 (d, J = 2.80 Hz, 1H), 3.30 (dd, J = 4.40, 25.40 Hz, 1H), 3.18 (d, J = 7.20 Hz, 1H), 2.81-2.86 (m, 2H), 2.65-2.74 (m, 2H), 1.24 (dd, J = 6.80, 27.60 Hz, 3H), 0.77-0.81 (m, 4H), 0.61-0.65 (m, 2H). |
| 330 | 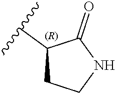 | 530.1 1.86 min (B) | — |

### LCMS Conditions:

Method A: Column: Kinetex XB-C18, 3.0 mm×75 mm, 2.6 μm particles; Mobile Phase A: 02:98 acetonitrile:water with 5 mM Ammonium formate (pH 3.3); Mobile Phase B: 98:02 acetonitrile:water with 5 mm Ammonium formate; Temperature: 25° C.; Gradient: 20% B to 100% B over 4 min, then a 0.60 min hold at 100% B; Flow Rate: 1.0 ml/min; Detection: MS and UV (220 nm). Method B: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm).

### Example 331

### Methyl (3R,4R)-3-[4-(2-chloro-5-cyano-3-{[8-cyano-4-(cyclopropylamino)pyrazolo[1,5-a][1,3,5]triazin-2-yl]amino}phenyl)piperazin-1-yl]-4-hydroxypyrrolidine-1-carboxylate

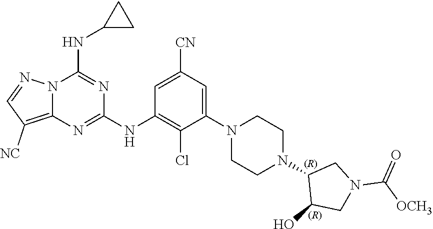

### Step 1. Benzyl 4-((3R,4S)-1-(tert-butoxycarbonyl)-4-hydroxypyrrolidin-3-yl)piperazine-1-carboxylate

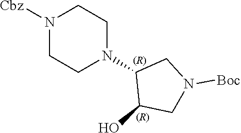

Tert-butyl 6-oxa-3-azabicyclo[3.1.0]hexane-3-carboxylate (10.51 g, 56.7 mmol) was added to a stirred solution of benzyl piperazine-1-carboxylate (5 g, 22.70 mmol) in THF (50 mL) and water (50 mL) at ambient temperature. The resulting mixture was heated to 80° C. and stirred there for 16 h. Another lot of tert-butyl 6-oxa-3-azabicyclo[3.1.0]hexane-3-carboxylate (5.26 g, 28.4 mmol) was added to the reaction mixture at rt, and the heating continued at 80° C. for 24 h. The reaction mixture was diluted with water, and extracted with ethyl acetate. The combined organic layer was dried over anhydrous Na₂SO₄, filtered, and concentrated under reduced pressure. The crude product obtained was purified by column chromatography (Biotage isolera, 100 g snap) by eluting with 80-100% ethyl acetate in petroleum ether afford benzyl 4-(1-(tert-butoxycarbonyl)-4-hydroxypyrrolidin-3-yl)piperazine-1-carboxylate (8.5 g, 20.77 mmol, 92% yield) as a pale-yellow gummy. Further the individual enantiomers were separated by SFC chiral purification to get first eluting isomer benzyl 4-((3R,4R)-1-(tert-butoxycarbonyl)-4-hydroxypyrrolidin-3-yl)piperazine-1-carboxylate (2.6 g, 28.2% yield) as a pale-yellow gummy. ¹H NMR (400 MHz, DMSO-d₆) δ 7.40-7.31 (m, 5H), 5.15 (d, J=5.20 Hz, 1H), 5.08 (s, 2H), 4.17-4.10 (m, 1H), 3.50-3.42 (m, 2H), 3.39-3.32 (m, 3H), 3.14-3.06 (m, 1H), 3.04-2.95 (m, 1H), 2.77-2.67 (m, 1H), 2.59-2.52 (m, 2H), 2.39-2.30 (m, 2H), 1.39 (s, 9H). Assignment of the absolute configuration for the first eluting isomer is tentative.

### Step 2. Tert-butyl (3R,4R)-3-hydroxy-4-(piperazin-1-yl)pyrrolidine-1-carboxylate

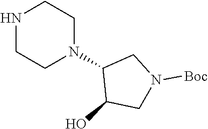 [SMILES: CC(C)(C)OC(=O)N1C[C@@H](O)[C@H](N2CCNCC2)C1]

To a solution of benzyl 4-((3R,4R)-1-(tert-butoxycarbonyl)-4-hydroxypyrrolidin-3-yal)piperazine-1-carboxylate (2.6 g, 6.41 mmol) in methanol (50 mL) was taken in miniclave. Pd—C (1 g, 0.940 mmol) was added under N₂ atmosphere. The reaction mixture was kept under hydrogen atmosphere (30 psi pressure) for 16 h at rt. The reaction mixture was filtered through celite pad and washed with methanol. The filtrate collected was concentrated under reduced pressure to afford tert-butyl (3R,4R)-3-hydroxy-4-(piperazin-1-yl)pyrrolidine-1-carboxylate (1.7 g, 91% yield) as pale brown gummy. LCMS (M+H)+=272.2.

### Step 3. tert-butyl (3R,4R)-3-(4-(2-chloro-5-cyano-3-((8-cyano-4-(cyclopropyl(4-methoxybenzyl)amino)pyrazolo[1,5-a][1,3,5]triazin-2-yl)amino)phenyl)piperazin-1-yl)-4-hydroxypyrrolidine-1-carboxylate

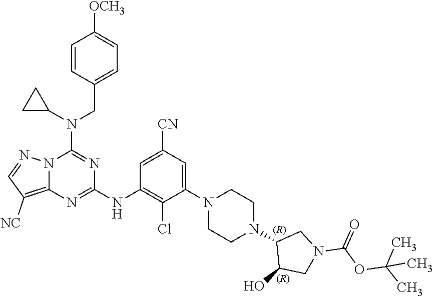

A 100 mL three neck RB flask was charged dioxane (35 mL), 2-((3-bromo-2-chloro-5-cyanophenyl)amino)-4-(cyclopropyl(4-methoxybenzyl) amino)pyrazolo[1,5-a][1,3,5]triazine-8-carbonitrile (1.7 g, 3.09 mmol), tert-butyl (3R,4R)-3-hydroxy-4-(piperazin-1-yl)pyrrolidine-1-carboxylate (1.636 g, 6.03 mmol), Cs₂CO₃ (3.02 g, 9.28 mmol), BINAP (0.578 g, 0.928 mmol) and finally Pd₂(dba)₃ (0.283 g, 0.309 mmol) added sequentially under nitrogen atmosphere. The resulting mixture was heated to 110° C. and stirred for 40 h. The reaction mixture was cooled to rt, filtered through celite pad and washed with DCM (100 mL). The filtrate was concentrated under reduced pressure. The crude product obtained was purified by column chromatography (Biotage isolera) by eluting with 30-40% ethyl acetate in petroleum ether. The desired fractions were concentrated under reduced pressure to afford tert-butyl (3R,4R)-3-(4-(2-chloro-5-cyano-3-((8-cyano-4-(cyclopropyl(4-methoxybenzyl) amino)pyrazolo[1,5-a][1,3,5]triazin-2-yl)amino)phenyl)piperazin-1-yl)-4-hydroxypyrrolidine-1-carboxylate (900 mg, 33% yield) as pale yellow solid. LCMS (M-56)⁺=640.4.

### Step 4. 2-((2-chloro-5-cyano-3-(4-((3R,4R)-4-hydroxypyrrolidin-3-yl)piperazin-1-yl)phenyl)amino)-4-(cyclopropyl(4-methoxybenzyl)amino)pyrazolo[1,5-a][1,3,5]triazine-8-carbonitrile, TFA

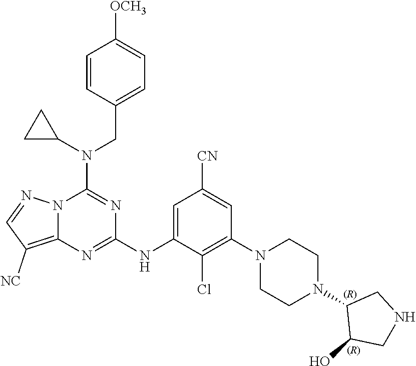

To a stirred solution of tert-butyl (3R,4R)-3-(4-(2-chloro-5-cyano-3-((8-cyano-4-(cyclopropyl(4-methoxybenzyl)amino)pyrazolo[1,5-a][1,3,5]triazin-2-yl)amino)phenyl)piperazin-1-yl)-4-hydroxypyrrolidine-1-carboxylate (700 mg, 0.946 mmol) in DCE (14 mL) TFA (1.457 mL, 18.91 mmol) was added at 0° C. The resulting mixture was stirred at ambient temperature for 1.5 h. Reaction mixture was concentrated under reduced pressure to afford 2-((2-chloro-5-cyano-3-(4-((3R,4R)-4-hydroxypyrrolidin-3-yl)piperazin-1-yl)phenyl)amino)-4-(cyclopropyl(4-methoxybenzyl)amino)pyrazolo[1,5-a][1,3,5]triazine-8-carbonitrile, TFA (1 g, 105% yield) as pale brown gummy solid. The residue obtained was taken in DCE (15 mL). TFA (4.81 mL, 62.5 mmol) and anisole (0.683 mL, 6.25 mmol) was added, and the contents stirred at 50° C. for 2 h, cooled to rt and concentrated. To the residue was added DCM and concentrated for 3 times. 2 mL of 7N ammonia in methanol was added and the precipitate obtained was filtered and washed with methanol and dried under vacuum to afford 2-((2-chloro-5-cyano-3-(4-((3R,4R)-4-hydroxy pyrrolidin-3-yl)piperazin-1-yl)phenyl)amino)-4-(cyclopropylamino)pyrazolo[1,5-a][1,3,5]triazine-8-carbonitrile (240 mg, 29.5% yield) as an off white solid. LC/MS [M−H]⁺=520.2.

### Step 5. Example 331

To a solution of 2-((2-chloro-5-cyano-3-(4-((3R,4R)-4-hydroxypyrrolidin-3-yl)piperazin-1-yl)phenyl)amino)-4-(cyclopropylamino)pyrazolo[1,5-a][1,3,5]triazine-8-carbonitrile (25 mg, 0.048 mmol) in DMSO (1 mL) was added methyl chloroformate (4.47 μl, 0.058 mmol) and DIPEA (0.025 mL, 0.144 mmol) at ambient temperature. The resulting mixture was stirred at ambient temperature for 16 h. The reaction mixture was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation afford methyl (3R,4R)-3-[4-(2-chloro-5-cyano-3-{[8-cyano-4-(cyclopropylamino)pyrazolo[1,5-a][1,3,5]triazin-2-yl]amino}phenyl)piperazin-1-yl]-4-hydroxypyrrolidine-1-carboxylate (7.4 mg, 0.013 mmol, 26.5% yield) as an off white solid. LC/MS [M+H]⁺=578.2. ¹H NMR (400 MHz, DMSO-d₆) δ 9.58 (s, 1H), 9.23 (s, 1H), 8.42 (s, 1H), 8.08 (d, J=1.60 Hz, 1H), 7.38 (d, J=1.60 Hz, 1H), 5.24-5.20 (m, 1H), 4.25-4.18 (m, 1H), 3.60-3.51 (m, 5H), 3.24-3.19 (m, 1H), 3.11-3.04 (m, 1H), 3.03-2.98 (m, 4H), 2.83-2.72 (m, 4H), 2.61-2.53 (m, 3H), 0.81-69.00 (m, 4H).

### Example 332

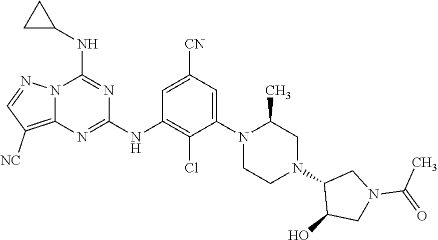 [SMILES: CC(=O)N1C[C@@H](O)[C@H](N2CCN(c3cc(C#N)cc(Nc4nc(NC5CC5)n5ncc(C#N)c5n4)c3Cl)[C@@H](C)C2)C1]

Example 332 as a mixture of diastereomers was prepared from 2-((2-chloro-5-cyano-3-((2S)-2-methyl-4-(pyrrolidin-3-yl)piperazin-1-yl)phenyl) amino)-4-(cyclopropylamino)pyrazolo[1,5-a][1,3,5]triazine-8-carbonitrile in a similar procedure as described for Example 334. Analytical Reverse Phase chromatography was used to determine the final purity. Injection conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+). Injection 6 results: Purity: 100%; Observed Mass: 576.1; RT: 1.56 min.

### Example 333

### 2-({2-Chloro-5-cyano-3-[(3R)-3-(hydroxymethyl)-4-[1-(oxetan-3-yl)piperidin-4-yl]piperazin-1-yl]phenyl}amino)-4-(cyclopropylamino)pyrazolo[1,5-a][1,3,5]triazine-8-carbonitrile

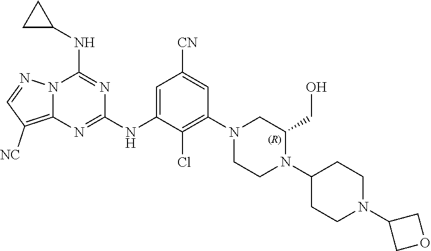

### Step 1. tert-butyl (R)-4-(1-((benzyloxy)carbonyl)piperidin-4-yl)-3-(hydroxymethyl)piperazine-1-carboxylate

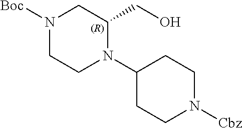

To a stirred solution of tert-butyl (R)-3-(hydroxymethyl)piperazine-1-carboxylate (15 g, 69.4 mmol) in DCE (70 mL) under N₂ atmosphere, benzyl 4-oxopiperidine-1-carboxylate (24.27 g, 104 mmol) was added, followed by acetic acid (15.88 mL, 277 mmol) and the contents stirred for 30 min at rt. Sodium triacetoxyborohydride (30 g) was added at 0° C. The reaction mixture was allowed to reach rt and stirred for 40 h. The reaction mixture was quenched with 10% NaOH solution and extracted with DCM (2×). The combined organic layers were washed with water, brine, dried over anhydrous sodium sulphate, filtered, and concentrated under vacuum. The crude product obtained was purified by Biotage isolera, eluting with 7% MeOH/EtOAc afforded tert-butyl (R)-4-(1-((benzyloxy)carbonyl)piperidin-4-yl)-3-(hydroxymethyl)piperazine-1-carboxylate (11.5 g, 23.87 mmol, 34.4% yield) as a pale-yellow oil. LCMS (M+H)+=434.2.

### Step 2. tert-butyl (R)-4-(1-((benzyloxy)carbonyl)piperidin-4-yl)-3-(((tert-butyldimethylsilyl)oxy)methyl)piperazine-1-carboxylate

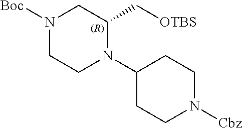

To a stirred solution of tert-butyl (R)-4-(1-((benzyloxy) carbonyl) piperidin-4-yl)-3-(hydroxymethyl) piperazine-1-carboxylate (9.5 g, 21.91 mmol) in DCM (200 mL) was cooled to 0° C. under N₂ atmosphere. Triethylamine (21.63 mL, 150 mmol) was added, followed by TBS-Cl (4.95 g, 32.9 mmol). The reaction mixture was allowed to stir at RT for 16 h. The reaction mixture was diluted with cold water and DCM. The DCM layer was separated and washed with 10% aq.NaHCO₃ solution. The organic layer was separated, washed with saturated brine solution, dried over anhydrous Na₂SO₄, filtered and concentrated under reduced pressure to afford tert-butyl (R)-4-(1-((benzyloxy)carbonyl)piperidin-4-yl)-3-(((tert-butyldimethylsilyl)oxy)methyl) piperazine-1-carboxylate (11 g, 20.08 mmol, 92% yield) as a pale-yellow liquid. LCMS (M+H)+=548.7.

### Step 3. benzyl (R)-4-(2-(((tert-butyldimethylsilyl) oxy)methyl)piperazin-1-yl)piperidine-1-carboxylate

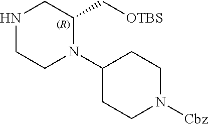

A solution of tert-butyl (R)-4-(1-((benzyloxy)carbonyl) piperidin-4-yl)-3-(((tert-butyldimethylsilyl)oxy)methyl)piperazine-1-carboxylate (9.0 g, 16.43 mmol) in DCM (120 mL) was cooled to 0° C. under N₂ atmosphere. TFA (5.03 mL, 65.7 mmol) was added, and the reaction mixture was allowed to stir at rt for 16 h. The reaction mixture was diluted with cold water. The DCM layer was separated and washed with saturated NaHCO₃ solution, brine, dried over anhydrous Na₂SO₄, filtered, and concentrated under reduced pressure to afford benzyl (R)-4-(2-(((tert-butyldimethylsilyl) oxy)methyl)piperazin-1-yl)piperidine-1-carboxylate (6.250 g, quantitative) as a yellow gummy liquid. LCMS (M+H)+=448.3.

### Step 4. benzyl (R)-4-(2-(((tert-butyldimethylsilyl)oxy)methyl)-4-(2-chloro-5-cyano-3-((8-cyano-4-(cyclopropyl(4-methoxybenzyl)amino)pyrazolo[1,5-a][1,3,5]triazin-2-yl)amino)phenyl)piperazin-1-yl)piperidine-1-carboxylate

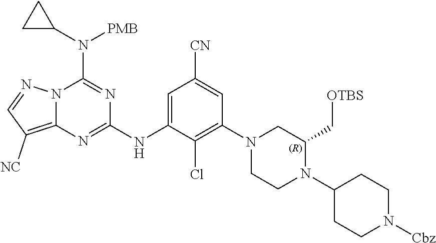

To a stirred solution of 2-((3-bromo-2-chloro-5-cyanophenyl)amino)-4-(cyclopropyl(4-methoxybenzyl)amino)pyrazolo[1,5-a][1,3,5]triazine-8-carbonitrile (1.2 g, 2.183 mmol) in dioxane (40 mL) were added benzyl (R)-4-(2-(((tert-butyldimethylsilyl)oxy)methyl)piperazin-1-yl)piperidine-1-carboxylate (1.954 g, 4.37 mmol) and cesium carbonate (4.27 g, 13.10 mmol). The reaction mixture was purged with nitrogen for 5 min followed by the addition of Pd₂dba₃ (0.200 g, 0.218 mmol) and BINAP (0.408 g, 0.655 mmol). The reaction mixture was heated to 105° C. and stirred for 16 h. The reaction mixture was cooled to room temperature, filtered through celite and concentrated. The crude was purified by Biotage Isolera, eluting with 40 to 50% EtOAc/hexane afforded benzyl (R)-4-(2-(((tert-butyldimethylsilyl)oxy) methyl)-4-(2-chloro-5-cyano-3-((8-cyano-4-(cyclopropyl(4-methoxybenzyl)amino)pyrazolo[1,5-a][1,3,5]triazin-2-yl)amino)phenyl)piperazin-1-yl)piperidine-1-carboxylate (1 g, 0.993 mmol, 45.5% yield) as a pale yellow solid. LC/MS [M+H]⁺=916.2.

### Step 5. (R)-2-((3-(3-(((tert-butyldimethylsilyl)oxy)methyl)-4-(piperidin-4-yl)piperazin-1-yl)-2-chloro-5-cyanophenyl)amino)-4-(cyclopropylamino)pyrazolo[1,5-a][1,3,5]triazine-8-carbonitrile

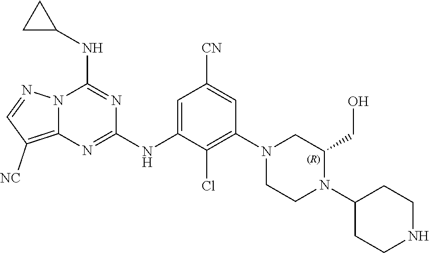

Benzyl (R)-4-(2-(((tert-butyldimethylsilyl)oxy)methyl)-4-(2-chloro-5-cyano-3-((8-cyano-4-(cyclopropyl(4-methoxybenzyl)amino)pyrazolo[1,5-a][1,3,5]triazin-2-yl)amino)phenyl)piperazin-1-yl)piperidine-1-carboxylate (550 mg, 0.600 mmol) in DCM (10 mL) cooled to 0° C. was added TFA (0.533 mL, 6.00 mmol) and the reaction mixture was stirred at rt for 16 h. It was then concentrated, and the crude was triturated with Et₂O, and the resulting solid was dried under high vacuum. To the solid, 7N ammonia in methanol (1 mL) was added, filtered, and washed with MeOH. The solid obtained was purified by reverse phase silica column eluting with water/ACN to afford (R)-2-((2-chloro-5-cyano-3-(3-(hydroxymethyl)-4-(piperidin-4-yl)piperazin-1-yl)phenyl)amino)-4-(cyclopropylamino)pyrazolo[1,5-a][1,3,5]triazine-8-carbonitrile (230 mg, 0.298 mmol, 49.7% yield) as an off white solid. LC/MS [M−H]⁺=548.2. ¹H-NMR (400 MHz, DMSO-d₆): δ 9.56 (s, 1H), 9.23 (s, 1H), 8.41 (s, 1H), 8.08 (s, 1H), 7.37 (d, J=1.60 Hz, 1H), 4.52 (t, J=5.20 Hz, 1H), 3.64-3.65 (m, 1H), 3.52-3.54 (m, 1H), 3.27 (d, J=20.40 Hz, 1H), 3.20-3.00 (m, 3H), 2.94 (t, J=8.80 Hz, 1H), 2.83-2.79 (m, 5H), 2.64-2.61 (m, 2H), 2.48 (t, J=26.00 Hz, 1H), 1.80-1.76 (m, 1H), 1.61-1.58 (m, 1H), 1.50-1.40 (m, 1H), 1.20-1.30 (m, 1H), 0.74-0.77 (m, 4H).

### Step 6. Example 333

A mixture of (R)-2-((3-(3-(((tert-butyldimethylsilyl)oxy)methyl)-4-(piperidin-4-yl)piperazin-1-yl)-2-chloro-5-cyanophenyl)amino)-4-(cyclopropylamino)pyrazolo[1,5-a][1,3,5]triazine-8-carbonitrile (40 mg, 0.073 mmol), in THF (0.3 mL) and DMSO (0.1 mL) was added acetic acid (0.013 mL, 0.219 mmol) and oxetan-3-one (10.52 mg, 0.146 mmol) and the contents stirred at rt for 0.5 h. Sodium triacetoxyborohydride (46.4 mg, 0.219 mmol) was added and the reaction mixture was stirred at rt for 16 h. The reaction was quenched with MeOH (0.5 mL) and purified via preparative LC/MS employing the following conditions: Column: XBridge C18, 150 mm×19 mm, 5-μm particles; Mobile Phase A: 10 mM Ammonium bicarbonate; Mobile Phase B: acetonitrile: Gradient: a 0-min hold at 20% B, 20-60% B over 12 min, then a 0-min hold at 100% B; Flow Rate: 15 mL/min; Column Temperature: 25° C. Fraction collection was triggered by MS signals. Fractions containing the desired product were combined and dried via centrifugal evaporation gave 2-({2-chloro-5-cyano-3-[(3R)-3-(hydroxymethyl)-4-[1-(oxetan-3-yl)piperidin-4-yl]piperazin-1-yl]phenyl}amino)-4-(cyclopropylamino)pyrazolo[1,5-a][1,3,5]triazine-8-carbonitrile as an off white solid. LC/MS [M+H]⁺=604.2. LCMS Injection condition: Column: XBridge C8, 4.6 mm×50 mm, 5 μm particles; Mobile Phase A: Water with 0.1% TFA; Mobile Phase B: Acetonitrile with 0.1% TFA; Temperature: 25° C.; Gradient: 5% B to 95% B over 2.5 min, then a 1.5 min hold at 95% B; Flow Rate: 1.5 ml/min; Detection: MS and UV (220 nm). ¹H-NMR (400 MHz, DMSO-d₆): δ 9.58 (s, 1H), 9.24 (s, 1H), 8.42 (s, 1H), 8.08 (d, J=1.60 Hz, 1H), 7.37 (d, J=2.00 Hz, 1H), 4.52 (t, J=6.40 Hz, 3H), 4.41-4.43 (m, 2H), 3.70-3.60 (m, 1H), 3.55-3.56 (m, 1H), 3.20-3.30 (m, 1H), 3.18-3.20 (m, 1H), 2.93-3.03 (m, 1H), 2.83-2.84 (m, 4H), 2.73-2.75 (m, 3H), 2.67-2.68 (m, 1H), 1.76-1.82 (m, 3H), 1.63-1.74 (m, 2H), 1.45-1.60 (m, 1H), 0.74-0.77 (m, 4H). δ 9.51 (s, 1H), 9.20 (s, 1H), 8.41 (s, 1H), 7.73 (s, 1H), 7.16 (s, 1H), 4.54 (t, J=6.80 Hz, 2H), 4.45 (s, 2H), 4.35 (t, J=6.00 Hz, 1H), 3.84 (t, J=6.00 Hz, 1H), 3.70 (d, J=2.00 Hz, 1H), 3.49 (s, 1H), 3.09 (d, J=9.60 Hz, 1H), 2.93 (d, J=10.00 Hz, 1H), 2.86 (br s, 1H), 2.80 (d, J=1.60 Hz, 1H), 1.83 (d, J=9.60 Hz, 1H), 1.75 (d, J=8.80 Hz, 1H), 0.81-0.76 (m, 4H).

### Example 334 and Example 335

### 2-((2-chloro-5-cyano-3-((S)-4-((R)-1,1-dioxidotetrahydrothiophen-3-yl)-2-methylpiperazin-1-yl)phenyl)amino)-4-(cyclopropylamino)pyrazolo[1,5-a][1,3,5]triazine-8-carbonitrile and 2-((2-chloro-5-cyano-3-((S)-4-((S)-1,1-dioxidotetrahydrothiophen-3-yl)-2-methylpiperazin-1-yl)phenyl)amino)-4-(cyclopropylamino)pyrazolo[1,5-a][1,3,5]triazine-8-carbonitrile

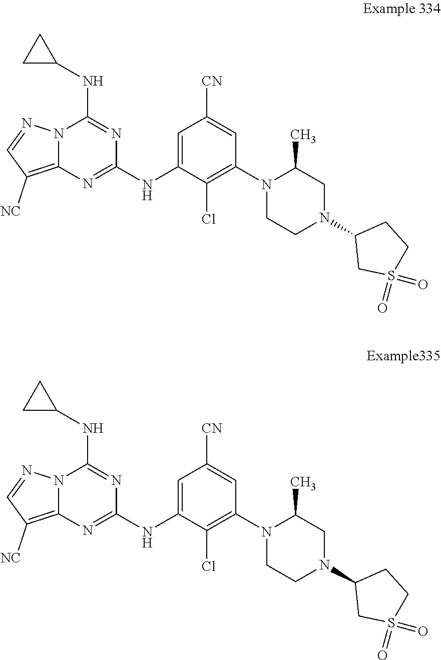 [SMILES: C[C@H]1CN([C@@H]2CCS(=O)(=O)C2)CCN1c1cc(C#N)cc(Nc2nc(NC3CC3)n3ncc(C#N)c3n2)c1Cl ; C[C@H]1CN([C@H]2CCS(=O)(=O)C2)CCN1c1cc(C#N)cc(Nc2nc(NC3CC3)n3ncc(C#N)c3n2)c1Cl]

### Step 1. Tert-butyl (S)-4-(3-((tert-butoxycarbonyl)amino)-2-chloro-5-cyanophenyl)-3-methylpiperazine-1-carboxylate

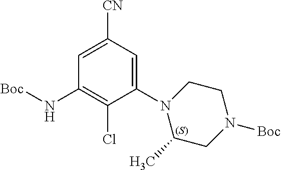

A mixture of tert-butyl (3-bromo-2-chloro-5-cyanophenyl)carbamate (3.8 g, 11.46 mmol), tert-butyl (S)-3-methylpiperazine-1-carboxylate (3.44 g, 17.19 mmol), BINAP (2.141 g, 3.44 mmol), Cs₂CO₃ (13.07 g, 40.1 mmol) in toluene (90 mL) was purged with nitrogen and then Pd₂(dba)₃ (1.049 g, 1.146 mmol) was added and purged with nitrogen for 5 min. The mixture was stirred at 115° C. for 16 h. It was then filtered and concentrated. The residue was purified by ISCO silica gel column chromatography eluting with 20% EtOAc in hexane to offer Tert-butyl (S)-4-(3-((tert-butoxycarbonyl)amino)-2-chloro-5-cyanophenyl)-3-methylpiperazine-1-carboxylate (2.2 g, 42.6% yield) as a pale-yellow solid. ¹H-NMR (400 MHz, DMSO-d₆): δ 8.90 (s, 1H), 7.79 (d, J=1.60 Hz, 1H), 7.51 (d, J=1.20 Hz, 1H), 3.63-3.59 (m, 1H), 3.47-3.41 (m, 3H), 3.15-3.12 (m, 2H), 2.65-2.61 (m, 1H), 1.47 (s, 9H), 1.41 (s, 9H), 1.80 (d, J=8.00 Hz, 3H). LC/MS [M+H]⁺=451.2

### Step 2. Tert-butyl (S)-4-(3-amino-2-chloro-5-cyanophenyl)-3-methylpiperazine-1-carboxylate

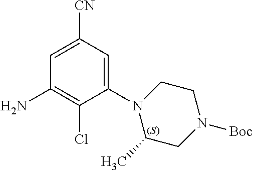

To a solution of tert-butyl (S)-4-(3-((tert-butoxycarbonyl)amino)-2-chloro-5-cyanophenyl)-3-methylpiperazine-1-carboxylate (30.8 g, 68.3 mmol) in DCM (300 mL) at 0° C. was added TFA (79 mL, 1024 mmol) over 5 min. and then stirred at rt for 3 h. The reaction mixture was concentrated under reduced pressure and azeotroped with toluene to afford a pale brown gummy liquid. The crude was dissolved in DCM (300 mL) and cooled to 0° C. and then TEA (57.1 mL, 410 mmol) was added followed by Boc₂O (14.91 g, 68.3 mmol). After stirring for 0.5 h, the reaction mixture was concentrated, and the crude was purified on silica column by using 18% ethyl acetate in petroleum ethe to give tert-butyl (S)-4-(3-amino-2-chloro-5-cyanophenyl)-3-methyl piperazine-1-carboxylate (18.7 g, 76% yield) as a pale brown solid. LC/MS [M−H]⁺=351.2. ¹H-NMR (400 MHz, DMSO-D₆): δ 6.88 (d, J=1.6 Hz, 1H), 6.82 (d, J=1.6 Hz, 1H), 5.83 (br s, 2H), 3.61-3.58 (m, 1H), 3.44-3.34 (m, 3H), 3.20-3.06 (m, 2H), 2.59-2.53 (m, 1H), 1.42 (s, 9H), 0.8 (d, J=6 Hz, 3H). LC/MS [M+H]⁺=351.0.

### Step 3. tert-Butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate

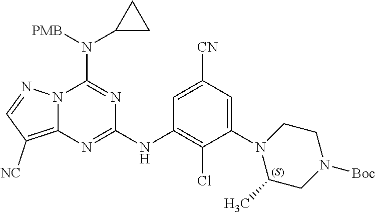

### Method 1:

To a stirred solution of (4-(Cyclopropyl(4-methoxybenzyl)amino)-2-(methylsulfonyl)pyrazolo[1,5-a][1,3,5]triazine-8-carbonitrile) (28 g, 70.3 mmol) and tert-butyl (S)-4-(3-amino-2-chloro-5-cyanophenyl)-3-methylpiperazine-1-carboxylate (24.66 g, 70.3 mmol) in THF (750 mL) at −78° C. was added LiHMDS (141 mL, 141 mmol) in THF dropwise over 15 min. After stirring for another 30 min at −78° C., the reaction mixture was quenched with 300 mL of ice water and 750 mL of EA. The organic layer was washed with brine, dried over Na₂SO₄ and concentrated. The crude was then treated with 210 mL of MeOH followed by stirring for 15 min at rt. The precipitate was filtrated washed with 30 mL of MeOH to offer tert-Butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate (40 g, 85% yield) as a cream color solid. LC/MS [M−H]⁺=667.2. ¹H-NMR (400 MHz, CDCl₃): δ 9.76 (s, 1H), 8.42 (s, 1H), 7.94 (br s, 1H), 7.53 (s, 1H), 7.16 (d, J=8.40 Hz, 2H), 6.86 (d, J=8.80 Hz, 2H), 5.14-5.11 (m, 2H), 3.72 (s, 3H), 3.55-3.50 (m, 2H), 3.18-3.14 (m, 2H), 2.88 (br s, 1H), 2.62-2.55 (m, 2H), 1.43 (s, 9H), 0.85-0.77 (m, 8H). LC/MS [M+H]⁺=669.2.

### Method 2:

### Step a. 2-Amino-4-(cyclopropyl(4-methoxybenzyl)amino)pyrazolo[1,5-a][1,3,5]triazine-8-carbonitrile

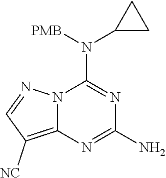

A mixture of 4-(cyclopropyl(4-methoxybenzyl)amino)-2-(methylsulfonyl)pyrazolo[1,5-a][1,3,5]triazine-8-carbonitrile (598 mg, 1.5 mmol) and 0.5 M ammonia in dioxane (9 mL, 4.50 mmol) was stirred at 80° C. for 2 h. After removal of most of the solvent, the formed precipitate was collected washing with MeOH to give 2-Amino-4-(cyclopropyl(4-methoxy benzyl)amino)pyrazolo[1,5-a][1,3,5]triazine-8-carbonitrile (460 mg, 91%) as a solid. ¹H NMR (499 MHz, DMSO-d₆) δ 8.27 (s, 1H), 7.47-7.33 (m, 2H), 7.19 (d, J=8.6 Hz, 2H), 6.88 (d, J=8.7 Hz, 2H), 5.10 (s, 2H), 3.72 (s, 3H), 3.09-2.82 (m, 1H), 0.86-0.68 (m, 4H). LC/MS [M+H]⁺=336.2.

### Step b. tert-butyl (S)-4-(3-bromo-2-chloro-5-cyanophenyl)-3-methylpiperazine-1-carboxylate

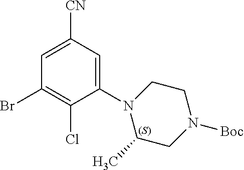

To a stirred solution of 3,5-dibromo-4-chlorobenzonitrile (2 g, 6.77 mmol) in toluene (20 mL), tert-butyl (S)-3-methylpiperazine-1-carboxylate (1.492 g, 7.45 mmol), and cesium carbonate (6.62 g, 20.31 mmol) were added at under N₂ atm. After degassing for 10 min, Pd₂(dba)₃ (0.248 g, 0.271 mmol) was added followed by degassing for another 10 min. The resulting reaction mixture was heated to 95° C. for 24 h. After cooling to rt, the reaction mixture was filtered through a pad of celite and washed with ethyl acetate. The combined organic layers were washed with water and brine, dried over anhydrous sodium sulfate, filtered, concentrated, and purified by silica gel ISCO chromatography eluting with 20% ethyl acetate in hexane to get tert-butyl (S)-4-(3-bromo-2-chloro-5-cyanophenyl)-3-methylpiperazine-1-carboxylate (1.9 g, 67.7% yield) as a white solid. ¹H NMR (499 MHz, CDCl₃) δ 7.68 (d, J=1.9 Hz, 1H), 7.25 (d, J=1.7 Hz, 1H), 3.89-3.52 (m, 3H), 3.51-3.42 (m, 1H), 3.40-3.18 (m, 2H), 2.74-2.63 (m, 1H), 1.51 (s, 9H), 0.95 (d, J=6.3 Hz, 3H). LC/MS [M+H]⁺=414.1.

### Step c. tert-Butyl (S)-4-(2-chloro-5-cyano-3-((8-cyano-4-(cyclopropyl(4-methoxybenzyl)amino)pyrazolo[1,5-a][1,3,5]triazin-2-yl)amino)phenyl)-3-methylpiperazine-1-carboxylate

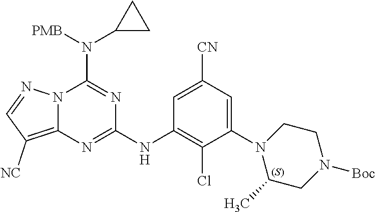

A mixture of 2-Amino-4-(cyclopropyl(4-methoxybenzyl)amino)pyrazolo [1,5-a][1,3,5]triazine-8-carbonitrile (1.4 g, 4.17 mmol), tert-butyl (S)-4-(3-bromo-2-chloro-5-cyanophenyl)-3-methylpiperazine-1-carboxylate (2.078 g, 5.01 mmol), XANTPHOS (0.242 g, 0.417 mmol) and Cs₂CO₃ (4.08 g, 12.52 mmol) in dioxane (20 mL) was purged with nitrogen for 10 min, then Pd₂(dba)₃ (0.191 g, 0.209 mmol) was added followed by purging with nitrogen for another 10 min, finally the resulting mixture was stirred at 90° C. for 16 h. The brown slurry was cooled to rt, celite was added and diluted with ethyl acetate. The reaction mixture was filtered through Celite washing with ethyl acetate. The filtrate was concentrated in vacuo and the residue was purified by silica gel ISCO chromatography, eluting with 30-60% EtOAc/hexane to obtain tert-Butyl (S)-4-(2-chloro-5-cyano-3-((8-cyano-4-(cyclopropyl(4-methoxybenzyl) amino)pyrazolo[1,5-a][1,3,5]triazin-2-yl)amino)phenyl)-3-methylpiperazine-1-carboxylate (2.5 g, 89%) as a solid. ¹H NMR (499 MHz, DMSO-d₆): δ 9.73 (s, 1H), 8.41 (s, 1H), 7.94 (br s, 1H), 7.52 (s, 1H), 7.16 (d, J=8.6 Hz, 2H), 6.96-6.78 (m, 2H), 5.32-4.92 (m, 2H), 3.72 (s, 3H), 3.61-3.34 (m, 4H), 3.22-3.06 (m, 2H), 3.00-2.82 (m, 1H), 2.69-2.58 (m, 1H), 1.43 (s, 9H), 0.91-0.65 (m, 8H). LC/MS [M+H]⁺=669.3.

### Step 4. (S)-2-((2-chloro-5-cyano-3-(2-methylpiperazin-1-yl)phenyl)amino)-4-(cyclopropylamino)pyrazolo[1,5-a][1,3,5]triazine-8-carbonitrile

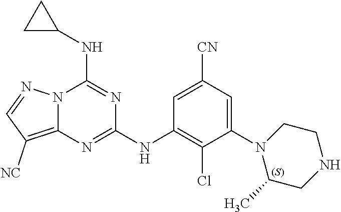

To a solution tert-butyl (S)-4-(2-chloro-5-cyano-3-((8-cyano-4-(cyclopropyl(4-methoxybenzyl)amino)pyrazolo[1,5-a][1,3,5]triazin-2-yl)amino) phenyl)-3-methylpiperazine-1-carboxylate (2.4 g, 3.59 mmol) in 3 mL of DCM was added 2 mL of TFA and then stirred for 0.5 h. After concentration, the residue was taken into DCE (23.91 ml) followed by addition of TFA (11.05 ml, 143 mmol) and anisole (1.567 mL, 14.35 mmol), and the reaction mixture was heated at 50° C. for 1 h. The solvent was removed in vacuo and the material azeotroped with DCM 3 times to remove the excess TFA. 2 ml of 7N NH₃ in MeOH was added to the residue. The resulting solid was filtered and washed with MeOH to give (S)-2-((2-chloro-5-cyano-3-(2-methylpiperazin-1-yl)phenyl) amino)-4-(cyclopropylamino)pyrazolo[1,5-a][1,3,5]triazine-8-carbonitrile (1.5 g, 93% yield) as white solid. ¹H NMR (499 MHz, DMSO-d₆) δ 9.5 (br s, 1H), 8.41 (s, 1H), 8.14 (s, 1H), 7.48 (d, J=1.8 Hz, 1H), 3.42-3.26 (m, 2H), 3.14-3.06 (m, 1H), 2.98 (dd, J=11.9, 3.1 Hz, 1H), 2.90-2.77 (m, 3H), 2.61-2.52 (m, 2H), 0.85 (d, J=6.2 Hz, 3H), 0.83-0.74 (m, 4H). LC/MS [M+H]⁺=449.3.

### Step 5. Example 334 and Example 335

A mixture of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (600 mg, 1.337 mmol), acetic acid (0.383 mL, 6.68 mmol) and dihydrothiophen-3(2H)-one 1,1-dioxide (538 mg, 4.01 mmol) was stirred at rt for 3 h. Sodium cyanoborohydride (252 mg, 4.01 mmol) was added and the contents heated at 50° C. for 48 h. The reaction mixture was concentrated, and the residue partitioned between ethyl acetate and sat. aq. NaHCO₃. The ethyl acetate layer was dried over sodium sulfate, concentrated and the residue was subjected to SFC purification (SFC column: Chiralpak AS-H, 30 mm×250 mm, 5 μm particles; 45% cosolvent (MeOH/MeCN (50:50)—55% CO₂; flow rate: 90 mL/min; column temperature: 45° C.; UV monitored at 210 nm) to give the first peak 2-((2-chloro-5-cyano-3-((S)-4-((R)-1,1-dioxidotetrahydrothiophen-3-yl)-2-methylpiperazin-1-yl)phenyl)amino)-4-(cyclopropylamino)pyrazolo[1,5-a][1,3,5]triazine-8-carbonitrile (303 mg, 40%) and the second peak 2-((2-chloro-5-cyano-3-((S)-4-((S)-1,1-dioxidotetrahydrothiophen-3-yl)-2-methylpiperazin-1-yl)phenyl)amino)-4-(cyclopropylamino)pyrazolo[1,5-a][1,3,5]triazine-8-carbonitrile (237 mg, 31%). Analytical data for Example 334: ¹H NMR (499 MHz, DMSO-d₆) δ 9.50 (br s, 1H), 9.22 (br s, 1H), 8.41 (s, 1H), 8.16 (s, 1H), 7.53 (d, J=1.2 Hz, 1H), 3.56-3.46 (m, 1H), 3.39-3.24 (m, 2H), 3.22-3.14 (m, 1H), 3.12-3.04 (m, 1H), 2.98 (dd, J=12.6, 8.4 Hz, 1H), 2.88-2.80 (m, 2H), 2.74-2.64 (m, 2H), 2.62-2.56 (m, 1H), 2.45-2.32 (m, 2H), 2.31-2.23 (m, 1H), 2.07-1.93 (m, 1H), 0.88 (d, J=6.2 Hz, 3H), 0.82-0.70 (m, 4H). LC/MS [M+H]⁺=567.3. Analytical data for Example 335: ¹H NMR (499 MHz, DMSO-d₆) δ 9.50 (br s, 1H), 9.23 (br s, 1H), 8.41 (s, 1H), 8.16 (s, 1H), 7.54 (s, 1H), 3.50 (br s, 1H), 3.37 (dd, J=12.9, 7.5 Hz, 1H), 3.30-3.23 (m, 1H), 3.21-3.14 (m, 1H), 3.13-3.04 (m, 1H), 3.00 (dd, J=12.8, 9.0 Hz, 1H), 2.88-2.77 (m, 2H), 2.75-2.66 (m, 2H), 2.57-2.52 (m, 2H), 2.39-2.25 (m, 2H), 2.08-1.97 (m, 1H), 0.88 (d, J=6.2 Hz, 3H), 0.83-0.70 (m, 4H). LC/MS [M+H]⁺=567.3.

### Example 336

### (S)-2-((S)-4-(5-cyano-3-((8-cyano-4-(cyclopropylamino)pyrazolo[1,5-a][1,3,5]triazin-2-yl)amino)-2-methoxyphenyl)-3-methylpiperazin-1-yl)propanamide, TFA

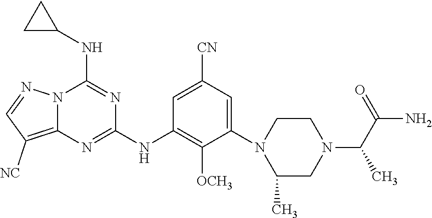 [SMILES: COc1c(Nc2nc(NC3CC3)n3ncc(C#N)c3n2)cc(C#N)cc1N1CCN([C@@H](C)C(N)=O)C[C@@H]1C]

### Step 1. tert-butyl (S)-4-(5-cyano-2-iodophenyl)-3-methylpiperazine-1-carboxylate

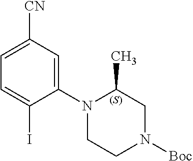

To a mixture of copper(I) iodide (10.59 g, 55.6 mmol and tert-butyl (S)-4-(2-amino-5-cyanophenyl)-3-methylpiperazine-1-carboxylate (1.1 g, 3.01 mmol) in acetonitrile (50 mL) was added tert-butyl nitrite (5.21 g, 50.6 mmol) in acetonitrile (10 mL) dropwise at 0° C. Then the reaction mixture was allowed to stir at rt for 16 h. The reaction mixture was quenched with 10% aq.NaHCO₃ solution and stirred for 15 min and extracted with EtOAc. The organic layer was separated and washed with water, dried over Na₂SO₄, filtered, and concentrated. It was then purified by Biotage isolera, eluting with 12% EtOAc/hexane afford tert-butyl (S)-4-(5-cyano-2-iodophenyl)-3-methylpiperazine-1-carboxylate (5 g, 11.70 mmol, 46.3% yield) as a gummy liquid. LC/MS [M+H]⁺=428.2.

### Step 2. tert-butyl (S)-4-(5-cyano-2-((4-methoxybenzyl)oxy)phenyl)-3-methylpiperazine-1-carboxylate

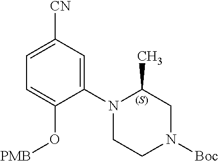

To a 250 mL sealed tube tert-butyl (S)-4-(5-cyano-2-iodophenyl)-3-methylpiperazine-1-carboxylate (5 g, 11.70 mmol) and (4-methoxyphenyl) methanol (12.93 g, 94 mmol) were added, followed by toluene (40 mL) copper(I) iodide (0.669 g, 3.51 mmol), 1,10-phenanthroline (1.265 g, 7.02 mmol) and cesium carbonate (8.39 g, 25.7 mmol). The contents were heated at 110° C., and stirred for 16 h. The reaction mixture was diluted with EtOAc and water. The EtOAc layer was separated and dried over Na₂SO₄ and concentrated. The residue obtained was purified using reverse phase silica column purification eluting with 80% ACN in 0.1% aq. ammonium formate. The pure fractions were collected and concentrated to get the tert-butyl (S)-4-(5-cyano-2-((4-methoxybenzyl)oxy)phenyl)-3-methylpiperazine-1-carboxylate (3.5 g, 8.00 mmol, 68.4% yield) as a gummy liquid. LC/MS [M+H]⁺=438.3.

### Step 3. tert-butyl (S)-4-(5-cyano-2-hydroxyphenyl)-3-methylpiperazine-1-carboxylate

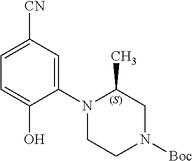

To a stirred solution of tert-butyl (S)-4-(5-cyano-2-((4-methoxybenzyl) oxy)phenyl)-3-methylpiperazine-1-carboxylate (3.2 g, 7.31 mmol) was taken in EtOH/EtOAc (50 ml) (1:1). The reaction mixture was degassed with N₂, and then Pd—C (2 g, 1.879 mmol) was added under N₂ atm. The reaction mixture was stirred under H₂ pressure (Bladder) for 1-5 h, filtered through a celite pad, and the filtrate was concentrated. The residue obtained was purified by Biotage Isolera eluting with 20% EtOAc in hexane gave the desired product tert-butyl (S)-4-(5-cyano-2-hydroxyphenyl)-3-methylpiperazine-1-carboxylate (1.75 g, 5.51 mmol, 75% yield) as white solid. LC/MS [M+H]⁺=318.3.

### Step 4. tert-butyl (S)-4-(3-bromo-5-cyano-2-hydroxyphenyl)-3-methylpiperazine-1-carboxylate

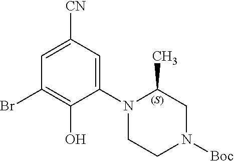

To a stirred solution of tert-butyl (S)-4-(5-cyano-2-hydroxyphenyl)-3-methylpiperazine-1-carboxylate (1.7 g, 5.36 mmol) in dry ACN (25 mL), cooled to 0° C., NBS (1.239 g, 6.96 mmol) was added and stirred for 1 h at 0° C. Then the reaction mixture was allowed to stir at rt for 5-16 h. The reaction mixture was diluted with DCM and washed with aq. NaHCO₃ solution. The DCM layer was separated and dried over sodium sulfate and concentrated. The crude obtained was purified by Biotage Isolera eluting at 15% EtOAc gave the desired product tert-butyl (S)-4-(3-bromo-5-cyano-2-hydroxyphenyl)-3-methylpiperazine-1-carboxylate (700 mg, 33% yield). LC/MS [M+2H]⁺=395.3, 397.3.

### Step 5. tert-butyl (S)-4-(3-bromo-5-cyano-2-methoxyphenyl)-3-methylpiperazine-1-carboxylate

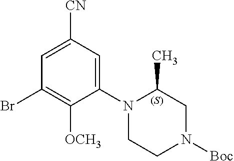

To a stirred solution of tert-butyl (S)-4-(3-bromo-5-cyano-2-hydroxyphenyl)-3-methylpiperazine-1-carboxylate (800 mg, 2.019 mmol) in ACN (15 mL) was added K₂CO₃ (558 mg, 4.04 mmol) and iodomethane (373 mg, 2.62 mmol) and the contents heated at 60° C. for 16 h. The reaction mixture was diluted with EtOAc and water. The EtOAc layer was separated and dried over sodium sulfate and concentrated. The crude product (800 mg, 1.813 mmol, 90% yield, gummy liquid) was taken to the next step without further purification. LC/MS [M+2H]⁺=410.2, 412.2.

### Step 6. tert-butyl (S)-4-(5-cyano-3-((8-cyano-4-(cyclopropyl(4-methoxybenzyl)amino)pyrazolo[1,5-a][1,3,5]triazin-2-yl)amino)-2-methoxyphenyl)-3-methylpiperazine-1-carboxylate

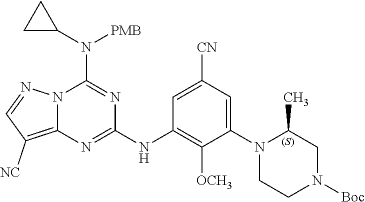

A 100 mL sealed tube was charged with dioxane (50 mL), tert-butyl (S)-4-(3-bromo-5-cyano-2-methoxyphenyl)-3-methylpiperazine-1-carboxylate (800 mg, 1.950 mmol), 2-amino-4-(cyclopropyl(4-methoxybenzyl)amino) pyrazolo[1,5-a][1,3,5]triazine-8-carbonitrile (654 mg, 1.950 mmol), Pd₂(dba)₃ (179 mg, 0.195 mmol), Xantphos (243 mg, 0.390 mmol) and cesium carbonate (1588 mg, 4.87 mmol) sequentially under nitrogen purging. The resulting mixture was heated at 110° C. and stirred for 16 h. The reaction mixture was cooled to rt, filtered through celite pad and washed with DCM (100 mL). The filtrate was concentrated under reduced pressure. The crude product obtained was purified by column chromatography (Biotage isolera) by eluting with 33% ethyl acetate in hexane afford tert-butyl (S)-4-(5-cyano-3-((8-cyano-4-(cyclopropyl(4-methoxybenzyl)amino)pyrazolo[1,5-a][1,3,5]triazin-2-yl)amino)-2-methoxyphenyl)-3-methylpiperazine-1-carboxylate (800 mg, 1.203 mmol, 61.7% yield) as a pale-yellow solid. LCMS (M+H)+=665.3.

### Step 7. (S)-2-((5-cyano-2-methoxy-3-(2-methylpiperazin-1-yl)phenyl)amino)-4-(cyclopropylamino)pyrazolo[1,5-a][1,3,5]triazine-8-carbonitrile

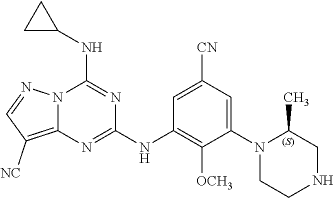

To a stirred solution of tert-butyl (S)-4-(5-cyano-3-((8-cyano-4-(cyclopropyl(4-methoxybenzyl)amino)pyrazolo[1,5-a][1,3,5]triazin-2-yl)amino)-2-methoxyphenyl)-3-methylpiperazine-1-carboxylate (800 mg, 1.203 mmol) in CH₂Cl₂ (20 mL) was added TFA (1.854 mL, 24.07 mmol) at 0° C. and then allowed to stir at rt for 5 h. The reaction mixture was concentrated, and the residue was azeotroped with DCM and three times to yield a light brown solid. The solid obtained was dissolved in ClCH₂CH₂Cl (10 mL) followed by the addition of TFA (3.54 mL, 46.0 mmol) and anisole (0.377 mL, 3.45 mmol) and the contents heated at 55° C. for 2 h. The reaction mixture was cooled to rt and concentrated. MeOH (2 mL) was added to the residue and cooled to 0° C. A few drops of 7 N NH₃ in MeOH were added. The white ppt was filtered and washed with cold MeOH to yield (S)-2-((5-cyano-2-methoxy-3-(2-methylpiperazin-1-yl)phenyl)amino)-4-(cyclopropylamino)pyrazolo[1,5-a][1,3,5]triazine-8-carbonitrile (320 mg, 0.561 mmol, 48.9% yield) as an off-white solid. LC/MS [M+H]⁺=445.3.

### Step 8. Example 336

To a stirred solution of (S)-2-((5-cyano-2-methoxy-3-(2-methyl piperazin-1-yl)phenyl)amino)-4-(cyclopropylamino)pyrazolo[1,5-a][1,3,5]triazine-8-carbonitrile (20 mg, 0.045 mmol) in dry DMSO (0.5 mL) in a 4 mL vial was added DIPEA (0.024 mL, 0.135 mmol) and (R)-1-amino-1-oxopropan-2-yl methanesulfonate (11.28 mg, 0.067 mmol) and the contents heated for 16 h at 80° C. The reaction mixture was cooled to rt, quenched with MeOH (0.5 mL) and purified with reverse phase preparative HPLC to give (S)-2-((S)-4-(5-cyano-3-((8-cyano-4-(cyclopropylamino)pyrazolo[1,5-a][1,3,5]triazin-2-yl)amino)-2-methoxyphenyl)-3-methylpiperazin-1-yl) propanamide·TFA (10 mg, 0.015 mmol, 33.2% yield) as white solid. ¹H NMR 400 MHz, DMSO-d₆) δ 10.18-9.85 (m, 1H), 9.32 (br s, 1H), 9.04 (s, 1H), 8.62-8.40 (m, 2H), 8.16-7.99 (m, 1H), 7.86-7.69 (m, 1H), 7.57-7.38 (m, 1H), 4.12-3.86 (m, 3H), 3.15-2.83 (m, 3H), 2.54 (s, 1H), 1.49 (br d, J=2.0 Hz, 2H), 1.00-0.89 (m, 2H), 0.84 (d, J=5.5 Hz, 3H). LC/MS [M+H]⁺=516.2.

### Example 337

### (S)-2-(4-(2-chloro-5-cyano-3-((8-cyano-4-(ethylamino)pyrazolo[1,5-a][1,3,5]triazin-2-yl)amino)phenyl)piperazin-1-yl)propanamide

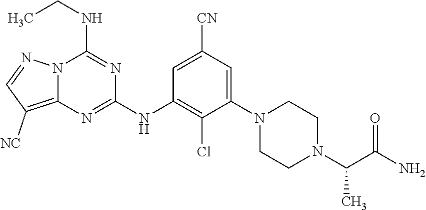 [SMILES: CCNc1nc(Nc2cc(C#N)cc(N3CCN([C@@H](C)C(N)=O)CC3)c2Cl)nc2c(C#N)cnn12]

Example 337 was synthesized based on the protocol outlined for the synthesis of Example 336, Step 8. ¹H NMR (499 MHz, DMSO-d₆) δ 9.56 (br s, 1H), 9.10-9.00 (m, 1H), 8.40 (s, 1H), 7.91 (d, J=1.8 Hz, 1H), 7.39 (d, J=1.9 Hz, 1H), 7.24 (br d, J=2.4 Hz, 1H), 7.02 (br d, J=2.2 Hz, 1H), 3.47-3.37 (m, 2H), 3.11-3.03 (m, 5H), 2.74-2.60 (m, 4H), 1.18 (t, J=7.1 Hz, 3H), 1.14 (d, J=6.9 Hz, 3H). LCMS (Method B): RT=1.62 min; LCMS (ESI) m/z calcd for C₂₂H₂₄ClN₁₁O: 493.1; found: 494.0 [M+H]⁺.

### Example 338

### (S)-2-((R)-4-(2-chloro-5-cyano-3-((8-cyano-4-(cyclopropylamino)pyrazolo[1,5-a][1,3,5]triazin-2-yl)amino)phenyl)-3-methylpiperazin-1-yl)propanamide

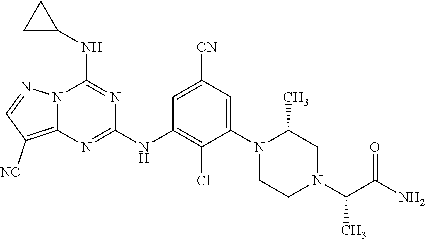 [SMILES: C[C@@H]1CN([C@@H](C)C(N)=O)CCN1c1cc(C#N)cc(Nc2nc(NC3CC3)n3ncc(C#N)c3n2)c1Cl]

Example 338 was synthesized based on the protocol outlined for the synthesis of Example 336, Step 8. ¹HNMR (400 MHz, DMSO-d₆): δ 9.55 (s, 1H), 9.25 (d, J=2.80 Hz, 1H), 8.42 (s, 1H), 8.15 (d, J=1.60 Hz, 1H), 7.52 (d, J=1.60 Hz, 1H), 7.24 (d, J=2.00 Hz, 1H), 7.07 (d, J=2.80 Hz, 1H), 3.48-3.55 (m, 1H), 3.30 (d, J=6.00 Hz, 2H), 3.17-3.18 (m, 1H), 2.80-2.87 (m, 1H), 2.67-2.78 (m, 1H), 2.59-2.62 (m, 2H), 2.24-2.31 (m, 1H), 1.12 (d, J=−6.80 Hz, 3H), 0.88 (d, J=6.00 Hz, 3H), 0.74-0.80 (m, 4H). LCMS [M+H]⁺=520.2.

### Example 339

### (S)-2-((S)-4-(2-chloro-5-cyano-3-((8-cyano-4-(cyclopropylamino)pyrazolo[1,5-a][1,3,5]triazin-2-yl)amino)phenyl)-3-cyclopropylpiperazin-1-yl)propanamide

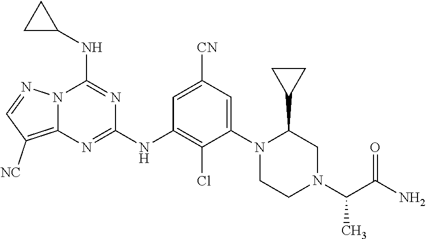 [SMILES: C[C@@H](C(N)=O)N1CCN(c2cc(C#N)cc(Nc3nc(NC4CC4)n4ncc(C#N)c4n3)c2Cl)[C@@H](C2CC2)C1]

Example 339 was synthesized based on the protocol outlined for the synthesis of Example 336, Step 8. ¹H NMR (499 MHz, DMSO-d₆) δ 9.45 (br s, 1H), 9.19 (br s, 1H), 8.40 (s, 1H), 8.10 (br s, 1H), 7.54 (s, 1H), 7.21 (d, J=2.9 Hz, 1H), 7.07 (d, J=2.6 Hz, 1H), 3.46-3.35 (m, 1H), 3.11 (q, J=6.9 Hz, 1H), 2.92-2.83 (m, 2H), 2.74-2.56 (m, 5H), 1.13 (d, J=6.9 Hz, 3H), 0.85-0.70 (m, 5H), 0.26 (tt, J=8.9, 4.5 Hz, 1H), 0.15 (tt, J=8.7, 4.5 Hz, 1H), −0.07 (dq, J=9.5, 4.7 Hz, 1H), −0.42 (br dd, J=8.9, 4.3 Hz, 1H). LCMS [M+H]⁺=546.1.

### Example 340

### (S)-2-((2-chloro-5-cyano-3-(4-((3-hydroxyoxetan-3-yl)methyl)-2-methylpiperazin-1-yl)phenyl)amino)-4-(cyclopropylamino)pyrazolo[1,5-a][1,3,5]triazine-8-carbonitrile

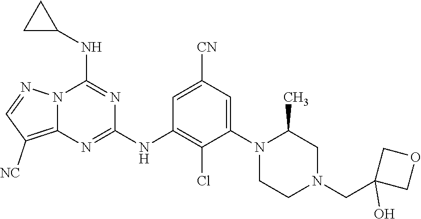 [SMILES: C[C@H]1CN(CC2(O)COC2)CCN1c1cc(C#N)cc(Nc2nc(NC3CC3)n3ncc(C#N)c3n2)c1Cl]

Example 340 was synthesized based on the protocol outlined for the synthesis of Example 336, Step 8. ¹HNMR (400 MHz, DMSO-d₆): δ 9.47 (s, 1H), 9.20 (d, J=2.8 Hz, 1H), 8.39 (s, 1H), 8.12 (s, 1H), 7.49 (s, 1H), 5.25 (s, 1H), 4.41 (d, J=9.6 Hz, 4H), 3.49-3.45 (m, 1H), 3.13-3.08 (m, 1H), 2.82-2.79 (m, 1H), 2.82-2.79 (m, 2H), 2.68-2.58 (m, 5H), 2.33-2.27 (m, 1H), 0.84 (d, J=6 Hz, 3H), 0.77-0.70 (m, 4H). LCMS [M+H]⁺=found: 535.1.

### Example 341

### 2-((3-((S)-4-(((S)-1-acetylpyrrolidin-3-yl)methyl)-2-methylpiperazin-1-yl)-2-chloro-5-cyanophenyl)amino)-4-(cyclopropylamino)pyrazolo[1,5-a][1,3,5]triazine-8-carbonitrile

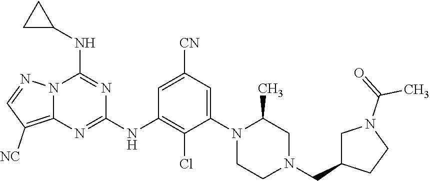 [SMILES: CC(=O)N1CC[C@@H](CN2CCN(c3cc(C#N)cc(Nc4nc(NC5CC5)n5ncc(C#N)c5n4)c3Cl)[C@@H](C)C2)C1]

Example 341 was synthesized based on the protocol outlined for the synthesis of Example 336, Step 8. ¹H NMR (499 MHz, DMSO-d₆) δ 9.59 (br s, 1H), 9.26 (br s, 1H), 8.43 (s, 1H), 8.31 (br s, 1H), 7.70 (br s, 1H), 3.77-3.11 (m, 8H), 3.06-2.81 (m, 4H), 2.78-2.61 (m, 2H), 2.21-1.91 (m, 5H), 1.79-1.58 (m, 1H), 0.91 (br d, J=5.7 Hz, 3H), 0.85-0.73 (m, 4H). LCMS [M+H]⁺=574.1.

### Example 342

### 2-((3-((S)-4-(((R)-1-acetylpyrrolidin-3-yl)methyl)-2-methylpiperazin-1-yl)-2-chloro-5-cyanophenyl)amino)-4-(cyclopropylamino)pyrazolo[1,5-a][1,3,5]triazine-8-carbonitrile

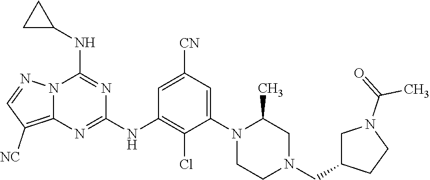 [SMILES: CC(=O)N1CC[C@H](CN2CCN(c3cc(C#N)cc(Nc4nc(NC5CC5)n5ncc(C#N)c5n4)c3Cl)[C@@H](C)C2)C1]

Example 342 was synthesized based on the protocol outlined for the synthesis of Example 336, Step 8. ¹H NMR (499 MHz, DMSO-d₆) δ 9.50 (s, 1H), 9.22 (d, J=3.1 Hz, 1H), 8.41 (s, 1H), 8.15 (s, 1H), 7.53 (br s, 1H), 3.63-3.35 (m, 4H), 3.26-3.10 (m, 2H), 3.06-2.92 (m, 1H), 2.89-2.80 (m, 1H), 2.77-2.62 (m, 2H), 2.47-2.27 (m, 4H), 2.08-1.87 (m, 5H), 1.70-1.47 (m, 1H), 0.89 (br d, J=5.8 Hz, 3H), 0.84-0.69 (m, 4H). LCMS [M+H]⁺=574.1.

### Example 343

### 2-((2-chloro-5-cyano-3-((S)-2-methyl-4-(((S)-5-oxopyrrolidin-3-yl)methyl)piperazin-1-yl)phenyl)amino)-4-(cyclopropylamino)pyrazolo[1,5-a][1,3,5]triazine-8-carbonitrile

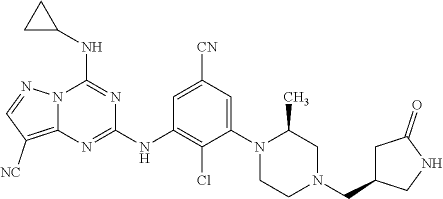 [SMILES: C[C@H]1CN(C[C@@H]2CNC(=O)C2)CCN1c1cc(C#N)cc(Nc2nc(NC3CC3)n3ncc(C#N)c3n2)c1Cl]

Example 343 was synthesized based on the protocol outlined for the synthesis of Example 336, Step 8. ¹H NMR (499 MHz, DMSO-d₆) δ 9.28 (br s, 2H), 8.35 (br s, 1H), 8.31-8.14 (m, 1H), 7.49 (br d, J=9.7 Hz, 2H), 3.51 (br s, 1H), 3.18 (br d, J=4.3 Hz, 1H), 3.00 (dd, J=9.5, 5.3 Hz, 1H), 2.89 (br s, 1H), 2.74-2.59 (m, 4H), 2.48-2.41 (m, 1H), 2.40-2.21 (m, 5H), 1.92 (dd, J=16.6, 6.5 Hz, 1H), 0.88 (d, J=6.1 Hz, 3H), 0.78-0.71 (m, 4H). LCMS [M+H]⁺=546.2.

### Example 344

### 2-((2-chloro-5-cyano-3-((S)-2-methyl-4-(((R)-5-oxopyrrolidin-3-yl)methyl)piperazin-1-yl)phenyl)amino)-4-(cyclopropylamino)pyrazolo[1,5-a][1,3,5]triazine-8-carbonitrile

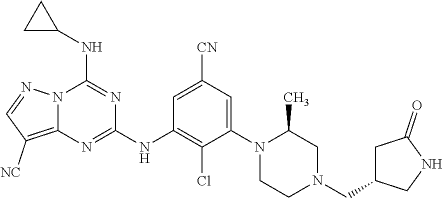 [SMILES: C[C@H]1CN(C[C@H]2CNC(=O)C2)CCN1c1cc(C#N)cc(Nc2nc(NC3CC3)n3ncc(C#N)c3n2)c1Cl]

Example 344 was synthesized based on the protocol outlined for the synthesis of Example 336, Step 8. ¹H NMR (499 MHz, DMSO-d₆) δ 9.33 (br s, 2H), 8.34 (br s, 1H), 8.31-8.15 (m, 1H), 7.50-7.46 (m, 2H), 3.51 (br d, J=6.9 Hz, 1H), 3.18 (br d, J=4.4 Hz, 1H), 2.98 (dd, J=9.6, 5.3 Hz, 1H), 2.90 (br s, 1H), 2.77-2.60 (m, 4H), 2.47 (br d, J=2.1 Hz, 1H), 2.39-2.30 (m, 3H), 2.28-2.15 (m, 2H), 1.93 (dd, J=16.5, 6.4 Hz, 1H), 0.88 (d, J=6.1 Hz, 3H), 0.77-0.71 (m, 4H). LCMS [M+H]⁺=546.2.

### Example 345

### 2-((2-chloro-5-cyano-3-((2S)-2-methyl-4-(2-oxoazepan-3-yl)piperazin-1-yl)phenyl)amino)-4-(cyclopropylamino)pyrazolo[1,5-a][1,3,5]triazine-8-carbonitrile

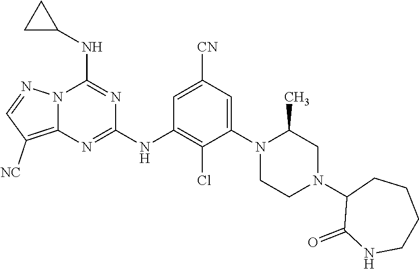 [SMILES: C[C@H]1CN(C2CCCCNC2=O)CCN1c1cc(C#N)cc(Nc2nc(NC3CC3)n3ncc(C#N)c3n2)c1Cl]

Example 345 was synthesized based on the protocol outlined for the synthesis of Example 336, Step 8. ¹H NMR (499 MHz, DMSO-d₆) δ 9.50 (s, 1H), 9.22 (br s, 1H), 8.42 (s, 1H), 8.14 (br s, 1H), 7.52-7.45 (m, 2H), 3.65-3.42 (m, 2H), 3.27-3.18 (m, 1H), 3.05-2.78 (m, 4H), 2.77-2.48 (m, 4H), 1.83 (br d, J=8.3 Hz, 2H), 1.60 (br d, J=8.5 Hz, 3H), 1.51-1.38 (m, 1H), 0.91 (br t, J=6.8 Hz, 3H), 0.84-0.78 (m, 2H), 0.78-0.72 (m, 2H). LCMS (Method B): t_{R}=1.96 min; LCMS (ESI) m/z calcd for C₂₇H₃₀ClN₁₁O: 559.2; found: 560.1 [M+H]⁺.

### Example 346

### 2-((2-chloro-5-cyano-3-((S)-2-methyl-4-(((R)-2-oxopiperidin-4-yl)methyl)piperazin-1-yl)phenyl)amino)-4-(cyclopropylamino)pyrazolo[1,5-a][1,3,5]triazine-8-carbonitrile

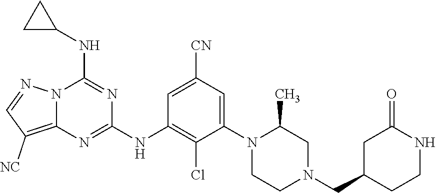 [SMILES: C[C@H]1CN(C[C@@H]2CCNC(=O)C2)CCN1c1cc(C#N)cc(Nc2nc(NC3CC3)n3ncc(C#N)c3n2)c1Cl]

Example 346 was synthesized based on the protocol outlined for the synthesis of Example 336, Step 8. 1H NMR (499 MHz, DMSO-d₆) δ 9.40 (br s, 1H), 9.24 (br s, 1H), 8.38 (s, 1H), 8.19 (br s, 1H), 7.51 (s, 1H), 7.41 (br s, 1H), 3.52 (br d, J=5.5 Hz, 1H), 3.25-3.11 (m, 4H), 2.87 (br s, 1H), 2.75-2.62 (m, 3H), 2.48-2.41 (m, 1H), 2.28-2.20 (m, 4H), 2.11-2.02 (m, 1H), 1.89-1.82 (m, 2H), 1.37-1.29 (m, 1H), 0.89 (d, J=6.1 Hz, 3H), 0.80-0.73 (m, 4H). LCMS [M+H]⁺=560.0.

### Example 347

### 2-((2-chloro-5-cyano-3-((S)-2-methyl-4-(((S)-2-oxopiperidin-4-yl)methyl)piperazin-1-yl)phenyl)amino)-4-(cyclopropylamino)pyrazolo[1,5-a][1,3,5]triazine-8-carbonitrile

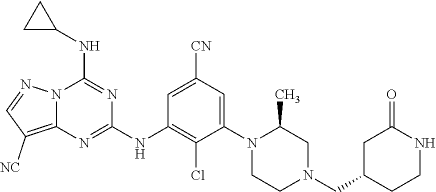 [SMILES: C[C@H]1CN(C[C@H]2CCNC(=O)C2)CCN1c1cc(C#N)cc(Nc2nc(NC3CC3)n3ncc(C#N)c3n2)c1Cl]

Example 347 was synthesized based on the protocol outlined for the synthesis of Example 336, Step 8. 1H NMR (499 MHz, DMSO-d₆) δ 9.3 (br s, 2H), 8.36 (br s, 1H), 8.22 (br d, J=1.2 Hz, 1H), 7.51 (br s, 1H), 7.41 (br s, 1H), 3.52 (br s, 1H), 3.23-3.06 (m, 4H), 2.88 (br s, 1H), 2.70 (br t, J=7.6 Hz, 2H), 2.48-2.41 (m, 1H), 2.31-2.14 (m, 4H), 2.13-2.01 (m, 1H), 1.89-1.78 (m, 2H), 1.37-1.28 (m, 1H), 0.89 (br d, J=6.1 Hz, 3H), 0.81-0.72 (m, 4H). LCMS [M+H]⁺=560.1.

### Example 348

### 2-((2-chloro-5-cyano-3-((2S)-2-methyl-4-((2-oxoazepan-3-yl)methyl)piperazin-1-yl)phenyl)amino)-4-(cyclopropylamino)pyrazolo[1,5-a][1,3,5]triazine-8-carbonitrile, TFA

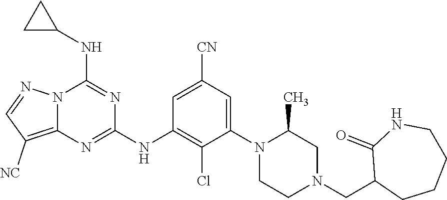 [SMILES: C[C@H]1CN(CC2CCCCNC2=O)CCN1c1cc(C#N)cc(Nc2nc(NC3CC3)n3ncc(C#N)c3n2)c1Cl]

Example 348 was synthesized based on the protocol outlined for the synthesis of Example 336, Step 8. ¹H NMR (499 MHz, DMSO-d₆) δ 9.50 (s, 1H), 9.23 (br s, 1H), 8.42 (s, 1H), 8.14 (br s, 1H), 7.53 (br d, J=10.4 Hz, 1H), 7.41 (br s, 1H), 3.59-3.45 (m, 1H), 3.30-3.12 (m, 2H), 3.07-2.97 (m, 1H), 2.84 (br dd, J=6.3, 3.3 Hz, 1H), 2.73-2.58 (m, 3H), 2.39-2.25 (m, 2H), 2.11-2.02 (m, 1H), 1.94-1.88 (m, 1H), 1.79-1.69 (m, 1H), 1.59-1.49 (m, 1H), 1.31-1.07 (m, 5H), 0.88 (br dd, J=5.8, 2.1 Hz, 3H), 0.83-0.74 (m, 4H). LCMS [M+H]⁺=574.2.

### Example 349

### 2-((2-Chloro-5-cyano-3-((S)-2-methyl-4-(((1s,3R)-3-(methylsulfonyl)cyclobutyl)methyl)piperazin-1-yl)phenyl)amino)-4-(cyclopropylamino)pyrazolo[1,5-a][1,3,5]triazine-8-carbonitrile

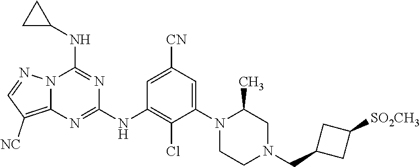 [SMILES: C[C@H]1CN(C[C@H]2C[C@@H](S(C)(=O)=O)C2)CCN1c1cc(C#N)cc(Nc2nc(NC3CC3)n3ncc(C#N)c3n2)c1Cl]

Example 349 was synthesized based on the protocol outlined for the synthesis of Example 336, Step 8. ¹H NMR (599 MHz, DMSO-d₆) δ 9.49 (s, 1H), 9.21 (br d, J=2.7 Hz, 1H), 8.40 (s, 1H), 8.18 (br s, 1H), 7.53 (br s, 1H), 3.94-3.84 (m, 1H), 3.56-3.40 (m, 2H), 3.21-3.09 (m, 1H), 2.84 (s, 5H), 2.73 (br d, J=5.4 Hz, 1H), 2.65-2.51 (m, 3H), 2.43-2.24 (m, 3H), 2.09-1.95 (m, 3H), 0.87 (br d, J=5.1 Hz, 3H), 0.83-0.74 (m, 4H). LCMS [M+H]⁺=595.1.

### Example 350

### 2-((2-chloro-5-cyano-3-((S)-2-methyl-4-(((1 r,3S)-3-(methylsulfonyl)cyclobutyl)methyl)piperazin-1-yl)phenyl)amino)-4-(cyclopropylamino)pyrazolo[1,5-a][1,3,5]triazine-8-carbonitrile

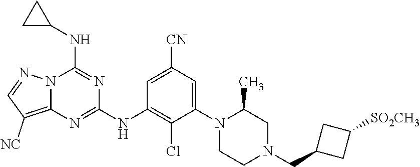 [SMILES: C[C@H]1CN(C[C@H]2C[C@H](S(C)(=O)=O)C2)CCN1c1cc(C#N)cc(Nc2nc(NC3CC3)n3ncc(C#N)c3n2)c1Cl]

Example 350 was synthesized based on the protocol outlined for the synthesis of Example 336, Step 8. ¹H NMR (500 MHz, DMSO-d₆) δ 9.49 (s, 1H), 9.22 (br s, 1H), 8.41 (s, 1H), 8.15 (s, 1H), 7.52 (s, 1H), 3.98-3.87 (m, 1H), 3.55-3.44 (m, 2H), 3.21-3.11 (m, 1H), 2.88 (s, 3H), 2.84 (br s, 1H), 2.72-2.57 (m, 3H), 2.48-2.38 (m, 5H), 2.22-2.11 (m, 1H), 2.10-1.98 (m, 2H), 0.87 (d, J=6.2 Hz, 3H), 0.83-0.71 (m, 4H). LCMS [M+H]⁺=595.1.

### Example 351

### (S)-2-((5-cyano-2-cyclopropyl-3-(4-(1,1-dioxidotetrahydro-2H-thiopyran-4-yl)-2-methylpiperazin-1-yl)phenyl)amino)-4-(cyclopropylamino)pyrazolo[1,5-a][1,3,5]triazine-8-carbonitrile

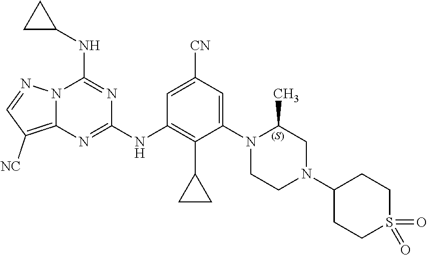

### Step 1. tert-butyl (S)-4-(5-cyano-2-nitrophenyl)-3-methylpiperazine-1-carboxylate

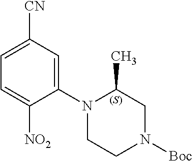

3-fluoro-4-nitrobenzonitrile (100 g, 602 mmol) was added to a stirred solution of tert-butyl (S)-3-methylpiperazine-1-carboxylate (133 g, 662 mmol) and DIPEA (315 mL, 1806 mmol) in DMSO (500 mL) at ambient temperature. The resulting mixture was stirred at 100° C. for 16 h. Reaction mixture was diluted with water (3000 mL) and extracted with ethyl acetate (2000 mL×2). Combined organic layer was washed with water (3000 mL×2), brine solution (2000 mL), dried over anhydrous Na₂SO₄, filtered, and concentrated under reduced pressure to afford tert-butyl (S)-4-(5-cyano-2-nitrophenyl)-3-methylpiperazine-1-carboxylate (230 g, 636 mmol, 106% yield) as pale brown gummy. The crude product obtained was taken for next step without purification. LC/MS [M+H]⁺=347.3.

### Step 2. tert-butyl (S)-4-(2-amino-5-cyanophenyl)-3-methylpiperazine-1-carboxylate

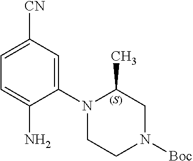

Zinc (152 g, 2324 mmol) was added to a stirred suspension of tert-butyl (S)-4-(5-cyano-2-nitrophenyl)-3-methylpiperazine-1-carboxylate (115 g, 332 mmol) and ammonium chloride (266 g, 4980 mmol) in THF (1000 mL) and methanol (1000 mL) 0° C. The resulting mixture was stirred at ambient temperature for 16 h. The reaction mixture was diluted with ethyl acetate (2 L), stirred for 15 min, the resulting mixture was filtered through celite pad, washed with 2 L of ethyl acetate, and the filtrate was concentrated under reduced pressure. The crude product obtained was purified by column chromatography over silica gel eluting with ethyl acetate in petroleum ether. The desired fractions were pooled together and concentrated under reduced pressure to afford tert-butyl (S)-4-(2-amino-5-cyanophenyl)-3-methylpiperazine-1-carboxylate (95 g, 278 mmol, 84% yield) as pale brown solid. LC/MS [M+H]⁺=317.3. ¹H NMR (400 MHz, DMSO-d₆) 5=7.37 (s, 1H), 7.25 (dd, J=2.0, 8.5 Hz, 1H), 6.73 (d, J=8.0 Hz, 1H), 5.96 (brs, 2H), 3.88-3.67 (m, 2H), 3.27-3.14 (m, 1H), 3.07-2.98 (m, 1H), 2.88-2.72 (m, 2H), 1.43 (s, 9H), 0.75 (d, J=6.0 Hz, 3H).

### Step 3. tert-butyl (S)-4-(2-amino-3-chloro-5-cyanophenyl)-3-methylpiperazine-1-carboxylate

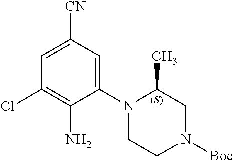

NCS (57.0 g, 427 mmol) was added to a stirred solution of tert-butyl (S)-4-(2-amino-5-cyanophenyl)-3-methylpiperazine-1-carboxylate (90 g, 284 mmol) in tetrahydrofuran (1000 mL) at rt. The resulting mixture was stirred at ambient temperature for 16h. Reaction mixture was quenched with saturated sodium thiosulfate solution and extracted with ethyl acetate (1500 mL×2). The combined organic layer was washed with saturated brine solution (1500 mL), dried over anhydrous Na₂SO₄, filtered, and concentrated under reduced pressure. The crude product obtained was purified by column chromatography over silica gel eluting with ethyl acetate in petroleum ether. The desired fractions were pooled together and concentrated under reduce to afford tert-butyl (S)-4-(2-amino-3-chloro-5-cyanophenyl)-3-methylpiperazine-1-carboxylate (67 g, 180 mmol, 63.4% yield) as pale brown solid. LC/MS [M+H]⁺=351.3. 1H NMR (400 MHz, DMSO-d₆) 5=7.57 (d, J=2.0 Hz, 1H), 7.51-7.48 (br s, 1H), 6.09 (br s, 2H), 3.95-3.65 (m, 2H), 3.27-3.12 (m, 1H), 3.09-2.95 (m, 1H), 2.90-2.78 (m, 2H), 2.58-2.52 (m, 2H), 1.43 (s, 9H), 0.75 (s, 3H).

### Step 4. tert-butyl (S)-4-(2-bromo-3-chloro-5-cyanophenyl)-3-methylpiperazine-1-carboxylate

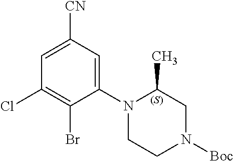

Copper(I) bromide (40.9 g, 285 mmol) was added to a stirred solution of tert-butyl (S)-4-(2-amino-3-chloro-5-cyanophenyl)-3-methylpiperazine-1-carboxylate (25 g, 71.3 mmol) in acetonitrile (400 mL) at 0° C. It was stirred for 20 min. Tert-butyl nitrite (16.95 mL, 143 mmol) was added at 0° C. over 30 min and stirred at ambient temperature for 24 h. Reaction mixture was diluted with water (1000 mL). Ethyl acetate (1000 mL) was added, the contents were stirred for 15 min, the resulting mixture was filtered through celite pad and washed with 500 mL of ethyl acetate. The organic layer was separated and aqueous layer was extracted with ethyl acetate (750 mL). Combined organic layers were washed with water (1000 mL), saturated brine solution (1000 mL), dried over anhydrous Na₂SO₄, filtered, and concentrated under reduced pressure. The desired fractions were pooled together and concentrated under reduced pressure to afford tert-butyl (S)-4-(2-bromo-3-chloro-5-cyanophenyl)-3-methylpiperazine-1-carboxylate (25 g, 58.3 mmol, 93% yield) as off white solid.

### Step 5. tert-butyl (S)-4-(3-chloro-5-cyano-2-cyclopropylphenyl)-3-methylpiperazine-1-carboxylate

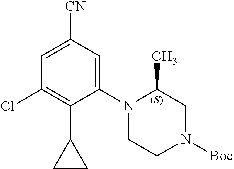

Into a 250 mL sealed tube, 1,4-Dioxane (60 mL), water (12 mL), tert-butyl (S)-4-(2-bromo-3-chloro-5-cyanophenyl)-3-methylpiperazine-1-carboxylate (4.1 g, 9.89 mmol), cyclopropylboronic acid (1.104 g, 12.85 mmol), K₂CO₃ (4.10 g, 29.7 mmol) and finally PdCl₂(dppf)-CH₂Cl₂ adduct (0.807 g, 0.989 mmol) were added under nitrogen purging at ambient temperature. The reaction mixture was stirred at 90° C. for 10 h. (Reaction was conducted in two separate tubes), The reaction mixtures were mixed and concentrated under reduced pressure. The crude product obtained was purified by column chromatography over silica gel by eluting with ethyl acetate in petroleum ether. The desired fractions were pooled together and concentrated under reduced pressure to afford tert-butyl (S)-4-(3-chloro-5-cyano-2-cyclopropylphenyl)-3-methylpiperazine-1-carboxylate (3 g, 7.84 mmol, 79% yield) as a pale-yellow gummy solid. LC/MS [M+H]⁺=376.2. ¹H-NMR (400 MHz, DMSO-d₆): δ 7.62 (s, 1H), 7.37 (s, 1H), 3.73-3.62 (m, 2H), 3.53-3.39 (m, 2H), 3.23-3.17 (m, 2H), 2.78-2.68 (m, 1H), 1.86-1.78 (m, 1H), 1.20-1.08 (m, 2H), 1.05-0.98 (m, 1H), 0.80 (d, J=6.40 Hz, 3H), 0.52-0.43 (m, 1H).

### Step 6. tert-butyl (S)-4-(5-cyano-3-((8-cyano-4-(cyclopropyl(4-methoxybenzyl)amino)pyrazolo[1,5-a][1,3,5]triazin-2-yl)amino)-2-cyclopropylphenyl)-3-methylpiperazine-1-carboxylate

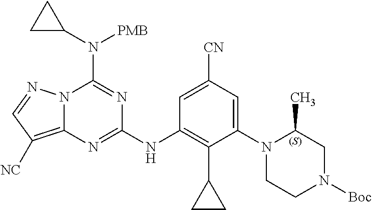

Into a 20 mL microwave vial, 1,4-Dioxane (8 mL), tert-butyl (S)-4-(3-chloro-5-cyano-2-cyclopropylphenyl)-3-methylpiperazine-1-carboxylate (673 mg, 1.789 mmol), 2-amino-4-(cyclopropyl(4-methoxybenzyl)amino) pyrazolo[1,5-a][1,3,5]triazine-8-carbonitrile (500 mg, 1.491 mmol), Cs₂CO₃ (1457 mg, 4.47 mmol), xantphos (345 mg, 0.596 mmol) and finally Pd₂(dba)₃ (273 mg, 0.298 mmol) were charged sequentially under nitrogen purging at ambient temperature. The reaction mixture was stirred at 140° C. for 5 h in Biotage microwave reactor. [The reaction was carried out in two more batches (2×500 mg)]. Reaction mixtures were mixed and concentrated under reduced pressure. Crude product obtained was purified by column chromatography over silica gel by eluting with ethyl acetate in petroleum ether. The desired fractions were pooled together and concentrated under reduced pressure to afford tert-butyl (S)-4-(5-cyano-3-((8-cyano-4-(cyclopropyl(4-methoxybenzyl) amino)pyrazolo[1,5-a][1,3,5]triazin-2-yl)amino)-2-cyclopropylphenyl)-3-methylpiperazine-1-carboxylate (300 mg, 8% yield) as pale brown solid. LC/MS [M+H]⁺=673.2.

### Step 7. (S)-2-((5-cyano-2-cyclopropyl-3-(2-methylpiperazin-1-yl)phenyl)amino)-4-(cyclopropylamino)pyrazolo[1,5-a][1,3,5]triazine-8-carbonitrile

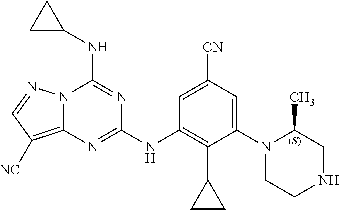

TFA (0.685 mL, 8.89 mmol) was added to a stirred solution of tert-butyl (S)-4-(5-cyano-3-((8-cyano-4-(cyclopropyl(4-methoxybenzyl)amino) pyrazolo[1,5-a][1,3,5]triazin-2-yl)amino)-2-cyclopropylphenyl)-3-methylpiperazine-1-carboxylate (300 mg, 0.445 mmol) in dichloromethane (10 mL) at 0° C. The resulting mixture was stirred at ambient temperature for 1h. Reaction mixture was concentrated under reduced pressure to afford (S)-2-((5-cyano-2-cyclopropyl-3-(2-methylpiperazin-1-yl)phenyl)amino)-4-(cyclopropyl(4-methoxybenzyl)amino)pyrazolo[1,5-a][1,3,5]triazine-8-carbonitrile·TFA (400 mg, 0.460 mmol) as pale brown gummy salt. The solid obtained was taken in 1,2-dichloroethane (10 mL) at 0° C. TFA (1.342 mL, 17.42 mmol) and anisole (0.254 mL, 2.323 mmol) were added, and the resulting mixture was stirred at 55° C. for 1.5 h. The reaction mixture was concentrated under reduced pressure. The residue was diluted with dichloromethane (5 mL) and 20 mL of diethyl ether was added and the contents stirred for 10 min. The resulting solid was filtered, washed with diethyl ether (10 mL) to afford (S)-2-((5-cyano-2-cyclopropyl-3-(2-methylpiperazin-1-yl)phenyl)amino)-4-(cyclopropylamino)pyrazolo[1,5-a][1,3,5]triazine-8-carbonitrile·TFA (150 mg) as pale brown solid. LC/MS [M+H]⁺=455.2. The solid obtained was taken to next step. The filtrate was purified by reverse phase preparative HPLC (Ammonium formate method, Column: Xbridge C18 (150×19) mm, 5 micron. The product fractions were pooled together and lyophilized to afford (S)-2-((5-cyano-2-cyclopropyl-3-(2-methylpiperazin-1-yl)phenyl)amino)-4-(cyclopropylamino)pyrazolo[1,5-a][1,3,5]triazine-8-carbonitrile, formic acid salt (10.3 mg) as off white solid. LC/MS [M+H]⁺=455.2. ¹H-NMR (400 MHz, DMSO-d₆): δ 9.41 (br s, 1H), 8.39 (s, 1H), 8.25 (s, 1H), 7.90 (br s, 1H), 7.14 (s, 1H), 3.56-3.51 (m, 2H), 3.17-3.08 (m, 3H), 2.98-2.82 (m, 5H), 2.70-2.63 (m, 3H), 1.86-1.79 (m, 1H), 1.08-0.96 (m, 3H), 0.88 (d, J=6.0 Hz, 3H), 0.83-0.73 (m, 6H), 0.32-0.24 (m, 1H).

### Step 8. Example 351

Tetrahydro-4H-thiopyran-4-one 1,1-dioxide (11.73 mg, 0.079 mmol), sodium triacetoxyborohydride (55.9 mg, 0.264 mmol) and acetic acid (9.06 μL, 0.158 mmol) were added to a solution of (S)-2-((5-cyano-2-cyclopropyl-3-(2-methylpiperazin-1-yl)phenyl)amino)-4-(cyclopropylamino)pyrazolo[1,5-a][1,3,5]triazine-8-carbonitrile, TFA (30 mg, 0.053 mmol) in DMSO (0.5 mL) at ambient temperature. The resulting mixture was stirred at ambient temperature for 16 h. The reaction mixture was purified by reverse phase preparative HPLC (Ammonium formate method), Column: Xselect C18 (150×19) mm, 5 micron. The product fractions were pooled together and lyophilized to afford (S)-2-((5-cyano-2-cyclopropyl-3-(4-(1,1-dioxidotetrahydro-2H-thiopyran-4-yl)-2-methylpiperazin-1-yl)phenyl)amino)-4-(cyclopropylamino) pyrazolo[1,5-a][1,3,5]triazine-8-carbonitrile (12.7 mg, 39.8% yield) as off white solid. LC/MS [M+H]⁺=587.3. ¹H-NMR (400 MHz, DMSO-d₆): δ9.40 (s, 1H), 9.17-9.14 (m, 1H), 8.39 (s, 1H), 7.89 (s, 1H), 7.14 (s, 1H), 3.68-3.62 (m, 1H), 3.27-3.23 (m, 1H), 3.18-3.08 (m, 5H), 2.91-2.84 (m, 1H), 2.80-2.73 (m, 3H), 2.65-2.59 (m, 2H), 2.11-1.99 (m, 4H), 1.87-1.79 (m, 1H), 1.27-1.15 (m, 1H), 1.07-0.96 (m, 2H), 0.91 (d, J=6.5 Hz, 3H), 0.84-0.72 (m, 6H), 0.33-0.23 (m, 1H).

### Example 352

### (S)-2-((3-(4-(1-acetylazetidin-3-yl)-2-methylpiperazin-1-yl)-5-cyano-2-methoxyphenyl)amino)-4-(cyclopropylamino)pyrazolo[1,5-a][1,3,5]triazine-8-carbonitrile, TFA

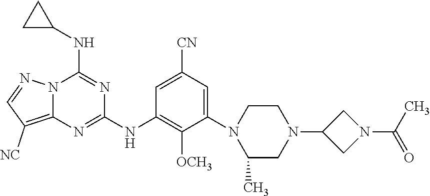 [SMILES: COc1c(Nc2nc(NC3CC3)n3ncc(C#N)c3n2)cc(C#N)cc1N1CCN(C2CN(C(C)=O)C2)C[C@@H]1C]

The title compound was synthesized in a similar procedure as described for Example 336. ¹H NMR (400 MHz, DMSO-d₆) 5=9.31 (br s, 1H), 9.04 (s, 1H), 8.57-8.38 (m, 1H), 7.55-7.33 (m, 1H), 4.47-4.27 (m, 2H), 4.11-3.92 (m, 3H), 3.17 (s, 1H), 2.98-2.83 (m, 2H), 2.54 (s, 1H), 1.81 (d, J=2.5 Hz, 2H), 1.23 (s, 1H), 0.99 (br d, J=4.3 Hz, 2H), 0.84 (d, J=5.5 Hz, 2H). LC/MS [M+H]⁺=542.2.

### Example 353

### 2-((5-cyano-2-(difluoromethoxy)-3-((2S)-4-(1,1-dioxidotetrahydrothiophen-3-yl)-2-methylpiperazin-1-yl)phenyl)amino)-4-(cyclopropylamino)pyrazolo[1,5-a][1,3,5]triazine-8-carbonitrile

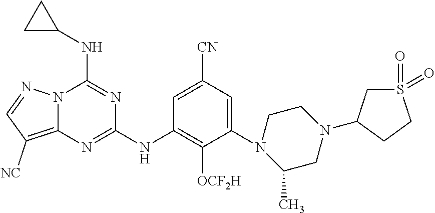

### Step 1. tert-butyl (S)-4-(3-bromo-5-cyano-2-(difluoromethoxy)phenyl)-3-methylpiperazine-1-carboxylate

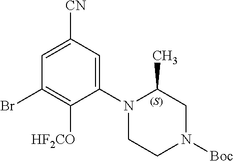

To a 50 mL sealed tube were added tert-butyl (S)-4-(3-bromo-5-cyano-2-hydroxyphenyl)-3-methylpiperazine-1-carboxylate (350 mg, 0.883 mmol), DMF (5 mL), K₂CO₃ (366 mg, 2.65 mmol) and 2-chloro-2,2-difluoroacetic acid (230 mg, 1.766 mmol) and the contents heated at 100° C. for 5 h. The reaction mixture was diluted with EtOAc and water. The EtOAc layer was separated and dried over sodium sulfate and concentrated. The crude product obtained was purified by silica column purification eluting at 15% EtOAc in hexane gave (S)-2-((5-cyano-2-(hydroxymethyl)-3-(2-methylpiperazin-1-yl)phenyl) amino)-4-(cyclopropylamino)pyrazolo[1,5-a][1,3,5]triazine-8-carbonitrile (110 mg, 0.238 mmol, 44.7% yield) as pale yellow solid. LC/MS [M+2H]⁺=445.2. ¹H NMR (400 MHz, CDCl₃) 5=7.64 (d, J=2.0 Hz, 1H), 7.23 (d, J=1.5 Hz, 1H), 6.76 (t, J=73.0 Hz, 1H), 3.61 (br d, J=1.5 Hz, 4H), 3.51-3.21 (m, 3H), 2.78-2.68 (m, 1H), 1.60 (br s, 1H), 1.51 (s, 9H), 1.28 (t, J=7.3 Hz, 2H), 0.98 (d, J=6.5 Hz, 3H).

### Step 2. tert-butyl (S)-4-(5-cyano-3-((8-cyano-4-(cyclopropyl(4-methoxybenzyl)amino)pyrazolo[1,5-a][1,3,5]triazin-2-yl)amino)-2-(difluoromethoxy)phenyl)-3-methylpiperazine-1-carboxylate

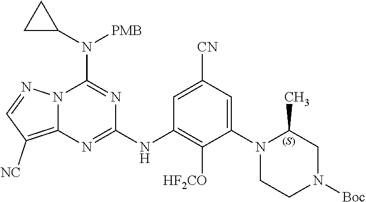

A 100 mL sealed tube was charged dioxane (20 mL), tert-butyl (S)-4-(3-bromo-5-cyano-2-(difluoromethoxy)phenyl)-3-methylpiperazine-1-carboxylate (450 mg, 1.008 mmol) and 2-amino-4-(cyclopropyl(4-methoxy benzyl)amino)pyrazolo[1,5-a][1,3,5]triazine-8-carbonitrile (338 mg, 1.008 mmol). The reaction mixture was purged with N₂ for 5 min. followed by the addition of Pd₂(dba)₃ (92 mg, 0.101 mmol), Xantphos (243 mg, 0.390 mmol) and cesium carbonate (821 mg, 2.52 mmol) sequentially under nitrogen purging. The resulting mixture was heated to 110° C. for 16 h. The reaction mixture was cooled to rt, filtered through a celite pad, washed with DCM (50 mL) and concentrated under reduced pressure. The crude product obtained was purified by column chromatography (Biotage isolera) eluting with 19% ethyl acetate in hexane afford tert-butyl (S)-4-(5-cyano-3-((8-cyano-4-(cyclopropyl(4-methoxybenzyl)amino)pyrazolo[1,5-a][1,3,5]triazin-2-yl)amino)-2-(difluoromethoxy)phenyl)-3-methylpiperazine-1-carboxylate (480 mg, 0.685 mmol, 67.9% yield) as pale-yellow solid. LCMS (M+1)+=701.2.

### Step 3. (S)-2-((5-cyano-2-(difluoromethoxy)-3-(2-methylpiperazin-1-yl)phenyl)amino)-4-(cyclopropylamino)pyrazolo[1,5-a][1,3,5]triazine-8-carbonitrile

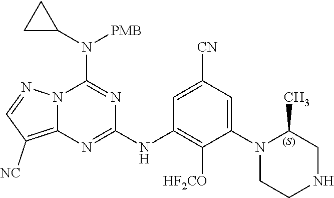

To a stirred solution of tert-butyl (S)-4-(5-cyano-3-((8-cyano-4-(cyclopropyl(4-methoxybenzyl)amino)pyrazolo[1,5-a][1,3,5]triazin-2-yl)amino)-2-(difluoromethoxy)phenyl)-3-methylpiperazine-1-carboxylate (480 mg, 0.685 mmol) in CH₂Cl₂ (10 mL) was added TFA (1.055 mL, 13.70 mmol) at 0° C. The reaction mixture was stirred at RT for 5 h and concentrated. To the residue was added DCM and the contents azeotroped three times to yield a light brown solid. The solid obtained was dissolved in ClCH₂CH₂Cl (10 mL) followed by the addition of TFA (2.052 mL, 26.6 mmol) and anisole (0.218 mL, 1.998 mmol). The contents were heated at 50° C. for 2 h, cooled to rt and concentrated. The residue was azeotroped twice with DCM and the contents dried under high vacuum. MeOH (2 mL) was added to the residue and cooled to 0° C. A few drops of 7 N NH₃ in MeOH were added. The white precipitate was filtered and washed with chilled MeOH to yield (S)-2-((5-cyano-2-(difluoromethoxy)-3-(2-methylpiperazin-1-yl)phenyl)amino)-4-(cyclopropylamino)pyrazolo[1,5-a][1,3,5]triazine-8-carbonitrile (280 mg, 0.553 mmol, 83% yield) as an off-white solid. LC/MS [M+H]⁺=481.2.

### Step 4. Example 353

To a stirred solution of (S)-2-((5-cyano-2-(difluoromethoxy)-3-(2-methylpiperazin-1-yl)phenyl)amino)-4-(cyclopropylamino)pyrazolo[1,5-a][1,3,5]triazine-8-carbonitrile (40 mg, 0.083 mmol) and dihydrothiophen-3(2H)-one 1,1-dioxide (16.75 mg, 0.125 mmol) in dry THF (0.5 mL) was added. acetic acid (0.014 mL, 0.250 mmol) and sodium cyanoborohydride (10.46 mg, 0.166 mmol). The contents were stirred for 16 h at rt and stirred at 50° C. overnight. The reaction mixture was cooled to rt, quenched with MeOH (0.5 mL) and purified using reverse phase preparative HPLC to give the title compound (27 mg, 0.045 mmol, 54.2% yield) as white solid. ¹HNMR (400 MHz, DMSO-d₆) δ=9.43-9.07 (m, 2H), 8.43 (s, 1H), 8.31 (s, 1H), 7.43 (s, 1H), 7.34-6.84 (m, 2H), 3.58-3.44 (m, 1H), 3.15-2.88 (m, 3H), 2.84-2.54 (m, 4H), 2.09-1.91 (m, 1H), 0.93 (d, J=6.5 Hz, 3H), 0.86-0.76 (m, 4H). LC/MS [M+H]⁺=599.

### Example 354

### (2S)-2-[(3R)-4-(2-chloro-5-cyano-3-{[8-cyano-4-(cyclopropylamino)pyrazolo[1,5-a][1,3,5]triazin-2-yl]amino}phenyl)-3-(trifluoromethyl)piperazin-1-yl]propanamide

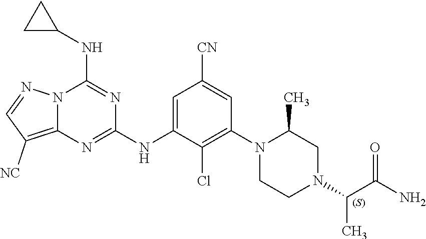

Example 357 was prepared in a similar procedure as described for Example 66. LC/MS [M+H]⁺=574.0. RT=1.75 min. LC/MS condition: Column: Waters XBridge C18, 2.1 mm×50 mm, 1.7 μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0% B to 100% B over 3 min, then a 0.50 min hold at 100% B; Flow: 1 mL/min; Detection: MS and UV (220 nm). ¹H NMR (500 MHz, DMSO-d₆) δ 9.49-9.33 (m, 1H), 9.11-8.94 (m, 1H), 8.28-8.19 (m, 1H), 8.01-7.82 (m, 1H), 7.53-7.39 (m, 1H), 7.01-6.78 (m, 2H), 4.29-4.08 (m, 1H), 3.59-3.43 (m, 1H), 3.03-2.95 (m, 1H), 2.95-2.89 (m, 1H), 2.86-2.79 (m, 1H), 2.74-2.61 (m, 3H), 2.31-2.24 (m, 1H), 1.00-0.94 (m, 3H), 0.66-0.51 (m, 4H).

### Example 355

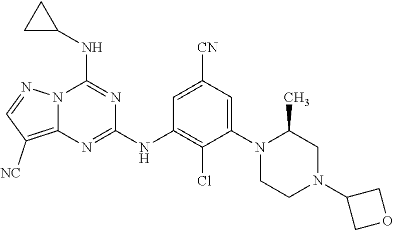 [SMILES: C[C@H]1CN(C2COC2)CCN1c1cc(C#N)cc(Nc2nc(NC3CC3)n3ncc(C#N)c3n2)c1Cl]

¹H NMR (499 MHz, DMSO-d₆) δ 9.69-9.46 (m, 1H), 9.31-9.08 (m, 1H), 8.50-8.39 (m, 1H), 8.24-8.05 (m, 1H), 7.67-7.46 (m, 1H), 4.61-4.52 (m, 2H), 4.52-4.42 (m, 2H), 3.61-3.41 (m, 2H), 3.25-3.09 (m, 1H), 2.90-2.80 (m, 1H), 2.80-2.66 (m, 1H), 2.66-2.54 (m, 1H), 2.49-2.44 (m, 1H), 2.40-2.27 (m, 1H), 2.21-1.62 (m, 1H), 0.99-0.85 (m, 3H), 0.85-0.65 (m, 4H). LCMS (M+1)=505.1.

### Example 356

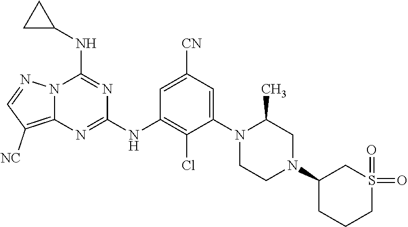 [SMILES: C[C@H]1CN([C@@H]2CCCS(=O)(=O)C2)CCN1c1cc(C#N)cc(Nc2nc(NC3CC3)n3ncc(C#N)c3n2)c1Cl]

¹H NMR (499 MHz, DMSO-d₆) δ 9.88-8.69 (m, 2H), 8.75-8.30 (m, 1H), 8.30-7.88 (m, 1H), 7.72-7.26 (m, 1H), 3.57-3.43 (m, 1H), 3.28-3.21 (m, 1H), 3.21-3.09 (m, 2H), 3.07-2.91 (m, 3H), 2.91-2.80 (m, 2H), 2.74-2.61 (m, 3H), 2.39-2.27 (m, 1H), 2.11-2.01 (m, 1H), 1.92-1.85 (m, 1H), 1.79-1.66 (m, 1H), 1.64-1.52 (m, 1H), 0.92-0.84 (m, 3H), 0.84-0.70 (m, 4H). LCMS (M+1)=581.1.

### Example 357

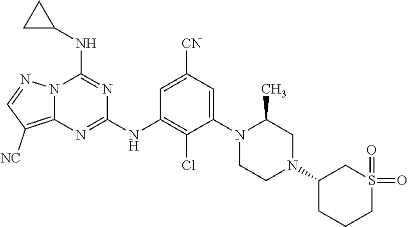 [SMILES: C[C@H]1CN([C@H]2CCCS(=O)(=O)C2)CCN1c1cc(C#N)cc(Nc2nc(NC3CC3)n3ncc(C#N)c3n2)c1Cl]

¹H NMR (499 MHz, DMSO-d₆) δ 9.70-9.35 (m, 1H), 9.35-9.00 (m, 1H), 8.57-8.34 (m, 1H), 8.28-8.12 (m, 1H), 7.63-7.36 (m, 1H), 3.59-3.43 (m, 1H), 3.28-3.12 (m, 3H), 3.07-2.92 (m, 3H), 2.89-2.80 (m, 1H), 2.80-2.70 (m, 2H), 2.70-2.58 (m, 2H), 2.48-2.39 (m, 1H), 2.10-2.01 (m, 1H), 1.91-1.79 (m, 1H), 1.79-1.67 (m, 1H), 1.67-1.52 (m, 1H), 0.92-0.84 (m, 3H), 0.85-0.69 (m, 4H). LCMS (M+1)=581.2.

### Example 358

### 2-((2-chloro-5-cyano-3-(7-(1,1-dioxidotetrahydrothiophen-3-yl)-3-methyl-2,7-diazaspiro[4.4]nonan-2-yl)phenyl)amino)-4-(cyclopropylamino)pyrazolo[1,5-a][1,3,5]triazine-8-carbonitrile

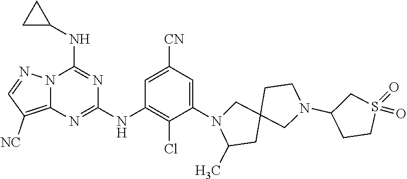 [SMILES: CC1CC2(CCN(C3CCS(=O)(=O)C3)C2)CN1c1cc(C#N)cc(Nc2nc(NC3CC3)n3ncc(C#N)c3n2)c1Cl]

### Step 1. 1-(tert-butyl) 3-ethyl 3-(2-methylallyl)pyrrolidine-1,3-dicarboxylate

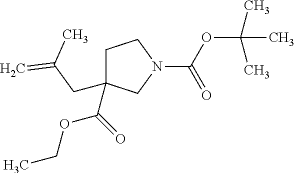 [SMILES: C=C(C)CC1(C(=O)OCC)CCN(C(=O)OC(C)(C)C)C1]

To a solution of LDA (92 mL, 185 mmol) in THF (500 mL) was added 1-(tert-butyl) 3-ethyl pyrrolidine-1,3-dicarboxylate (30.0 g, 123 mmol) at −78° C., and stirred at −78° C., for 1 h. A solution of 3-bromo-2-methylprop-1-ene (18.65 mL, 185 mmol) in THF was added dropwise at −78° C. The resulting solution was further stirred at ambient temperature for 2 h. The reaction mixture was cooled to 0° C. quenched with ammonium chloride solution (300 mL) and extracted with ethyl acetate (2×500 mL). The combined extracts were washed with brine (500 mL), dried over sodium sulphate and concentrated under reduced pressure to yield the crude product, which was further purified by combi-flash using redisep, silica gel, 120 g and Ethyl acetate: Pet ether (15-20%) to afford 1-(tert-butyl) 3-ethyl 3-(2-methylallyl)pyrrolidine-1,3-dicarboxylate (35 g, 118 mmol, 95% yield) (1-3) as a pale yellow liquid. LCMS: RT=3.20 min MS (ES): m/z=242.05 [M-56]+. 1H NMR (400 MHz, CDCl₃) δ ppm 4.79-4.85 (m, 1H), 4.66-4.71 (m, 1H), 4.11-4.20 (m, 2H), 3.73-3.91 (m, 1H), 3.30-3.49 (m, 2H), 3.21-3.29 (m, 1H), 2.42-2.48 (m, 2H), 2.29-2.40 (m, 1H), 1.80-1.89 (m, 1H), 1.67-1.71 (m, 3H), 1.44-1.48 (m, 9H), 1.24-1.29 (m, 3H).

### Step 2. 1-(tert-butyl) 3-ethyl 3-(2-oxopropyl)pyrrolidine-1,3-dicarboxylate

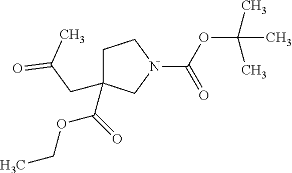 [SMILES: CCOC(=O)C1(CC(C)=O)CCN(C(=O)OC(C)(C)C)C1]

To a solution of 1-(tert-butyl) 3-ethyl 3-(2-methylallyl)pyrrolidine-1,3-dicarboxylate (35 g, 118 mmol) in acetone (500 mL) and H₂O (100 mL) at rt was added potassium osmium(VI) oxide dihydrate (1.735 g, 4.71 mmol) and the reaction mixture was cooled to 0° C. Sodium metaperiodate (101 g, 471 mmol) in H₂O (400 mL) was added dropwise at 0° C. over 20 min. The resulting solution was stirred at ambient temperature for 1 h. The reaction mixture was quenched with H₂O (300 mL) and extracted with ethyl acetate (2×500 mL). The combined extracts were washed with brine (500 mL), dried over sodium sulphate, and concentrated under reduced pressure to afford 1-(tert-butyl) 3-ethyl 3-(2-oxopropyl)pyrrolidine-1,3-dicarboxylate (35 g, 117 mmol, 99% yield) (1-4) as a brown liquid. The crude was further used for next step without further purification. LCMS: RT=2.321 min MS (ES): m/z=200.10 [M-100]+. 1H NMR (400 MHz, CHLOROFORM-d) δ ppm 4.13 (q, J=7.00 Hz, 2H) 3.81-3.90 (m, 1H) 3.34-3.48 (m, 2H) 3.13-3.24 (m, 1H) 2.97-3.05 (m, 1H) 2.70-2.92 (m, 2H) 2.12 (s, 3H) 1.75-1.84 (m, 1H) 1.41-1.44 (m, 9H) 1.18-1.23 (m, 3H).

### Step 3. tert-butyl 8-methyl-6-oxo-2,7-diazaspiro[4.4]nonane-2-carboxylate

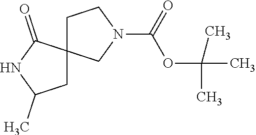 [SMILES: CC1CC2(CCN(C(=O)OC(C)(C)C)C2)C(=O)N1]

To a solution of 1-(tert-butyl) 3-ethyl 3-(2-oxopropyl)pyrrolidine-1,3-dicarboxylate (35 g, 117 mmol) in EtOH (1000 mL) at rt were added ammonium acetate (135 g, 1754 mmol), sodium cyanoborohydride (29.4 g, 468 mmol) and magnesium sulphate (anhydrous) (99 g, 818 mmol). The resulting solution was further stirred at 80° C. for 16 h. The reaction mixture was cooled to rt. The resulting solution was concentrated under reduced pressure, quenched with H₂O (300 mL) and extracted with ethyl acetate (2×500 mL). The combined extracts were washed with 10% NaHCO₃ solution (500 mL), brine (500 mL), dried over sodium sulphate and concentrated under reduced pressure to afford the crude product. The crude was further purified by ISCO (basic alumina, 120 g) with 2-5% MeOH/DCM to afford tert-butyl 8-methyl-6-oxo-2,7-diazaspiro[4.4]nonane-2-carboxylate (25 g, 98 mmol, 84% yield) (1-5) as a pale-yellow gummy solid. LCMS: RT=1.71 min MS (ES): m/z=199.05 [M-56]+. 1H NMR (400 MHz, CHLOROFORM-d) δ ppm 6.01-6.20 (m, 1H) 3.61-3.81 (m, 2H) 3.29-3.50 (m, 2H) 2.26-2.39 (m, 1H) 1.78-2.20 (m, 1H) 1.53-1.87 (m, 3H) 1.44-1.47 (m, 9H) 1.26 (br d, J=6.00 Hz, 3H).

### Step 4. tert-butyl 8-methyl-2,7-diazaspiro[4.4]nonane-2-carboxylate

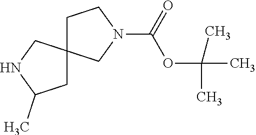 [SMILES: CC1CC2(CCN(C(=O)OC(C)(C)C)C2)CN1]

To a solution of tert-butyl 8-methyl-6-oxo-2,7-diazaspiro[4.4]nonane-2-carboxylate (25 g, 98 mmol) in THF (300 mL) was added borane dimethyl sulfide complex (28.0 mL, 295 mmol) at 0° C. The resulting solution was further stirred at 70° C. for 12 h. The reaction was cooled to 0° C., quenched with water (70 mL) and refluxed. After 2 h, the reaction was cooled to 0° C. and pH of the solution was adjusted to strongly alkaline by dropwise addition of 6N NaOH solution. The resulting solution was extracted with ethyl acetate (2×700 mL). The combined organic extracts were washed with brine (500 mL), dried over sodium sulphate, and concentrated under reduced pressure to afford the crude product tert-butyl 8-methyl-2,7-diazaspiro[4.4]nonane-2-carboxylate (20 g, 83 mmol, 85% yield) as a gummy solid. The crude was used for next step without any purification. LCMS: RT=1.078 min MS (ES): m/z=241.15 [M+1]⁺.

### Step 5. 2-benzyl 7-(tert-butyl) 3-methyl-2,7-diazaspiro[4.4]nonane-2,7-dicarboxylate

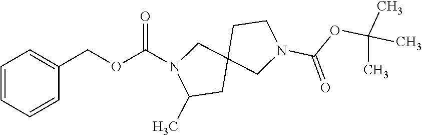 [SMILES: CC1CC2(CCN(C(=O)OC(C)(C)C)C2)CN1C(=O)OCc1ccccc1]

To a solution of tert-butyl 8-methyl-2,7-diazaspiro[4.4]nonane-2-carboxylate (20 g, 83 mmol) in DCM (250 mL) were added benzyl chloroformate (13.07 mL, 92 mmol) and triethylamine (34.8 mL, 250 mmol) at 0° C. The resulting solution was further stirred at rt for 2 h. The reaction mixture was concentrated in vacuo, extracted with ethyl acetate (2×450 mL).

The combined extracts were washed with NaHCO₃ solution (500 mL), saturated brine (500 ml), dried over Na₂SO₄ and concentrated under reduced pressure. The crude was purified by ISCO eluting with 25-30% EtOAc/pet. ether to afford 2-benzyl 7-(tert-butyl) 3-methyl-2,7-diazaspiro[4.4]nonane-2,7-dicarboxylate (14 g, 37.4 mmol, 44.9% yield) as colorless liquid. LCMS: RT=3.255 min Column: MS (ES): m/z=392.15 [M+NH₄]⁺. ¹H NMR (400 MHz, CHLOROFORM-d) δ ppm 7.34-7.39 (m, 5H) 5.07-5.19 (m, 2H) 3.90-4.02 (m, 1H) 3.25-3.38 (m, 4H) 1.84-1.94 (m, 2H) 1.69-1.80 (m, 2H) 1.46 (s, 9H) 1.39 (br d, J=6.50 Hz, 2H) 1.23-1.31 (m, 3H).

### Step 6. tert-butyl 8-methyl-2,7-diazaspiro[4.4]nonane-2-carboxylate

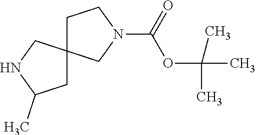 [SMILES: CC1CC2(CCN(C(=O)OC(C)(C)C)C2)CN1]

A solution of 2-benzyl 7-(tert-butyl) 3-methyl-2,7-diazaspiro[4.4]nonane-2,7-dicarboxylate (14 g, 37.4 mmol) in MeOH (300 mL) was evacuated and back filled with nitrogen 3 times. Palladium on carbon (10% by weight, 14 g, 13.16 mmol) was added and reaction mixture was evacuated and back filled with hydrogen. The reaction was stirred under the atmosphere of hydrogen for 2 h at rt. The reaction mixture was filtered through celite pad. The filtrate was concentrated in vacuo to obtain tert-butyl 8-methyl-2,7-diazaspiro[4.4]nonane-2-carboxylate (8.5 g, 35.4 mmol, 95% yield) as colorless liquid. The crude was taken to next step without further purification. LC/MS: RT=1.704 min MS (ES): m/z=241.15 [M+1]⁺. NMR: ¹H NMR (400 MHz, DMSO-d₆) δ ppm 3.23 (br d, J=7.00 Hz, 2H), 3.04-3.13 (m, 3H), 2.72-2.78 (m, 1H), 2.53-2.62 (m, 1H), 1.68-1.83 (m, 3H), 1.38-1.39 (m, 9H), 1.07-1.24 (m, 2H), 1.03-1.06 (m, 3H).

### Step 7. tert-butyl 7-(3-bromo-2-chloro-5-cyanophenyl)-8-methyl-2,7-diazaspiro[4.4]nonane-2-carboxylate

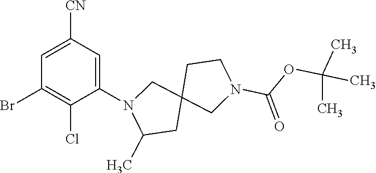 [SMILES: CC1CC2(CCN(C(=O)OC(C)(C)C)C2)CN1c1cc(C#N)cc(Br)c1Cl]

To a mixture of 3,5-dibromo-4-chlorobenzonitrile (500 mg, 1.693 mmol) in toluene (10 mL) were added tert-butyl 8-methyl-2,7-diazaspiro[4.4]nonane-2-carboxylate (488 mg, 2.031 mmol) and cesium carbonate (1655 mg, 5.08 mmol) and the contents purged with argon for 5 min. Tris(dibenzylideneacetone)dipalladium (0) (78 mg, 0.085 mmol) and Bis(2-diphenylphosphinophenyl)ether (91 mg, 0.169 mmol) were added under argon. The resulting mixture was stirred at 110° C. for 16 h. The reaction mixture was cooled to rt, filtered through celite pad. Then the filtrate was extracted with ethyl acetate (2×50 mL). The combined extracts were washed with saturated brine (50 ml), dried over Na₂SO₄, concentrated under reduced pressure and purified by ISCO with 25-30% EtOAc/pet. ether to yield tert-butyl 7-(3-bromo-2-chloro-5-cyanophenyl)-8-methyl-2,7-diazaspiro[4.4]nonane-2-carboxylate (260 mg, 0.572 mmol, 33.8% yield) as a gummy yellow liquid. LC/MS: RT=1.38 min; MS (ES): m/z=397.9 [M-56]⁺. ¹H NMR (400 MHz, CHLOROFORM-d) δ ppm 7.35-7.41 (m, 1H) 7.08-7.13 (m, 1H) 4.07-4.17 (m, 1H) 3.74-3.86 (m, 1H) 3.23-3.54 (m, 4H) 2.87-3.00 (m, 1H) 2.10-2.23 (m, 1H) 1.92-2.02 (m, 1H) 1.76-1.83 (m, 1H) 1.63-1.72 (m, 1H) 1.44-1.50 (m, 9H) 1.07-1.11 (m, 3H).

### Step 8. tert-butyl 7-(2-chloro-5-cyano-3-((8-cyano-4-(cyclopropyl(4-methoxybenzyl)amino)pyrazolo[1,5-a][1,3,5]triazin-2-yl)amino)phenyl)-8-methyl-2,7-diazaspiro[4.4]nonane-2-carboxylate

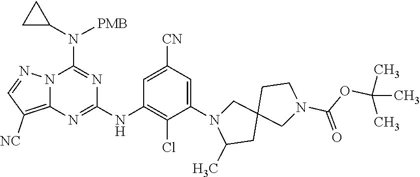

To a stirred solution of tert-butyl 7-(3-bromo-2-chloro-5-cyanophenyl)-8-methyl-2,7-diazaspiro[4.4]nonane-2-carboxylate (4.8 g, 10.55 mmol) in 1,4-Dioxane (20 mL) were added 2-amino-4-(cyclopropyl(4-methoxybenzyl) amino)pyrazolo[1,5-a][1,3,5]triazine-8-carbonitrile (3.54 g, 10.55 mmol) and cesium carbonate (10.32 g, 31.7 mmol). The reaction was purged with argon for 5 min. Tris(dibenzylideneacetone)dipalladium (0) (0.966 g, 1.055 mmol) and Xantphos (1.221 g, 2.111 mmol) were added under inert atmosphere and stirred at 110° C. for 16 h. The reaction mixture was cooled to room temperature and filtered through celite pad. The filtrate was extracted with EtOAc (2×250 mL). The combined extracts were washed with brine (250 mL), dried over Na₂SO₄, and concentrated under reduced pressure to yield crude product. The crude was purified using ISCO with 25-30% EtOAc/pet. ether to obtain tert-butyl 7-(2-chloro-5-cyano-3-((8-cyano-4-(cyclopropyl(4-methoxy benzyl)amino)pyrazolo[1,5-a][1,3,5]triazin-2-yl)amino)phenyl)-8-methyl-2,7-diazaspiro[4.4]nonane-2-carboxylate (330 mg, 0.465 mmol, 4.41% yield) as pale yellow solid. LC/MS: RT=3.821 min MS (ES): m/z=709.30 [M+1]⁺. ¹H NMR (400 MHz, CHLOROFORM-d) δ ppm 8.53-8.60 (m, 1H) 8.07-8.09 (m, 1H) 7.79-7.82 (m, 1H) 7.10-7.15 (m, 2H) 6.89-6.92 (m, 1H) 6.82-6.86 (m, 2H) 5.37-5.45 (m, 2H) 4.02-4.14 (m, 1H) 3.78 (s, 3H) 3.50 (s, 1H) 3.28-3.41 (m, 2H) 3.06-3.15 (m, 1H) 2.81-2.90 (m, 1H) 2.09-2.18 (m, 1H) 1.78-1.83 (m, 2H) 1.57-1.62 (m, 3H) 1.46-1.50 (m, 9H) 1.11-1.16 (m, 2H) 1.09 (d, J=6.00 Hz, 3H) 0.87-0.91 (m, 2H).

### Step 9. 2-((2-chloro-5-cyano-3-(3-methyl-2,7-diazaspiro[4.4]nonan-2-yl)phenyl)amino)-4-(cyclopropylamino)pyrazolo[1,5-a][1,3,5]triazine-8-carbonitrile

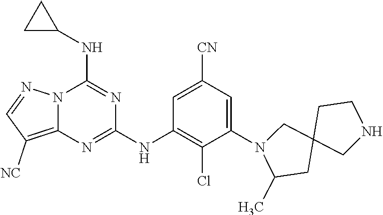 [SMILES: CC1CC2(CCNC2)CN1c1cc(C#N)cc(Nc2nc(NC3CC3)n3ncc(C#N)c3n2)c1Cl]

To a stirred solution of tert-butyl 7-(2-chloro-5-cyano-3-((8-cyano-4-(cyclopropyl(4-methoxybenzyl)amino)pyrazolo[1,5-a][1,3,5]triazin-2-yl)amino)phenyl)-8-methyl-2,7-diazaspiro[4.4]nonane-2-carboxylate (330 mg, 0.465 mmol) in DCE (2 mL) were added TFA (0.3 mL, 3.89 mmol) and the reaction mixture was stirred at 60° C. for 2 h. The reaction mixture was cooled to rt and concentrated under reduced pressure to obtain 2-((2-chloro-5-cyano-3-(3-methyl-2,7-diazaspiro[4.4]nonan-2-yl)phenyl)amino)-4-(cyclopropylamino)pyrazolo[1,5-a][1,3,5]triazine-8-carbonitrile (220 mg, 0.450 mmol, 97% yield) (1-14) as off white solid. LCMS: RT=0.618 min; MS (ES): m/z=489.20 [M+1]⁺. ¹H NMR (400 MHz, DMSO-d₆) δ ppm 8.35-8.39 (m, 1H) 7.80-7.85 (m, 1H) 7.21-7.25 (m, 1H) 4.11-4.19 (m, 1H) 3.72-3.78 (m, 2H) 2.31-2.34 (m, 1H) 2.04-2.09 (m, 1H) 1.80-1.83 (m, 5H) 1.52-1.64 (m, 3H) 1.16-1.25 (m, 2H) 1.00-1.04 (m, 3H) 0.74-0.80 (m, 4H).

### Step 10. Example 358

To a solution of 2-((2-chloro-5-cyano-3-(3-methyl-2,7-diazaspiro[4.4]nonan-2-yl)phenyl)amino)-4-(cyclopropylamino)pyrazolo[1,5-a][1,3,5]triazine-8-carbonitrile (100 mg, 0.205 mmol.) in THF (2.5 mL) and DMSO (0.5 mL) were added acetic acid (1.171 μL, 0.020 mmol) and dihydrothiophen-3(2H)-one 1,1-dioxide (54.9 mg, 0.409 mmol) at rt for 1 h. Sodium cyanoborohydride (51.4 mg, 0.818 mmol) was added portion wise and the reaction was stirred for 18 h at rt. The reaction mixture was concentrated in vacuo and diluted with ethyl acetate (30 mL). The resulting mixture was washed with saturated NaHCO₃ (30 mL), brine (30 mL), dried over Na₂SO₄ and concentrated under reduced pressure. The residue was purified by preparative HPLC using following conditions.

Column: SunFire OBD C18 (150*19) Sum, Mobile Phase A: 10 mM Ammonium Acetate in Water pH-4.5, Mobile Phase B: ACN, Flow rate: 20.0 ml/min and then isomers were separated by SFC Method Info: Column & Mobile Phase: WELK (RR) (250×4.6) mm-5p, 0.2% Methanolic Ammonia in MeOH, Total Flow: 4.0000 mL/min, Co-Solvent %: 45.0%, Oven Temperature: 40, ABPR Pressure: 100.0 bar, ABPR Temperature: 50 C to afford the eight isomers of 2-((2-chloro-5-cyano-3-(7-(1,1-dioxidotetrahydrothiophen-3-yl)-3-methyl-2,7-diazaspiro[4.4]nonan-2-yl)phenyl)amino)-4-(cyclopropylamino)pyrazolo[1,5-a][1,3,5]triazine-8-carbonitrile.
Isomer 1: LC/MS [M+NH3]=607.15 RT=5.5 min ¹H NMR (400 MHz, DMSO-d₆) δ 9.58-9.44 (m, 1H), 9.23-9.05 (m, 1H), 8.40 (s, 1H), 7.80 (s, 1H), 7.26 (d, J=1.6 Hz, 1H), 4.15 (td, J=6.2, 9.4 Hz, 1H), 3.77 (d, J=9.5 Hz, 1H), 3.26-3.20 (m, 2H), 3.16-3.03 (m, 2H), 2.92-2.81 (m, 2H), 2.76-2.66 (m, 2H), 2.36-2.24 (m, 2H), 2.14-1.93 (m, 2H), 1.74-1.57 (m, 3H), 1.02 (d, J=5.9 Hz, 3H), 0.84-0.73 (m, 4H).
Isomer 2: LC/MS [M+NH3]=607.15 RT=7.46 min ¹H NMR (400 MHz, DMSO-d₆) δ 9.56-9.20 (m, 1H), 8.39 (s, 1H), 7.79 (s, 1H), 7.22 (s, 1H), 4.13 (td, J=6.1, 8.9 Hz, 1H), 3.73 (br d, J=9.5 Hz, 2H), 3.22-2.80 (m, 3H), 2.70-2.55 (m, 2H), 2.43-2.30 (m, 2H), 2.30-2.10 (m, 2H), 2.02-1.74 (m, 4H), 1.60 (dd, J=9.8, 11.8 Hz, 1H), 1.01 (d, J=5.9 Hz, 3H), 0.85-0.67 (m, 4H) (1 exchangeable proton not found).
Isomer 3: LC/MS [M+NH3]=607.15 RT=4.59 min ¹H NMR (400 MHz, DMSO-d₆) δ 9.60-9.36 (m, 1H), 8.39 (s, 1H), 7.80 (s, 1H), 7.21 (d, J=1.6 Hz, 1H), 4.13 (td, J=6.0, 9.2 Hz, 1H), 3.74 (d, J=9.5 Hz, 2H), 3.27-2.84 (m, 3H), 2.72-2.62 (m, 2H), 2.44 (br d, J=5.6 Hz, 2H), 2.31-2.14 (m, 2H), 1.87 (s, 4H), 1.60 (dd, J=9.6, 12.1 Hz, 1H), 1.01 (d, J=5.9 Hz, 3H), 0.86-0.72 (m, 4H) (1 exchangeable proton not found).
Isomer 4: LC/MS [M+NH3]=607.15 RT=4.49 min ¹H NMR (400 MHz, DMSO-d₆) δ 9.56-9.20 (m, 1H), 8.38 (s, 1H), 7.81 (br s, 1H), 7.21 (d, J=1.6 Hz, 1H), 4.24-3.99 (m, 1H), 3.74 (d, J=9.5 Hz, 2H), 3.24-2.84 (m, 3H), 2.68 (s, 2H), 2.48-2.37 (m, 2H), 2.30-2.13 (m, 2H), 2.06-1.72 (m, 4H), 1.60 (dd, J=9.7, 11.8 Hz, 1H), 1.01 (d, J=5.9 Hz, 3H), 0.87-0.64 (m, 4H) (1 exchangeable proton not found).
Isomer 5: LC/MS [M+NH3]=607.15 RT=4.31 min ¹H NMR (400 MHz, DMSO-d₆) δ 9.60-9.36 (m, 1H), 8.39 (s, 1H), 7.80 (s, 1H), 7.21 (d, J=1.6 Hz, 1H), 4.13 (td, J=6.0, 9.2 Hz, 1H), 3.74 (d, J=9.5 Hz, 2H), 3.27-2.84 (m, 3H), 2.72-2.62 (m, 2H), 2.44 (br d, J=5.6 Hz, 2H), 2.31-2.14 (m, 2H), 1.87 (s, 4H), 1.60 (dd, J=9.6, 12.1 Hz, 1H), 1.01 (d, J=5.9 Hz, 3H), 0.86-0.72 (m, 4H) (1 exchangeable proton not found).
Isomer 6: LC/MS [M+NH3]=607.15 RT=5.84 min ¹H NMR (400 MHz, DMSO-d₆) δ 9.57-9.44 (m, 1H), 9.24-9.07 (m, 1H), 8.39 (s, 1H), 7.80 (br s, 1H), 7.25 (d, J=1.8 Hz, 1H), 4.25-4.08 (m, 1H), 3.78 (d, J=9.9 Hz, 1H), 3.27-3.18 (m, 2H), 3.16-2.79 (m, 4H), 2.74-2.61 (m, 2H), 2.39-2.24 (m, 2H), 2.19-1.91 (m, 2H), 1.74-1.53 (m, 3H), 1.03 (d, J=5.9 Hz, 3H), 0.88-0.70 (m, 4H).
Isomer 7: LC/MS [M+NH3]=607.15 RT=7.92 min ¹H NMR (400 MHz, DMSO-d₆) δ 8.30-8.00 (m, 2H), 7.16 (s, 1H), 4.12 (td, J=5.9, 9.5 Hz, 1H), 3.77 (br d, J=9.5 Hz, 1H), 3.16-2.89 (m, 4H), 2.75-2.53 (m, 4H), 2.37-2.21 (m, 2H), 2.17-1.91 (m, 2H), 1.76-1.52 (m, 3H), 1.01 (d, J=5.9 Hz, 3H), 0.74-0.58 (m, 4H) (2 exchangeable protons not found).
Isomer 8: LC/MS [M+NH3]=607.15 RT=8.95 min ¹H NMR (400 MHz, DMSO-d₆) δ 8.22 (br dd, J=2.1, 4.0 Hz, 1H), 8.12 (br s, 1H), 7.15 (s, 1H), 4.12 (td, J=6.1, 9.0 Hz, 1H), 3.76 (br d, J=9.4 Hz, 1H), 3.17-3.03 (m, 2H), 2.96 (br dd, J=7.8, 12.9 Hz, 2H), 2.83 (br d, J=9.4 Hz, 1H), 2.74-2.65 (m, 2H), 2.61 (br t, J=7.0 Hz, 1H), 2.36-2.27 (m, 2H), 2.13-1.94 (m, 2H), 1.71-1.56 (m, 3H), 1.01 (d, J=5.9 Hz, 3H), 0.73-0.57 (m, 4H) (2 exchangeable protons not found).

### Example 359

### 2-((2-chloro-5-cyano-3-((2S)-2-methyl-4-((2-oxooxazolidin-4-yl)methyl)piperazin-1-yl)phenyl)amino)-4-(cyclopropylamino)pyrazolo[1,5-a][1,3,5]triazine-8-carbonitrile

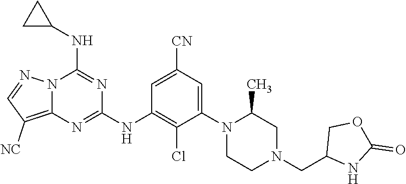 [SMILES: C[C@H]1CN(CC2COC(=O)N2)CCN1c1cc(C#N)cc(Nc2nc(NC3CC3)n3ncc(C#N)c3n2)c1Cl]

### Step 1. (2-oxooxazolidin-4-yl)methyl 4-methylbenzenesulfonate

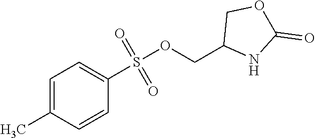 [SMILES: Cc1ccc(S(=O)(=O)OCC2COC(=O)N2)cc1]

To a stirred solution of 4-(hydroxymethyl)oxazolidin-2-one (200 mg, 1.708 mmol) in DCM (10 mL) was added 1-methylimidazole (0.204 mL, 2.56 mmol) at rt followed by p-toluenesulfonyl chloride (358 mg, 1.879 mmol) at 0° C. The resulting mixture was stirred at room temperature for 16 hours. The reaction mixture was extracted with DCM (2×10 mL), washed with NaHCO₃ solution (10 mL), 1N HCl solution (10 mL), brine (10 mL), dried over Na₂SO₄, and concentrated in vacuo to obtain (2-oxooxazolidin-4-yl)methyl 4-methylbenzenesulfonate (140 mg, 0.516 mmol, 30.2% yield) as an off-white solid. LC/MS [M+NH_{3]=289.25} RT=1.661 min. ¹H NMR (400 MHz, DMSO-d₆) 5 ppm 7.85-7.89 (m, 1H), 7.79-7.83 (m, 2H), 7.48-7.53 (m, 2H), 4.28-4.35 (m, 1H), 3.91-4.06 (m, 4H), 2.42-2.44 (m, 3H).

### Step 2. Example 359

To a stirred solution of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (100 mg, 0.223 mmol) in DMSO (2 mL) was added (2-oxooxazolidin-4-yl)methyl 4-methylbenzenesulfonate (91 mg, 0.334 mmol), and the reaction mixture was stirred at 85° C. for 6 h. The reaction was diluted with EtOAc (70 mL), washed with saturated NaHCO₃ (50 mL), brine (70 mL), dried over Na₂SO₄ and concentrated under reduced pressure to yield the crude product which was purified using preparative HPLC employing the following conditions. Column: X-Select CSHI (250*19) 5; mobile phase A: 10 mM ammonium acetate in water; mobile phase B: ACN:Methanol (1:1); Flow: 20 mL\Min. Fractions containing desired product were concentrated under reduced pressure and individual isomers were separated using preparatory SFC employing the following conditions. Chiralpak IH (250×4.6) mm, 5u, % CO₂: 50%, % co solvent: 50% OF 5 mM ammonium acetate in ACN:MeOH (50:50), total Flow: 4.0 g/min, back Pressure: 100 bar, temperature: 35° C., UV: 220 nm. The fractions containing desired product were concentrated under reduced pressure to yield.

Isomer 1: 2-((2-chloro-5-cyano-3-((2S)-2-methyl-4-((2-oxooxazolidin-4-yl)methyl)piperazin-1-yl)phenyl)amino)-4-(cyclopropylamino)pyrazolo[1,5-a][1,3,5]triazine-8-carbonitrile (4.4 mg, 3.60% yield) as off white solid. LC/MS [M+H]=585.25 RT=1.88 min. ¹H NMR (400 MHz, DMSO-d₆) δ ppm 9.30-9.43 (m, 1H) 8.35-8.41 (m, 1H) 8.15-8.23 (m, 1H) 7.64-7.73 (m, 1H) 7.46-7.54 (m, 1H) 4.35-4.44 (m, 1H) 3.96-4.06 (m, 2H) 3.48 (br s, 1H) 3.13-3.18 (m, 1H) 2.77-2.89 (m, 2H) 2.66-2.70 (m, 1H) 2.29-2.43 (m, 3H) 2.16-2.27 (m, 1H) 1.76-1.84 (m, 1H) 0.84-0.95 (m, 3H) 0.69-0.81 (m, 4H).
Isomer-2: 2-((2-chloro-5-cyano-3-((2S)-2-methyl-4-((2-oxooxazolidin-4-yl)methyl)piperazin-1-yl)phenyl)amino)-4-(cyclopropylamino)pyrazolo[1,5-a][1,3,5]triazine-8-carbonitrile (1) (41 mg, 16.79% yield). LC/MS [M+H]=585.2 RT=1.88 min. ¹H NMR (400 MHz, DMSO-d₆) δ ppm 9.21-9.43 (m, 1H), 8.37-8.42 (m, 1H), 8.12-8.17 (m, 1H), 7.65-7.70 (m, 1H), 7.46-7.52 (m, 1H), 4.33-4.41 (m, 1H), 4.01-4.09 (m, 1H), 3.93-4.02 (m, 1H), 3.46-3.56 (m, 1H), 3.16 (br d, J=5.63 Hz, 1H) 2.79-2.86 (m, 1H) 2.58-2.75 (m, 4H) 2.42 (br d, J=6.25 Hz, 3H) 2.24-2.36 (m, 2H) 0.87 (d, J=6.13 Hz, 3H) 0.65-0.79 (m, 3H).

### Example 360

### 2-((2-chloro-5-cyano-3-((2S)-2-methyl-4-((4-methyl-2-oxooxazolidin-4-yl)methyl)piperazin-1-yl)phenyl)amino)-4-(cyclopropylamino)pyrazolo[1,5-a][1,3,5]triazine-8-carbonitrile

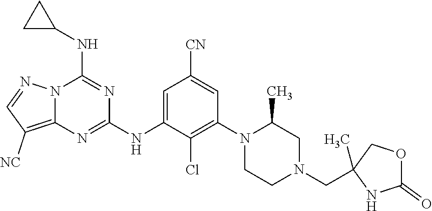 [SMILES: C[C@H]1CN(CC2(C)COC(=O)N2)CCN1c1cc(C#N)cc(Nc2nc(NC3CC3)n3ncc(C#N)c3n2)c1Cl]

Example 360 were prepared from 4-(hydroxymethyl)-4-methyloxazolidin-2-one (120 mg, 0.184 mmol) in the same general manner as described for Example 359, followed by purification by prep HPLC.

Isomer 1: LC/MS [M+H]=562.2 RT=1.78 min. ¹H NMR (400 MHz, DMSO-d6) δ=9.51 (s, 1H), 9.22 (d, J=3.8 Hz, 1H), 8.41 (s, 1H), 8.12 (s, 1H), 7.63 (s, 1H), 7.49 (s, 1H), 4.20 (d, J=8.5 Hz, 1H), 3.94 (d, J=8.3 Hz, 1H), 3.51 (br s, 1H), 3.17 (s, 1H), 2.88-2.80 (m, 2H), 2.76-2.69 (m, 1H), 2.63 (s, 1H), 2.39 (br d, J=1.8 Hz, 3H), 1.20 (s, 3H), 0.90 (d, J=6.3 Hz, 3H), 0.82-0.70 (m, 4H).
Isomer 2: LC/MS [M+H]=562.2 RT=1.79 min. 1H NMR (400 MHz, DMSO-d6) δ=9.50 (br s, 1H), 9.24 (br s, 1H), 8.41 (s, 1H), 8.12 (d, J=1.5 Hz, 1H), 7.64 (s, 1H), 7.48 (s, 1H), 4.22 (d, J=8.3 Hz, 1H), 3.95 (d, J=8.5 Hz, 1H), 3.57-3.47 (m, 1H), 3.24 (br s, 1H), 2.88-2.80 (m, 1H), 2.77-2.61 (m, 4H), 2.47 (br s, 1H), 2.39 (s, 2H), 1.21 (s, 3H), 0.89 (d, J=6.3 Hz, 3H), 0.82-0.71 (m, 4H).

### Example 361

### (S)-2-((2-chloro-5-cyano-3-(2-methyl-4-(2-morpholino-2-oxoethyl)piperazin-1-yl)phenyl)amino)-4-(cyclopropylamino)pyrazolo[1,5-a][1,3,5]triazine-8-carbonitrile

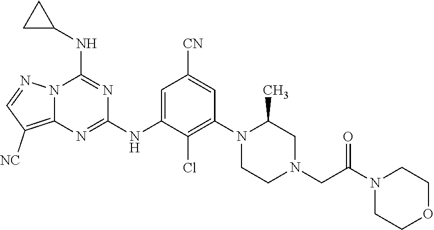 [SMILES: C[C@H]1CN(CC(=O)N2CCOCC2)CCN1c1cc(C#N)cc(Nc2nc(NC3CC3)n3ncc(C#N)c3n2)c1Cl]

### Step 1. tert-butyl (S)-2-(4-(2-chloro-5-cyano-3-((8-cyano-4-(cyclopropylamino) pyrazolo[1,5-a][1,3,5]triazin-2-yl) amino) phenyl)-3-methylpiperazin-1-yl) acetate

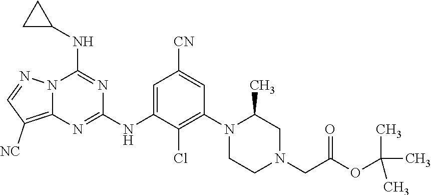 [SMILES: C[C@H]1CN(CC(=O)OC(C)(C)C)CCN1c1cc(C#N)cc(Nc2nc(NC3CC3)n3ncc(C#N)c3n2)c1Cl]

To a mixture of (S)-2-((2-chloro-5-cyano-3-(2-methylpiperazin-1-yl) phenyl) amino)-4-(cyclopropylamino) pyrazolo[1,5-a][1,3,5]triazine-8-carbonitrile (300 mg, 0.668 mmol), tert-butyl 2-bromoacetate (156 mg, 0.802 mmol) in DMSO (1.0 mL) and THF (10.0 mL) was added at 0° C. followed by DIPEA (0.350 mL, 2.005 mmol). The reaction was stirred at 60° C. for 14 h.

Volatiles were removed in vacuo. Reaction mixture was diluted with ethyl acetate, washed with saturated NaHCO₃ (50 mL), brine (70 mL), dried over Na₂SO₄ and concentrated under reduced pressure to yield tert-butyl (S)-2-(4-(2-chloro-5-cyano-3-((8-cyano-4-(cyclopropylamino) pyrazolo[1,5-a][1,3,5]triazin-2-yl) amino) phenyl)-3-methylpiperazin-1-yl) acetate (300 mg, 0.533 mmol, 80% yield) LC/MS, [M+H]=563.2, RT=1.08 min.

### Step 2. (S)-2-(4-(2-chloro-5-cyano-3-((8-cyano-4-(cyclopropylamino) pyrazolo[1,5-a][1,3,5]triazin-2-yl) amino) phenyl)-3-methylpiperazin-1-yl) acetic acid

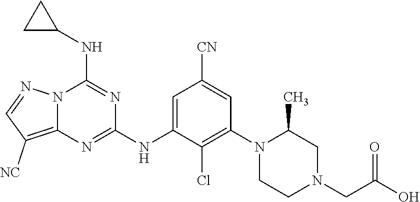 [SMILES: C[C@H]1CN(CC(=O)O)CCN1c1cc(C#N)cc(Nc2nc(NC3CC3)n3ncc(C#N)c3n2)c1Cl]

To a mixture of tert-butyl (S)-2-(4-(2-chloro-5-cyano-3-((8-cyano-4-(cyclopropylamino) pyrazolo[1,5-a][1,3,5]triazin-2-yl) amino) phenyl)-3-methylpiperazin-1-yl) acetate (480 mg, 0.852 mmol) in DCM (10.0 mL) at 0° C. was added TFA (1.314 mL, 17.05 mmol). The reaction was stirred at rt for 14 h. Volatiles were evaporated. Resulting mixture was triturated with diethyl ether for 15 min followed by filtration to obtain (S)-2-(4-(2-chloro-5-cyano-3-((8-cyano-4-(cyclopropylamino) pyrazolo[1,5-a][1,3,5]triazin-2-yl) amino) phenyl)-3-methylpiperazin-1-yl) acetic acid (420 mg, 0.828 mmol, 97% yield) as a light yellow solid. LC/MS [M+H]=507.2, RT=1.85 min.

### Step 3. Example 361

To a mixture of (S)-2-(4-(2-chloro-5-cyano-3-((8-cyano-4-(cyclopropylamino)pyrazolo[1,5-a][1,3,5]triazin-2-yl)amino)phenyl)-3-methylpiperazin-1-yl)acetic acid (35.0 mg, 0.069 mmol) in DMF (2.0 mL) was added morpholine (7.22 mg, 0.083 mmol) at 0° C. followed by HATU (31.5 mg, 0.083 mmol) and DIPEA (0.036 mL, 0.207 mmol). The reaction was stirred at rt for 2 h. Reaction mixture was slowly quenched with cold water and stirred for 30 min. The reaction mixture is extracted with ethyl Acetate, washed with saturated NaHCO₃ (50 mL), brine (70 mL), dried over Na₂SO₄ and concentrated under reduced pressure to obtain crude product. Crude product was purified using preparative HPLC using following conditions. Waters XBridge C18, 19×150 mm, 5-μm particles; Mobile Phase A: 10-mM ammonium acetate; Mobile Phase B: acetonitrile; Gradient: 10-45% B over 20 min, then a 5-min hold at 100% B; Flow: 20 mL/min. Fractions containing the desired product were combined and dried via centrifugal evaporation to obtain (S)-2-((2-chloro-5-cyano-3-(2-methyl-4-(2-morpholino-2-oxoethyl)piperazin-1-yl)phenyl)amino)-4-(cyclopropylamino)pyrazolo[1,5-a][1,3,5]triazine-8-carbonitrile (2.0 mg, 3.47 μmol, 5.03% yield) LC/MS, [M+H]=576.2, RT=1.67 min. ¹H NMR (400 MHz, DMSO-d₆) δ ppm 9.58 (s, 1H), 9.26 (d, J=3.3 Hz, 1H), 8.43 (s, 1H), 8.28 (br s, 1H), 7.69 (br s, 1H), 4.44 (br s, 1H), 3.72-3.50 (m, 7H), 3.46-3.40 (m, 2H), 3.17 (s, 1H), 2.98-2.77 (m, 2H), 1.05-0.67 (m, 7H).

### Example 362

### 2-((2-chloro-5-cyano-3-(7-(1,1-dioxidotetrahydrothiophen-3-yl)-2,7-diazaspiro[4.4]nonan-2-yl)phenyl)amino)-4-(cyclopropylamino)pyrazolo[1,5-a][1,3,5]triazine-8-carbonitrile

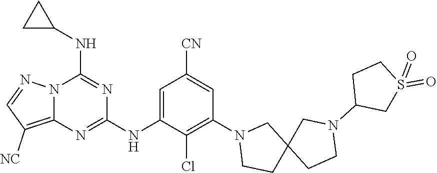 [SMILES: N#Cc1cc(Nc2nc(NC3CC3)n3ncc(C#N)c3n2)c(Cl)c(N2CCC3(CCN(C4CCS(=O)(=O)C4)C3)C2)c1]

### Step 1. 2-((2-chloro-5-cyano-3-(2,7-diazaspiro[4.4]nonan-2-yl)phenyl)amino)-4-(cyclopropylamino)pyrazolo[1,5-a][1,3,5]triazine-8-carbonitrile

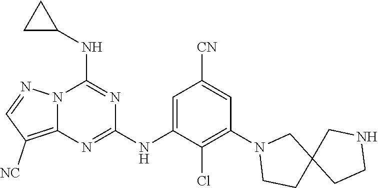 [SMILES: N#Cc1cc(Nc2nc(NC3CC3)n3ncc(C#N)c3n2)c(Cl)c(N2CCC3(CCNC3)C2)c1]

This compound was made employing steps 7 to 9 as outlined for Example 359 starting from commercially available tert-butyl 2,7-diazaspiro[4.4]nonane-2-carboxylate and 3,5-dibromo-4-chlorobenzonitrile.

### Step 2. Example 362

To a solution of 2-((2-chloro-5-cyano-3-(2,7-diazaspiro[4.4]nonan-2-yl)phenyl)amino)-4-(cyclopropylamino)pyrazolo[1,5-a][1,3,5]triazine-8-carbonitrile (200 mg, 0.421 mmol) in THF (8 mL) and DMSO (2 mL) at rt was added acetic acid (4.82 μL, 0.084 mmol) and dihydrothiophen-3(2H)-one 1,1-dioxide (85 mg, 0.632 mmol). The reaction mixture was stirred at rt for 1 h. Sodium cyanoborohydride (106 mg, 1.684 mmol) was added and the reaction was stirred at rt for 18 h. Volatiles were removed in vacuo. The reaction mixture was diluted with ethyl acetate, washed with saturated NaHCO₃ (50 mL), brine (70 mL), dried over Na₂SO₄ and concentrated under reduced pressure to obtain crude product. Crude product was purified using preparative HPLC (Kinetex EVO C18 (4.6×100) mm, 2.6 micron Buffer: 0.05% TFA in Water, Mobile Phase C: Buffer:Acetonitrile (95:5), Mobile Phase D: Acetonitrile:Buffer (95:5)) to obtain 2-((2-chloro-5-cyano-3-(2,7-diazaspiro[4.4]nonan-2-yl)phenyl)amino)-4-(cyclopropylamino) pyrazolo[1,5-a][1,3,5]triazine-8-carbonitrile as a mixture of diastereomers. LC/MS [M+H]=593.2, RT=1.79 min. ¹H NMR (400 MHz, DMSO-d₆) δ=9.50 (s, 1H), 9.20 (br s, 1H), 8.41 (s, 1H), 7.83 (s, 1H), 7.48-6.95 (m, 3H), 4.21-4.12 (m, 1H), 3.78 (s, 3H), 3.51-3.46 (m, 3H), 2.89-2.75 (m, 1H), 2.65-2.56 (m, 1H), 2.08 (s, 4H), 0.88-0.69 (m, 6H).

The mixture of diastereomers were further separated using chiral SFC (Column Name: Chiralpak ADH (250*4.6) mm. 5p, Co-Solvent Name: 0.2% Ammonia in methanol, Flow Rate: 4 ml/min, Co-Solvent Percentage: 50%, Back Pressure: 100 Bar) into 4 isomers.

Isomer 1: LC/MS [M+H]=593.2, RT=10.2 min. 1H NMR (400 MHz, DMSO-d6) δ ppm 9.40-9.54 (m, 1H) 9.13-9.27 (m, 1H) 8.33-8.43 (m, 1H) 7.75-7.88 (m, 1H) 7.10-7.16 (m, 1H) 4.07-4.30 (m, 1H) 3.43-3.92 (m, 6H) 3.18-3.23 (m, 1H) 2.74-2.95 (m, 2H) 2.63-2.73 (m, 1H) 2.26-2.35 (m, 1H) 1.82-2.23 (m, 6H) 1.17-1.34 (m, 2H) 0.70-0.91 (m, 4H).
Isomer 2: LC/MS [M+H]=593.2, RT=6.88 min. 1H NMR (400 MHz, DMSO-d6) δ ppm 9.46-9.53 (m, 1H) 9.16-9.24 (m, 1H) 8.34-8.43 (m, 1H) 7.76-7.86 (m, 1H) 7.19-7.25 (m, 1H) 7.06-7.15 (m, 2H) 6.93-6.98 (m, 1H) 3.44-3.56 (m, 4H) 2.80-2.97 (m, 2H) 2.64-2.72 (m, 1H) 2.30-2.35 (m, 1H) 1.85-2.12 (m, 5H) 1.36-1.46 (m, 1H) 1.10-1.29 (m, 3H) 0.71-0.85 (m, 4H).
Isomer 3: LC/MS [M+H]=593.2, RT=9.04 min. ¹H NMR (400 MHz, DMSO-d6) δ ppm 9.43-9.57 (m, 1H) 9.20 (br d, J=3.00 Hz, 1H) 8.33-8.49 (m, 1H) 7.76-7.91 (m, 1H) 6.86-7.34 (m, 1H) 6.84-7.28 (m, 1H) 4.12-4.39 (m, 1H) 3.70-3.99 (m, 2H) 3.46-3.64 (m, 4H) 3.09-3.25 (m, 2H) 2.83-2.94 (m, 1H) 2.17-2.27 (m, 1H) 1.83-2.11 (m, 3H) 1.39-1.46 (m, 1H) 1.13-1.28 (m, 1H) 0.63-0.90 (m, 4H)
Isomer 4: LC/MS [M+H]=593.2, RT=8.48 min. 1H NMR (400 MHz, DMSO-d6) δ ppm 9.44-9.58 (m, 1H) 9.12-9.26 (m, 1H) 8.35-8.46 (m, 1H) 7.76-7.91 (m, 1H) 7.10-7.18 (m, 1H) 4.07-4.29 (m, 1H) 3.63-3.82 (m, 2H) 3.42-3.56 (m, 4H) 2.80-2.99 (m, 2H) 2.67-2.75 (m, 1H) 1.75-2.18 (m, 5H) 0.66-0.89 (m, 4H).

### Example 363

### (S)-2-((2-chloro-5-cyano-3-(2-(1,1-dioxidotetrahydro-2H-thiopyran-4-yl)-6-methyl-2,6-dihydropyrrolo[3,4-c]pyrazol-5(4H)-yl)phenyl)amino)-4-(cyclopropylamino)pyrazolo[1,5-a][1,3,5]triazine-8-carbonitrile

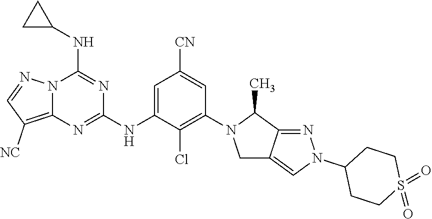 [SMILES: C[C@H]1c2nn(C3CCS(=O)(=O)CC3)cc2CN1c1cc(C#N)cc(Nc2nc(NC3CC3)n3ncc(C#N)c3n2)c1Cl]

### Step 1. tert-butyl (S)-3-hydroxy-2-methyl-5-oxo-2,5-dihydro-1H-pyrrole-1-carboxylate

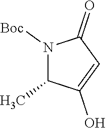

To a solution of (tert-butoxycarbonyl)-L-alanine (10 g, 52.9 mmol) in DCM (100 mL) at 0° C. was added DCC (16.36 g, 79 mmol) and the contents stirred at rt for 30 min. DMAP (8.39 g, 68.7 mmol) and 2,2-dimethyl-1,3-dioxane-4,6-dione (9.14 g, 63.4 mmol) were added and the reaction was stirred at rt for 16 h. The reaction mixture was transferred into a separatory funnel, washed with water and 1.5N HCl. The DCM layer was separated and concentrated. The reaction mixture was dissolved in ethyl acetate and heated at 85° C. for 2 h. Reaction mixture was cooled to room temperature, washed with sodium bicarbonate solution, dried over sodium sulphate and concentrated to afford crude product. Crude product was purified using ISCO to isolate tert-butyl (S)-3-hydroxy-2-methyl-5-oxo-2,5-dihydro-1H-pyrrole-1-carboxylate (8.2 g, 38.5 mmol, 72.8% yield). LC/MS [M+H]=214.2, RT=1.53 min.

### Step 2. tert-butyl (2S)-3-hydroxy-2-methyl-5-oxopyrrolidine-1-carboxylate

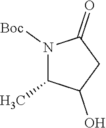

To a stirred solution of tert-butyl (S)-3-hydroxy-2-methyl-5-oxo-2,5-dihydro-1H-pyrrole-1-carboxylate (5 g, 23.45 mmol) in DCM (50 mL) at 0° C. was added NaBH₄ (2.66 g, 70.3 mmol) and the contents stirred at rt for 16 h. The reaction mixture was diluted with water, extracted with DCM, dried over sodium sulphate, and concentrated to afford crude product. The crude product was purified by ISCO to obtain tert-butyl (2S)-3-hydroxy-2-methyl-5-oxopyrrolidine-1-carboxylate (2.2 g, 10.22 mmol, 43.6% yield). LC/MS [M+H]10=215.3, RT=3.32 min.

### Step 3. tert-butyl (2S)-3-hydroxy-2-methylpyrrolidine-1-carboxylate

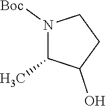

To a solution of tert-butyl (2S)-3-hydroxy-2-methyl-5-oxopyrrolidine-1-carboxylate (4 g, 18.58 mmol) in THF (10 mL) was added borane dimethyl sulfide complex (27.9 mL, 55.7 mmol) and the contents stirred at 75° C. for 16 h. The reaction was quenched with water, extracted with EtOAc, dried over sodium sulphate and concentrated to afford the crude product, which was further purified using ISCO to yield tert-butyl (2S)-3-hydroxy-2-methylpyrrolidine-1-carboxylate (2 g, 9.94 mmol, 53.5% yield)¹H NMR (400 MHz, CDCl₃) δ ppm 1.16-1.24 (m, 4H), 1.43-1.52 (m, 9H), 1.77-2.10 (m, 1H), 3.30-3.58 (m, 4H), 3.80-3.94 (m, 2H).

### Step 4. tert-butyl (S)-2-methyl-3-oxopyrrolidine-1-carboxylate

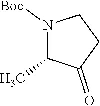

To a solution of tert-butyl (2S)-3-hydroxy-2-methylpyrrolidine-1-carboxylate (700 mg, 3.48 mmol) in DCM (20 mL) at 0° C. was added DMP (1475 mg, 3.48 mmol) and the contents stirred at rt for 16 h. The reaction mixture was extracted with DCM, washed with brine, dried over Na₂SO₄, and concentrated in vacuo to yield the crude product which was purified using ISCO to afford tert-butyl (S)-2-methyl-3-oxopyrrolidine-1-carboxylate (450 mg, 2.258 mmol, 64.9% yield). ¹H NMR (300 MHz, DMSO-d₆) δ ppm 1.21-1.47 (m, 9H) 2.55-2.94 (m, 4H) 3.30-3.58 (m, 3H) 4.09-4.31 (m, 2H).

### Step 5. tert-butyl (S,E)-4-((dimethylamino)methylene)-2-methyl-3-oxopyrrolidine-1-carboxylate

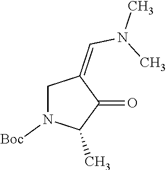 [SMILES: C[C@H]1C(=O)/C(=C\N(C)C)CN1C(=O)OC(C)(C)C]

A solution of tert-butyl (S)-2-methyl-3-oxopyrrolidine-1-carboxylate (200 mg, 1.004 mmol) in N,N-dimethylformamide dimethylacetal (120 mg, 1.004 mmol) was stirred at 110° C. After 3 h, all the starting material was consumed. Volatiles were evaporated in vacuo to afford crude tert-butyl (S,E)-4-((dimethylamino)methylene)-2-methyl-3-oxopyrrolidine-1-carboxylate (200 mg, 0.786 mmol, 78% yield), which was directly taken to next step without further purification.

### Step 6. tert-butyl (S)-6-methyl-2,6-dihydropyrrolo[3,4-c]pyrazole-5(4H)-carboxylate

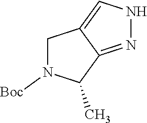 [SMILES: C[C@H]1c2n[nH]cc2CN1C(=O)OC(C)(C)C]

To a solution of tert-butyl (S,E)-4-((dimethylamino)methylene)-2-methyl-3-oxopyrrolidine-1-carboxylate (300 mg, 1.180 mmol) in EtOH (10 mL) was added acetic acid (0.068 mL, 1.180 mmol) and the contents stirred at 80° C. for 3 h. Reaction was cooled to rt, quenched with water, extracted with ethyl acetate, washed with brine, dried over Na₂SO₄, and concentrated in vacuo to yield crude product, which was purified by ISCO to isolate tert-butyl (S)-6-methyl-2,6-dihydropyrrolo[3,4-c]pyrazole-5(4H)-carboxylate (200 mg, 0.896 mmol, 76% yield). MS [M+H]=224.3.

### Step 7. tert-butyl(S)-2-(1,1-dioxidotetrahydro-2H-thiopyran-4-yl)-6-methyl-2,6-dihydropyrrolo[3,4-c]pyrazole-5(4H)-carboxylate

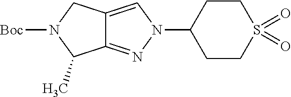 [SMILES: C[C@H]1c2nn(C3CCS(=O)(=O)CC3)cc2CN1C(=O)OC(C)(C)C]

A mixture of tert-butyl (S)-6-methyl-2,6-dihydropyrrolo[3,4-c]pyrazole-5(4H)-carboxylate (80 mg, 0.358 mmol), Cs₂CO₃ (350 mg, 1.075 mmol), and 1,1-dioxidotetrahydro-2H-thiopyran-4-yl 4-methylbenzenesulfonate (164 mg, 0.537 mmol) in DMF (2 mL) was stirred at 80° C. for 3 h. The reaction was cooled to rt, quenched with water, extracted with ethyl acetate, washed with brine, dried over Na₂SO₄, and concentrated in vacuo to yield crude product, which was purified by ISCO to afford tert-butyl (S)-2-(1,1-dioxidotetrahydro-2H-thiopyran-4-yl)-6-methyl-2,6-dihydropyrrolo[3,4-c]pyrazole-5(4H)-carboxylate (72 mg, 0.203 mmol, 56.5% yield), MS (E+) m/z: 356.4 (M+H).

### Step 8. (S)-4-(6-methyl-5,6-dihydropyrrolo[3,4-c]pyrazol-2(4H)-yl)tetrahydro-2H-thiopyran 1,1-dioxide

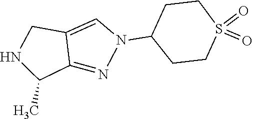 [SMILES: C[C@@H]1NCc2cn(C3CCS(=O)(=O)CC3)nc21]

To a stirred solution of tert-butyl (S)-2-(1,1-dioxidotetrahydro-2H-thiopyran-4-yl)-6-methyl-2,6-dihydropyrrolo[3,4-c]pyrazole-5(4H)-carboxylate (31 mg, 0.087 mmol) in DCM (5 mL) was added TFA (6.72 μL, 0.087 mmol) and the contents stirred at rt for 2 h. Volatiles were evaporated in vacuo to obtain (S)-4-(6-methyl-5,6-dihydropyrrolo[3,4-c]pyrazol-2(4H)-yl)tetrahydro-2H-thiopyran 1,1-dioxide (220 mg, 0.862 mmol, 98% yield). MS (E+) m/z: 256.3 (M+H).

### Step 9. (S)-2-((2-chloro-5-cyano-3-(2-(1,1-dioxidotetrahydro-2H-thiopyran-4-yl)-6-methyl-2,6-dihydropyrrolo[3,4-c]pyrazol-5(4H)-yl)phenyl)amino)-4-(cyclopropyl(4-methoxybenzyl)amino)pyrazolo[1,5-a][1,3,5]triazine-8-carbonitrile

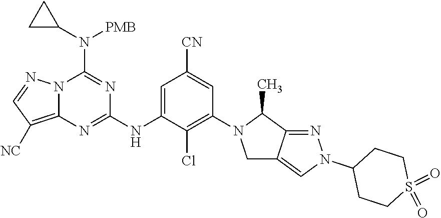

A mixture of 2-((3-bromo-2-chloro-5-cyanophenyl)amino)-4-(cyclopropyl(4-methoxybenzyl)amino)pyrazolo[1,5-a][1,3,5]triazine-8-carbonitrile (50 mg, 0.091 mmol), (S)-4-(6-methyl-5,6-dihydropyrrolo[3,4-c]pyrazol-2(4H)-yl)tetrahydro-2H-thiopyran 1,1-dioxide (23.22 mg, 0.091 mmol) and dioxane (4 mL) was degassed with nitrogen for 15 min. Tris(dibenzylideneacetone)dipalladium(0) (8.33 mg, 9.09 μmol) and (R/S)-BINAP (8.49 mg, 0.014 mmol) were added, and the reaction is stirred at 115° C. for 16 h. The Reaction was quenched with water, extracted with ethyl acetate, washed with brine, dried over Na₂SO₄, and concentrated in vacuo to obtain the crude product, which was purified by ISCO to afford (S)-2-((2-chloro-5-cyano-3-(2-(1,1-dioxidotetrahydro-2H-thiopyran-4-yl)-6-methyl-2,6-dihydropyrrolo[3,4-c]pyrazol-5(4H)-yl)phenyl)amino)-4-(cyclopropyl(4-methoxybenzyl)amino)pyrazolo[1,5-a][1,3,5]triazine-8-carbonitrile (30 mg, 0.041 mmol, 45.5% yield). MS (E+) m/z: 723.2 (M+H).

### Step 10. Example 366

To a stirred solution of (S)-2-((2-chloro-5-cyano-3-(2-(1,1-dioxidotetrahydro-2H-thiopyran-4-yl)-6-methyl-2,6-dihydropyrrolo[3,4-c]pyrazol-5(4H)-yl)phenyl)amino)-4-(cyclopropyl(4-methoxybenzyl)amino) pyrazolo[1,5-a][1,3,5]triazine-8-carbonitrile (30 mg, 0.041 mmol) in DCE (3 mL) was added TFA (3.19 μL, 0.041 mmol) and the contents stirred at 60° C. for 1 h. Volatiles were evaporated to obtain the crude product, which was purified using preparatory HPLC (Column: Waters XBridge C18, 150 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-min hold at 20% B, 20-40% B over 20 min, then a 5-min hold at 100% B; Flow Rate: 20 mL/min; Column Temperature: 25 C) to obtain (S)-2-((2-chloro-5-cyano-3-(2-(1,1-dioxidotetrahydro-2H-thiopyran-4-yl)-6-methyl-2,6-dihydropyrrolo[3,4-c]pyrazol-5(4H)-yl)phenyl) amino)-4-(cyclopropylamino)pyrazolo[1,5-a][1,3,5]triazine-8-carbonitrile (20 mg, 0.033 mmol, 77% yield). LC/MS [M+H]=605.2, RT=1.46 min. ¹H NMR (400 MHz, DMSO-d₆) δ=9.45 (s, 1H), 9.17 (br s, 1H), 8.40 (s, 1H), 7.69 (d, J=1.5 Hz, 1H), 7.05 (d, J=2.0 Hz, 1H), 4.11 (br s, 1H), 3.84-3.75 (m, 1H), 3.19 (s, 2H), 2.96 (br d, J=9.0 Hz, 1H), 2.90-2.81 (m, 1H), 2.47 (br s, 2H), 1.95-1.86 (m, 1H), 1.84-1.73 (m, 1H), 1.73-1.62 (m, 2H), 1.58 (br d, J=8.8 Hz, 1H), 1.50-1.39 (m, 1H), 0.94-0.69 (m, 4H).

### Example 364

### ((R)-2-((2-chloro-5-cyano-3-(4-methyl-2,6-dihydropyrrolo[3,4-c]pyrazol-5(4H)-yl)phenyl)amino)-4-(cyclopropylamino)pyrazolo[1,5-a][1,3,5]triazine-8-carbonitrile)

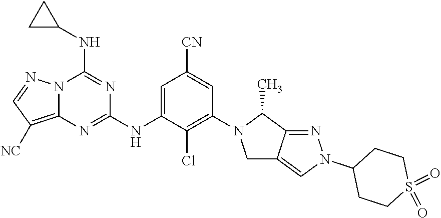 [SMILES: C[C@@H]1c2nn(C3CCS(=O)(=O)CC3)cc2CN1c1cc(C#N)cc(Nc2nc(NC3CC3)n3ncc(C#N)c3n2)c1Cl]

Example 364 was prepared from (tert-butoxycarbonyl)-D-alanine in the same general manner as described for Example 363, followed by purification by prep HPLC. LC/MS [M+H]=604.2, RT=1.72 min. ¹H NMR (400 MHz, DMSO-d6) δ=9.60 (s, 1H), 9.20 (br d, J=2.8 Hz, 1H), 8.41 (s, 1H), 7.90 (s, 1H), 7.56 (d, J=1.8 Hz, 1H), 7.36 (s, 1H), 5.72-5.60 (m, 1H), 4.98 (dd, J=3.0, 11.5 Hz, 1H), 4.69-4.56 (m, 1H), 3.99 (d, J=11.5 Hz, 1H), 3.51-3.38 (m, 2H), 3.30-3.19 (m, 3H), 2.89 (dt, J=4.3, 6.6 Hz, 1H), 2.45-2.38 (m, 1H), 2.32-2.17 (m, 2H), 1.26 (d, J=6.3 Hz, 3H), 0.84-0.76 (m, 4H).

### Example 365

### 2-((2-chloro-5-cyano-3-((S)-2-methyl-4-(((R)-5-oxopyrrolidin-2-yl)methyl)piperazin-1-yl)phenyl)amino)-4-(cyclopropylamino)pyrazolo[1,5-a][1,3,5]triazine-8-carbonitrile

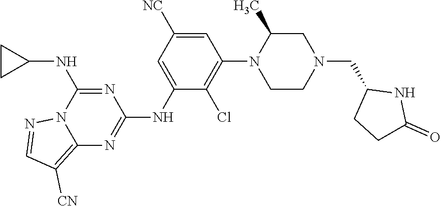 [SMILES: C[C@H]1CN(C[C@H]2CCC(=O)N2)CCN1c1cc(C#N)cc(Nc2nc(NC3CC3)n3ncc(C#N)c3n2)c1Cl]

A mixture of (S)-2-((2-chloro-5-cyano-3-(2-methylpiperazin-1-yl)phenyl)amino)-4-(cyclopropylamino)pyrazolo[1,5-a][1,3,5]triazine-8-carbonitrile (20 mg, 0.045 mmol), (R)-5-(bromomethyl)pyrrolidin-2-one (7.93 mg, 0.045 mmol), N,N-Diisopropylethylamine (0.038 mL, 0.223 mmol), and DMSO (1 mL) was stirred at 80° C. overnight. The reaction mixture was cooled to rt and loaded onto preparative HPLC (Column: Waters XBridge C18, 19×150 mm, 5-μm particles; Mobile Phase A: 10-mM ammonium acetate; Mobile Phase B: acetonitrile; Gradient: 15-52% B over 23 min, then a 5-min hold at 100% B; Flow: 20 mL/min.) to isolate 2-((2-chloro-5-cyano-3-((S)-2-methyl-4-(((R)-5-oxopyrrolidin-2-yl)methyl)piperazin-1-yl)phenyl)amino)-4-(cyclopropylamino)pyrazolo[1,5-a][1,3,5]triazine-8-carbonitrile (4.7 mg, 8.44 μmol, 18.93% yield). LC/MS [M+H]=547.2, RT=1.20 min. ¹H NMR (400 MHz, DMSO-d₆) δ=9.51 (br s, 1H), 9.27-9.14 (m, 1H), 8.42 (s, 1H), 8.12 (br s, 1H), 7.63-7.30 (m, 2H), 3.82-3.46 (m, 2H), 3.22-3.16 (m, 1H), 2.92-2.81 (m, 1H), 2.78-2.69 (m, 1H), 2.63-2.55 (m, 1H), 2.35-2.03 (m, 5H), 1.81-1.60 (m, 1H), 0.90 (br d, J=5.8 Hz, 3H), 0.84-0.74 (m, 4H).

### Example 366

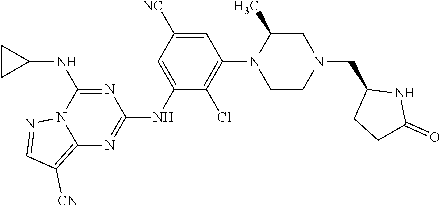 [SMILES: C[C@H]1CN(C[C@@H]2CCC(=O)N2)CCN1c1cc(C#N)cc(Nc2nc(NC3CC3)n3ncc(C#N)c3n2)c1Cl]

Example 366 was prepared from (S)-5-(Bromomethyl)-2-pyrrolidinone) in the same general manner as described for Example 365, followed by purification by prep HPLC LC/MS [M+H]=547.2, RT=1.68 min. ¹H NMR (400 MHz, DMSO-d₆) δ=9.49 (s, 1H), 9.24-9.16 (m, 1H), 8.42-8.38 (m, 1H), 8.14 (d, J=1.0 Hz, 1H), 7.51 (br d, J=10.3 Hz, 2H), 3.16 (d, J=5.3 Hz, 3H), 2.87-2.80 (m, 1H), 2.75-2.69 (m, 1H), 2.14-2.04 (m, 3H), 0.89-0.85 (m, 3H), 0.81-0.73 (m, 4H).

### Biological Assay

CK2 Kinase Assay: The effectiveness of compounds of the present invention as inhibitors of protein kinases can be readily evaluated by assays known to those skilled in the art.

### CK2A1 and CK2A2 HTRF Probe Displacement Assay

A time resolved FRET-based competition binding assay was used to assess compound binding to CK2A1 (C70-10G, SignalChem) and CK2A2 (C71-10G, SignalChem). The reaction solution was prepared containing 0.2 nM Anti GST-Tb (Cisbio, 61 GSTTLA), an appropriate fluorescein-labeled ATP competitive probe and 1.5 nM of CK2A1 or CK2A2 in a Buffer (20 mM HEPES, 10 mM MgCl2, 0.015% Brij-35, 4 mM DTT, 0.05 mg/mL BSA). Using Formulatrix Tempest, 2 μL of the reaction solution was dispensed into wells of a 1536 plate (Black Low Binding Polystyrene 1536 Plate, Corning, 3724) containing 10 nL of compounds of interest at appropriate concentrations in DMSO. The plate was incubated at room temperature for 1 h. FRET was measured using an EnVision plate reader (Excitation: 340 nM, Emission: 520 nM/495 nM). Total signal (0% inhibition) was calculated from wells containing nL DMSO only. Blank signal (100% inhibition) calculated from wells containing 10 nL of 15 nM staurosporine and internal controls. The IC50, defined as the concentration of competing test compound needed to reduce specific binding of the probe by 50%, was calculated using the 4 parameter logistic equation y=A+((B−A)/(1+((C/x)\hat{ }D))), where A and B denotes minimal and maximal % inhibition, respectively, C is the IC50, D is hill slope and x represents compound concentration.

The exemplified compounds of the present invention had IC₅₀ values of <300 nM in the CK2 kinase assay described above.

1. A compound of Formula (I): 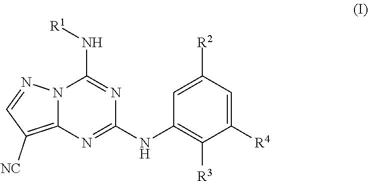
or a stereoisomer, a tautomer, or a pharmaceutically acceptable salt thereof, wherein:
R¹ is independently C₁₋₄ alkyl, C₃₋₄ cycloalkyl substituted with 0-1 R^{1a};
R^{1a} is independently F or C₁₋₄ alkyl;
R² and R³ are independently Cl, cyano, C₁₋₄ alkoxy, C₁₋₄ fluoroalkoxy, or C₃₋₄ cycloalkyl;
R⁴ is independently 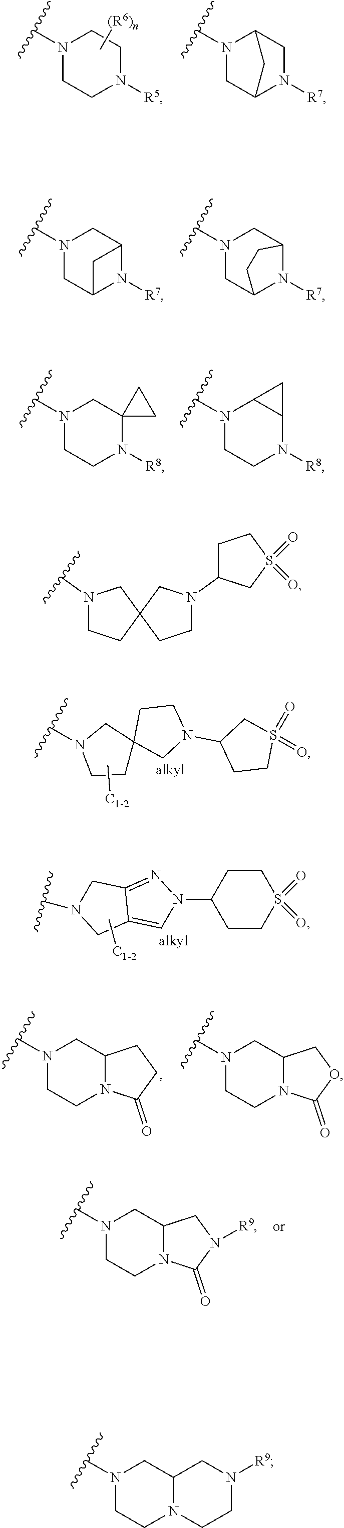
R⁵ is independently H, C₁₋₄ fluoroalkyl, —C(═O)(C₁₋₄ alkyl), —C(O)(C₁₋₄ fluoroalkyl), —C(═O)C(C₁₋₂ alkyl)(C₁₋₂ fluoroalkyl)(OH), —C(═O)O(C₁₋₄ alkyl), —C(═O)CH₂O(C₁₋₂ alkyl), —C(═O)CH₂OC(═O)(C₁₋₂ alkyl), —C(═O)NHR¹⁰, —C(═O)C(═O)NHR¹¹, —SO₂(C₁₋₄ alkyl), C₁₋₄ alkyl substituted with 0-2 R^{a}, —(CH₂)₀₋₁—C₃₋₆ cycloalkyl substituted with 0-2 R^{d}, —X-(4- to 7-membered heterocyclic ring including 1 to 2 ring heteroatoms which are independently N, N(R^{b}), O, or S(O)ₚ, and wherein the heterocyclic ring is partially saturated or fully saturated, and is substituted with 0 to 2 R^{e}), or —X-(5-membered heteroaryl wherein from 1 to 4 ring atoms are each independently N, or N(R^{b}), wherein the heteroaryl is substituted with 0 to 1 R^{d});
X is independently a bond, —CH₂—, —C(═O)—, —C(═O)CH₂—, —CH₂C(═O)—, or —C(═O)O—;
R⁶ is independently C₁₋₄ alkyl substituted with 0-1 OH, C₁₋₄ fluoroalkyl, or C₃₋₄ cycloalkyl;
R⁷ and R⁸ are independently H, C₁₋₄ alkyl substituted with 0-2 R^{e}, —C(═O)(C₁₋₄ alkyl), —C(═O)O(C₁₋₄ alkyl), —CH₂C(═O)NH₂, —SO₂(C₁₋₂ alkyl), 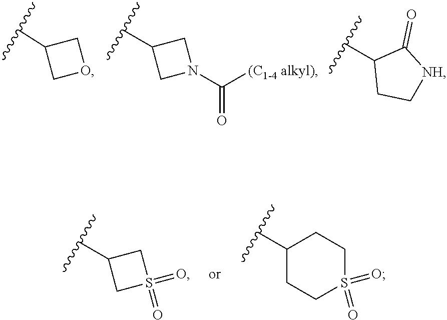
R⁹ is independently H, C₁₋₄ alkyl substituted with 0-2 R^{e}, —C(═O)(C₁₋₄ alkyl), —C(═O)O(C₁₋₄ alkyl), or;
R¹⁰ is independently C₁₋₄ alkyl substituted with 0-2 R^{c}, C₁₋₄ fluoroalkyl, or C₃₋₄ cycloalkyl;
R¹¹ is independently H, C₁₋₄ alkyl, or C₃₋₄ cycloalkyl;
R^{a} is independently halogen, OH, C₁₋₄alkoxy, —C(═O)NH₂, —C(═O)O(C₁₋₄ alkyl), C₃₋₄ cycloalkyl substituted with 0-1 R^{d}, or 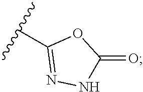 [SMILES: *c1n[nH]c(=O)o1]
R^{b} is independently H, C₁₋₄ alkyl substituted with 0-2 R^{e}, —C(═O)(C₁₋₄ alkyl), —C(═O)C(C₁₋₂ alkyl)(C₁₋₂ fluoroalkyl)(OH), —C(═O)O(C₁₋₄ alkyl), —C(═O)C(═O)NH₂, —SO₂(C₁₋₄ alkyl substituted with 0-1 R^{e}), C₃₋₄ cycloalkyl substituted with 0-1 R^{d}, 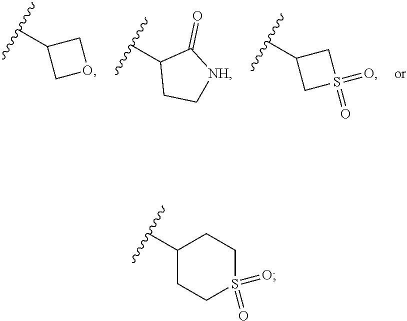 [SMILES: *C1COC1 ; *C1CCNC1=O ; *C1CS(=O)(=O)C1 ; *C1CCS(=O)(=O)CC1]
R^{c} is independently C₁₋₂ alkoxy;
R^{d} is independently D, halogen, OH, NH₂, C₁₋₄alkyl, or —SO₂(C₁₋₄alkyl);
R^{e} is independently oxo or R^{d};
R^{f} is independently F, OH, C₁₋₄ alkoxy, or —C(═O)NH₂.
n is independently 0, 1 or 2; and
p is independently 0, 1 or 2.

2. The compound of claim 1, or a stereoisomer, a tautomer, or a pharmaceutically acceptable salt thereof; wherein:
R¹ is independently C₁₋₂ alkyl, C₁₋₂fluoroalkyl, or C₃₋₄ cycloalkyl substituted with 0-1 R^{1a};
R^{1a} is independently F or C₁₋₂ alkyl;
R² is independently cyano, C₁₋₄ fluoroalkoxy, or C₃₋₄ cycloalkyl;
R³ is independently C₁, C₁₋₄ alkoxy, C₁₋₄ fluoroalkoxy, or C₃₋₄ cycloalkyl;
R⁶ is independently C₁₋₄ alkyl substituted with 0-1 OH, CF₃, or C₃₋₄ cycloalkyl;
R¹⁰ is independently C₁₋₄ alkyl substituted with 0-2 R^{c}, C₁₋₄ fluoroalkyl, or C₃₋₄ cycloalkyl; and
R¹¹ is independently H, C₁₋₄ alkyl, or C₃₋₄ cycloalkyl.

3. The compound of claim 2, or a stereoisomer, a tautomer, or a pharmaceutically acceptable salt thereof; wherein:
R⁴ is independently

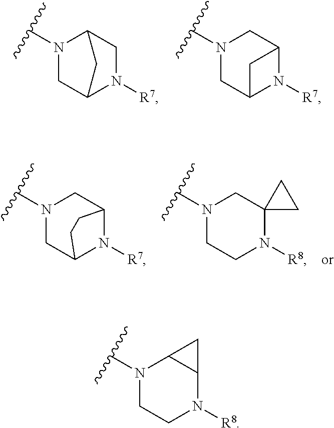

4. The compound of claim 2, or a stereoisomer, a tautomer, or a pharmaceutically acceptable salt thereof; wherein:
R⁴ is independently

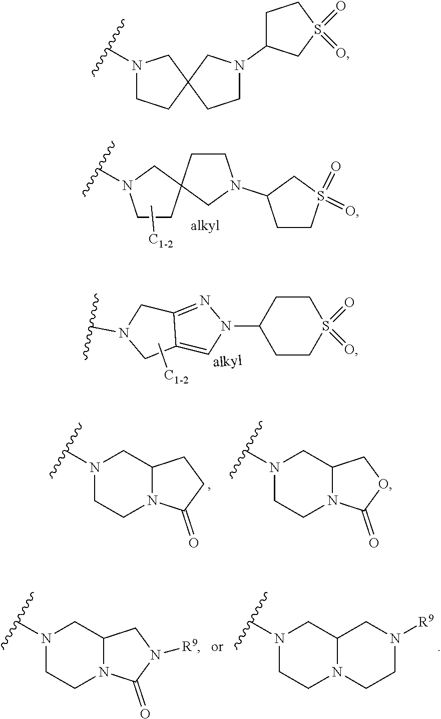

5. The compound of claim 2, wherein the compound is represented by Formula (II): 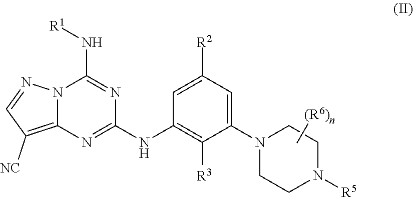
or a stereoisomer, a tautomer, or pharmaceutically acceptable salt thereof.

6. A compound of Formula (III): 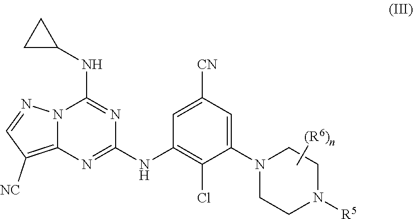
or a stereoisomer, a tautomer, or pharmaceutically acceptable salt thereof, wherein:
R⁵ is independently H, C₁₋₄ fluoroalkyl, —C(═O)(C₁₋₂ alkyl), —C(═O)(C₁₋₂ fluoroalkyl), —C(═O)C(CH₃)(CF₃)(OH), —C(═O)O(C₁₋₄ alkyl), —C(═O)CH₂O(C₁₋₂ alkyl), —C(═O)CH₂OC(═O)(C₁₋₂ alkyl), —C(═O)NHR¹⁰, —C(═O)C(═O)NHR¹¹, —SO₂(C₁₋₂ alkyl), C₁₋₄ alkyl substituted with 0-2 R^{a}, —(CH₂)₀₋₁—C₃₋₄ cycloalkyl substituted with 0-1 R^{d}, 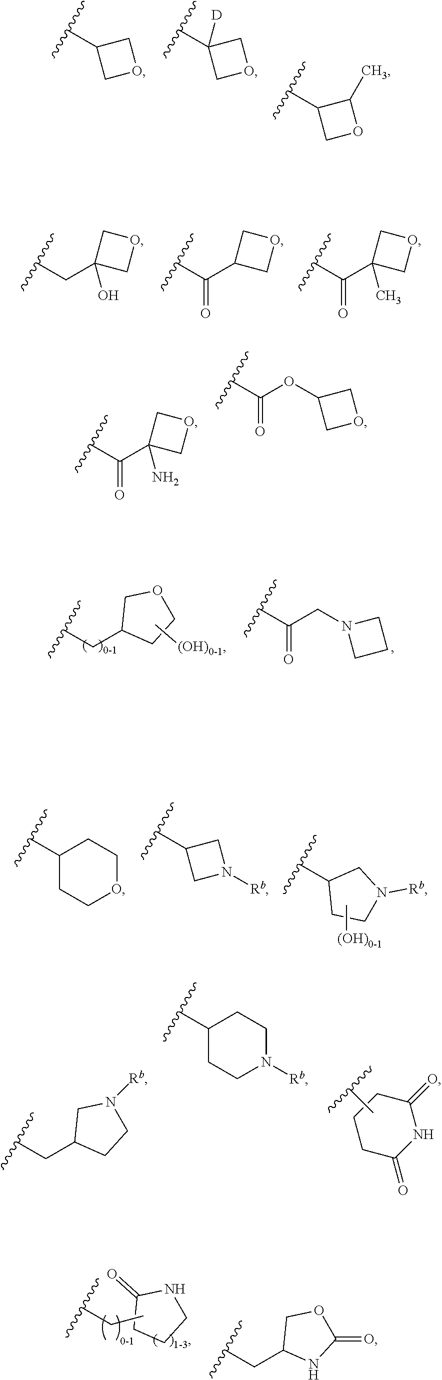 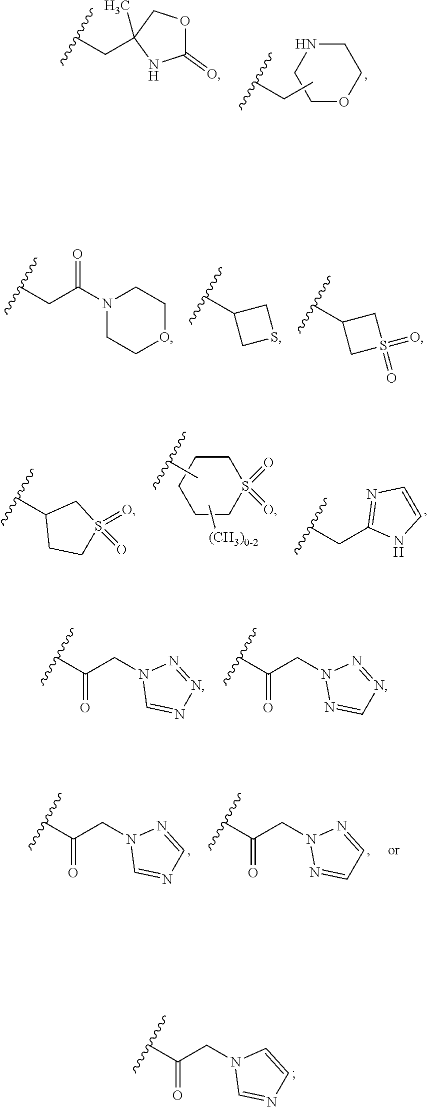
R⁶ is independently C₁₋₂ alkyl substituted with 0-1 OH, CF₃, or cyclopropyl;
R¹⁰ is independently C₁₋₄ alkyl substituted with 0-2 R^{c}, C₁₋₄ fluoroalkyl, or C₃₋₄ cycloalkyl;
R¹¹ is independently H, C₁₋₄ alkyl, or C₃₋₄ cycloalkyl;
R^{a} is independently F, OH, C₁₋₂alkoxy, —C(═O)NH₂, —C(═O)O(C₁₋₂ alkyl), or C₃₋₄ cycloalkyl substituted with 0-1 R^{d};
R^{b} is independently H, C₁₋₄ alkyl substituted with 0-2 R^{f}, —C(═O)(C₁₋₂ alkyl), —C(═O)C(CH₃)(CF₃)(OH), —C(═O)O(C₁₋₂ alkyl), —C(═O)C(═O)NH₂, —SO₂(C₁₋₂ alkyl substituted with 0-1 Rₑ), C₃₋₄ cycloalkyl substituted with 0-1 R^{d}, 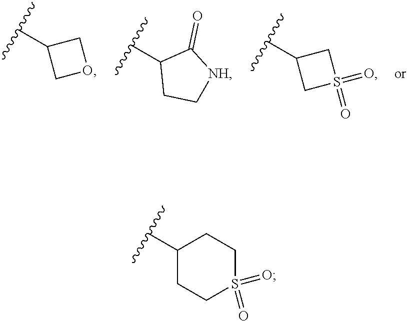 [SMILES: *C1COC1 ; *C1CCNC1=O ; *C1CS(=O)(=O)C1 ; *C1CCS(=O)(=O)CC1]
R^{c} is independently C₁₋₂ alkoxy;
R^{d} is independently F, OH, or —SO₂(C₁₋₂ alkyl);
R^{f} is independently F, OH, C₁₋₂ alkoxy, or —C(═O)NH₂; and
n is independently 0, 1 or 2.

7. The compound of claim 6, wherein the compound is represented by Formula (IV): 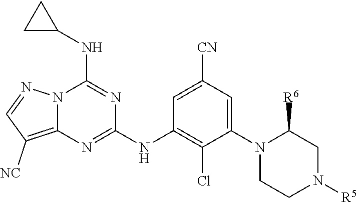
or a stereoisomer, a tautomer, or pharmaceutically acceptable salt thereof.

8. The compound according to claim 1, which is selected from any one of the Examples as described in the specification, or a stereoisomer, or a pharmaceutically acceptable salt thereof.

9. A composition comprising a compound of claim 1, or a pharmaceutically acceptable salt thereof, and one or more a pharmaceutically acceptable carriers, diluents, or excipients.

10. A method for treating cancer, psoriasis or rheumatoid arthritis, comprising administering to a mammalian species in need thereof, a therapeutically effective amount of one or more compounds according to claim 1.

11. The method according to claim 10 wherein the cancer is carcinoma of the prostate, pancreatic ductal adenocarcinoma, breast, colon, lung, ovary, pancreas and thyroid, neuroblastoma, glioblastoma, medulloblastoma, melanoma, multiple myeloma, and/or acute myelogenous leukemia (AML).

12. A composition comprising a compound of claim 5, or a pharmaceutically acceptable salt thereof, and one or more a pharmaceutically acceptable carriers, diluents, or excipients.

13. A composition comprising a compound of claim 6, or a pharmaceutically acceptable salt thereof, and one or more a pharmaceutically acceptable carriers, diluents, or excipients.

14. A composition comprising a compound of claim 7, or a pharmaceutically acceptable salt thereof, and one or more a pharmaceutically acceptable carriers, diluents, or excipients.

15. A composition comprising a compound of claim 8, or a pharmaceutically acceptable salt thereof, and one or more a pharmaceutically acceptable carriers, diluents, or excipients.
